(19) United States
(12) Patent Application Publication
(10) Pub. No.: US 2016/0176867 A1
(43) Pub. Date: Jun. 23, 2016

(54) MODIFIED BET-PROTEIN-INHIBITING DIHYDROQUINOXALINONES AND DIHYDROPYRIDOPYRAZINONES

(71) Applicant: BAYER PHARMA AKTIENGESELLSCHAFT, Berlin (DE)

(72) Inventors: Norbert SCHMEES, Berlin (DE); Bernard HAENDLER, Berlin (DE); Detlef STÖCKIGT, Potsdam (DE); Daniel GALLENKAMP, Wuppertal (DE); Richard BISSELL, Stockport (DE); Richard Alexander BOUGLAS, Buxton (DE)

(21) Appl. No.: 14/903,549

(22) PCT Filed: Jul. 7, 2014
(86) PCT No.: PCT/EP2014/064486

§ 371 Date: Jan. 7, 2016

(30) Foreign Application Priority Data
Jul. 9, 2013 (EP) 13175767.6

(51) Int. Cl. C07D 471/04; C07D 405/04; A61K 31/498; C07D 405/14; C07D 241/44; C07D 519/00; C07D 401/04; A61K 31/4985; A61K 31/5377

(52) U.S. Cl. CPC C07D 471/04; A61K 31/4985; C07D 405/04; A61K 31/498; A61K 31/5377; C07D 241/44; C07D 519/00; C07D 401/04; C07D 405/14

(57) ABSTRACT

The present invention relates to BET protein-inhibitory, especially BRD4-inhibitory, dihydroquinoxalinones and dihydropyridopyrazinones of the general formula (I)

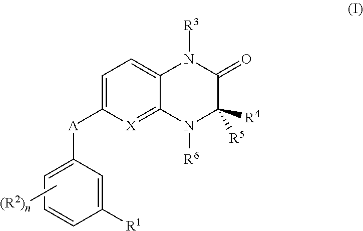

in which A, X, R¹, R², R³, R⁴, R⁵, R⁶ and n are each as defined in the description,
to intermediates for preparation of the inventive compounds, to pharmaceutical compositions comprising the inventive compounds, and to the prophylactic and therapeutic use thereof in the case of hyperproliferative disorders, especially in the case of neoplastic disorders.

This invention further relates to the use of BET protein inhibitors in viral infections, in neurodegenerative disorders, in inflammation diseases, in atherosclerotic disorders and in male fertility control.

The present invention relates to BET protein-inhibitory, especially BRD4-inhibitory, dihydroquinoxalinones and dihydropyridopyrazinones, to intermediates for preparation of the inventive compounds, to pharmaceutical compositions comprising the inventive compounds, and to the prophylactic and therapeutic use thereof in the case of hyperproliferative disorders, especially in the case of neoplastic disorders. This invention further relates to the use of BET protein inhibitors in viral infections, in neurodegenerative disorders, in inflammation diseases, in atherosclerotic disorders and in male fertility control.

The human BET family (bromo domain and extra C-terminal domain family) has four members (BRD2, BRD3, BRD4 and BRDT) containing two related bromo domains and one extraterminal domain (Wu and Chiang, J. Biol. Chem., 2007, 282:13141-13145). The bromo domains are protein regions which recognize acetylated lysine residues. Such acetylated lysines are often found at the N-terminal end of histones (e.g. histone 3 or histone 4) and are features of an open chromatin structure and active gene transcription (Kuo and Allis, Bioessays, 1998, 20:615-626). The various acetylation patterns which have been recognized by BET proteins in histones have been studied in detail (Umehara et al., J. Biol. Chem., 2010, 285:7610-7618; Filippakopoulos et al., Cell, 2012, 149:214-231). In addition, bromo domains can recognize further acetylated proteins. For example, BRD4 binds to RelA, which leads to stimulation of NF-κB and transcriptional activity of inflammatory genes (Huang et al., Mol. Cell. Biol., 2009, 29:1375-1387; Zhang et al., J. Biol. Chem., 2012, 287: 28840-28851; Zou et al., Oncogene, 2013, doi:10.1038/onc.2013.179). BRD4 also binds to cyclin T1 and forms an active complex which is important for transcription elongation (Schröder et al., J. Biol. Chem., 2012, 287:1090-1099). The extraterminal domain of BRD2, BRD3 and BRD4 interacts with several proteins involved in chromatin modulation and the regulation of gene expression (Rahman et al., Mol. Cell. Biol., 2011, 31:2641-2652).

In mechanistic terms, BET proteins play an important role in cell growth and in the cell cycle. They are associated with mitotic chromosomes, suggesting a role in epigenetic memory (Dey et al., Mol. Biol. Cell, 2009, 20:4899-4909; Yang et al., Mol. Cell. Biol., 2008, 28:967-976). Involvement of BRD4 in the post-mitotic reactivation of gene transcription has been demonstrated (Zhao et al., Nat. Cell. Biol., 2011, 13:1295-1304). BRD4 is essential for transcription elongation and recruits the elongation complex P-TEFb consisting of CDK9 and cyclin T1, which leads to activation of RNA polymerase II (Yang et al., Mol. Cell, 2005, 19:535-545; Schröder et al., J. Biol. Chem., 2012, 287:1090-1099). Consequently, the expression of genes involved in cell proliferation is stimulated, for example of c-Myc, cyclin D1 and aurora B (You et al., Mol. Cell. Biol., 2009, 29:5094-5103; Zuber et al., Nature, 2011, doi:10.1038). BRD2 is involved in the regulation of target genes of the androgen receptor (Draker et al., PLOS Genetics, 2012, 8, e1003047). BRD2 and BRD3 bind to transcribed genes in hyperacetylated chromatin regions and promote transcription by RNA polymerase II (LeRoy et al., Mol. Cell, 2008, 30:51-60).

Knock-down of BRD4 or the inhibition of the interaction with acetylated histones in various cell lines leads to G1 arrest (Mochizuki et al., J. Biol. Chem., 2008, 283:9040-9048; Mertz et al., Proc. Natl. Acad. Sci. USA, 2011, 108:16669-16674). It has also been shown that BRD4 binds to promoter regions of several genes which are activated in the G1 phase, for example cyclin D1 and D2 (Mochizuki et al., J. Biol. Chem., 2008, 283:9040-9048). In addition, inhibition of the expression of c-Myc, an essential factor in cell proliferation, after BRD4 inhibition has been demonstrated (Dawson et al., Nature, 2011, 478:529-533; Delmore et al., Cell, 2011, 146:1-14; Mertz et al., Proc. Natl. Acad. Sci. USA, 2011, 108:16669-16674). Inhibition of the expression of androgen-regulated genes and binding of BRD2 to corresponding regulatory regions has also been demonstrated (Draker et al., PLOS Genetics, 2012, 8, e1003047).

BRD2 and BRD4 knockout mice die early in embryogenesis (Gyuris et al., Biochim. Biophys. Acta, 2009, 1789:413-421; Houzelstein et al., Mol. Cell. Biol., 2002, 22:3794-3802). Heterozygotic BRD4 mice have various growth defects attributable to reduced cell proliferation (Houzelstein et al., Mol. Cell. Biol., 2002, 22:3794-3802).

BET proteins play an important role in various tumour types. Fusion between the BET proteins BRD3 or BRD4 and NUT, a protein which is normally expressed only in the testes, leads to an aggressive form of squamous cell carcinoma, called NUT midline carcinoma (French, Cancer Genet. Cytogenet., 2010, 203:16-20). The fusion protein prevents cell differentiation and promotes proliferation (Yan et al., J. Biol. Chem., 2011, 286:27663-27675, Grayson et al., 2013, doi:10-1038/onc.2013.126). The growth of in vivo models derived therefrom is inhibited by a BRD4 inhibitor (Filippakopoulos et al., Nature, 2010, 468:1067-1073). Screening for therapeutic targets in an acute myeloid leukaemia cell line (AML) showed that BRD4 plays an important role in this tumour (Zuber et al., Nature, 2011, 478, 524-528). Reduction in BRD4 expression leads to a selective arrest of the cell cycle and to apoptosis. Treatment with a BRD4 inhibitor prevents the proliferation of an AML xenograft in vivo. Further experiments with a BRD4 inhibitor show that BRD4 is involved in various haematological tumours, for example multiple myeloma (Delmore et al., Cell, 2011, 146, 904-917) and Burkitt's lymphoma (Mertz et al., Proc. Natl. Acad. Sci. USA, 2011, 108, 16669-16674). In solid tumours too, for example lung cancer, BRD4 plays an important role (Lockwood et al., Proc. Natl. Acad. Sci. USA, 2012, 109, 19408-19413). Elevated expression of BRD4 has been detected in multiple myeloma, and amplification of the BRD4 gene has also been found in patients having multiple myeloma (Delmore et al., Cell, 2011, 146, 904-917). Amplification of the DNA region containing the BRD4 gene was detected in primary breast tumours (Kadota et al., Cancer Res, 2009, 69:7357-7365). For BRD2 too, there are data relating to a role in tumours. A transgenic mouse which overexpresses BRD2 selectively in B cells develops B cell lymphoma and leukaemia (Greenwall et al., Blood, 2005, 103:1475-1484).

BET proteins are also involved in viral infections. BRD4 binds to the E2 protein of various papillomaviruses and is important for the survival of the viruses in latently infected cells (Wu et al., Genes Dev., 2006, 20:2383-2396; Vosa et al., J. Virol., 2006, 80:8909-8919). The herpes virus, which is responsible for Kaposi's sarcoma, also interacts with various BET proteins, which is important for disease survival (Viejo-Borbolla et al., J. Virol., 2005, 79:13618-13629; You et al., J. Virol., 2006, 80:8909-8919). Through binding to P-TEFb, BRD4 also plays an important role in the replication of HIV-1 (Bisgrove et al., Proc. Natl. Acad. Sci. USA, 2007, 104:13690-13695). Treatment with a BRD4 inhibitor leads to stimulation of the dormant, untreatable reservoir of HIV-1 viruses in T cells (Banerjee et al., J. Leukoc. Biol., 2012, 92, 1147-1154). This reactivation could enable new therapeutic methods for AIDS treatment (Zinchenko et al., J. Leukoc. Biol., 2012, 92, 1127-1129). A critical role of BRD4 in DNA replication of polyomaviruses has also been reported (Wang et al., PLoS Pathog., 2012, 8, doi:10.1371).

BET proteins are additionally involved in inflammation processes. BRD2-hypomorphic mice show reduced inflammation in adipose tissue (Wang et al., Biochem. J., 2009, 425:71-83). Infiltration of macrophages in white adipose tissue is also reduced in BRD2-deficient mice (Wang et al., Biochem. J., 2009, 425:71-83). It has also been shown that BRD4 regulates a number of genes involved in inflammation. In LPS-stimulated macrophages, a BRD4 inhibitor prevents the expression of inflammatory genes, for example IL-1 or IL-6 (Nicodeme et al., Nature, 2010, 468:1119-1123).

BET proteins are also involved in the regulation of the ApoA1 gene (Mirguet et al., Bioorg. Med. Chem. Lett., 2012, 22:2963-2967). The corresponding protein is part of high-density lipoprotein (HDL), which plays an important role in atherosclerosis (Smith, Arterioscler. Thromb. Vasc. Biol., 2010, 30:151-155). Through the stimulation of ApoA1 expression, BET protein inhibitors can increase the concentrations of cholesterol HDL and hence may potentially be useful for the treatment of atherosclerosis (Mirguet et al., Bioorg. Med. Chem. Lett., 2012, 22:2963-2967). The BET protein BRDT plays an essential role in spermatogenesis through the regulation of the expression of several genes important during and after meiosis (Shang et al., Development, 2007, 134:3507-3515; Matzuk et al., Cell, 2012, 150:673-684). In addition, BRDT is involved in the post-meiotic organization of chromatin (Dhar et al., J. Biol. Chem., 2012, 287:6387-6405). In vivo experiments in mice show that treatment with a BET inhibitor which also inhibits BRDT leads to a decrease in sperm production and infertility (Matzuk et al., Cell, 2012, 150:673-684).

All these studies show that the BET proteins play an essential role in various pathologies, and also in male fertility. It would therefore be desirable to find potent and selective inhibitors which prevent the interaction between the BET proteins and acetylated proteins. These novel inhibitors should also have suitable pharmacokinetic properties which allow inhibition of these interactions in vivo, i.e. in patients.

It has now been found that substituted dihydroquinoxalinones and pyridopyrazinones have the desired properties, i.e. show BET-inhibitory, especially BRD4-inhibitory, action. The inventive compounds are thus valuable active ingredients for prophylactic and therapeutic use in the case of hyperproliferative disorders, especially in the case of neoplastic disorders. In addition, the inventive compounds can be used in the case of viral infections, in the case of neurodegenerative disorders, in the case of inflammation diseases, in the case of atherosclerotic disorders and in male fertility control.

### PRIOR ART

The nomenclature applied in the assessment of the prior art (derived from the nomenclature software ACD Name batch, Version 12.01, from Advanced Chemical Development, Inc.) is illustrated by the following diagrams:

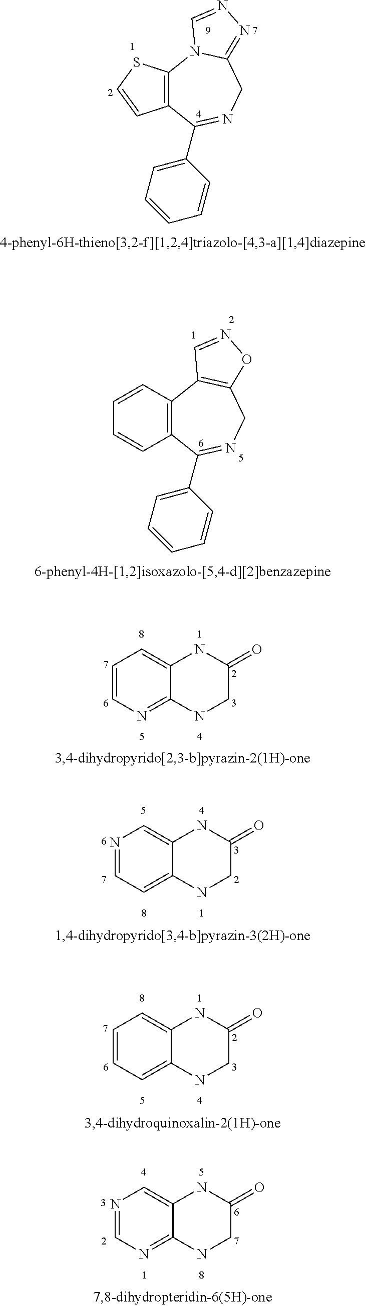 [SMILES: c1ccc(C2=NCc3nncn3-c3sccc32)cc1 ; c1ccc(C2=NCc3oncc3-c3ccccc32)cc1 ; O=C1CNc2ncccc2N1 ; O=C1CNc2ccncc2N1 ; O=C1CNc2ccccc2N1 ; O=C1CNc2ncncc2N1]

Based on the chemical structure, only very few types of BRD4 inhibitors have been described to date (Chun-Wa Chung et al., Progress in Medicinal Chemistry 2012, 51, 1-55).

The first published BRD4 inhibitors were diazepines. For example, phenylthienotriazolo-1,4-diazepines (4-phenyl-6H-thieno[3,2-f][1,2,4]triazolo[4,3-a][1,4]diazepines) are described in WO2009/084693 (Mitsubishi Tanabe Pharma Corporation) and as compound JQ1 in WO2011/143669 (Dana Farber Cancer Institute).

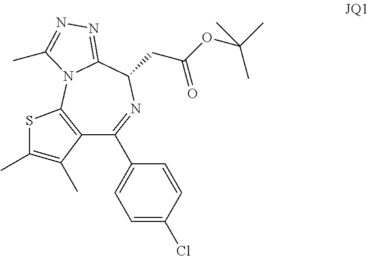 [SMILES: Cc1sc2c(c1C)C(c1ccc(Cl)cc1)=N[C@@H](CC(=O)OC(C)(C)C)c1nnc(C)n1-2]

Replacement of the thieno moiety by a benzo moiety also leads to active inhibitors (J. Med. Chem. 2011, 54, 3827-3838; E. Nicodeme et al., Nature 2010, 468, 1119). Further 4-phenyl-6H-thieno[3,2-f][1,2,4]triazolo[4,3-a][1,4]diazepines and related compounds having alternative rings as a fusion partner rather than the benzo moiety are claimed generically or described explicitly in WO2012/075456 (Constellation Pharmaceuticals).

Azepines as BRD4 inhibitors are described in WO2012/075383 (Constellation Pharmaceuticals). This application relates to 6-substituted 4H-isoxazolo[5,4-d][2]benzazepines and 4H-isoxazolo[3,4-d][2]benzazepines, including those compounds which have optionally substituted phenyl at position 6, and also to analogues with alternative heterocyclic fusion partners rather than the benzo moiety, for example thieno- or pyridoazepines. Another structural class of BRD4 inhibitors described is that of 7-isoxazoloquinolines and related quinolone derivatives (Bioorganic & Medicinal Chemistry Letters 22 (2012) 2963-2967). WO2011/054845 (GlaxoSmithKline) describes further benzodiazepines as BRD4 inhibitors.

The inventive compounds, in contrast, are substituted 3,4-dihydroquinoxalin-2(1H)-one derivatives and 3,4-dihydropyrido[2,3-b]pyrazin-2(1H)-one derivatives which differ structurally in various ways from the above-discussed chemotypes of BRD4 inhibitors. Because of the significant structural differences, it was not to be expected that the compounds claimed here would also have BRD4-inhibitory action. It is therefore surprising that the inventive compounds have good inhibitory action in spite of the considerable structural differences.

Some documents include compounds which are structurally similar but are aimed at completely different mechanisms of action, and in some cases also other indications. Dihydroquinoxalinones and dihydropyridopyrazinones and related bicyclic systems have been described in a series of patent applications.

WO 2010/085570 (Takeda Pharmaceutical Company) describes inhibitors of poly-ADP-ribose polymerase (PARP) which are derived from a series of bi- and tricyclic skeletons, and which include 3,4-dihydropyrido[2,3-b]pyrazin-2(1H)-one derivatives, as medicaments for treatment of various diseases. The exemplary compounds disclosed therein differ from the inventive compounds, for example, by the type and position of the substitution on the pyrido moiety of the dihydropyridopyrazinone skeleton.

WO 2006/005510 (Boehringer Ingelheim) describes 1,4-dihydropyrido[3,4-b]pyrazin-3 (2H)-one derivatives as inhibitors of PLK-1 for treatment of hyperproliferative disorders. The position of the pyrido nitrogen distinguishes the substances disclosed in that publication from the inventive compounds. The substances claimed are characterized by an anilinic group which is bonded via —NH— to C-7 of the dihydropyridopyrazinone skeleton and which is itself substituted in the para position by a carboxamide.

WO 2008/117061 (Sterix Ltd) describes a series of bicyclic chemotypes, including 3,4-dihydroquinoxalin-2(1H)-one derivatives, as inhibitors of steroid sulphatase, for uses including inhibition of the growth of tumours. The substances claimed in the application mentioned differ from the substances disclosed in this present invention, for example, by the substitution at N-1.

US 2006/0019961 (P. E. Mahaney et al.) describes substituted 3,4-dihydroquinoxalin-2(1H)-one derivatives as modulators of the oestrogen receptor for treatment of various inflammation disorders, cardiovascular disorders and autoimmune disorders. The example substances disclosed in this application have only small substituents (such as halogen or methyl) at C-6, but a substituent which necessarily has a hydroxylated aromatic system at N-4, by virtue of which the substances differ from the compounds of this present invention.

WO 2006/050054, WO 2007/134169 and US 2009/0264384 (Nuada LLC) describe a series of bicyclic chemotypes, including 3,4-dihydroquinoxalin-2(1H)-one derivatives, as inhibitors of tumour necrosis factor alpha (TNF-α) and various isoforms of phosphodiesterase for treatment of inflammation disorders among others. N-1 in the structures claimed is substituted by a group characterized, for example, by a carboxamide or a terminal group derived from the boronic acid, which differ from the compounds of this present invention.

WO 2003/020722 and WO 2004/076454 (Boehringer Ingelheim) disclose 7,8-dihydropteridin-6(5H)-ones as inhibitors of specific cell cycle kinases for treatment of hyperproliferative disorders.

WO 2006/018182 (Boehringer Ingelheim) describes pharmaceutical preparations of 7,8-dihydropteridin-6(5H)-ones in combination inter alia with various cytostatics for treatment of neoplastic disorders.

WO 2006/018185 (Boehringer Ingelheim) describes the use of 7,8-dihydropteridin-6(5H)-ones for treatment of various neoplastic disorders.

WO 2011/101369 (Boehringer Ingelheim), WO 2011/113293 (Jiangsu Hengrui Medicine), WO 2009/141575 (Chroma Therapeutics), WO 2009/071480 (Nerviano Medical Sciences) and also WO 2006/021378, WO 2006/021379 and WO 2006/021548 (likewise Boehringer Ingelheim) disclose further 7,8-dihydropteridin-6(5H)-one derivatives as inhibitors of PLK-1 for treating hyperproliferative disorders.

U.S. Pat. No. 6,369,057 describes various quinoxaline and quinoxalinone derivatives as antivirally active compounds; EP 0657166 and EP 728481 describe combinations of such compounds with nucleosides or protease inhibitors having antiviral action.

WO 2007/022638 (Methylgene Inc.) discloses, in quite general terms, HDAC inhibitors of several chemotypes, dihydro-quinoxalinone derivatives inter alia, but the structures of the example compounds disclosed differ distinctly from the compounds of the present invention.

WO 1999/050254 (Pfizer) describes, among other compounds, quinoxalinones and dihydroquinoxalinones as inhibitors of serine proteases for antithrombotic therapy, but these compounds differ distinctly by the type and position of the substituents from the inventive compounds.

Some 3,4-dihydroquinoxalin-2(1H)-one derivatives substituted at C-6 by an aromatic amino group, in which the phenyl group is in turn substituted by apara-amide group (corresponding to 2-oxo-1,2,3,4-tetrahydroquinoxaline derivatives), are indexed by Chemical Abstracts as “Chemical Library” substances without a literature reference [see 4-{[(3R)-4-cyclopentyl-3-ethyl-1-methyl-2-oxo-1,2,3,4-tetrahydroquinoxalin-6-yl]amino}-3-methoxy-N-[2-methyl-1-(pyrrolidin-1-yl)propan-2-yl]benzamide, CAS Registry No. 1026451-60-4, N-(1-benzylpiperidin-4-yl)-4-{[(3R)-4-cyclopentyl-1,3-dimethyl-2-oxo-1,2,3,4-tetrahydroquinoxalin-6-yl]amino}-3-methoxybenzamide, CAS Registry No. 1026961-36-3,4-{[(3R)-4-cyclohexyl-1,3-dimethyl-2-oxo-1,2,3,4-tetrahydroquinoxalin-6-yl]amino}-N-[1-(dimethylamino)-2-methylpropan-2-yl]-3-methoxybenzamide, CAS Registry No. 1025882-57-8]. No therapeutic use for these compounds has been described to date.

Nevertheless, there is still a great need for active compounds for prophylaxis and treatment of disorders, especially of hyperproliferative disorders, and very particularly of neoplastic disorders.

It has now been found that compounds of the general formula (I)

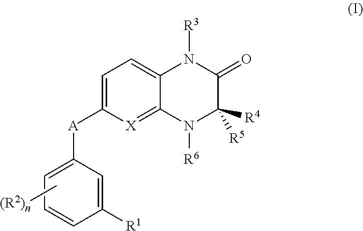

in which
A is —NH—, —N(C₁-C₃-alkyl)- or —O—,
X is —N— or —CH—,
n is 0.1 or 2,
R¹ is a —C(═O)NR⁷R⁸ or —S(═O)₂NR⁷R⁸ group,
   or
   is 5-membered monocyclic heteroaryl- which may optionally be mono-, di- or trisubstituted identically or differently by halogen, cyano, C₁-C₄-alkyl-, C₂-C₄-alkenyl-, C₂-C₄-alkynyl-, halo-C₁-C₄-alkyl-, C₁-C₄-alkoxy-, halo-C₁-C₄-alkoxy-, C₁-C₄-alkylthio-, halo-C₁-C₄-alkylthio-,
   —NR⁹R¹⁰, —C(═O)OR¹¹, —C(═O)N⁹R¹¹, —C(═O)R¹¹, —S(═O)₂R¹¹, —S(═O)₂NR⁹R¹⁰,
R² is hydrogen, halogen, cyano, C₁-C₃-alkyl-, C₂-C₄-alkenyl-, C₂-C₄-alkynyl-, halo-C₁-C₄-alkyl-, C₁-C₄-alkoxy-, halo-C₁-C₄-alkoxy-, C₁-C₄-alkylthio- or halo-C₁-C₄-alkylthio-, and, if n is 2, R² may be the same or different,
   or
R¹ and R² together are a *—S(═O)₂—NR⁸—CH₂—**, *—S(═O)₂—NR⁸—CH₂—CH₂—**, *—C(═O)—NR⁸—CH₂—** or *—C(═O)—NR⁸—CH₂—CH₂—** group in which “*” denotes the attachment point of R¹ to the phenyl ring in formula (I), and in which “**” denotes a carbon atom of this phenyl ring adjacent to this attachment point,
R³ is methyl- or ethyl-,
R⁴ is hydrogen or C₁-C₃-alkyl-,
R⁵ is hydrogen or C₁-C₃-alkyl-,
   or
R⁴ and R⁵ together with the carbon atom to which they are bonded are C₃-C₆-cycloalkylene,
R⁶ is C₁-C₆-alkyl- which may optionally be monosubstituted by C₁-C₃-alkoxy-, phenyl-, C₃-C₈-cycloalkyl-, or 4- to 8-membered heterocycloalkyl-,
   in which phenyl- may itself optionally be mono-, di- or trisubstituted identically or differently by: halogen, cyano, C₁-C₄-alkyl-, C₂-C₄-alkenyl-, C₂-C₄-alkynyl-, C₁-C₄-alkoxy-, halo-C₁-C₄-alkyl-, halo-C₁-C₄-alkoxy-, and in which C₃-C₈-cycloalkyl- and 4- to 8-membered heterocycloalkyl- may themselves optionally be mono- or disubstituted identically or differently by C₁-C₃-alkyl-,
   or
   is C₃-C₈-cycloalkyl- or 4- to 8-membered heterocycloalkyl-, which may optionally be mono- or disubstituted identically or differently by C₁-C₃-alkyl- or C₁-C₄-alkoxycarbonyl-,
   or
   is phenyl which may optionally be mono- or disubstituted identically or differently by halogen, C₁-C₃-alkyl- or 4-8-membered heterocycloalkyl-,
      in which the 4-8-membered heterocycloalkyl- may itself optionally be mono- or disubstituted identically or differently by C₁-C₃-alkyl or C₁-C₄-alkoxycarbonyl-,
R⁷ is hydrogen,
   or
   is C₁-C₆-alkyl- which may optionally be mono-, di- or trisubstituted identically or differently by: hydroxyl, oxo, fluorine, cyano, C₁-C₄-alkoxy-, halo-C₁-C₄-alkoxy-, —NR⁹R¹⁰, C₃-C₈-cycloalkyl-, C₄-C₈-cycloalkenyl-, 4- to 8-membered heterocycloalkyl-, 4-bis 8-membered heterocycloalkenyl-, C₅-C₁₁-spirocycloalkyl-, C₅-C₁₁-heterospirocycloalkyl-, bridged C₆-C₁₂-cycloalkyl-, bridged C₆-C₁₂-heterocycloalkyl-, C₆-C₁₂-bicycloalkyl-, C₆-C₁₂-heterobicycloalkyl-, phenyl-, 5- to 6-membered heteroaryl-,
      in which C₃-C₈-cycloalkyl-, C₄-C₈-cycloalkenyl-, 4- to 8-membered heterocycloalkyl-, 4- to 8-membered heterocycloalkenyl-, C₅-C₁₁-spirocycloalkyl-, C₅-C₁₁-heterospirocycloalkyl-, bridged C₆-C₁₂-cycloalkyl-, bridged C₆-C₁₂-heterocycloalkyl-, C₆-C₁₂-bicycloalkyl-, C₆-C₁₂-heterobicycloalkyl- may itself optionally be mono- or disubstituted identically or differently by: hydroxyl, fluorine, oxo, cyano, C₁-C₃-alkyl-, fluoro-C₁-C₃-alkyl-, C₃-C₆-cycloalkyl-, cyclopropylmethyl-, C₁-C₃-alkylcarbonyl-, C₁-C₄-alkoxycarbonyl- or —NR⁹R¹⁰, and
      in which phenyl and 5- to 6-membered heteroaryl may optionally be mono- or disubstituted identically or differently by: halogen, cyano, trifluoromethyl-, C₁-C₃-alkyl-, C₁-C₃-alkoxy-,
   or
   is C₃-C₆-alkenyl- or C₃-C₆-alkynyl-,
   or
   is C₃-C₈-cycloalkyl-, C₄-C₈-cycloalkenyl-, C₅-C₁₁-spirocycloalkyl-, bridged C₆-C₁₂-cycloalkyl- or C₆-C₁₂-bicycloalkyl-, which may optionally be mono- or disubstituted identically or differently by: hydroxyl, oxo, cyano, fluorine, C₁-C₃-alkyl, C₁-C₃-alkoxy, trifluoromethyl, —NR⁹R¹⁰,
   or
   is 4- to 8-membered heterocycloalkyl-, 4- to 8-membered heterocycloalkenyl-, C₅-C₁₁-heterospirocycloalkyl-, bridged C₆-C₁₂-heterocycloalkyl- or C₆-C₁₂-heterobicycloalkyl-, which may optionally be mono- or disubstituted identically or differently by: hydroxyl, fluorine, oxo, cyano, C₁-C₃-alkyl-, fluoro-C₁-C₃-alkyl-, C₃-C₆-cycloalkyl-, cyclopropylmethyl-, C₁-C₃-alkylcarbonyl-, C₁-C₄-alkoxycarbonyl- or —NR⁹R¹⁰,
R⁸ is hydrogen or optionally singly or doubly, identically or differently hydroxyl-, oxo- or C₁-C₃-alkoxy-substituted C₁-C₃-alkyl-, or fluoro-C₁-C₃-alkyl,
   or
R⁷ and R⁸ together with the nitrogen atom to which they are bonded are 4- to 8-membered heterocycloalkyl, 4- to 8-membered heterocycloalkenyl-, C₅-C₁₁-heterospirocycloalkyl-, bridged C₆-C₁₂-heterocycloalkyl- or C₆-C₁₂-heterobicycloalkyl-, which may optionally be mono- or disubstituted identically or differently by: hydroxyl, fluorine, oxo, cyano, C₁-C₃-alkyl-, fluoro-C₁-C₃-alkyl-, C₃-C₆-cycloalkyl-, cyclopropylmethyl-, C₁-C₃-alkylcarbonyl-, C₁-C₄-alkoxycarbonyl- or —NR⁹R¹⁰,
R⁹ and R¹⁰ are each independently hydrogen or optionally singly or doubly, identically or differently hydroxyl-, oxo- or C₁-C₃-alkoxy-substituted C₁-C₃-alkyl, or fluoro-C₁-C₃-alkyl, or 4- to 8-membered heterocycloalkyl,
   in which the 4- to 8-membered heterocycloalkyl- may itself optionally be mono- or disubstituted identically or differently by C₁-C₃-alkyl,
   or
R⁹ and R¹⁰ together with the nitrogen atom to which they are attached are 4- to
8-membered heterocycloalkyl- which may optionally be mono- or disubstituted identically or differently by: hydroxyl, fluorine, oxo, cyano, C₁-C₃-alkyl-, fluoro-C₁-C₃-alkyl-, C₃-C₆-cycloalkyl-, cyclopropylmethyl-, C₁-C₃-alkylcarbonyl- or C₁-C₄-alkoxycarbonyl-,
R¹¹ is C₁-C₆-alkyl- or phenyl-C₁-C₃-alkyl-,
and the diastereomers, racemates, polymorphs and physiologically acceptable salts thereof, surprisingly inhibit the interaction between BET proteins, especially BRD4, and an acetylated histone 4 peptide and hence inhibit the growth of cancer cells.

Preference is given to those compounds of the general formula (I)

in which
A is —NH— or —N(C₁-C₃-alkyl)-,
X is —N— or —CH—,
n is 0.1 or 2,
R¹ is a —C(═O)NR⁷R⁸ or —S(═O)₂NR⁷R⁸ group,
   or
   is oxazolyl-, thiazolyl-, oxadiazolyl- or thiadiazolyl-, which may optionally be mono- or disubstituted identically or differently by halogen, cyano, C₁-C₃-alkyl-, trifluoromethyl-, C₁-C₃-alkoxy-, trifluoromethoxy- or —NR⁹R¹⁰,
R² is hydrogen, fluorine, chlorine, cyano, methyl-, methoxy-, ethyl- or ethoxy-, and if n is 2, R² may be the same or different,
R¹ and R² together are a *—S(═O)₂—NR⁸—CH₂—** or *—C(═O)—NR⁸—CH₂—** group in which “*” denotes the attachment point of R¹ to the phenyl ring in formula (I), and in which “**” denotes a carbon atom of this phenyl ring adjacent to this attachment point,
R³ is methyl- or ethyl-,
R⁴ is hydrogen, methyl- or ethyl-,
R⁵ is hydrogen, methyl- or ethyl-,
R⁶ is C₂-C₅-alkyl-,
   or
   is methyl- or ethyl-monosubstituted by C₁-C₃-alkoxy-, phenyl- or 4- to 8-membered heterocycloalkyl-,
      in which phenyl- may itself optionally be mono-, di- or trisubstituted identically or differently by: fluorine, chlorine, bromine, cyano, C₁-C₃-alkyl-, C₁-C₃-alkoxy-, and
      in which the 4- to 8-membered heterocycloalkyl- may itself optionally be mono- or disubstituted identically or differently by methyl-,
   or
   is C₃-C₈-cycloalkyl- or 4- to 8-membered heterocycloalkyl-, which may optionally be mono- or disubstituted identically or differently by C₁-C₃-alkyl- or C₁-C₄-alkoxycarbonyl-,
   or
   is phenyl which may optionally be mono- or disubstituted identically or differently by fluorine, chlorine, methyl- or 6-membered heterocycloalkyl-,
      in which the 6-membered heterocycloalkyl- may itself optionally be monosubstituted by methyl- or tert-butoxycarbonyl-,
R⁷ is hydrogen,
   or
   is C₁-C₆-alkyl- which may optionally be mono-, di- or trisubstituted identically or differently by: hydroxyl, oxo, fluorine, cyano, C₁-C₃-alkoxy-, fluoro-C₁-C₃-alkoxy-, —NR⁹R¹⁰, 4- to 8-membered heterocycloalkyl-, phenyl-, 5- to 6-membered heteroaryl-,
      in which the 4- to 8-membered heterocycloalkyl- may itself optionally be monosubstituted by: hydroxyl, oxo, C₁-C₃-alkyl-, fluoro-C₁-C₃-alkyl-, cyclopropyl-, cyclopropylmethyl-, acetyl- or tert-butoxycarbonyl-,
   or
   is C₃-C₆-cycloalkyl- which may optionally be mono- or disubstituted identically or differently by: hydroxyl, oxo, cyano, fluorine, —NR⁹R¹⁰,
   or
   is 4- to 8-membered heterocycloalkyl-, C₆-C₈-heterospirocycloalkyl-, bridged C₆-C₁₀-heterocycloalkyl- or C₆-C₁₀-heterobicycloalkyl-, which may optionally be mono- or disubstituted identically or differently by: hydroxyl, oxo, C₁-C₃-alkyl-, fluoro-C₁-C₃-alkyl-, cyclopropyl-, cyclopropylmethyl-, acetyl- or tert-butoxycarbonyl-,
R⁸ is hydrogen or C₁-C₃-alkyl-,
   or
R⁷ and R⁸ together with the nitrogen atom to which they are bonded are 4- to 8-membered heterocycloalkyl-, C₆-C₈-heterospirocycloalkyl-, bridged C₆-C₁₀-heterocycloalkyl- or C₆-C₁₀-heterobicycloalkyl-,
   which may optionally be mono- or disubstituted identically or differently by: hydroxyl, fluorine, oxo, C₁-C₃-alkyl-, fluoro-C₁-C₃-alkyl-, cyclopropyl-, cyclopropylmethyl-, acetyl- or tert-butoxycarbonyl-,
R⁹ and R¹⁰ are each independently hydrogen or optionally mono-hydroxyl- or -oxo-substituted C₁-C₃-alkyl- or trifluoromethyl-, or 6-membered heterocycloalkyl-,
   in which the 6-membered heterocycloalkyl- may itself optionally be mono- or disubstituted identically or differently by C₁-C₃-alkyl,
   or
R⁹ and R¹⁰ together with the nitrogen atom to which they are bonded are 4- to 7-membered heterocycloalkyl- which may optionally be mono- or disubstituted identically or differently by: hydroxyl, fluorine, oxo, C₁-C₃-alkyl-, fluoro-C₁-C₃-alkyl-, cyclopropyl-, cyclopropylmethyl-, acetyl- or tert-butoxycarbonyl-,
   and the diastereomers, racemates, polymorphs and physiologically acceptable salts thereof.

Particular preference is given to those compounds of the general formula (I)

in which
A is —NH— or —N(methyl)-,
X is —N— or —CH—,
n is 0 or 1,
R¹ is a —C(═O)NR⁷R⁸ or —S(═O)₂NR⁷R⁸ group,
   or
   is oxazolyl- or oxadiazolyl- which may optionally be mono- or disubstituted identically or differently by C₁-C₃-alkyl-,
R² is hydrogen, fluorine, chlorine, methyl- or methoxy-, or
R¹ and R² together are a *—S(═O)₂—NR⁸—CH₂—** group in which “*” denotes the attachment point of R¹ to the phenyl ring in formula (I), and in which “**” denotes a carbon atom of this phenyl ring adjacent to this attachment point,
R³ is methyl-,
R⁴ is methyl- or ethyl-,
R⁵ is hydrogen,
R⁶ is C₃-C₅-alkyl- or 2-methoxyethyl-,
   or
   is methyl-monosubstituted by phenyl- or 4- to 6-membered heterocycloalkyl-, in which phenyl- may itself optionally be mono- or disubstituted identically or differently by: fluorine, chlorine, cyano, methyl-, methoxy-, and in which the 4- to 6-membered heterocycloalkyl- may itself optionally be monosubstituted by methyl-,
   or
   is C₃-C₈-cycloalkyl- or is 4- to 6-membered heterocycloalkyl-, which may optionally be mono- or disubstituted identically or differently by C₁-C₃-alkyl- or C₁-C₄-alkoxycarbonyl-,
   or
   is phenyl which may optionally be mono- or disubstituted identically or differently by fluorine, chlorine, methyl- or N-tert-butoxycarbonylpiperazinyl-,
R⁷ is hydrogen,
   or
   is C₁-C₄-alkyl which may optionally be monosubstituted by
   —NR⁹R¹⁰ or 4- to 8-membered heterocycloalkyl-,
      in which the 4- to 8-membered heterocycloalkyl- may itself optionally be monosubstituted by: oxo, C₁-C₃-alkyl-, fluoro-C₁-C₃-alkyl-, cyclopropyl- or cyclopropylmethyl-,
   or is C₃-C₆-cycloalkyl- which may optionally be monosubstituted by hydroxyl, fluorine or —NR⁹R¹⁰,
   or
   is 4- to 8-membered heterocycloalkyl- which may optionally be mono- or disubstituted identically or differently by: oxo, C₁-C₃-alkyl-, fluoro-C₁-C₃-alkyl-, cyclopropyl- or cyclopropylmethyl-,
R⁸ is hydrogen, methyl- or ethyl-,
   or
R⁷ and R⁸ together with the nitrogen atom to which they are bonded are 4- to 6-membered heterocycloalkyl- or C₆-C₈-heterospirocycloalkyl-, which may optionally be mono- or disubstituted identically or differently by: fluorine, oxo, C₁-C₃-alkyl-, fluoro-C₁-C₃-alkyl-, cyclopropyl- or cyclopropylmethyl-,
R⁹ and R¹⁰ are each independently hydrogen or optionally mono-hydroxyl- or -oxo-substituted C₁-C₃-alkyl-, trifluoromethyl-, or is
N-methylpiperidinyl-,
   or
R⁹ and R¹⁰ together with the nitrogen atom to which they are bonded are 4- to 7-membered heterocycloalkyl- which may optionally be mono- or disubstituted identically or differently by: fluorine, oxo, C₁-C₃-alkyl-, fluoro-C₁-C₃-alkyl-, cyclopropyl- or cyclopropylmethyl-,
   and the diastereomers, racemates, polymorphs and physiologically acceptable salts thereof.

Particular preference is further given to those compounds of the general formula (I)

in which
A is —NH— or —N(methyl)-,
X is —N—,
n is 0 or 1,
R¹ is a —C(═O)NR⁷R⁸ or —S(═O)₂NR⁷R⁸ group,
   or
   is oxazolyl- or oxadiazolyl- which may optionally be mono- or disubstituted identically or differently by C₁-C₃-alkyl-,
R² is hydrogen, fluorine, chlorine, methyl- or methoxy-, or
R¹ and R² together are a *—S(═O)₂—NR⁸—CH₂—** group in which “*” denotes the attachment point of R¹ to the phenyl ring in formula (I), and in which “**” denotes a carbon atom of this phenyl ring adjacent to this attachment point,
R³ is methyl-,
R⁴ is methyl- or ethyl-,
R⁵ is hydrogen,
R⁶ is C₃-C₅-alkyl- or 2-methoxyethyl-,
   or
   is methyl- monosubstituted by phenyl- or 4- to 6-membered heterocycloalkyl-,
      in which phenyl- may itself optionally be mono- or disubstituted identically or differently by: fluorine, chlorine, cyano, methyl-, methoxy-, and
      in which the 4- to 6-membered heterocycloalkyl- may itself optionally be monosubstituted by methyl-,
   or
   is C₃-C₈-cycloalkyl- or is 4- to 6-membered heterocycloalkyl-, which may optionally be mono- or disubstituted identically or differently by C₁-C₃-alkyl- or C₁-C₄-alkoxycarbonyl-,
   or
   is phenyl which may optionally be mono- or disubstituted identically or differently by fluorine, chlorine, methyl- or N-tert-butoxycarbonylpiperazinyl-,
R⁷ is hydrogen,
   or
   is C₁-C₄-alkyl which may optionally be monosubstituted by
   —NR⁹R¹⁰ or 4- to 8-membered heterocycloalkyl-,
      in which the 4- to 8-membered heterocycloalkyl- may itself optionally be monosubstituted by: oxo, C₁-C₃-alkyl-, fluoro-C₁-C₃-alkyl-, cyclopropyl- or cyclopropylmethyl-,
   or
   is C₃-C₆-cycloalkyl- which may optionally be monosubstituted by hydroxyl, fluorine or NR⁹R¹⁰,
   or
   is 4- to 8-membered heterocycloalkyl- which may optionally be mono- or disubstituted identically or differently by: oxo, C₁-C₃-alkyl-, fluoro-C₁-C₃-alkyl-, cyclopropyl- or cyclopropylmethyl-,
R⁸ is hydrogen, methyl- or ethyl-,
   or
R⁷ and R⁸ together with the nitrogen atom to which they are bonded are 4- to 6-membered heterocycloalkyl- or C₆-C₈-heterospirocycloalkyl-, which may optionally be mono- or disubstituted identically or differently by: fluorine, oxo, C₁-C₃-alkyl-, fluoro-C₁-C₃-alkyl-, cyclopropyl- or cyclopropylmethyl-,
R⁹ and R¹⁰ are each independently hydrogen or optionally mono-hydroxyl- or -oxo-substituted C₁-C₃-alkyl-, trifluoromethyl-, or is N-methylpiperidinyl-,
   or
R⁹ and R¹⁰ together with the nitrogen atom to which they are bonded are 4- to 7-membered heterocycloalkyl- which may optionally be mono- or disubstituted identically or differently by: fluorine, oxo, C₁-C₃-alkyl-, fluoro-C₁-C₃-alkyl-, cyclopropyl- or cyclopropylmethyl-,
   and the diastereomers, racemates, polymorphs and physiologically acceptable salts thereof.

Particular preference is further given to those compounds of the general formula (I)

in which
A is —NH— or —N(methyl)-,
X is —CH—,
n is 0 or 1,
R¹ is a —C(═O)NR⁷R⁸ or —S(═O)₂NR⁷R⁸ group,
   or
   is oxazolyl- or oxadiazolyl- which may optionally be mono- or disubstituted identically or differently by C₁-C₃-alkyl-,
R² is hydrogen, fluorine, chlorine, methyl- or methoxy-, or
R¹ and R² together are a *—S(═O)₂—NR⁸—CH₂—** group in which “*” denotes the attachment point of R¹ to the phenyl ring in formula (I), and in which “**” denotes a carbon atom of this phenyl ring adjacent to this attachment point,
R³ is methyl-,
R⁴ is methyl- or ethyl-,
R⁵ is hydrogen,
R⁶ is C₃-C₅-alkyl- or 2-methoxyethyl-,
   or
   is methyl-monosubstituted by phenyl- or 4- to 6-membered heterocycloalkyl-, in which phenyl- may itself optionally be mono- or disubstituted identically or differently by: fluorine, chlorine, cyano, methyl-, methoxy-, and in which the 4- to 6-membered heterocycloalkyl- may itself optionally be monosubstituted by methyl-,
   or
   is C₃-C₈-cycloalkyl- or is 4- to 6-membered heterocycloalkyl-, which may optionally be mono- or disubstituted identically or differently by C₁-C₃-alkyl- or C₁-C₄-alkoxycarbonyl-,
   or
   is phenyl which may optionally be mono- or disubstituted identically or differently by fluorine, chlorine, methyl- or N-tert-butoxycarbonylpiperazinyl-,
R⁷ is hydrogen,
   or
   is C₁-C₄-alkyl which may optionally be monosubstituted by
   —NR⁹R¹⁰ or 4- to 8-membered heterocycloalkyl-,
      in which the 4- to 8-membered heterocycloalkyl- may itself optionally be monosubstituted by: oxo, C₁-C₃-alkyl-, fluoro-C₁-C₃-alkyl-, cyclopropyl- or cyclopropylmethyl-,
   or
   is C₃-C₆-cycloalkyl- which may optionally be monosubstituted by hydroxyl, fluorine or NR⁹R¹⁰,
   or
   is 4- to 8-membered heterocycloalkyl- which may optionally be mono- or disubstituted identically or differently by: oxo, C₁-C₃-alkyl-, fluoro-C₁-C₃-alkyl-, cyclopropyl- or cyclopropylmethyl-,
R⁸ is hydrogen, methyl- or ethyl-,
   or
R⁷ and R⁸ together with the nitrogen atom to which they are bonded are 4- to 6-membered heterocycloalkyl- or C₆-C₈-heterospirocycloalkyl-, which may optionally be mono- or disubstituted identically or differently by: fluorine, oxo, C₁-C₃-alkyl-, fluoro-C₁-C₃-alkyl-, cyclopropyl- or cyclopropylmethyl-,
R⁹ and R¹⁰ are each independently hydrogen or optionally mono-hydroxyl- or -oxo-substituted C₁-C₃-alkyl-, trifluoromethyl-, or is N-methylpiperidinyl-,
   or
R⁹ and R¹⁰ together with the nitrogen atom to which they are bonded are 4- to 7-membered heterocycloalkyl- which may optionally be mono- or disubstituted identically or differently by: fluorine, oxo, C₁-C₃-alkyl-, fluoro-C₁-C₃-alkyl-, cyclopropyl- or cyclopropylmethyl-,
   and the diastereomers, racemates, polymorphs and physiologically acceptable salts thereof.

Very particular preference is given to those compounds of the general formula (I)

in which
A is —NH— or —N(methyl)-,
X is —N— or —CH—,
n is 0 or 1,
R¹ is a —C(═O)NR⁷R⁸ or —S(═O)₂NR⁷R⁸ group,
   or
   is oxazolyl- or oxadiazolyl- which may optionally be mono- or disubstituted by methyl-,
R² is hydrogen, methyl- or methoxy-, or
R¹ and R² together are a *—S(═O)₂—NH—CH₂—** group in which “*” denotes the attachment point of R¹ to the phenyl ring in formula (I), and in which “**” denotes a carbon atom of this phenyl ring adjacent to this attachment point,
R³ is methyl-,
R⁴ is methyl-,
R⁵ is hydrogen,
R⁶ is isopropyl-, isobutyl- or 2-methoxyethyl-,
   or
   is benzyl wherein the phenyl moiety may optionally be mono- or disubstituted identically or differently by: fluorine, methoxy-,
   or
   is C₅-C₇-cycloalkyl- which may optionally be mono- or disubstituted by methyl-,
   or
   is tetrahydrofuranyl-, tetrahydropyranyl- or piperidinyl-,
      in which piperidinyl- may optionally be monosubstituted by methyl- or tert-butoxycarbonyl-,
   or
   is phenyl which may optionally be monosubstituted by fluorine, methyl- or N-tert-butoxycarbonylpiperazinyl-,
R⁷ is hydrogen,
   or
   is C₁-C₃-alkyl which may optionally be monosubstituted by —NR⁹R¹⁰ or N-methylpiperidinyl-,
   or
   is cyclopropyl-, or is cyclohexyl-,
      in which cyclohexyl- may optionally be monosubstituted by hydroxyl- or —NR⁹R¹⁰,
   or
   is 4- to 6-membered heterocycloalkyl which may optionally be monosubstituted by methyl-,
R⁸ is hydrogen, methyl- or ethyl-,
   or
R⁷ and R⁸ together with the nitrogen atom to which they are bonded are 4- to 6-membered heterocycloalkyl- which may optionally be mono- or disubstituted by fluorine, or which may optionally be monosubstituted by methyl-, isopropyl-, 2,2,2-trifluoroethyl- or cyclopropylmethyl-, or is 6-azaspiro[3.3]heptyl- or is 2-oxa-6-azaspiro[3.3]heptyl-,
R⁹ and R¹⁰ are each independently hydrogen, C₁-C₃-alkyl- or N-methylpiperidinyl-,
   or
R⁹ and R¹⁰ together with the nitrogen atom to which they are bonded are 6-membered heterocycloalkyl- which may optionally be mono- or disubstituted by fluorine, or which may optionally be monosubstituted by methyl-, 2,2,2-trifluoroethyl-, cyclopropyl- or cyclopropylmethyl-,
   and the diastereomers, racemates, polymorphs and physiologically acceptable salts thereof.

Exceptional preference is given to those compounds of the general formula (I)

in which
A is —NH— or —N(methyl)-,
X is —N— or —CH—,
n is 0 or 1,
R¹ is a —C(═O)NR⁷R⁸ or —S(═O)₂NR⁷R⁸ group,
   or is

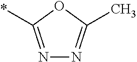 [SMILES: *c1nnc(C)o1]

in which “*” denotes the attachment point to the rest of the molecule,
R² is hydrogen, methyl- or methoxy-,
   or
R¹ and R² together with the phenyl ring to which they are bonded are

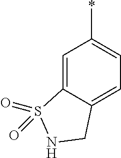 [SMILES: *c1ccc2c(c1)S(=O)(=O)NC2]

in which “*” denotes the attachment point to the rest of the molecule,
R³ is methyl-,
R⁴ is methyl-,
R⁵ is hydrogen,
R⁶ is isopropyl-, isobutyl-, 2-methoxyethyl-, benzyl-, 4-methoxybenzyl-, 2,6-difluorobenzyl-, cyclopentyl-, cyclohexyl-, cycloheptyl-, tetrahydropyran-4-yl-, phenyl-, 3-methylphenyl- or 4-fluorophenyl-,
   or is

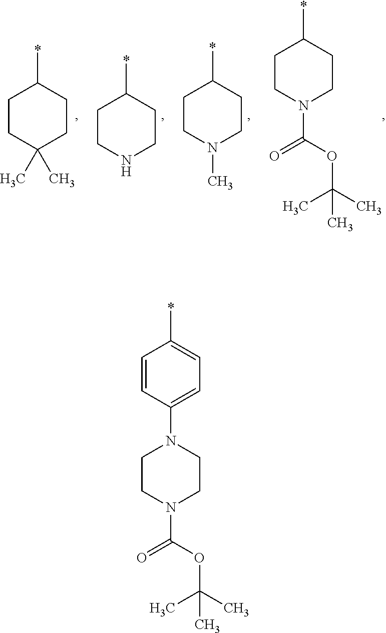

in which “*” denotes the attachment point to the rest of the molecule,
R⁷ is hydrogen, methyl-, ethyl-, isopropyl- or cyclopropyl-,
   or is

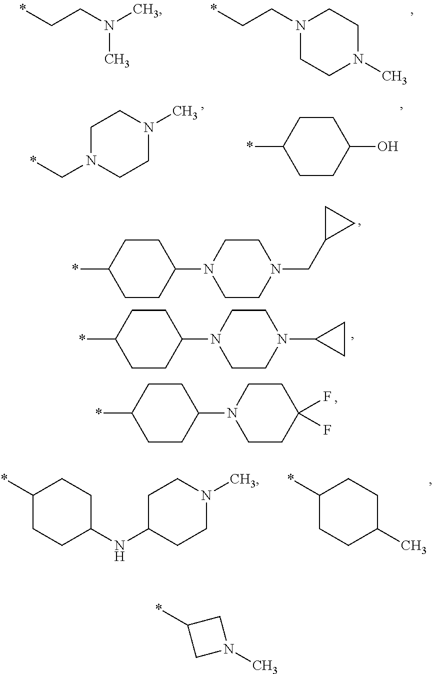

where “*” in each case denotes the attachment point to the rest of the molecule,
R⁸ is hydrogen, methyl- or ethyl-,
   or
R⁷ and R⁸ together with the nitrogen atom to which they are bonded are

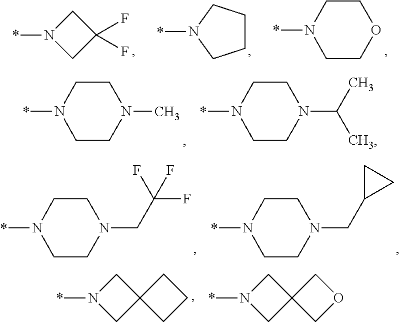

where “*” in each case denotes the attachment point to the rest of the molecule, and the diastereomers, racemates, polymorphs and physiologically acceptable salts thereof.

Likewise of interest are those compounds of the general formula I in which
A is —NH— or —O—,
X is —N— or —CH—,
n is 0.1 or 2,
R¹ is a —C(═O)NR⁷R⁸ or —S(═O)₂NR⁷R⁸ group,
   or
   is 5-membered monocyclic heteroaryl- which may optionally be mono-, di- or trisubstituted identically or differently by halogen, cyano, C₁-C₄-alkyl-, C₂-C₄-alkenyl-, C₂-C₄-alkynyl-, halo-C₁-C₄-alkyl-, C₁-C₄-alkoxy-, halo-C₁-C₄-alkoxy-, C₁-C₄-alkylthio-, halo-C₁-C₄-alkylthio-,
   —NR⁹R¹⁰, —C(═O)OR¹¹, —C(═O)N⁹R¹⁰, —C(═O)R¹¹, —S(═O)₂R¹¹, —S(═O)₂NR⁹R¹⁰,
R² is hydrogen, halogen, cyano, C₁-C₃-alkyl-, C₂-C₄-alkenyl-, C₂-C₄-alkynyl-, halo-C₁-C₄-alkyl-, C₁-C₄-alkoxy-, halo-C₁-C₄-alkoxy-, C₁-C₄-alkylthio- or halo-C₁-C₄-alkylthio-, and, if n is 2, R² may be the same or different,
R³ is methyl- or ethyl-,
R⁴ is hydrogen or C₁-C₃-alkyl-,
R⁵ is hydrogen or C₁-C₃-alkyl-,
   or
R⁴ and R⁵ together with the carbon atom to which they are bonded are C₃-C₆-cycloalkylene,
R⁶ is C₁-C₆-alkyl- which may optionally be monosubstituted by phenyl-, C₃-C₈-cycloalkyl-, or 4- to 8-membered heterocycloalkyl-,
   in which phenyl- may itself optionally be mono-, di- or trisubstituted identically or differently by: halogen, cyano, C₁-C₄-alkyl-, C₂-C₄-alkenyl-, C₂-C₄-alkynyl-, C₁-C₄-alkoxy-, halo-C₁-C₄-alkyl-, halo-C₁-C₄-alkoxy-,
   and
   in which C₃-C₈-cycloalkyl- and 4- to 8-membered heterocycloalkyl- may themselves optionally be mono- or disubstituted identically or differently by C₁-C₃-alkyl-,
   or
   is C₃-C₈-cycloalkyl- or 4- to 8-membered heterocycloalkyl- which may optionally be mono- or disubstituted identically or differently by C₁-C₃-alkyl-,
R⁷ is C₁-C₆-alkyl- which may optionally be mono-, di- or trisubstituted identically or differently by: hydroxyl, oxo, fluorine, cyano, C₁-C₄-alkoxy-, halo-C₁-C₄-alkoxy-, —NR⁹R¹⁰, C₃-C₈-cycloalkyl-, C₄-C₈-cycloalkenyl-, 4- to 8-membered heterocycloalkyl-, 4- to 8-membered heterocycloalkenyl-, C₅-C₁₁-spirocycloalkyl-, C₅-C₁₁-heterospirocycloalkyl-, bridged C₆-C₁₂-cycloalkyl-, bridged C₆-C₁₂-heterocycloalkyl-, C₆-C₁₂-bicycloalkyl-, C₆-C₁₂-heterobicycloalkyl-, phenyl-, 5- to 6-membered heteroaryl-,
   and in which C₃-C₈-cycloalkyl-, C₄-C₈-cycloalkenyl-, 4- to 8-membered heterocycloalkyl-, 4- to 8-membered heterocycloalkenyl-, C₅-C₁₁-spirocycloalkyl-, C₅-C₁₁-heterospirocycloalkyl-, bridged C₆-C₁₂-cycloalkyl-, bridged C₆-C₁₂-heterocycloalkyl-, C₆-C₁₂-bicycloalkyl-, C₆-C₁₂-heterobicycloalkyl- may optionally be mono- or disubstituted identically or differently by: hydroxyl, fluorine, oxo, cyano, C₁-C₃-alkyl-, fluoro-C₁-C₃-alkyl-, C₃-C₆-cycloalkyl-, cyclopropylmethyl-, C₁-C₃-alkylcarbonyl-, C₁-C₄-alkoxycarbonyl- or —NR⁹R¹⁰,
   and
   in which phenyl- and 5- to 6-membered heteroaryl- may optionally be mono- or disubstituted identically or differently by: halogen, cyano, trifluoromethyl-, C₁-C₃-alkyl-, C₁-C₃-alkoxy-,
   or is C₃-C₆-alkenyl- or C₃-C₆-alkynyl-,
   or is C₃-C₈-cycloalkyl-, C₄-C₈-cycloalkenyl-, C₅-C₁₁-spirocycloalkyl-, bridged C₆-C₁₂-cycloalkyl- or C₆-C₁₂-bicycloalkyl-, which may optionally be mono- or disubstituted identically or differently by: hydroxyl, oxo, cyano, fluorine, C₁-C₃-alkyl, C₁-C₃-alkoxy, trifluoromethyl, —NR⁹R¹⁰,
   or is 4- to 8-membered heterocycloalkyl-, 4- to 8-membered heterocycloalkenyl-, C₅-C₁₁-heterospirocycloalkyl-, bridged C₆-C₁₂-heterocycloalkyl- or C₆-C₁₂-heterobicycloalkyl-, which may optionally be mono- or disubstituted identically or differently by: hydroxyl, fluorine, oxo, cyano, C₁-C₃-alkyl-, fluoro-C₁-C₃-alkyl-, C₃-C₆-cycloalkyl-, cyclopropylmethyl-, C₁-C₃-alkylcarbonyl-, C₁-C₄-alkoxycarbonyl- or —NR⁹R¹⁰,
R⁸ is hydrogen or optionally singly or doubly, identically or differently hydroxyl-, oxo- or C₁-C₃-alkoxy-substituted C₁-C₃-alkyl-, or fluoro-C₁-C₃-alkyl,
   or
R⁷ and R⁸ together with the nitrogen atom to which they are bonded are 4- to 8-membered heterocycloalkyl, 4- to 8-membered heterocycloalkenyl-, C₅-C₁-heterospirocycloalkyl-, bridged C₆-C₁₂-heterocycloalkyl- or C₆-C₁₂-heterobicycloalkyl-, which may optionally be mono- or disubstituted identically or differently by: hydroxyl, fluorine, oxo, cyano, C₁-C₃-alkyl-, fluoro-C₁-C₃-alkyl-, C₃-C₆-cycloalkyl-, cyclopropylmethyl-, C₁-C₃-alkylcarbonyl-, C₁-C₄-alkoxycarbonyl- or —NR⁹R¹⁰,
R⁹ and R¹⁰ are each independently hydrogen or optionally singly or doubly, identically or differently hydroxyl-, oxo- or C₁-C₃-alkoxy-substituted C₁-C₃-alkyl, or fluoro-C₁-C₃-alkyl,
   or
R⁹ and R¹⁰ together with the nitrogen atom to which they are bonded are 4-8-membered heterocycloalkyl- which may optionally be mono- or disubstituted identically or differently by: hydroxyl, fluorine, oxo, cyano, C₁-C₃-alkyl-, fluoro-C₁-C₃-alkyl-, C₃-C₆-cycloalkyl-, cyclopropylmethyl-, C₁-C₃-alkylcarbonyl- or C₁-C₄-alkoxycarbonyl- and
R¹¹ is C₁-C₆-alkyl- or phenyl-C₁-C₃-alkyl-,
   and the diastereomers, racemates, polymorphs and physiologically acceptable salts thereof, surprisingly inhibit the interaction between BET proteins, especially BRD4, and an acetylated histone 4 peptide and hence inhibit the growth of cancer cells.

Preferably of interest are those compounds of the general formula (I) in which

in which
A is —NH—,
X is —N— or —CH—,
n is 0.1 or 2,
R¹ is a —C(═O)NR⁷R⁸ or —S(═O)₂NR⁷R⁸ group,
   or
   is oxazolyl-, thiazolyl-, oxadiazolyl- or thiadiazolyl-, which may optionally be mono- or disubstituted identically or differently by halogen, cyano, C₁-C₃-alkyl-, trifluoromethyl-, C₁-C₃-alkoxy-, trifluoromethoxy- or —NR⁹R¹⁰,
R² is hydrogen, fluorine, chlorine, cyano, methoxy- or ethoxy-, and ifn is 2, R² may be the same or different,
R³ is methyl- or ethyl-,
R⁴ is hydrogen, methyl- or ethyl-,
R⁵ is hydrogen, methyl- or ethyl-,
R⁶ is unsubstituted C₂-C₅-alkyl-,
   or
   is methyl- or ethyl-monosubstituted by phenyl- or 4- to 8-membered heterocycloalkyl-,
   in which phenyl- may itself optionally be mono- or disubstituted identically or differently by: fluorine, chlorine, bromine, cyano, C₁-C₃-alkyl-, C₁-C₃-alkoxy-, trifluoromethyl-,
   and
   in which 4- to 8-membered heterocycloalkyl- may itself optionally be mono- or disubstituted by methyl-,
   or
   is C₃-C₆-cycloalkyl- or 4- to 8-membered heterocycloalkyl- which may optionally be mono- or disubstituted by methyl-,
R⁷ is C₁-C₆-alkyl- which may optionally be mono-, di- or trisubstituted identically or differently by: hydroxyl, oxo, fluorine, cyano, C₁-C₃-alkoxy-, fluoro-C₁-C₃-alkoxy-, —NR⁹R¹⁰, 4- to 8-membered heterocycloalkyl-, phenyl-, 5- to 6-membered heteroaryl-,
   and in which the 4- to 8-membered heterocycloalkyl- may optionally be monosubstituted by: hydroxyl, oxo, C₁-C₃-alkyl-, fluoro-C₁-C₃-alkyl-, cyclopropyl-cyclopropylmethyl-, acetyl- or tert-butoxycarbonyl-,
   or is C₃-C₆-cycloalkyl- which may optionally be mono- or disubstituted identically or differently by: hydroxyl, oxo, cyano, fluorine, —NR⁹R¹⁰,
   or is 4- to 8-membered heterocycloalkyl-, C₆-C₈-heterospirocycloalkyl-, bridged C₆-C₁₀-heterocycloalkyl- or C₆-C₁₀-heterobicycloalkyl-, which may optionally be mono- or disubstituted identically or differently by: hydroxyl, oxo, C₁-C₃-alkyl-, fluoro-C₁-C₃-alkyl-, cyclopropyl-, cyclopropylmethyl-, acetyl- or tert-butoxycarbonyl-,
R⁸ is hydrogen or C₁-C₃-alkyl,
   or
R⁷ and R⁸ together with the nitrogen atom to which they are bonded are 4- to 8-membered heterocycloalkyl-, C₆-C₈-heterospirocycloalkyl-, bridged C₆-C₁₀-heterocycloalkyl- or C₆-C₁₀-heterobicycloalkyl-,
   which may optionally be mono- or disubstituted identically or differently by: hydroxyl, oxo, C₁-C₃-alkyl-, fluoro-C₁-C₃-alkyl-, cyclopropyl-, cyclopropylmethyl-, acetyl- or tert-butoxycarbonyl-,
R⁹ and R¹⁰ are each independently hydrogen or optionally mono-hydroxyl- or -oxo-substituted C₁-C₃-alkyl-, or trifluoromethyl-,
   or
R⁹ and R¹⁰ together with the nitrogen atom to which they are bonded are 4-7-membered heterocycloalkyl- which may optionally be mono- or disubstituted identically or differently by: hydroxyl, oxo, C₁-C₃-alkyl-, fluoro-C₁-C₃-alkyl-, cyclopropyl-, cyclopropylmethyl-, acetyl- or tert-butoxycarbonyl-,
   and the diastereomers, racemates, polymorphs and physiologically acceptable salts thereof.

Of particularly preferred interest are also those compounds of the general formula (I)

in which
A is —NH—,
X is —N— or —CH—,
n is 0 or 1,
R¹ is a —C(═O)NR⁷R⁸ or —S(═O)₂NR⁷R⁸ group,
R² is fluorine, chlorine or methoxy-,
R³ is methyl-,
R⁴ is methyl- or ethyl-,
R⁵ is hydrogen,
R⁶ is unsubstituted C₃-C₅-alkyl-,
   or
   is methyl-monosubstituted by phenyl- or 4- to 6-membered heterocycloalkyl-, in which phenyl- may itself optionally be mono- or disubstituted identically or differently by: fluorine, chlorine, cyano, methyl-, methoxy-,
   and
   in which 4- to 6-membered heterocycloalkyl- may itself optionally be monosubstituted by methyl-,
   or
   is C₃-C₆-cycloalkyl-, or 4- to 6-membered heterocycloalkyl-,
R⁷ is C₁-C₄-alkyl- which may optionally be monosubstituted by —NR⁹R¹⁰ or 4- to 8-membered heterocycloalkyl-,
   in which the 4- to 8-membered heterocycloalkyl- may optionally be monosubstituted by: oxo, C₁-C₃-alkyl-, fluoro-C₁-C₃-alkyl-, cyclopropyl- or cyclopropylmethyl-,
   or is C₃-C₆-cycloalkyl- which may optionally be monosubstituted by hydroxyl, fluorine or —NR⁹R¹⁰,
   or is 4- to 8-membered heterocycloalkyl- which may optionally be mono- or disubstituted identically or differently by: oxo, C₁-C₃-alkyl-, fluoro-C₁-C₃-alkyl-, cyclopropyl- or cyclopropylmethyl-,
R⁸ is hydrogen or methyl-,
   or
R⁷ and R⁸ together with the nitrogen atom to which they are bonded are 5- to 6-membered heterocycloalkyl- or C₆-C₈-heterospirocycloalkyl-, which may optionally be mono- or disubstituted identically or differently by: oxo, C₁-C₃-alkyl-, fluoro-C₁-C₃-alkyl-, cyclopropyl- or cyclopropylmethyl-,
R⁹ and R¹⁰ are each independently hydrogen or optionally mono-hydroxyl- or -oxo-substituted C₁-C₃-alkyl, or trifluoromethyl-,
   or
R⁹ and R¹⁰ together with the nitrogen atom to which they are bonded are 4-7-membered heterocycloalkyl- which may optionally be mono- or disubstituted identically or differently by: oxo, C₁-C₃-alkyl-, fluoro-C₁-C₃-alkyl-, cyclopropyl- or cyclopropylmethyl-,
   and the diastereomers, racemates, polymorphs and physiologically acceptable salts thereof.

Of particularly preferred interest are additionally also those compounds of the general formula (I)

in which
A is —NH—,
X is —N—,
n is 0 or 1,
R¹ is a —C(═O)NR⁷R⁸ or —S(═O)₂NR⁷R⁸ group,
R² is fluorine, chlorine or methoxy-,
R³ is methyl-,
R⁴ is methyl- or ethyl-,
R⁵ is hydrogen,
R⁶ is unsubstituted C₃-C₅-alkyl-,
   or
   is methyl-monosubstituted by phenyl- or 4- to 6-membered heterocycloalkyl-, in which phenyl- may itself optionally be mono- or disubstituted identically or differently by: fluorine, chlorine, cyano, methyl-, methoxy-,
   and
   in which 4- to 6-membered heterocycloalkyl- may itself optionally be monosubstituted by methyl-,
   or
   is C₃-C₆-cycloalkyl-, or 4- to 6-membered heterocycloalkyl-,
R⁷ is C₁-C₄-alkyl- which may optionally be monosubstituted by —NR⁹R¹⁰ or 4- to 8-membered heterocycloalkyl-,
   in which the 4- to 8-membered heterocycloalkyl- may optionally be monosubstituted by: oxo, C₁-C₃-alkyl-, fluoro-C₁-C₃-alkyl-, cyclopropyl- or cyclopropylmethyl-,
   or is C₃-C₆-cycloalkyl- which may optionally be monosubstituted by hydroxyl, fluorine or —NR⁹R¹⁰,
   or is 4- to 8-membered heterocycloalkyl- which may optionally be mono- or disubstituted identically or differently by: oxo, C₁-C₃-alkyl-, fluoro-C₁-C₃-alkyl-, cyclopropyl- or cyclopropylmethyl-,
R⁸ is hydrogen or methyl-,
   or
R⁷ and R⁸ together with the nitrogen atom to which they are bonded are 5- to 6-membered heterocycloalkyl- or C₆-C₈-heterospirocycloalkyl-, which may optionally be mono- or disubstituted identically or differently by: oxo, C₁-C₃-alkyl-, fluoro-C₁-C₃-alkyl-, cyclopropyl- or cyclopropylmethyl-,
R⁹ and R¹⁰ are each independently hydrogen or optionally mono-hydroxyl- or -oxo-substituted C₁-C₃-alkyl, or trifluoromethyl-,
   or
R⁹ and R¹⁰ together with the nitrogen atom to which they are bonded are 4-7-membered heterocycloalkyl- which may optionally be mono- or disubstituted identically or differently by: oxo, C₁-C₃-alkyl-, fluoro-C₁-C₃-alkyl-, cyclopropyl- or cyclopropylmethyl-,
   and the diastereomers, racemates, polymorphs and physiologically acceptable salts thereof.

Of particularly preferred interest are additionally also those compounds of the general formula (I)

in which
A is —NH—,
X is —CH—,
n is 0 or 1,
R¹ is a —C(═O)NR⁷R⁸ or —S(═O)₂NR⁷R⁸ group,
R² is fluorine, chlorine or methoxy-,
R³ is methyl-,
R⁴ is methyl- or ethyl-,
R⁵ is hydrogen,
R⁶ is unsubstituted C₃-C₅-alkyl-,
   or
   is methyl-monosubstituted by phenyl- or 4- to 6-membered heterocycloalkyl-, in which phenyl- may itself optionally be mono- or disubstituted identically or differently by: fluorine, chlorine, cyano, methyl-, methoxy-,
   and
   in which 4- to 6-membered heterocycloalkyl- may itself optionally be monosubstituted by methyl-,
   or
   is C₃-C₆-cycloalkyl-, or 4- to 6-membered heterocycloalkyl-,
R⁷ is C₁-C₄-alkyl- which may optionally be monosubstituted by —NR⁹R¹⁰ or 4- to 8-membered heterocycloalkyl-,
   in which the 4- to 8-membered heterocycloalkyl- may optionally be monosubstituted by: oxo, C₁-C₃-alkyl-, fluoro-C₁-C₃-alkyl-, cyclopropyl- or cyclopropylmethyl-,
   or is C₃-C₆-cycloalkyl- which may optionally be monosubstituted by hydroxyl, fluorine or —NR⁹R¹⁰,
   or is 4- to 8-membered heterocycloalkyl- which may optionally be mono- or disubstituted identically or differently by: oxo, C₁-C₃-alkyl-, fluoro-C₁-C₃-alkyl-, cyclopropyl- or cyclopropylmethyl-,
R⁸ is hydrogen or methyl-,
   or
R⁷ and R⁸ together with the nitrogen atom to which they are bonded are 5- to 6-membered heterocycloalkyl- or C₆-C₈-heterospirocycloalkyl-, which may optionally be mono- or disubstituted identically or differently by: oxo, C₁-C₃-alkyl-, fluoro-C₁-C₃-alkyl-, cyclopropyl- or cyclopropylmethyl-,
R⁹ and R¹⁰ are each independently hydrogen or optionally mono-hydroxyl- or -oxo-substituted C₁-C₃-alkyl, or trifluoromethyl-,
   or
R⁹ and R¹⁰ together with the nitrogen atom to which they are bonded are 4-7-membered heterocycloalkyl- which may optionally be mono- or disubstituted identically or differently by: oxo, C₁-C₃-alkyl-, fluoro-C₁-C₃-alkyl-, cyclopropyl- or cyclopropylmethyl-,
and the diastereomers, racemates, polymorphs and physiologically acceptable salts thereof.

Of very particularly preferred interest are those compounds of the general formula (I)

in which
A is —NH—,
X is —N— or —CH—,
n is 0 or 1,
R¹ is a —C(═O)NR⁷R⁸ or —S(═O)₂NR⁷R⁸ group,
R² is methoxy-,
R³ is methyl-,
R⁴ is methyl-,
R⁵ is hydrogen,
R⁶ is isopropyl-,
   or
   is benzyl, wherein the phenyl moiety may optionally be mono- or disubstituted identically or differently by: fluorine, chlorine, methoxy-,
   or
   is cyclopentyl- or cyclohexyl-,
   or
   is tetrahydrofuranyl- or tetrahydropyranyl-,
R⁷ is C₁-C₃-alkyl- which may optionally be monosubstituted by —NR⁹R¹⁰,
   or is C₅-C₆-cycloalkyl- which may optionally be monosubstituted by —NR⁹R¹⁰,
   or is 4- to 6-membered heterocycloalkyl- which may optionally be monosubstituted by methyl-,
R⁸ is hydrogen or methyl-,
   or
R⁷ and R⁸ together with the nitrogen atom to which they are bonded are 6-membered heterocycloalkyl- which may optionally be monosubstituted by methyl-,
R⁹ and R¹⁰ are each independently hydrogen or C₁-C₃-alkyl-,
   or
R⁹ and R¹⁰ together with the nitrogen atom to which they are bonded are 6-membered heterocycloalkyl- which may optionally be monosubstituted by methyl-, 2,2,2-trifluoroethyl- or cyclopropylmethyl-,
   and the diastereomers, racemates, polymorphs and physiologically acceptable salts thereof.

Of exceptionally preferred interest are also those compounds of the general formula (I)

in which
A is —NH—,
X is —N— or —CH—,
n is 0 or 1,
R¹ is a —C(═O)NR⁷R⁸ or —S(═O)₂NR⁷R⁸ group,
R² is methoxy-,
R³ is methyl-,
R⁴ is methyl-,
R⁵ is hydrogen,
R⁶ is 4-methoxybenzyl-, 2,6-difluorobenzyl-, cyclopentyl- or tetrahydropyran-4-yl-,
R⁷ is

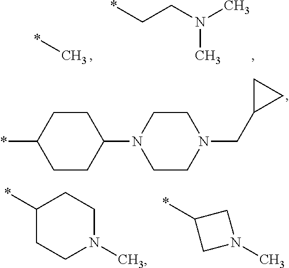

where “*” in each case denotes the attachment point to the rest of the molecule, and
R⁸ is hydrogen or methyl-,
   or
R⁷ and R⁸ together with the nitrogen atom to which they are bonded are

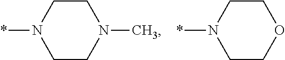

where “*” in each case denotes the attachment point to the rest of the molecule, and the diastereomers, racemates, polymorphs and physiologically acceptable salts thereof.

Preference is given to compounds of the general formula (I) in which A is —NH—.

Preference is given to compounds of the general formula (I) in which A is —O—.

Preference is given to compounds of the general formula (I) in which A is —NH— or is —N(C₁-C₃-alkyl)-.

Preference is given to compounds of the general formula (I) in which A is —N(C₁-C₃-alkyl)-.

Particular preference is given to compounds of the general formula (I) in which A is —NH— or is —N(methyl)-.

Particular preference is given to compounds of the general formula (I) in which A is —N(methyl)-.

Preference is given to compounds of the general formula (I) in which X is —N—.

Preference is given to compounds of the general formula (I) in which X is —CH—.

Preference is given to compounds of the general formula (I) in which n is the number 0 or the number 1.

Preference is given to compounds of the general formula (I) in which n is the number 0.

Preference is given to compounds of the general formula (I) in which n is the number 1.

Preference is given to compounds of the general formula (I) in which R¹ is —C(═O)NR⁷R⁸.

Preference is given to compounds of the general formula (I) in which R¹ is —S(═O)₂NR⁷R⁸.

Preference is given to compounds of the general formula (I) in which R¹ is 5-membered monocyclic hetaryl- which may optionally be mono-, di- or trisubstituted identically or differently by halogen, cyano, C₁-C₄-alkyl-, C₂-C₄-alkenyl-, C₂-C₄-alkynyl-, halo-C₁-C₄-alkyl-, C₁-C₄-alkoxy-, halo-C₁-C₄-alkoxy-, C₁-C₄-alkylthio-, halo-C₁-C₄-alkylthio-, —NR⁹R¹⁰, —C(═O)OR¹¹, —C(═O)N⁹R¹⁰, —C(═O)R¹¹, —S(═O)₂R¹¹, —S(═O)₂NR⁹R¹⁰.

Preference is given to compounds of the general formula (I) in which R¹ is oxazolyl-, thiazolyl-, oxadiazolyl- or thiadiazolyl-, which may optionally be mono- or disubstituted identically or differently by halogen, cyano, C₁-C₃-alkyl-, trifluoromethyl-, C₁-C₃-alkoxy-, trifluoromethoxy- or —NR⁹R¹⁰.

Preference is given to compounds of the general formula (I) in which R¹ is a —C(═O)NR⁷R⁸ or —S(═O)₂NR⁷R⁸ group, or

is oxazolyl-, thiazolyl-, oxadiazolyl- or thiadiazolyl-, which may optionally be mono- or disubstituted identically or differently by halogen, cyano, C₁-C₃-alkyl-, trifluoromethyl-, C₁-C₃-alkoxy-, trifluoromethoxy- or —NR⁹R¹⁰.

Preference is given to compounds of the formula (I) in which R¹ is oxazolyl-, thiazolyl-, oxadiazolyl- or thiadiazolyl-, which may optionally be mono- or disubstituted identically or differently by halogen, cyano, C₁-C₃-alkyl-, trifluoromethyl-, C₁-C₃-alkoxy-, trifluoromethoxy- or —NR⁹R¹⁰.

Particular preference is given to compounds of the general formula (I) in which R¹ is a —C(═O)NR⁷R⁸ or —S(═O)₂NR⁷R⁸ group, or

is oxazolyl- or oxadiazolyl- which may optionally be mono- or disubstituted identically or differently by C₁-C₃-alkyl-.

Particular preference is given to compounds of the general formula (I) in which R¹ is oxazolyl- or oxadiazolyl- which may optionally be mono- or disubstituted identically or differently by C₁-C₃-alkyl-.

Very particular preference is given to compounds of the general formula (I) in which R¹ is a —C(═O)NR⁷R⁸ or —S(═O)₂NR⁷R⁸ group, or

is oxazolyl- or oxadiazolyl- which may optionally be mono- or disubstituted by methyl-.

Very particular preference is given to compounds of the general formula (I) in which R¹ is oxazolyl- or oxadiazolyl- which may optionally be mono- or disubstituted by methyl-.

Exceptional preference is given to compounds of the general formula (I) in which R¹ is a —C(═O)NR⁷R⁸ or —S(═O)₂NR⁷R⁸ group, or is

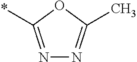

in which “*” denotes the attachment point to the rest of the molecule,

Exceptional preference is given to compounds of the general formula (I) in which R¹ is

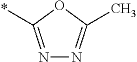

in which “*” denotes the attachment point to the rest of the molecule.

Preference is given to compounds of the general formula (I) in which R² is hydrogen, fluorine, chlorine, cyano, methyl-, methoxy-, ethyl- or ethoxy-.

Preference is given to compounds of the general formula (I) in which R² is C₁-C₃-alkoxy-.

Preference is given to compounds of the general formula (I) in which R² is ethoxy-.

Preference is given to compounds of the general formula (I) in which R² is fluorine.

Preference is given to compounds of the general formula (I) in which R² is chlorine.

Particular preference is given to compounds of the general formula (I) in which R² is hydrogen, fluorine, chlorine, methyl- or methoxy-.

Particular preference is given to compounds of the general formula (I) in which R² is hydrogen, methyl- or methoxy-.

Particular preference is given to compounds of the general formula (I) in which R² is methoxy-.

Particular preference is given to compounds of the general formula (I) in which R² is methyl-.

Particular preference is given to compounds of the general formula (I) in which R² is hydrogen.

Preference is given to compounds of the general formula (I) in which R¹ and R² together are a *—S(═O)₂—NR⁸—CH₂—** or *—C(═O)—NR⁸—CH₂—* group in which “*” denotes the attachment point of R¹ to the phenyl ring in formula (I), and in which “**” denotes a carbon atom of this phenyl ring adjacent to this attachment point.

Particular preference is given to compounds of the general formula (I) in which R¹ and R² together are a *—S(═O)₂—NR⁸—CH₂—** group in which “*” denotes the attachment point of R¹ to the phenyl ring in formula (I), and in which “**” denotes a carbon atom of this phenyl ring adjacent to this attachment point.

Very particular preference is given to compounds of the general formula (I) in which R¹ and R² together are a *—S(═O)₂—NH—CH₂—** group in which “*” denotes the attachment point of R1 to the phenyl ring in formula (I), and in which “**” denotes a carbon atom of this phenyl ring adjacent to this attachment point.

Particular preference is given to compounds of the general formula (I) in which R¹ is a —C(═O)NR⁷R⁸ or —S(═O)₂NR⁷R⁸ group,

or
is oxazolyl- or oxadiazolyl- which may optionally be mono- or disubstituted identically or differently by C₁-C₃-alkyl-,
and in which R² is hydrogen, fluorine, chlorine, methyl- or methoxy-,
or in which R¹ and R² together are a *—S(═O)₂—NR⁸—CH₂—** group in which “*” denotes the attachment point of R¹ to the phenyl ring in formula (I), and in which “**” denotes a carbon atom of this phenyl ring adjacent to this attachment point.

Very particular preference is given to compounds of the general formula (I) in which R¹ is a —C(═O)NR⁷R⁸ or —S(═O)₂NR⁷R⁸ group,

or
is oxazolyl- or oxadiazolyl- which may optionally be mono- or disubstituted identically or differently by methyl-,
and in which R² is hydrogen, methyl- or methoxy-,
or in which R¹ and R² together are a *—S(═O)₂—NH—CH₂—** group in which “*” denotes the attachment point of R¹ to the phenyl ring in formula (I), and in which “**” denotes a carbon atom of this phenyl ring adjacent to this attachment point.

Exceptional preference is given to compounds of the general formula (I) in which R¹ is a —C(═O)NR⁷R⁸ or —S(═O)₂NR⁷R⁸ group,

or
or is

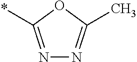 [SMILES: *c1nnc(C)o1]

in which “*” denotes the attachment point to the rest of the molecule,
and in which R² is hydrogen, methyl- or methoxy-,
or in which R¹ and R² together with the phenyl ring to which they are bonded are

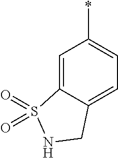 [SMILES: *c1ccc2c(c1)S(=O)(=O)NC2]

in which “*” denotes the attachment point to the rest of the molecule.

Preference is given to compounds of the general formula (I) in which R³ is methyl- or ethyl-.

Preference is given to compounds of the general formula (I) in which R³ is ethyl-.

Particular preference is given to compounds of the general formula (I) in which R³ is methyl-.

Preference is given to compounds of the general formula (I) in which R⁴ is hydrogen, methyl- or ethyl-.

Preference is given to compounds of the general formula (I) in which R⁴ is methyl- or ethyl-.

Preference is given to compounds of the general formula (I) in which R⁴ is ethyl-.

Particular preference is given to compounds of the general formula (I) in which R⁴ is methyl-.

Preference is given to compounds of the general formula (I) in which R⁴ is ethyl- and R⁵ is hydrogen.

Preference is given to compounds of the general formula (I) in which one substituent in each case from R⁴ and R⁵ is methyl- and one is hydrogen, so as to result in a racemate with respect to the stereocenter formed from R⁴, R⁵ and the carbon atom bonded to R⁴ and R⁵.

Particular preference is given to compounds of the general formula (I) in which one substituent in each case from R⁴ and R⁵ is methyl- and one is hydrogen, so as to result in an isomer mixture in which the (R) form predominates with respect to the stereocentre formed from R⁴, R⁵ and the carbon atom bonded to R⁴ and R.

Particular preference is given to compounds of the general formula (I) in which R⁴ is methyl- and R⁵ is hydrogen.

Preference is given to compounds of the general formula (I) in which R⁶ is unsubstituted C₃-C₅-alkyl-,

or
is methyl-monosubstituted by phenyl- or 4- to 6-membered heterocycloalkyl-,
in which phenyl- may itself optionally be mono- or disubstituted identically or differently by: fluorine, chlorine, cyano, methyl-, methoxy-,
and
in which 4- to 6-membered heterocycloalkyl- may itself optionally be monosubstituted by methyl-, or
is C₃-C₆-cycloalkyl-, or 4- to 6-membered heterocycloalkyl-.

Preference is given to compounds of the general formula (I) in which R⁶ is unsubstituted C₃-C₅-alkyl.

Preference is given to compounds of the general formula (I) in which R⁶ is methyl-monosubstituted by phenyl-,

in which phenyl- may itself optionally be mono- or disubstituted identically or differently by: fluorine, chlorine, cyano, methyl-, methoxy-.

Preference is given to compounds of the general formula (I) in which R⁶ is methyl-monosubstituted by 4- to 6-membered heterocycloalkyl-,

in which 4- to 6-membered heterocycloalkyl- itself may optionally be monosubstituted by methyl-.

Preference is given to compounds of the general formula (I) in which R⁶ is C₃-C₆-cycloalkyl-.

Preference is given to compounds of the general formula (I) in which R⁶ is 4- to 6-membered heterocycloalkyl-.

Preference is further given to compounds of the general formula (I) in which R⁶ is C₂-C₅-alkyl-, or is methyl- or ethyl-monosubstituted by C₁-C₃-alkoxy-, phenyl- or 4- to 8-membered heterocycloalkyl-,
in which phenyl- may itself optionally be mono-, di- or trisubstituted identically or differently by: fluorine, chlorine, bromine, cyano, C₁-C₃-alkyl-, C₁-C₃-alkoxy-, and in which the 4- to 8-membered heterocycloalkyl- may itself optionally be mono- or disubstituted identically or differently by methyl-,
   or
   is C₃-C₈-cycloalkyl- or 4- to 8-membered heterocycloalkyl-, which may optionally be mono- or disubstituted identically or differently by C₁-C₃-alkyl- or C₁-C₄-alkoxycarbonyl-,
   or
   is phenyl which may optionally be mono- or disubstituted identically or differently by fluorine, chlorine, methyl- or 6-membered heterocycloalkyl-,
in which the 6-membered heterocycloalkyl- may itself optionally be monosubstituted by methyl- or tert-butoxycarbonyl-,

Preference is given to compounds of the general formula (I) in which R⁶ is C₂-C₅-alkyl-, or is methyl- or ethyl-monosubstituted by C₁-C₃-alkoxy-, phenyl- or 4- to 8-membered heterocycloalkyl-,
in which phenyl- may itself optionally be mono-, di- or trisubstituted identically or differently by: fluorine, chlorine, bromine, cyano, C₁-C₃-alkyl-, C₁-C₃-alkoxy-, and in which the 4- to 8-membered heterocycloalkyl- may itself optionally be mono- or disubstituted identically or differently by methyl-.

Preference is given to compounds of the general formula (I) in which R⁶ is C₂-C₅-alkyl-.

Preference is given to compounds of the general formula (I) in which R⁶ is methyl- or ethyl-monosubstituted by C₁-C₃-alkoxy-, phenyl- or 4- to 8-membered heterocycloalkyl-,
in which phenyl- may itself optionally be mono-, di- or trisubstituted identically or differently by: fluorine, chlorine, bromine, cyano, C₁-C₃-alkyl-, C₁-C₃-alkoxy-, and in which the 4- to 8-membered heterocycloalkyl- may itself optionally be mono- or disubstituted identically or differently by methyl-.

Preference is given to compounds of the general formula (I) in which R⁶ is C₃-C₈-cycloalkyl- or 4- to 8-membered heterocycloalkyl- which may optionally be mono- or disubstituted identically or differently by C₁-C₃-alkyl- or C₁-C₄-alkoxycarbonyl-.

Preference is given to compounds of the general formula (I) in which R⁶ is phenyl which may optionally be mono- or disubstituted identically or differently by fluorine, chlorine, methyl- or 6-membered heterocycloalkyl-,
in which the 6-membered heterocycloalkyl- may itself optionally be monosubstituted by methyl- or tert-butoxycarbonyl-.

Particular preference is given to compounds of the general formula (I) in which R⁶ is benzyl-, wherein the phenyl moiety may optionally be mono- or disubstituted identically or differently by: fluorine, chlorine, methoxy-.

Particular preference is given to compounds of the general formula (I) in which R⁶ is cyclopentyl- or cyclohexyl-.

Particular preference is given to compounds of the general formula (I) in which R⁶ is tetrahydrofuranyl- or tetrahydropyranyl-.

Particular preference is further given to compounds of the general formula (I) in which R⁶ is C₃-C₅-alkyl- or is 2-methoxyethyl-,

or is methyl-monosubstituted by phenyl- or 4- to 6-membered heterocycloalkyl-,
in which phenyl- may itself optionally be mono- or disubstituted identically or differently by: fluorine, chlorine, cyano, methyl-, methoxy-, and
in which the 4- to 6-membered heterocycloalkyl- may itself optionally be monosubstituted by methyl-,
   or is C₃-C₈-cycloalkyl- or is 4- to 6-membered heterocycloalkyl-, which may optionally be mono- or disubstituted identically or differently by C₁-C₃-alkyl- or C₁-C₄-alkoxycarbonyl-,
   or is phenyl which may optionally be mono- or disubstituted identically or differently by fluorine, chlorine, methyl- or N-tert-butoxycarbonylpiperazinyl-.

Particular preference is given to compounds of the general formula (I) in which R⁶ is C₃-C₅-alkyl- or is 2-methoxyethyl-,

or is methyl-monosubstituted by phenyl- or 4- to 6-membered heterocycloalkyl-,
in which phenyl- may itself optionally be mono- or disubstituted identically or differently by: fluorine, chlorine, cyano, methyl-, methoxy-, and
in which the 4- to 6-membered heterocycloalkyl- may itself optionally be monosubstituted by methyl-.

Particular preference is given to compounds of the general formula (I) in which R⁶ is C₃-C₅-alkyl- or is 2-methoxyethyl-.

Particular preference is given to compounds of the general formula (I) in which R⁶ is methyl-monosubstituted by phenyl- or 4- to 6-membered heterocycloalkyl-,
in which phenyl- may itself optionally be mono- or disubstituted identically or differently by: fluorine, chlorine, cyano, methyl-, methoxy-, and
in which the 4- to 6-membered heterocycloalkyl- may itself optionally be monosubstituted by methyl-.

Particular preference is given to compounds of the general formula (I) in which R⁶ is C₃-C₈-cycloalkyl- or is 4- to 6-membered heterocycloalkyl- which may optionally be mono- or disubstituted identically or differently by C₁-C₃-alkyl- or C₁-C₄-alkoxycarbonyl-.

Particular preference is given to compounds of the general formula (I) in which R⁶ is phenyl which may optionally be mono- or disubstituted identically or differently by fluorine, chlorine, methyl- or N-tert-butoxycarbonylpiperazinyl-.

Very particular preference is given to compounds of the general formula (I) in which R⁶ is isopropyl-, isobutyl- or 2-methoxyethyl-, or

is benzyl wherein the phenyl moiety may optionally be mono- or disubstituted identically or differently by: fluorine, methoxy-, or
is C₅-C₇-cycloalkyl- which may optionally be mono- or disubstituted by methyl-, or
is tetrahydrofuranyl-, tetrahydropyranyl- or piperidinyl-,
in which piperidinyl- may optionally be monosubstituted by methyl- or tert-butoxycarbonyl-, or
   is phenyl which may optionally be monosubstituted by fluorine, methyl- or N-tert-butoxycarbonylpiperazinyl-.

Very particular preference is given to compounds of the general formula (I) in which R⁶ is isopropyl-, isobutyl- or 2-methoxyethyl-, or

is benzyl wherein the phenyl moiety may optionally be mono- or disubstituted identically or differently by: fluorine, methoxy-.

Very particular preference is given to compounds of the general formula (I) in which R⁶ is isopropyl-, isobutyl- or 2-methoxyethyl-.

Very particular preference is given to compounds of the general formula (I) in which R⁶ is benzyl- wherein the phenyl moiety may optionally be mono- or disubstituted identically or differently by: fluorine, methoxy-.

Very particular preference is given to compounds of the general formula (I) in which R⁶ is tetrahydrofuranyl-, tetrahydropyranyl- or piperidinyl-,
in which piperidinyl- may optionally be monosubstituted by methyl- or tert-butoxycarbonyl-.

Very particular preference is given to compounds of the general formula (I) in which R⁶ is phenyl which may optionally be monosubstituted by fluorine, methyl- or N-tert-butoxycarbonylpiperazinyl-.

Exceptional preference is given to compounds of the general formula (I) in which R⁶ is isopropyl-, isobutyl- or 2-methoxyethyl-, benzyl-, 4-methoxybenzyl-, 2,6-difluorobenzyl-, cyclopentyl-, cyclohexyl-, cycloheptyl-, tetrahydropyran-4-yl-, phenyl-, 3-methylphenyl- or 4-fluorophenyl-,

or is

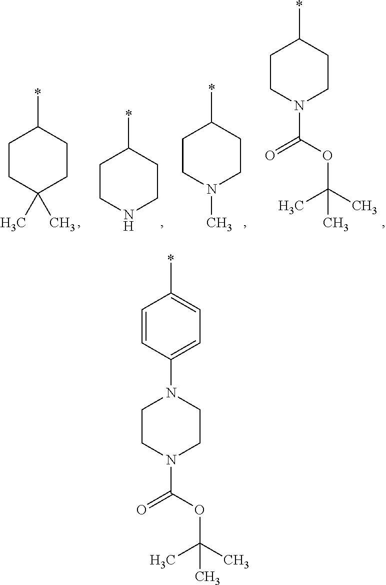

in which “*” in each case denotes the attachment point to the rest of the molecule.

Preference is given to compounds of the general formula (I) in which R⁷ is C₁-C₄-alkyl- which may optionally be monosubstituted by —NR⁹R¹⁰ or 4- to 8-membered heterocycloalkyl-,

in which the 4- to 8-membered heterocycloalkyl- may optionally be monosubstituted by: oxo, C₁-C₃-alkyl-, fluoro-C₁-C₃-alkyl-, cyclopropyl- or cyclopropylmethyl-,
or is C₃-C₆-cycloalkyl- which may optionally be monosubstituted by hydroxyl, fluorine or —NR⁹R¹⁰, or is 4- to 8-membered heterocycloalkyl- which may optionally be mono- or disubstituted identically or differently by: oxo, C₁-C₃-alkyl, fluoro-C₁-C₃-alkyl-, cyclopropyl- or cyclopropylmethyl-.

Preference is given to compounds of the general formula (I) in which R⁷ is C₁-C₄-alkyl- which may optionally be monosubstituted by —NR⁹R¹⁰ or 4- to 8-membered heterocycloalkyl-, in which the 4- to 8-membered heterocycloalkyl- may optionally be monosubstituted by: oxo, C₁-C₃-alkyl-, fluoro-C₁-C₃-alkyl-, cyclopropyl- or cyclopropylmethyl-.

Preference is given to compounds of the general formula (I) in which R⁷ is C₃-C₆-cycloalkyl- which may optionally be monosubstituted by hydroxyl, fluorine or —NR⁹R¹⁰.

Preference is given to compounds of the general formula (I) in which R⁷ is 4- to 8-membered heterocycloalkyl- which may optionally be mono- or disubstituted identically or differently by: oxo, C₁-C₃-alkyl, fluoro-C₁-C₃-alkyl-, cyclopropyl- or cyclopropylmethyl-.

Preference is further given to compounds of the general formula (I) in which R⁷ is hydrogen, or is C₁-C₆-alkyl- which may optionally be mono-, di- or trisubstituted identically or differently by: hydroxyl, oxo, fluorine, cyano, C₁-C₃-alkoxy-, fluoro-C₁-C₃-alkoxy-,

—NR⁹R¹⁰, 4- to 8-membered heterocycloalkyl-, phenyl-, 5- to 6-membered heteroaryl-,
in which the 4- to 8-membered heterocycloalkyl- may itself optionally be monosubstituted by: hydroxyl, oxo, C₁-C₃-alkyl-, fluoro-C₁-C₃-alkyl-, cyclopropyl-, cyclopropylmethyl-, acetyl- or tert-butoxycarbonyl-,
   or is C₃-C₆-cycloalkyl- which may optionally be mono- or disubstituted identically or differently by: hydroxyl, oxo, cyano, fluorine, —NR⁹R¹⁰,
   or is 4- to 8-membered heterocycloalkyl-, C₆-C₈-heterospirocycloalkyl-, bridged C₆-C₁₀-heterocycloalkyl- or C₆-C₁₀-heterobicycloalkyl-, which may optionally be mono- or disubstituted identically or differently by: hydroxyl, oxo, C₁-C₃-alkyl-, fluoro-C₁-C₃-alkyl-, cyclopropyl-, cyclopropylmethyl-, acetyl- or tert-butoxycarbonyl-.

Preference is given to compounds of the general formula (I) in which R⁷ is hydrogen.

Preference is given to compounds of the general formula (I) in which R⁷ is C₁-C₆-alkyl- which may optionally be mono-, di- or trisubstituted identically or differently by: hydroxyl, oxo, fluorine, cyano, C₁-C₃-alkoxy-, fluoro-C₁-C₃-alkoxy-, —NR⁹R¹⁰, 4- to 8-membered heterocycloalkyl-, phenyl-, 5- to 6-membered heteroaryl-,
in which the 4- to 8-membered heterocycloalkyl- may itself optionally be monosubstituted by: hydroxyl, oxo, C₁-C₃-alkyl-, fluoro-C₁-C₃-alkyl-, cyclopropyl-, cyclopropylmethyl-, acetyl- or tert-butoxycarbonyl-.

Preference is given to compounds of the general formula (I) in which R⁷ is C₃-C₆-cycloalkyl- which may optionally be mono- or disubstituted identically or differently by: hydroxyl, oxo, cyano, fluorine, —NR⁹R¹⁰.

Preference is given to compounds of the general formula (I) in which R¹ is 4- to 8-membered heterocycloalkyl-, C₆-C₈-heterospirocycloalkyl-, bridged C₆-C₁₀-heterocycloalkyl- or C₆-C₁₀-heterobicycloalkyl- which may optionally be mono- or disubstituted identically or differently by: hydroxyl, oxo, C₁-C₃-alkyl-, fluoro-C₁-C₃-alkyl-, cyclopropyl-, cyclopropylmethyl-, acetyl- or tert-butoxycarbonyl-.

Particular preference is given to compounds of the general formula (I) in which R⁷ is C₁-C₃-alkyl-which may optionally be monosubstituted by —NR⁹R¹⁰,

or is C₅-C₆-cycloalkyl- which may optionally be monosubstituted by —NR⁹R¹⁰,
or is 4- to 6-membered heterocycloalkyl- which may optionally be monosubstituted by methyl-.

Particular preference is given to compounds of the general formula (I) in which R⁷ is is C₁-C₃-alkyl- which may optionally be monosubstituted by —NR⁹R¹⁰.

Particular preference is given to compounds of the general formula (I) in which R⁷ is C₅-C₆-cycloalkyl- which may optionally be monosubstituted by —NR⁹R¹⁰.

Particular preference is given to compounds of the general formula (I) in which R⁷ is 4- to 6-membered heterocycloalkyl- which may optionally be monosubstituted by methyl-.

Particular preference is further given to compounds of the general formula (I) in which R⁷ is hydrogen,

or is C₁-C₄-alkyl which may optionally be monosubstituted by NR⁹R¹⁰ or
4- to 8-membered heterocycloalkyl-
in which the 4- to 8-membered heterocycloalkyl- may itself optionally be monosubstituted by: oxo, C₁-C₃-alkyl-, fluoro-C₁-C₃-alkyl-, cyclopropyl- or cyclopropylmethyl-,
   or is C₃-C₆-cycloalkyl- which may optionally be monosubstituted by hydroxyl, fluorine or —NR⁹R¹⁰, or is 4- to 8-membered heterocycloalkyl- which may optionally be mono- or disubstituted identically or differently by: oxo, C₁-C₃-alkyl-, fluoro-C₁-C₃-alkyl-, cyclopropyl- or cyclopropylmethyl-.

Particular preference is given to compounds of the general formula (I) in which R⁷ is C₁-C₄-alkyl-which may optionally be monosubstituted by —NR⁹R¹⁰ or 4- to 8-membered heterocycloalkyl-,
in which the 4- to 8-membered heterocycloalkyl- may itself optionally be monosubstituted by: oxo, C₁-C₃-alkyl-, fluoro-C₁-C₃-alkyl-, cyclopropyl- or cyclopropylmethyl-.

Particular preference is given to compounds of the general formula (I) in which R⁷ is C₃-C₆-cycloalkyl- which may optionally be monosubstituted by hydroxyl, fluorine or —NR⁹R¹⁰.

Particular preference is given to compounds of the general formula (I) in which R⁷ is 4- to 8-membered heterocycloalkyl which may optionally be mono- or disubstituted identically or differently by: oxo, C₁-C₃-alkyl-, fluoro-C₁-C₃-alkyl-, cyclopropyl- or cyclopropylmethyl-.

Very particular preference is given to compounds of the general formula (I) in which R⁷ is

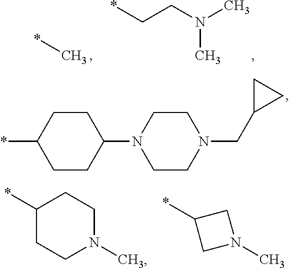

where “*” in each case denotes the attachment point to the rest of the molecule.

Very particular preference is further given to compounds of the general formula (I) in which R⁷ is hydrogen,

or is C₁-C₃-alkyl which may optionally be monosubstituted by —NR⁹R¹⁰ or N-methylpiperidinyl-,
or is cyclopropyl-, or is cyclohexyl-,
in which cyclohexyl- may optionally be monosubstituted by hydroxyl- or —NR⁹R¹⁰,
   or is 4- to 6-membered heterocycloalkyl which may optionally be monosubstituted by methyl-.

Very particular preference is given to compounds of the general formula (I) in which R⁷ is C₁-C₃-alkyl- which may optionally be monosubstituted by —NR⁹R¹⁰ or N-methylpiperidinyl-.

Very particular preference is given to compounds of the general formula (I) in which R⁷ is cyclopropyl-, or is cyclohexyl-,
in which cyclohexyl- may optionally be monosubstituted by hydroxyl- or —NR⁹R¹⁰.

Very particular preference is further given to compounds of the general formula (I) in which R⁷ is 4- to 6-membered heterocycloalkyl which may optionally be monosubstituted by methyl-.

Exceptional preference is further given to compounds of the general formula (I) in which R⁷ is hydrogen, methyl-, ethyl-, isopropyl- or cyclopropyl-,

or is

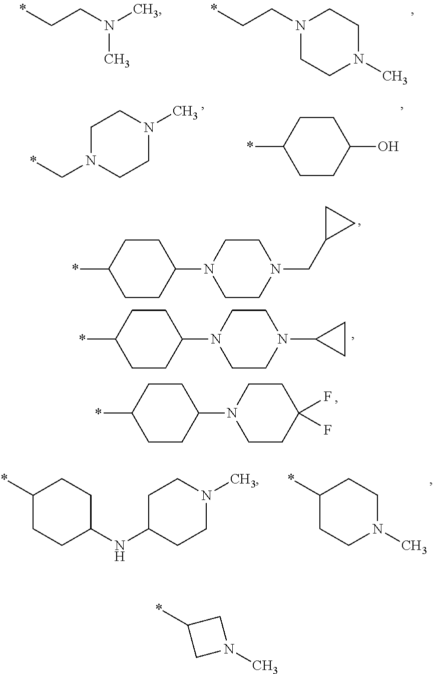

where “*” in each case denotes the attachment point to the rest of the molecule.

Preference is given to compounds of the general formula (I) in which R⁸ is hydrogen or methyl-.

Preference is given to compounds of the general formula (I) in which R⁸ is hydrogen, methyl- or ethyl-.

Preference is given to compounds of the general formula (I) in which R⁸ is hydrogen.

Preference is given to compounds of the general formula (I) in which R⁸ is methyl-.

Preference is given to compounds of the general formula (I) in which R⁸ is ethyl-.

Preference is given to compounds of the general formula (I) in which R⁷ and R⁸ together with the nitrogen atom to which they are bonded are 4- to 8-membered heterocycloalkyl-, C₆-C₈-heterospirocycloalkyl-, bridged C₆-C₁₀-heterocycloalkyl- or C₆-C₁₀-heterobicycloalkyl-, which may optionally be mono- or disubstituted identically or differently by: hydroxyl, oxo, C₁-C₃-alkyl-, fluoro-C₁-C₃-alkyl-, cyclopropyl-, cyclopropylmethyl-, acetyl- or tert-butoxycarbonyl-.

Preference is further given to compounds of the general formula (I) in which R¹ and R⁸ together with the nitrogen atom to which they are bonded are 4- to 8-membered heterocycloalkyl-, C₆-C₈-heterospirocycloalkyl-, bridged C₆-C₁₀-heterocycloalkyl- or C₆-C₁₀-heterobicycloalkyl-, which may optionally be mono- or disubstituted identically or differently by: hydroxyl, fluorine, oxo, C₁-C₃-alkyl-, fluoro-C₁-C₃-alkyl-, cyclopropyl-, cyclopropylmethyl-, acetyl- or tert-butoxycarbonyl-.

Preference is given to compounds of the general formula (I) in which R⁷ and R⁸ together with the nitrogen atom to which they are bonded are 4- to 8-membered heterocycloalkyl-, which may optionally be mono- or disubstituted identically or differently by: hydroxyl, oxo, C₁-C₃-alkyl-, fluoro-C₁-C₃-alkyl-, cyclopropyl-, cyclopropylmethyl-, acetyl- or tert-butoxycarbonyl-.

Particular preference is given to compounds of the general formula (I) in which R⁷ and R⁸ together with the nitrogen atom to which they are bonded are 5- to 6-membered heterocycloalkyl- or C₆-C₈-heterospirocycloalkyl-, which may optionally be mono- or disubstituted identically or differently by: oxo, C₁-C₃-alkyl, fluoro-C₁-C₃-alkyl-, cyclopropyl- or cyclopropylmethyl-.

Particular preference is given to compounds of the general formula (I) in which R⁷ and R⁸ together with the nitrogen atom to which they are bonded are 5- to 6-membered heterocycloalkyl- which may optionally be mono- or disubstituted identically or differently by: oxo, C₁-C₃-alkyl, fluoro-C₁-C₃-alkyl-, cyclopropyl- or cyclopropylmethyl-.

Particular preference is further given to compounds of the general formula (I) in which R⁷ and R⁸ together with the nitrogen atom to which they are bonded are 4- to 6-membered heterocycloalkyl or C₆-C₈-heterospirocycloalkyl, which may optionally be mono- or disubstituted identically or differently by: fluorine, oxo, C₁-C₃-alkyl-, fluoro-C₁-C₃-alkyl-, cyclopropyl- or cyclopropylmethyl-.

Very particular preference is given to compounds of the general formula (I) in which R⁷ and R⁸ together with the nitrogen atom to which they are bonded are 6-membered heterocycloalkyl- which may optionally be monosubstituted by methyl-.

Very particular preference is additionally given to compounds of the general formula (I) in which R⁷ and R⁸ together with the nitrogen atom to which they are bonded are

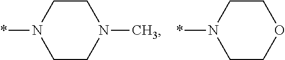

where “*” in each case denotes the attachment point to the rest of the molecule.

Very particular preference is further given to compounds of the general formula (I) in which R⁷ and R⁸ together with the nitrogen atom to which they are bonded are 4- to 6-membered heterocycloalkyl which may optionally be mono- or disubstituted by fluorine, or which may optionally be monosubstituted by methyl-, isopropyl-, 2,2,2-trifluoroethyl- or cyclopropylmethyl-, or are 6-azaspiro[3.3]heptyl- or are 2-oxa-6-azaspiro[3.3]heptyl-.

Exceptional preference is given to compounds of the general formula (I) in which R⁷ and R⁸ together with the nitrogen atom to which they are bonded are

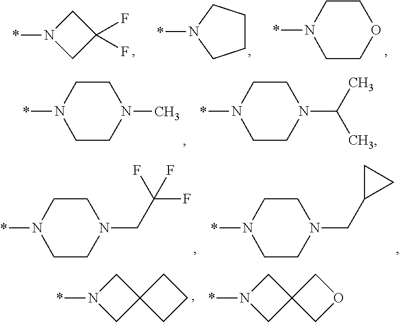

where “*” in each case denotes the attachment point to the rest of the molecule.

Preference is further given to compounds of the general formula (I) in which R⁷ is hydrogen, or is C₁-C₆-alkyl- which may optionally be mono-, di- or trisubstituted identically or differently by: hydroxyl, oxo, fluorine, cyano, C₁-C₃-alkoxy-, fluoro-C₁-C₃-alkoxy-,

—NR⁹R¹⁰, 4- to 8-membered heterocycloalkyl-, phenyl-, 5- to 6-membered heteroaryl-,
in which the 4- to 8-membered heterocycloalkyl- may itself optionally be monosubstituted by: hydroxyl, oxo, C₁-C₃-alkyl-, fluoro-C₁-C₃-alkyl-, cyclopropyl-, cyclopropylmethyl-, acetyl- or tert-butoxycarbonyl-,
   or is C₃-C₆-cycloalkyl- which may optionally be mono- or disubstituted identically or differently by: hydroxyl, oxo, cyano, fluorine, —NR⁹R¹⁰,
   or is 4- to 8-membered heterocycloalkyl-, C₆-C₈-heterospirocycloalkyl-, bridged C₆-C₁₀-heterocycloalkyl- or C₆-C₁₀-heterobicycloalkyl-, which may optionally be mono- or disubstituted identically or differently by: hydroxyl, oxo, C₁-C₃-alkyl-, fluoro-C₁-C₃-alkyl-, cyclopropyl-, cyclopropylmethyl-, acetyl- or tert-butoxycarbonyl-,
   and in which R⁸ is hydrogen or C₁-C₃-alkyl,
   or in which R⁷ and R⁸ together with the nitrogen atom to which they are bonded are 4- to 8-membered heterocycloalkyl-, C₆-C₈-heterospirocycloalkyl-, bridged C₆-C₁₀-heterocycloalkyl- or C₆-C₁₀-heterobicycloalkyl-,
   which may optionally be mono- or disubstituted identically or differently by: hydroxyl, fluorine, oxo, C₁-C₃-alkyl-, fluoro-C₁-C₃-alkyl-, cyclopropyl-, cyclopropylmethyl-, acetyl- or tert-butoxycarbonyl-.

Particular preference is given to compounds of the general formula (I) in which R⁷ is hydrogen, or is C₁-C₄-alkyl- which may optionally be monosubstituted by —NR⁹R¹⁰ or 4- to 8-membered heterocycloalkyl-,
in which the 4- to 8-membered heterocycloalkyl- may itself optionally be monosubstituted by: oxo, C₁-C₃-alkyl-, fluoro-C₁-C₃-alkyl-, cyclopropyl- or cyclopropylmethyl-,
   or is C₃-C₆-cycloalkyl- which may optionally be monosubstituted by hydroxyl, fluorine or —NR⁹R¹⁰, or is 4- to 8-membered heterocycloalkyl- which may optionally be mono- or disubstituted identically or differently by: oxo, C₁-C₃-alkyl-, fluoro-C₁-C₃-alkyl-, cyclopropyl- or cyclopropylmethyl-,
   and in which R⁸ is hydrogen, methyl- or ethyl-,
   or in which R⁷ and R⁸ together with the nitrogen atom to which they are bonded are 4- to 6-membered heterocycloalkyl- or C₆-C₈-heterospirocycloalkyl- which may optionally be mono- or disubstituted identically or differently by: fluorine, oxo, C₁-C₃-alkyl-, fluoro-C₁-C₃-alkyl-, cyclopropyl- or cyclopropylmethyl-.

Very particular preference is given to compounds of the general formula (I) in which R⁷ is hydrogen, or is C₁-C₃-alkyl- which may optionally be monosubstituted by —NR⁹R¹⁰ or N-methylpiperidinyl-,

or is cyclopropyl-, or is cyclohexyl-,
in which cyclohexyl- may optionally be monosubstituted by hydroxyl- or —NR⁹R¹⁰, or is 4- to 6-membered heterocycloalkyl which may optionally be monosubstituted by methyl-,
   and in which R⁸ is hydrogen, methyl- or ethyl-,
   or in which R⁷ and R⁸ together with the nitrogen atom to which they are bonded are 4- to 6-membered heterocycloalkyl- which may optionally be mono- or disubstituted by fluorine, or which may optionally be monosubstituted by methyl-, isopropyl-, 2,2,2-trifluoroethyl- or cyclopropylmethyl-, or are 6-azaspiro[3.3]heptyl- or are 2-oxa-6-azaspiro[3.3]heptyl-.

Preference is given to compounds of the general formula (I) in which R⁹ and R¹⁰ are each independently hydrogen or optionally mono-hydroxyl- or -oxo-substituted C₁-C₃-alkyl-, or trifluoromethyl-.

Preference is further given to compounds of the general formula (I) in which R⁹ and R¹⁰ are each independently hydrogen or optionally mono-hydroxyl- or -oxo-substituted C₁-C₃-alkyl, or are trifluoromethyl-, or are 6-membered heterocycloalkyl-,
in which the 6-membered heterocycloalkyl- may itself optionally be mono- or disubstituted identically or differently by C₁-C₃-alkyl.

Particular preference is given to compounds of the general formula (I) in which R⁹ and R¹⁰ are each independently hydrogen or C₁-C₃-alkyl-.

Particular preference is given to compounds of the general formula (I) in which R⁹ and R¹⁰ are each independently C₁-C₃-alkyl-.

Particular preference is given to compounds of the general formula (I) in which R⁹ and R¹⁰ are each methyl-.

Particular preference is further given to compounds of the general formula (I) in which R⁹ and R¹⁰ are each independently hydrogen or optionally mono-hydroxyl- or -oxo-substituted C₁-C₃-alkyl, trifluoromethyl-, or N-methylpiperidinyl-.

Particular preference is given to compounds of the general formula (I) in which R⁹ is optionally mono-hydroxyl- or -oxo-substituted C₁-C₃-alkyl-, trifluoromethyl-, or N-methylpiperidinyl-, and in which R¹⁰ is hydrogen.

Very particular preference is given to compounds of the general formula (I) in which R⁹ and R¹⁰ are each independently hydrogen, C₁-C₃-alkyl- or N-methylpiperidinyl-.

Very particular preference is given to compounds of the general formula (I) in which R⁹ is C₁-C₃-alkyl- or N-methylpiperidinyl-, and in which R¹⁰ is hydrogen.

Preference is given to compounds of the general formula (I) in which R⁹ and R¹⁰ together with the nitrogen atom to which they are bonded are 4-7-membered heterocycloalkyl- which may optionally be mono- or disubstituted identically or differently by: oxo, C₁-C₃-alkyl-, fluoro-C₁-C₃-alkyl-, cyclopropyl- or cyclopropylmethyl-.

Preference is further given to compounds of the general formula (I) in which R⁹ and R¹⁰ together with the nitrogen atom to which they are bonded are 4- to 7-membered heterocycloalkyl which may optionally be mono- or disubstituted identically or differently by: hydroxyl, fluorine, oxo, C₁-C₃-alkyl-, fluoro-C₁-C₃-alkyl-, cyclopropyl-, cyclopropylmethyl-, acetyl- or tert-butoxycarbonyl-.

Particular preference is given to compounds of the general formula (I) in which R⁹ and R¹⁰ together with the nitrogen atom to which they are bonded are 6-membered heterocycloalkyl- which may optionally be monosubstituted by methyl-, 2,2,2-trifluoroethyl- or cyclopropylmethyl-.

Particular preference is further given to compounds of the general formula (I) in which R⁹ and R¹⁰ together with the nitrogen atom to which they are bonded are 4- to 7-membered heterocycloalkyl which may optionally be mono- or disubstituted identically or differently by: fluorine, oxo, C₁-C₃-alkyl-, fluoro-C₁-C₃-alkyl-, cyclopropyl- or cyclopropylmethyl-.

Particular preference is given to compounds of the general formula (I) in which R⁹ and R¹⁰ together with the nitrogen atom to which they are bonded are N-cyclopropylmethylpiperazinyl-.

Very particular preference is given to compounds of the general formula (I) in which R⁹ and R¹⁰ together with the nitrogen atom to which they are bonded are 6-membered heterocycloalkyl which may optionally be mono- or disubstituted by fluorine, or which may optionally be monosubstituted by methyl-, 2,2,2-trifluoroethyl-, cyclopropyl- or cyclopropylmethyl-.

Preference is given to compounds of the general formula (I) in which R⁹ and R¹⁰ are each independently hydrogen or optionally mono-hydroxyl- or -oxo-substituted C₁-C₃-alkyl, or are trifluoromethyl-, or are 6-membered heterocycloalkyl-,
in which the 6-membered heterocycloalkyl- may itself optionally be mono- or disubstituted identically or differently by C₁-C₃-alkyl,
   or in which R⁹ and R¹⁰ together with the nitrogen atom to which they are bonded are 4- to 7-membered heterocycloalkyl- which may optionally be mono- or disubstituted identically or differently by: hydroxyl, fluorine, oxo, C₁-C₃-alkyl-, fluoro-C₁-C₃-alkyl-, cyclopropyl-, cyclopropylmethyl-, acetyl- or tert-butoxycarbonyl-.

Particular preference is given to compounds of the general formula (I) in which R⁹ and R¹⁰ are each independently hydrogen or optionally mono-hydroxyl- or -oxo-substituted C₁-C₃-alkyl, trifluoromethyl-, or N-methylpiperidinyl-,

or in which R⁹ and R¹⁰ together with the nitrogen atom to which they are bonded are 4- to 7-membered heterocycloalkyl- which may optionally be mono- or disubstituted identically or differently by: fluorine, oxo, C₁-C₃-alkyl-, fluoro-C₁-C₃-alkyl-, cyclopropyl- or cyclopropylmethyl-.

Very particular preference is given to compounds of the general formula (I) in which R⁹ and R¹⁰ are each independently hydrogen, C₁-C₃-alkyl- or

### N-methylpiperidinyl-,

or in which R⁹ and R¹⁰ together with the nitrogen atom to which they are bonded are 6-membered heterocycloalkyl- which may optionally be mono- or disubstituted by fluorine, or which may optionally be monosubstituted by methyl-, 2,2,2-trifluoroethyl-, cyclopropyl- or cyclopropylmethyl-.

Particular preference is given to compounds of the general formula (I) in which A is —NH— or —N(methyl)-, n is 0 or 1, R² is hydrogen, methyl- or methoxy-, R³ is methyl-, R⁴ is methyl- and R⁵ is hydrogen.

Particular preference is given to compounds of the general formula (I) in which A is —NH— or —N(methyl)-, X is —N—, n is 0 or 1, R² is hydrogen, methyl- or methoxy-, R³ is methyl-, R⁴ is methyl- and R⁵ is hydrogen.

Particular preference is given to compounds of the general formula (I) in which A is —NH— or —N(methyl)-, X is —CH—, n is 0 or 1, R² is hydrogen, methyl- or methoxy-, R³ is methyl-, R⁴ is methyl- and R⁵ is hydrogen.

The specific radical definitions given in the particular combinations or preferred combinations of radicals are, irrespective of the particular combinations of radicals specified, also replaced as desired by radical definitions of other combination.

Very particular preference is given to combinations of two or more of the abovementioned preferred ranges.

Very particular preference is given to the following compounds of the general formula (I): (3R)-4-cyclopentyl-1,3-dimethyl-6-({3-[(4-methylpiperazin-1-yl)carbonyl]phenyl}amino)-3,4-dihydropyrido[2,3-b]pyrazin-2(1H)-one;
3-{[(3R)-4-cyclopentyl-1,3-dimethyl-2-oxo-1,2,3,4-tetrahydropyrido[2,3-b]pyrazin-6-yl]amino}-N-(1-methylpiperidin-4-yl)benzamide;
3-{[(3R)-4-cyclopentyl-1,3-dimethyl-2-oxo-1,2,3,4-tetrahydropyrido[2,3-b]pyrazin-6-yl]amino}-N-(1-methylazetidin-3-yl)benzamide;
3-{[(3R)-4-cyclopentyl-1,3-dimethyl-2-oxo-1,2,3,4-tetrahydropyrido[2,3-b]pyrazin-6-yl]amino}-N-[2-(dimethylamino)ethyl]benzamide;
3-{[(3R)-4-cyclopentyl-1,3-dimethyl-2-oxo-1,2,3,4-tetrahydropyrido[2,3-b]pyrazin-6-yl]amino}-N-{trans-4-[4-(cyclopropylmethyl)piperazin-1-yl]cyclohexyl}benzamide;
3-{[(3R)-1,3-dimethyl-2-oxo-4-(tetrahydro-2H-pyran-4-yl)-1,2,3,4-tetrahydropyrido[2,3-b]pyrazin-6-yl]amino}-N,N-dimethylbenzenesulphonamide;
3-{[(3R)-1,3-dimethyl-2-oxo-4-(tetrahydro-2H-pyran-4-yl)-1,2,3,4-tetrahydroquinoxalin-6-yl]amino}-N,N-dimethylbenzenesulphonamide;
(3R)-1,3-dimethyl-6-{[3-(morpholin-4-ylsulphonyl)phenyl]amino}-4-(tetrahydro-2H-pyran-4-yl)-3,4-dihydroquinoxalin-2(1H)-one;
(3R)-1,3-dimethyl-6-{[3-(morpholin-4-ylsulphonyl)phenyl]amino}-4-(tetrahydro-2H-pyran-4-yl)-3,4-dihydropyrido[2,3-b]pyrazin-2(1H)-one;
3-{[(3R)-4-(4-methoxybenzyl)-1,3-dimethyl-2-oxo-1,2,3,4-tetrahydroquinoxalin-6-yl]amino}-N,N-dimethylbenzenesulphonamide;
(3R)-4-(4-methoxybenzyl)-1,3-dimethyl-6-{[3-(morpholin-4-ylsulphonyl)phenyl]amino}-3,4-dihydroquinoxalin-2(1H)-one;
(3R)-1,3-dimethyl-6-({3-[(4-methylpiperazin-1-yl)sulphonyl]phenyl}amino)-4-(tetrahydro-2H-pyran-4-yl)-3,4-dihydropyrido[2,3-b]pyrazin-2(1H)-one;
3-{[(3R)-1,3-dimethyl-2-oxo-4-(tetrahydro-2H-pyran-4-yl)-1,2,3,4-tetrahydropyrido[2,3-b]pyrazin-6-yl]amino}-N-(1-methylpiperidin-4-yl)benzamide;
N-{trans-4-[4-(cyclopropylmethyl)piperazin-1-yl]cyclohexyl}-3-{[(3R)-1,3-dimethyl-2-oxo-4-(tetrahydro-2H-pyran-4-yl)-1,2,3,4-tetrahydropyrido[2,3-b]pyrazin-6-yl]amino}benzamide;
(3R)-1,3-dimethyl-6-({3-[(4-methylpiperazin-1-yl)carbonyl]phenyl}amino)-4-(tetrahydro-2H-pyran-4-yl)-3,4-dihydropyrido[2,3-b]pyrazin-2(1H)-one;
3-{[(3R)-1,3-dimethyl-2-oxo-4-(tetrahydro-2H-pyran-4-yl)-1,2,3,4-tetrahydropyrido[2,3-b]pyrazin-6-yl]amino}-4-methoxy-N-(1-methylpiperidin-4-yl)benzamide;
(3R)-6-({2-methoxy-5-[(4-methylpiperazin-1-yl)carbonyl]phenyl}amino)-1,3-dimethyl-4-(tetrahydro-2H-pyran-4-yl)-3,4-dihydropyrido[2,3-b]pyrazin-2(1H)-one;
N-{trans-4-[4-(cyclopropylmethyl)piperazin-1-yl]cyclohexyl}-3-{[(3R)-1,3-dimethyl-2-oxo-4-(tetrahydro-2H-pyran-4-yl)-1,2,3,4-tetrahydropyrido[2,3-b]pyrazin-6-yl]amino}-4-methoxybenzamide;
N-[2-(dimethylamino)ethyl]-3-{[(3R)-1,3-dimethyl-2-oxo-4-(tetrahydro-2H-pyran-4-yl)-1,2,3,4-tetrahydropyrido[2,3-b]pyrazin-6-yl]amino}-4-methoxybenzamide;
3-{[(3R)-1,3-dimethyl-2-oxo-4-(tetrahydro-2H-pyran-4-yl)-1,2,3,4-tetrahydropyrido[2,3-b]pyrazin-6-yl]amino}-N-(1-methylpiperidin-4-yl)benzenesulphonamide;
N-[2-(dimethylamino)ethyl]-3-{[(3R)-1,3-dimethyl-2-oxo-4-(tetrahydro-2H-pyran-4-yl)-1,2,3,4-tetrahydropyrido[2,3-b]pyrazin-6-yl]amino}benzenesulphonamide;
N-{trans-4-[4-(cyclopropylmethyl)piperazin-1-yl]cyclohexyl}-3-{[4-(2,6-difluorobenzyl)-1,3-dimethyl-2-oxo-1,2,3,4-tetrahydroquinoxalin-6-yl]amino}benzamide;
3-{[(3R)-1,3-dimethyl-2-oxo-4-(tetrahydro-2H-pyran-4-yl)-1,2,3,4-tetrahydropyrido[2,3-b]pyrazin-6-yl]amino}-N-ethylbenzenesulphonamide;
(3R)-1,3-dimethyl-4-(1-methylpiperidin-4-yl)-6-{[3-(pyrrolidin-1-ylsulphonyl)phenyl]amino}-3,4-dihydropyrido[2,3-b]pyrazin-2(1H)-one;
(3R)-6-{[2-methoxy-5-(morpholin-4-ylsulphonyl)phenyl]amino}-1,3-dimethyl-4-(1-methylpiperidin-4-yl)-3,4-dihydropyrido[2,3-b]pyrazin-2(1H)-one;
N-cyclopropyl-3-{[(3R)-1,3-dimethyl-2-oxo-4-(tetrahydro-2H-pyran-4-yl)-1,2,3,4-tetrahydropyrido[2,3-b]pyrazin-6-yl]amino}benzenesulphonamide;
(3R)-1,3-dimethyl-6-{[3-(pyrrolidin-1-ylsulphonyl)phenyl]amino}-4-(tetrahydro-2H-pyran-4-yl)-3,4-dihydropyrido[2,3-b]pyrazin-2(1H)-one;
(3R)-6-{[2-methoxy-5-(morpholin-4-ylsulphonyl)phenyl]amino}-1,3-dimethyl-4-(tetrahydro-2H-pyran-4-yl)-3,4-dihydropyrido[2,3-b]pyrazin-2(1H)-one;
(3R)-6-({3-[(3,3-difluoroazetidin-1-yl)sulphonyl]phenyl}amino)-1,3-dimethyl-4-(1-methylpiperidin-4-yl)-3,4-dihydropyrido[2,3-b]pyrazin-2(1H)-one;
(3R)-6-{[2-methoxy-5-(2-oxa-6-azaspiro[3.3]hept-6-ylsulphonyl)phenyl]amino}-1,3-dimethyl-4-(tetrahydro-2H-pyran-4-yl)-3,4-dihydropyrido[2,3-b]pyrazin-2(1H)-one;
(3R)-6-({3-[(3,3-difluoroazetidin-1-yl)sulphonyl]phenyl}amino)-1,3-dimethyl-4-(tetrahydro-2H-pyran-4-yl)-3,4-dihydropyrido[2,3-b]pyrazin-2(1H)-one;
3-{[(3R)-1,3-dimethyl-2-oxo-4-(tetrahydro-2H-pyran-4-yl)-1,2,3,4-tetrahydropyrido[2,3-b]pyrazin-6-yl]amino}-N,N-diethylbenzenesulphonamide;
3-{[(3R)-4-benzyl-1,3-dimethyl-2-oxo-1,2,3,4-tetrahydropyrido[2,3-b]pyrazin-6-yl]amino}-N-(1-methylpiperidin-4-yl)benzenesulphonamide;
N-{trans-4-[4-(cyclopropylmethyl)piperazin-1-yl]cyclohexyl}-3-{[(3R)-4-isopropyl-1,3-dimethyl-2-oxo-1,2,3,4-tetrahydropyrido[2,3-b]pyrazin-6-yl]amino}benzenesulphonamide;
N-{trans-4-[4-(cyclopropylmethyl)piperazin-1-yl]cyclohexyl}-3-{[(3R)-1,3-dimethyl-2-oxo-4-(tetrahydro-2H-pyran-4-yl)-1,2,3,4-tetrahydropyrido[2,3-b]pyrazin-6-yl]amino}benzene sulphonamide;
N-{trans-4-[4-(cyclopropylmethyl)piperazin-1-yl]cyclohexyl}-3-{[(3S)-1,3-dimethyl-2-oxo-4-(tetrahydro-2H-pyran-4-yl)-1,2,3,4-tetrahydropyrido[2,3-b]pyrazin-6-yl]amino}benzenesulphonamide;
(3R)-1,3-dimethyl-4-(tetrahydro-2H-pyran-4-yl)-6-[(3-{[4-(2,2,2-trifluoroethyl)piperazin-1-yl]sulphonyl}phenyl)amino]-3,4-dihydropyrido[2,3-b]pyrazin-2(1H)-one;
3-{[(3R)-4-benzyl-1,3-dimethyl-2-oxo-1,2,3,4-tetrahydropyrido[2,3-b]pyrazin-6-yl]amino}-N-{trans-4-[4-(cyclopropylmethyl)piperazin-1-yl]cyclohexyl}benzenesulphonamide;
3-{[(3R)-1,3-dimethyl-2-oxo-4-(tetrahydro-2H-pyran-4-yl)-1,2,3,4-tetrahydropyrido[2,3-b]pyrazin-6-yl]amino}-N-methylbenzenesulphonamide;
(3R)-6-({2-methoxy-5-[(4-methylpiperazin-1-yl)sulphonyl]phenyl}amino)-1,3-dimethyl-4-(tetrahydro-2H-pyran-4-yl)-3,4-dihydropyrido[2,3-b]pyrazin-2(1H)-one;
3-{[(3R)-1,3-dimethyl-4-(1-methylpiperidin-4-yl)-2-oxo-1,2,3,4-tetrahydropyrido[2,3-b]pyrazin-6-yl]amino}-N,N-dimethylbenzenesulphonamide;
3-{[(3R)-1,3-dimethyl-2-oxo-4-(tetrahydro-2H-pyran-4-yl)-1,2,3,4-tetrahydropyrido[2,3-b]pyrazin-6-yl]amino}-N-isopropylbenzenesulphonamide;
(3R)-4-isopropyl-1,3-dimethyl-6-({3-[(4-methylpiperazin-1-yl)sulphonyl]phenyl}amino)-3,4-dihydropyrido[2,3-b]pyrazin-2(1H)-one;
3-{[(3R)-1,3-dimethyl-2-oxo-4-(tetrahydro-2H-pyran-4-yl)-1,2,3,4-tetrahydropyrido[2,3-b]pyrazin-6-yl]amino}-N-[(1-methylpiperidin-4-yl)methyl]benzenesulphonamide;
N-{trans-4-[4-(cyclopropylmethyl)piperazin-1-yl]cyclohexyl}-3-{[(3R)-4-isobutyl-1,3-dimethyl-2-oxo-1,2,3,4-tetrahydropyrido[2,3-b]pyrazin-6-yl]amino}benzenesulphonamide;
(3R)-6-({2-methoxy-5-[(4-methylpiperazin-1-yl)sulphonyl]phenyl}amino)-1,3-dimethyl-4-(1-methylpiperidin-4-yl)-3,4-dihydropyrido[2,3-b]pyrazin-2(1H)-one;
(3R)-1,3-dimethyl-4-(1-methylpiperidin-4-yl)-6-{[3-(morpholin-4-ylsulphonyl)phenyl]amino}-3,4-dihydropyrido[2,3-b]pyrazin-2(1H)-one;
3-{[(3R)-4-cycloheptyl-1,3-dimethyl-2-oxo-1,2,3,4-tetrahydropyrido[2,3-b]pyrazin-6-yl]amino}-N-(1-methylpiperidin-4-yl)benzenesulphonamide;
4-(2-methoxyethyl)-1,3-dimethyl-6-{[3-(morpholin-4-ylsulphonyl) phenyl]amino}-3,4-dihydropyrido[2,3-b]pyrazin-2(1H)-one;
(3R)-1,3-dimethyl-6-({3-[(4-methylpiperazin-1-yl)sulphonyl]phenyl}amino)-4-(1-methylpiperidin-4-yl)-3,4-dihydropyrido[2,3-b]pyrazin-2(1H)-one;
3-{[(3R)-4-isopropyl-1,3-dimethyl-2-oxo-1,2,3,4-tetrahydropyrido[2,3-b]pyrazin-6-yl]amino}-N-(1-methylpiperidin-4-yl)benzenesulphonamide;
tert-butyl 4-[(3R)-6-{[3-({trans-4-[4-(cyclopropylmethyl)piperazin-1-yl]cyclohexyl}sulphamoyl)phenyl]amino}-1,3-dimethyl-2-oxo-2,3-dihydropyrido[2,3-b]pyrazin-4(1H)-yl]piperidine-1-carboxylate;
4-(2-methoxyethyl)-1,3-dimethyl-6-({3-[(4-methylpiperazin-1-yl)sulphonyl]phenyl}amino)-3,4-dihydropyrido[2,3-b]pyrazin-2(1H)-one;
3-{[(3R)-1,3-dimethyl-2-oxo-4-(tetrahydro-2H-pyran-4-yl)-1,2,3,4-tetrahydropyrido[2,3-b]pyrazin-6-yl](methyl)amino}-N,N-dimethylbenzenesulphonamide;
N-[2-(dimethylamino)ethyl]-3-{[(3R)-4-isopropyl-1,3-dimethyl-2-oxo-1,2,3,4-tetrahydropyrido[2,3-b]pyrazin-6-yl]amino}benzenesulphonamide;
(3R)-6-[(1,1-dioxido-2,3-dihydro-1,2-benzothiazol-6-yl)amino]-1,3-dimethyl-4-(tetrahydro-2H-pyran-4-yl)-3,4-dihydropyrido[2,3-b]pyrazin-2(1H)-one;
tert-butyl 4-[(3R)-1,3-dimethyl-6-({3-[(1-methylpiperidin-4-yl)sulphamoyl]phenyl}amino)-2-oxo-2,3-dihydro-pyrido[2,3-b]pyrazin-4(1H)-yl]piperidine-1-carboxylate;
3-{[(3R)-1,3-dimethyl-2-oxo-4-(tetrahydro-2H-pyran-4-yl)-1,2,3,4-tetrahydropyrido[2,3-b]pyrazin-6-yl]amino}benzenesulphonamide;
tert-butyl 4-[(3R)-6-[(3-{[2-(dimethylamino)ethyl]sulphamoyl}phenyl)amino]-1,3-dimethyl-2-oxo-2,3-dihydropyrido[2,3-b]pyrazin-4(1H)-yl]piperidine-1-carboxylate;
tert-butyl 4-[(3R)-6-{[3-(2-azaspiro[3.3]hept-2-ylsulphonyl)phenyl]amino}-1,3-dimethyl-2-oxo-2,3-dihydropyrido[2,3-b]pyrazin-4(1H)-yl]piperidine-1-carboxylate;
tert-butyl 4-[(3R)-6-{[3-(dimethylsulphamoyl)phenyl]amino}-1,3-dimethyl-2-oxo-2,3-dihydropyrido[2,3-b]pyrazin-4(1H)-yl]piperidine-1-carboxylate;
tert-butyl 4-[(3R)-1,3-dimethyl-6-({3-[(4-methylpiperazin-1-yl)sulphonyl]phenyl}amino)-2-oxo-2,3-dihydropyrido[2,3-b]pyrazin-4(1H)-yl]piperidine-1-carboxylate;
5-{[(3R)-1,3-dimethyl-2-oxo-4-(tetrahydro-2H-pyran-4-yl)-1,2,3,4-tetrahydropyrido[2,3-b]pyrazin-6-yl]amino}-2-methylbenzenesulphonamide;
1,3-dimethyl-4-(3-methylphenyl)-6-{[3-(morpholin-4-ylsulphonyl)phenyl]amino}-3,4-dihydropyrido[2,3-b]pyrazin-2(1H)-one;
3-{[1,3-dimethyl-4-(3-methylphenyl)-2-oxo-1,2,3,4-tetrahydropyrido[2,3-b]pyrazin-6-yl]amino}-N-(1-methylpiperidin-4-yl)benzenesulphonamide;
tert-butyl 4-[(3R)-1,3-dimethyl-2-oxo-6-[(3-{[4-(2,2,2-trifluoroethyl)piperazin-1-yl]sulphonyl}phenyl)amino]-2,3-dihydropyrido[2,3-b]pyrazin-4(1H)-yl]piperidine-1-carboxylate;
tert-butyl 4-{4-[1,3-dimethyl-6-({3-[(4-methylpiperazin-1-yl)sulphonyl]phenyl}amino)-2-oxo-2,3-dihydropyrido[2,3-b]pyrazin-4(1H)-yl]phenyl}piperazien-1-carboxylate;
tert-butyl 4-[(2R)-7-{[3-(dimethylsulphamoyl)phenyl]amino}-2,4-dimethyl-3-oxo-3,4-dihydroquinoxalin-1(2H)-yl]piperidine-1-carboxylate;
3-{[(3R)-4-benzyl-1,3-dimethyl-2-oxo-1,2,3,4-tetrahydroquinoxalin-6-yl]amino}-N,N-dimethylbenzenesulphonamide;
tert-butyl 4-[(2R)-7-{[3-(dimethylsulphamoyl)phenyl](methyl)amino}-2,4-dimethyl-3-oxo-3,4-dihydroquinoxalin-1(2H)-yl]piperidine-1-carboxylate;
(3R)-1,3-dimethyl-6-({3-[(4-methylpiperazin-1-yl)sulphonyl]phenyl}amino)-4-(tetrahydro-2H-pyran-4-yl)-3,4-dihydroquinoxalin-2(1H)-one;
3-{[(3R)-4-benzyl-1,3-dimethyl-2-oxo-1,2,3,4-tetrahydropyrido[2,3-b]pyrazin-6-yl]amino}-N-{trans-4-[4-(cyclopropylmethyl)piperazin-1-yl]cyclohexyl}benzamide;
3-{[(3R)-4-benzyl-1,3-dimethyl-2-oxo-1,2,3,4-tetrahydropyrido[2,3-b]pyrazin-6-yl]amino}-N-(1-methylpiperidin-4-yl)benzamide;
3-{[(3R)-4-benzyl-1,3-dimethyl-2-oxo-1,2,3,4-tetrahydropyrido[2,3-b]pyrazin-6-yl]amino}-N-[2-(4-methylpiperazin-1-yl)ethyl]benzamide;
(3R)-4-benzyl-1,3-dimethyl-6-({3-[(4-methylpiperazin-1-yl)carbonyl]phenyl}amino)-3,4-dihydropyrido[2,3-b]pyrazin-2(1H)-one;
(3R)-4-benzyl-6-({3-[(4-isopropylpiperazin-1-yl)carbonyl]phenyl}amino)-1,3-dimethyl-3,4-dihydropyrido[2,3-b]pyrazin-2(1H)-one;
(3R)-4-isopropyl-1,3-dimethyl-6-({3-[(4-methylpiperazin-1-yl)carbonyl]phenyl}amino)-3,4-dihydropyrido[2,3-b]pyrazin-2(1H)-one;
tert-butyl 4-[(3R)-1,3-dimethyl-6-({3-[(4-methylpiperazin-1-yl)carbonyl]phenyl}amino)-2-oxo-2,3-dihydropyrido[2,3-b]pyrazin-4(1H)-yl]piperidine-1-carboxylate;
N-{trans-4-[4-(cyclopropylmethyl)piperazin-1-yl]cyclohexyl}-3-{[(3R)-4-isopropyl-1,3-dimethyl-2-oxo-1,2,3,4-tetrahydropyrido[2,3-b]pyrazin-6-yl]amino}benzamide;
3-{[(3R)-1,3-dimethyl-2-oxo-4-(tetrahydro-2H-pyran-4-yl)-1,2,3,4-tetrahydropyrido[2,3-b]pyrazin-6-yl]amino}-N-{4-[(1-methylpiperidin-4-yl)amino]cyclohexyl}benzamide;
5-{[(3R)-1,3-dimethyl-2-oxo-4-(tetrahydro-2H-pyran-4-yl)-1,2,3,4-tetrahydropyrido[2,3-b]pyrazin-6-yl]amino}-2-methoxy-N-(1-methylpiperidin-4-yl)benzamide;
N-{trans-4-[4-(cyclopropylmethyl)piperazin-1-yl]cyclohexyl}-5-{[(3R)-1,3-dimethyl-2-oxo-4-(tetrahydro-2H-pyran-4-yl)-1,2,3,4-tetrahydropyrido[2,3-b]pyrazin-6-yl]amino}-2-methoxybenzamide;
(3R)-6-({4-methoxy-3-[(4-methylpiperazin-1-yl)carbonyl]phenyl}amino)-1,3-dimethyl-4-(tetrahydro-2H-pyran-4-yl)-3,4-dihydropyrido[2,3-b]pyrazin-2(1H)-one;
3-{[(3R)-4-cyclohexyl-1,3-dimethyl-2-oxo-1,2,3,4-tetrahydropyrido[2,3-b]pyrazin-6-yl]amino}-N-[2-(4-methylpiperazin-1-yl)ethyl]benzamide;
(3R)-4-cyclohexyl-6-({3-[(4-isopropylpiperazin-1-yl)carbonyl]phenyl}amino)-1,3-dimethyl-3,4-dihydropyrido[2,3-b]pyrazin-2(1H)-one;
3-{[(3R)-4-cyclohexyl-1,3-dimethyl-2-oxo-1,2,3,4-tetrahydropyrido[2,3-b]pyrazin-6-yl]amino}-N-(4-hydroxycyclohexyl)benzamide;
N-{trans-4-[4-(cyclopropylmethyl)piperazin-1-yl]cyclohexyl}-3-{[(3R)-1,3-dimethyl-2-oxo-4-(tetrahydro-2H-pyran-4-yl)-1,2,3,4-tetrahydropyrido[2,3-b]pyrazin-6-yl]amino}-4-methoxybenzamide;
(3R)-6-[(3-{[4-(cyclopropylmethyl)piperazin-1-yl]carbonyl}phenyl) amino]-4-isopropyl-1,3-dimethyl-3,4-dihydropyrido[2,3-b]pyrazin-2(1H)-one;
N-[4-(4,4-difluoropiperidin-1-yl)cyclohexyl]-5-{[(3R)-1,3-dimethyl-2-oxo-4-(tetrahydro-2H-pyran-4-yl)-1,2,3,4-tetrahydropyrido[2,3-b]pyrazin-6-yl]amino}-2-methoxybenzamide;
N-[cis-4-(4-cyclopropylpiperazin-1-yl)cyclohexyl]-3-{[(3R)-1,3-dimethyl-2-oxo-4-(tetrahydro-2H-pyran-4-yl)-1,2,3,4-tetrahydropyrido[2,3-b]pyrazin-6-yl]amino}-4-methoxybenzamide;
N-{trans-4-[4-(cyclopropylmethyl)piperazin-1-yl]cyclohexyl}-3-{[(3R)-1,3-dimethyl-2-oxo-4-(tetrahydro-2H-pyran-4-yl)-1,2,3,4-tetrahydropyrido[2,3-b]pyrazin-6-yl]amino}-2-methoxybenzamide;
(3R)-6-({2-methoxy-3-[(4-methylpiperazin-1-yl)carbonyl]phenyl}amino)-1,3-dimethyl-4-(tetrahydro-2H-pyran-4-yl)-3,4-dihydropyrido[2,3-b]pyrazin-2(1H)-one;
N-{trans-4-[4-(cyclopropylmethyl)piperazin-1-yl]cyclohexyl}-3-{[(3R)-4-(4-fluorophenyl)-1,3-dimethyl-2-oxo-1,2,3,4-tetrahydropyrido[2,3-b]pyrazin-6-yl]amino}benzamide;
3-{[(3R)-4-isopropyl-1,3-dimethyl-2-oxo-1,2,3,4-tetrahydropyrido[2,3-b]pyrazin-6-yl]amino}-N-(1-methylpiperidin-4-yl)benzamide;
3-{[(3R)-4-cyclohexyl-1,3-dimethyl-2-oxo-1,2,3,4-tetrahydropyrido[2,3-b]pyrazin-6-yl]amino}-N-(1-methylpiperidin-4-yl)benzamide;
3-{[(3R)-4-(4,4-dimethylcyclohexyl)-1,3-dimethyl-2-oxo-1,2,3,4-tetrahydropyrido[2,3-b]pyrazin-6-yl]amino}-N-(1-methylpiperidin-4-yl)benzamide;
N-[2-(dimethylamino)ethyl]-3-{[(3R)-4-isopropyl-1,3-dimethyl-2-oxo-1,2,3,4-tetrahydropyrido[2,3-b]pyrazin-6-yl]amino}benzamide;
3-{[4-(2-methoxyethyl)-1,3-dimethyl-2-oxo-1,2,3,4-tetrahydropyrido[2,3-b]pyrazin-6-yl]amino}-N-(1-methylpiperidin-4-yl)benzamide;
3-{[(3R)-4-cyclohexyl-1,3-dimethyl-2-oxo-1,2,3,4-tetrahydropyrido[2,3-b]pyrazin-6-yl]amino}-N-{trans-4-[4-(cyclopropylmethyl)piperazin-1-yl]cyclohexyl}benzamide;
tert-butyl 4-[(3R)-1,3-dimethyl-6-({3-[(1-methylpiperidin-4-yl)carbamoyl]phenyl}amino)-2-oxo-2,3-dihydropyrido[2,3-b]pyrazin-4(1H)-yl]piperidine-1-carboxylate;
N-{trans-4-[4-(cyclopropylmethyl)piperazin-1-yl]cyclohexyl}-3-{[(3R)-4-(2-methoxyethyl)-1,3-dimethyl-2-oxo-1,2,3,4-tetrahydropyrido[2,3-b]pyrazin-6-yl]amino}benzamide;
tert-butyl 4-[(3R)-6-{[3-({trans-4-[4-(cyclopropylmethyl)piperazin-1-yl]cyclohexyl}carbamoyl)phenyl]amino}-1,3-dimethyl-2-oxo-2,3-dihydropyrido[2,3-b]pyrazin-4(1H)-yl]piperidine-1-carboxylate;
3-{[(3R)-1,3-dimethyl-2-oxo-4-(tetrahydro-2H-pyran-4-yl)-1,2,3,4-tetrahydropyrido[2,3-b]pyrazin-6-yl]amino}-2-methoxy-N-(1-methylpiperidin-4-yl)benzamide;
3-[(1,3-dimethyl-2-oxo-4-phenyl-1,2,3,4-tetrahydropyrido[2,3-b]pyrazin-6-yl)amino]-N-(1-methylpiperidin-4-yl)benzamide;
tert-butyl 4-[(2R)-7-{[3-(dimethylcarbamoyl)phenyl]amino}-2,4-dimethyl-3-oxo-3,4-dihydroquinoxalin-1(2H)-yl]piperidine-1-carboxylate;
(3R)-1,3-dimethyl-6-({3-[(4-methylpiperazin-1-yl)carbonyl]phenyl}amino)-4-(tetrahydro-2H-pyran-4-yl)-3,4-dihydroquinoxalin-2(1H)-one;
3-{[(3R)-4-benzyl-1,3-dimethyl-2-oxo-1,2,3,4-tetrahydroquinoxalin-6-yl]amino}-N,N-dimethylbenzamide;
N-{trans-4-[4-(cyclopropylmethyl)piperazin-1-yl]cyclohexyl}-3-{[(3R)-1,3-dimethyl-2-oxo-4-(tetrahydro-2H-pyran-4-yl)-1,2,3,4-tetrahydroquinoxalin-6-yl]amino}benzamide;
3-{[(3R)-1,3-dimethyl-2-oxo-4-(tetrahydro-2H-pyran-4-yl)-1,2,3,4-tetrahydroquinoxalin-6-yl]amino}-N-(1-methylpiperidin-4-yl)benzamide;
3-[(4-benzyl-1,3-dimethyl-2-oxo-1,2,3,4-tetrahydroquinoxalin-6-yl)(methyl)amino]-N,N-dimethylbenzenesulphonamide;
(3R)-4-(2-methoxyethyl)-1,3-dimethyl-6-{[3-(morpholin-4-ylsulphonyl) phenyl]amino}-3,4-dihydropyrido[2,3-b]pyrazin-2(1H)-one;
(3S)-4-(2-methoxyethyl)-1,3-dimethyl-6-{[3-(morpholin-4-ylsulphonyl) phenyl]amino}-3,4-dihydropyrido[2,3-b]pyrazin-2(1H)-one;
(3R)-4-(2-methoxyethyl)-1,3-dimethyl-6-({3-[(4-methylpiperazin-1-yl)sulphonyl]phenyl}amino)-3,4-dihydropyrido[2,3-b]pyrazin-2(1H)-one;
(3R)-4-(4-fluorophenyl)-1,3-dimethyl-6-{[3-(morpholin-4-ylsulphonyl)phenyl]amino}-3,4-dihydropyrido[2,3-b]pyrazin-2(1H)-one;
3-{[(3R)-4-(4-fluorophenyl)-1,3-dimethyl-2-oxo-1,2,3,4-tetrahydropyrido[2,3-b]pyrazin-6-yl]amino}-N-(1-methylpiperidin-4-yl)benzamide;
3-{[(3S)-4-(4-fluorophenyl)-1,3-dimethyl-2-oxo-1,2,3,4-tetrahydropyrido[2,3-b]pyrazin-6-yl]amino}-N-(1-methylpiperidin-4-yl)benzamide;
(3R)-4-(2-methoxyethyl)-1,3-dimethyl-6-({3-[(4-methylpiperazin-1-yl)carbonyl]phenyl}amino)-3,4-dihydropyrido[2,3-b]pyrazin-2(1H)-one;
(3S)-4-(2-methoxyethyl)-1,3-dimethyl-6-({3-[(4-methylpiperazin-1-yl)carbonyl]phenyl}amino)-3,4-dihydropyrido[2,3-b]pyrazin-2(1H)-one;
3-{[(3R)-4-(2-methoxyethyl)-1,3-dimethyl-2-oxo-1,2,3,4-tetrahydropyrido[2,3-b]pyrazin-6-yl]amino}-N-(1-methylpiperidin-4-yl)benzamide;
3-{[(3S)-4-(2-methoxyethyl)-1,3-dimethyl-2-oxo-1,2,3,4-tetrahydropyrido[2,3-b]pyrazin-6-yl]amino}-N-(1-methylpiperidin-4-yl)benzamide;
3-{[(3R)-1,3-dimethyl-2-oxo-4-phenyl-1,2,3,4-tetrahydropyrido[2,3-b]pyrazin-6-yl]amino}-N-(1-methylpiperidin-4-yl)benzamide;
(3R)-6-{[3-(2-azaspiro[3.3]hept-2-ylsulphonyl)phenyl]amino}-1,3-dimethyl-4-(piperidin-4-yl)-3,4-dihydropyrido[2,3-b]pyrazin-2(1H)-one;
3-{[(3R)-1,3-dimethyl-2-oxo-4-(piperidin-4-yl)-1,2,3,4-tetrahydropyrido[2,3-b]pyrazin-6-yl]amino}-N,N-dimethylbenzenesulphonamide;
3-{[(3R)-1,3-dimethyl-2-oxo-4-(piperidin-4-yl)-1,2,3,4-tetrahydropyrido[2,3-b]pyrazin-6-yl]amino}-N-(1-methylpiperidin-4-yl)benzenesulphonamide;
(3R)-1,3-dimethyl-6-({3-[(4-methylpiperazin-1-yl)sulphonyl]phenyl}amino)-4-(piperidin-4-yl)-3,4-dihydropyrido[2,3-b]pyrazin-2(1H)-one;
(3R)-1,3-dimethyl-4-(piperidin-4-yl)-6-[(3-{[4-(2,2,2-trifluoroethyl)piperazin-1-yl]sulphonyl}phenyl)amino]-3,4-dihydropyrido[2,3-b]pyrazin-2(1H)-one;
N-{trans-4-[4-(cyclopropylmethyl)piperazin-1-yl]cyclohexyl}-3-{[(3R)-1,3-dimethyl-2-oxo-4-(piperidin-4-yl)-1,2,3,4-tetrahydropyrido[2,3-b]pyrazin-6-yl]amino}benzenesulphonamide;
N-[2-(dimethylamino)ethyl]-3-{[(3R)-1,3-dimethyl-2-oxo-4-(piperidin-4-yl)-1,2,3,4-tetrahydropyrido[2,3-b]pyrazin-6-yl]amino}benzenesulphonamide;
3-{[(3R)-1,3-dimethyl-2-oxo-4-(piperidin-4-yl)-1,2,3,4-tetrahydroquinoxalin-6-yl]amino}-N,N-dimethylbenzenesulphonamide;
(3R)-1,3-dimethyl-6-({3-[(4-methylpiperazin-1-yl)carbonyl]phenyl}amino)-4-(piperidin-4-yl)-3,4-dihydropyrido[2,3-b]pyrazin-2(1H)-one;
N-{trans-4-[4-(cyclopropylmethyl)piperazin-1-yl]cyclohexyl}-3-{[(3R)-1,3-dimethyl-2-oxo-4-(piperidin-4-yl)-1,2,3,4-tetrahydropyrido[2,3-b]pyrazin-6-yl]amino}benzamide;
3-{[(3R)-1,3-dimethyl-2-oxo-4-(piperidin-2-yl)-1,2,3,4-tetrahydropyrido[2,3-b]pyrazin-6-yl]amino}-N-(1-methylpiperidin-4-yl)benzamide;
3-{[(3R)-1,3-dimethyl-2-oxo-4-(piperidin-4-yl)-1,2,3,4-tetrahydroquinoxalin-6-yl]amino}-N,N-dimethylbenzamide;
(3R)-1,3-dimethyl-6-{[3-(5-methyl-1,3,4-oxadiazol-2-yl)phenyl]amino}-4-(tetrahydro-2H-pyran-4-yl)-3,4-dihydropyrido[2,3-b]pyrazin-2(1H)-one;
4-(4-fluorophenyl)-1,3-dimethyl-6-{[3-(morpholin-4-ylsulphonyl) phenyl]amino}-3,4-dihydropyrido[2,3-b]pyrazin-2(1H)-one;
3-{[4-(4-fluorophenyl)-1,3-dimethyl-2-oxo-1,2,3,4-tetrahydropyrido[2,3-b]pyrazin-6-yl]amino}-N-(1-methylpiperidin-4-yl)benzamide
   and
4-(2-methoxyethyl)-1,3-dimethyl-6-({3-[(4-methylpiperazin-1-yl)carbonyl]phenyl}amino)-3,4-dihydropyrido[2,3-b]pyrazin-2(1H)-one.

### DEFINITIONS

C₁-C₆-Alkyl-, or a C₁-C₆-alkyl group, is understood to mean a linear or branched, saturated monovalent hydrocarbyl radical, for example a methyl, ethyl, propyl, butyl, pentyl, hexyl, iso-propyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl or 1,2-dimethylbutyl radical. Preferably, C₁-C₆-alkyl-, or a C₁-C₆-alkyl group, is understood to mean C₁-C₄-alkyl- or C₂-C₅-alkyl-, more preferably C₁-C₃-alkyl-, i.e. a methyl, ethyl, propyl or isopropyl radical.

C₂-C₆-Alkenyl-, or a C₂-C₆-alkenyl group, is understood to mean a straight-chain or branched, monovalent hydrocarbon radical having one or two C═C double bonds, for example an ethenyl, (E)-prop-2-enyl, (Z)-prop-2-enyl, allyl (prop-1-enyl), allenyl, buten-1-yl or buta-1,3-dienyl radical. Preference is given to C₃-C₆-alkenyl- and C₂-C₄-alkenyl-; particular preference is given to ethenyl- and allyl-.

C₂-C₆-Alkynyl, or a C₂-C₆-alkynyl group, is understood to mean a straight-chain or branched, monovalent hydrocarbon radical having one C≡C triple bond, for example an ethynyl, propargyl (prop-1-ynyl) or butyn-1-yl radical. Preference is given to C₃-C₆-alkynyl- and C₂-C₄-alkynyl-; particular preference is given to ethynyl and propargyl.

C₁-C₄-Alkoxy-, or a C₁-C₄-alkoxy group, is understood to mean a linear or branched, saturated alkyl ether radical —O-alkyl, for example a methoxy, ethoxy, n-propoxy, isopropoxy or tert-butoxy radical.

Preferably, C₁-C₄-alkoxy-, or a C₁-C₄-alkoxy group, is understood to mean C₁-C₃-alkoxy-, more preferably a methoxy or ethoxy radical.

C₁-C₄-Alkylthio-, or a C₁-C₄-alkylthio group, is understood to mean a linear or branched, saturated alkyl thioether radical —S-alkyl, for example a methylthio, ethylthio, n-propylthio, isopropylthio or tert-butylthio radical.

Preferably, C₁-C₄-alkylthio-, or a C₁-C₄-alkylthio group, is understood to mean C₁-C₃-alkylthio-, more preferably a methylthio or ethylthio radical.

A heteroatom is understood to mean —O—, NH—, ═N— or —S—. The heteroatom —NH— may optionally be substituted by C₁-C₃-alkyl, C₁-C₃-alkylcarbonyl, C₁-C₄-alkoxycarbonyl, or —S(═O)₂—C₁-C₃-alkyl. Preference is given to an oxygen or nitrogen atom.

Oxo, or an oxo substituent, is understood to mean a double-bonded oxygen atom ═O. Oxo may be bonded to atoms of suitable valency, for example to a saturated carbon atom or to sulphur. Preference is given to the bond to carbon to form a carbonyl group —(C═O)—. Preference is further given to the bond of two double-bonded oxygen atoms to sulphur, forming a sulphonyl group —(S═O)₂—.

Halogen is understood to mean fluorine, chlorine, bromine or iodine.

A halo-C₁-C₄-alkyl radical, or halo-C₁-C₄-alkyl-, is understood to mean a C₁-C₄-alkyl radical substituted by at least one halogen substituent, preferably by at least one fluorine substituent. Preference is given to fluoro-C₁-C₃-alkyl radicals, for example difluoromethyl-, trifluoromethyl-, 2,2,2-trifluoroethyl- or pentafluoroethyl-.

Particular preference is given to perfluorinated alkyl radicals such as trifluoromethyl- or pentafluoroethyl-.

Phenyl-C₁-C₃-alkyl- is understood to mean a group composed of an optionally substituted phenyl radical and a C₁-C₃-alkyl group, and bonded to the rest of the molecule via the C₁-C₃-alkyl group.

A halo-C₁-C₄-alkoxy radical, or halo-C₁-C₄-alkoxy-, is understood to mean a C₁-C₄-alkoxy radical substituted by at least one halogen substituent, preferably by at least one fluorine substituent. Preference is given to fluoro-C₁-C₃-alkoxy radicals, for example difluoromethoxy-, trifluoromethoxy- or 2,2,2-trifluoroethoxy-.

A halo-C₁-C₄-alkylthio radical, or halo-C₁-C₄-alkylthio-, is understood to mean a C₁-C₄-alkylthio radical substituted by at least one halogen substituent, preferably by at least one fluorine substituent. Preference is given to fluoro-C₁-C₃-alkylthio radicals, especially trifluoromethylthio-.

A C₁-C₄-alkylcarbonyl radical is understood to mean a C₁-C₄-alkyl-C(═O)— group. Preference is given to C₁-C₃-alkylcarbonyl-, particular preference to acetyl- or propanoyl-.

A C₁-C₄-alkoxycarbonyl radical is understood to mean a C₁-C₄-alkoxy-C(═O)— group. Preference is given to methoxycarbonyl-, ethoxycarbonyl- or tert-butoxycarbonyl-.

A C₁-C₄-alkoxy-C₁-C₄-alkyl radical is understood to mean a C₁-C₄-alkoxy-substituted C₁-C₄-alkyl radical, for example methoxymethyl-, methoxyethyl-, ethoxymethyl- and ethoxyethyl-.

Aryl is understood to mean an unsaturated, fully conjugated system which is formed from carbon atoms and has 3, 5 or 7 conjugated double bonds, for example phenyl-, naphthyl- or phenanthryl-. Preference is given to phenyl.

Heteroaryl- is understood to mean ring systems which have an aromatically conjugated ring system and contain at least one and up to five heteroatoms as defined above, contain. These ring systems may have 5, 6 or 7 ring atoms, or else, in the case of fused or benzofused ring systems, combinations of 5- and 6-membered ring systems, 5- and 5-membered ring systems, or else 6- and 6-membered ring systems. Examples include ring systems such as pyrrolyl-, pyrazolyl-, imidazolyl-triazolyl-, tetrazolyl-, furanyl-, thienyl-, oxazolyl-, thiazolyl-, isoxazolyl-, oxadiazolyl-, thiadiazolyl-, pyridinyl-, pyrimidinyl-, pyrazinyl-, triazinyl-,

oxazinyl-, indolyl-, benzimidazolyl-, indazolyl-, benzotriazolyl-, benzothiazolyl-, benzoxazolyl-, benzofuranyl-, benzothienyl-, quinolinyl-, isoquinolinyl-, cinnolinyl-, quinazolinyl-, quinoxalinyl-, imidazopyridinyl- or else benzoxazinyl-.

Preference is given to 5- to 6-membered monocyclic heteroaryl-, for example pyrrolyl-, pyrazolyl-, imidazolyl-, triazolyl-, tetrazolyl-, furanyl-, thienyl-, oxazolyl-, thiazolyl-, isoxazolyl-, oxadiazolyl-, thiadiazolyl-, pyridinyl-, pyrimidinyl-, pyrazinyl-, triazinyl-.

C₃-C₆-Cycloalkyl, C₃-C₈-cycloalkyl, and C₅-C₈-cycloalkyl are understood to mean a monocyclic, saturated ring system formed exclusively from carbon atoms and having, respectively, 3 to 6, 3 to 8, and 5 to 8 atoms. Examples are cyclopropyl-, cyclobutyl-, cyclopentyl-, cyclohexyl-, cycloheptyl- or cyclooctyl-.

Cycloalkylene, for example C₃-C₆-cycloalkylene, is understood to mean a bivalent cycloalkyl radical; preference is given to those C₃-C₆-cycloalkylene systems in which both bonds start from the same ring carbon atom.

C₄-C₆-Cycloalkenyl, C₄-C₈-cycloalkenyl, and C₅-C₈-cycloalkenyl are understood to mean a monocyclic, mono- or polyunsaturated, nonaromatic ring system formed exclusively from carbon atoms and having, respectively, 3 to 6, 3 to 8, and 5 to 8 atoms. Examples are cyclobuten-1-yl-, cyclopenten-1-yl-, cyclohexen-2-yl-, cyclohexen-1-yl- or cycloocta-2,5-dienyl-.

Heterocycloalkyl- is understood to mean a 4- to 8-membered monocyclic, saturated ring system having 1 to 3 heteroatoms as defined above in any combination. Preference is given to 4-7-membered heterocycloalkyl groups, particular preference to 5-6-membered heterocycloalkyl groups. Examples include pyrrolidinyl-, piperidinyl-, tetrahydrofuranyl-, tetrahydropyranyl-, oxetanyl-, azetidinyl-, azepanyl-, morpholinyl-, thiomorpholinyl- or piperazinyl-.

Heterocycloalkenyl is understood to mean a 4- to 8-membered monocyclic, mono- or polyunsaturated, nonaromatic ring system having 1 to 3 heteroatoms as defined above in any combination. Preference is given to 4-7-membered heterocycloalkenyl groups, particular preference to 5-6-membered heterocycloalkenyl groups. Examples include 4H-pyranyl-, 2H-pyranyl-, 2,5-dihydro-1H-pyrrolyl-, [1,3]dioxolyl-, 4H-[1,3,4]thiadiazinyl-, 2,5-dihydrofuranyl-, 2,3-dihydrofuranyl-, 2,5-dihydrothiophenyl-, 2,3-dihydrothiophenyl-, 4,5-dihydrooxazolyl-, or 4H-[1,4]thiazinyl-.

C₅-C₁₁-Spirocycloalkyl or C₅-C₁₁-heterospirocycloalkyl having a replacement of 1-4 carbon atoms by heteroatoms as defined above in any combination is understood to mean a fusion of two saturated ring systems which share a common atom. Examples are spiro[2.2]pentyl-, spiro[2.3]hexyl-, azaspiro[2.3]hexyl-, spiro[3.3]heptyl-, azaspiro[3.3]heptyl-, oxazaspiro[3.3]heptyl-, thiaazaspiro[3.3]heptyl-, oxaspiro[3.3]heptyl-, oxazaspiro[5.3]nonyl-, oxazaspiro[4.3]octyl-, oxazaspiro[5.5]undecyl-, diazaspiro[3.3]heptyl-, thiazaspiro[3.3]heptyl-, thiazaspiro[4.3]octyl-, azaspiro[5.5]decyl-, and the further homologous spiro[3.4], spiro[4.4], spiro[5.5], spiro[6.6], spiro[2.4], spiro[2.5], spiro[2.6], spiro[3.5], spiro[3.6], spiro[4.5], spiro[4.6] and spiro[5.6] systems including the variants modified by heteroatoms as per the definition. Preference is given to C₆-C₈-heterospirocycloalkyl.

C₆-C₁₂-Bicycloalkyl or C₆-C₁₂-heterobicycloalkyl having a replacement of 1-4 carbon atoms by heteroatoms as defined above in any combination is understood to mean a fusion of two saturated ring systems which share two directly adjacent atoms. Examples are bicyclo[2.2.0]hexyl-, bicyclo[3.3.0]octyl-, bicyclo[4.4.0]decyl-, bicyclo[5.4.0]undecyl-, bicyclo[3.2.0]heptyl-, bicyclo[4.2.0]octyl-, bicyclo[5.2.0]nonyl-, bicyclo[6.2.0]decyl-, bicyclo[4.3.0]nonyl-, bicyclo[5.3.0]decyl-, bicyclo[6.3.0]undecyl- and bicyclo[5.4.0]undecyl-, including the variants modified by heteroatoms, for example azabicyclo[3.3.0]octyl-, azabicyclo[4.3.0]nonyl-, diazabicyclo[4.3.0]nonyl-, oxazabicyclo[4.3.0]nonyl-, thiazabicyclo[4.3.0]nonyl- or azabicyclo[4.4.0]decyl-, and the further possible combinations as per the definition. Preference is given to C₆-C₁₀-heterobicycloalkyl.

A bridged C₆-C₁₂ ring system such as bridged C₆-C₁₂-cycloalkyl or bridged C₆-C₁₂-heterocycloalkyl is understood to mean a fusion of at least two saturated rings which share two atoms that are not directly adjacent to one another. This may give rise either to a bridged carbocycle (bridged cycloalkyl) or to a bridged heterocycle (bridged heterocycloalkyl) having a replacement of 1-4 carbon atoms by heteroatoms as defined above in any combination. Examples are bicyclo[2.2.1]heptyl-, azabicyclo[2.2.1]heptyl-, oxazabicyclo[2.2.1]heptyl-, thiazabicyclo[2.2.1]heptyl-, diazabicyclo[2.2.1]heptyl-, bicyclo[2.2.2]octyl-, azabicyclo[2.2.2]octyl-, diazabicyclo[2.2.2]octyl-, oxazabicyclo[2.2.2]octyl-, thiazabicyclo[2.2.2]octyl-, bicyclo[3.2.1]octyl-, azabicyclo[3.2.1]octyl-, diazabicyclo[3.2.1]octyl-, oxazabicyclo[3.2.1]octyl-, thiazabicyclo[3.2.1]octyl-, bicyclo[3.3.1]nonyl-, azabicyclo[3.3.1]nonyl-, diazabicyclo[3.3.1]nonyl-, oxazabicyclo[3.3.1]nonyl-, thiazabicyclo[3.3.1]nonyl-, bicyclo[4.2.1]nonyl-, azabicyclo[4.2.1]nonyl-, diazabicyclo[4.2.1]nonyl-, oxazabicyclo[4.2.1]nonyl-, thiazabicyclo[4.2.1]nonyl-, bicyclo[3.3.2]decyl-, azabicyclo[3.3.2]decyl-, diazabicyclo[3.3.2]decyl-, oxazabicyclo[3.3.2]decyl-, thiazabicyclo[3.3.2]decyl- or azabicyclo[4.2.2]decyl- and the further possible combinations as per the definition. Preference is given to bridged C₆-C₁₀-heterocycloalkyl.

Inventive compounds are the compounds of the general formula (I) and the salts, solvates and solvates of the salts thereof, the compounds, encompassed by the general formula (I), of the formulae specified hereinafter and the salts, solvates and solvates of the salts thereof, and the compounds encompassed by the general formula (I) and specified hereinafter as working examples and the salts, solvates and solvates of the salts thereof, to the extent that the compounds encompassed by the general formula (I) and specified hereinafter are not already salts, solvates and solvates of the salts.

The present invention is likewise considered to encompass the use of the salts of the inventive compounds.

In the context of the present invention, preferred salts are physiologically acceptable salts of the inventive compounds. The invention also encompasses salts which themselves are unsuitable for pharmaceutical applications but which can be used, for example, for the isolation or purification of the inventive compounds.

Physiologically acceptable salts of the inventive compounds include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

The present invention further provides all the possible crystalline and polymorphous forms of the inventive compounds, where the polymorphs may be present either as single polymorphs or as a mixture of a plurality of polymorphs in all concentration ranges.

The present invention also relates to medicaments comprising the inventive compounds together with at least one or more further active ingredients, especially for prophylaxis and/or treatment of neoplastic disorders.

In the context of the invention, solvates refer to those forms of the inventive compounds which, in the solid or liquid state, form a complex by coordination with solvent molecules. Hydrates are a specific form of solvates in which the coordination is with water. Preferred solvates in the context of the present invention are hydrates.

Depending on their structure, the inventive compounds may exist in different stereoisomeric forms, i.e. in the form of configurational isomers or if appropriate also as conformational isomers. The inventive compounds may have a centre of asymmetry at the carbon atom to which R⁴ and R⁵ are bonded. They may therefore take the form of pure enantiomers, racemates, or else of diastereomers or mixtures thereof when one or more of the substituents described in the formula (I) contains a further element of asymmetry, for example a chiral carbon atom. The present invention therefore also encompasses diastereomers and the respective mixtures thereof. The pure stereoisomers can be isolated from such mixtures in a known manner; chromatography processes are preferably used for this, in particular HPLC chromatography on a chiral or achiral phase.

In general, the inventive enantiomers inhibit the target to different degrees and have different activity in the cancer cell lines studied. The more active enantiomer is preferred, which is often that in which the centre of asymmetry represented by the carbon atom bonded to R⁴ and R⁵ has (R) configuration.

If the inventive compounds can occur in tautomeric forms, the present invention encompasses all the tautomeric forms.

The present invention also encompasses all suitable isotopic variants of the inventive compounds. An isotopic variant of an inventive compound is understood here to mean a compound in which at least one atom within the inventive compound has been exchanged for another atom of the same atomic number, but with a different atomic mass from the atomic mass which usually or predominantly occurs in nature. Examples of isotopes which can be incorporated into an inventive compound are those of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of an inventive compound, especially those in which one or more radioactive isotopes have been incorporated, may be beneficial, for example, for the examination of the mechanism of action or of the active ingredient distribution in the body; due to comparatively easy preparability and detectability, especially compounds labelled with ³H or ¹⁴C isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the inventive compounds may therefore in some cases also constitute a preferred embodiment of the present invention. Isotopic variants of the inventive compounds can be prepared by the processes known to those skilled in the art, for example by the methods described below and the instructions reproduced in the working examples, by using corresponding isotopic modifications of the particular reagents and/or starting compounds.

In addition, the present invention also encompasses prodrugs of the inventive compounds. The term “prodrugs” includes compounds which may themselves be biologically active or inactive but are converted to inventive compounds while resident in the body (for example metabolically or hydrolytically).

The inventive compounds may act systemically and/or locally. For this purpose, they can be administered in a suitable manner, for example by the oral, parenteral, pulmonary, nasal, sublingual, lingual, buccal, rectal, dermal, transdermal, conjunctival or otic route, or as implant or stent.

The inventive compounds can be administered in administration forms suitable for these administration routes.

Suitable administration forms for oral administration are those which function according to the prior art and deliver the inventive compounds rapidly and/or in modified fashion, and which contain the inventive compounds in crystalline and/or amorphized and/or dissolved form, for example tablets (uncoated or coated tablets, for example having enteric coatings or coatings which are insoluble or dissolve with a delay and control the release of the inventive compound), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can bypass an absorption step (for example intravenously, intraarterially, intracardially, intraspinally or intralumbally) or include an absorption (for example intramuscularly, subcutaneously, intracutaneously, percutaneously or intraperitoneally). Administration forms suitable for parenteral administration include preparations for injection and infusion in the form of solutions, suspensions, emulsions, lyophilizates or sterile powders.

For the other administration routes, suitable examples are inhalation medicaments (including powder inhalers, nebulizers), nasal drops, solutions or sprays; tablets for lingual, sublingual or buccal administration, films/wafers or capsules, suppositories, ear or eye preparations, vaginal capsules, aqueous suspensions (lotions, shaking mixtures), lipophilic suspensions, ointments, creams, transdermal therapeutic systems (for example patches), milk, pastes, foams, dusting powders, implants or stents.

The inventive compounds can be converted to the administration forms mentioned. This can be done in a manner known per se, by mixing with inert, nontoxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), dyes (e.g. inorganic pigments, for example iron oxides) and taste and/or odour correctors.

The present invention further provides medicaments comprising the inventive compounds, typically together with one or more inert, nontoxic, pharmaceutically suitable excipients, and for the use thereof for the aforementioned purposes.

The inventive compounds are formulated to give pharmaceutical preparations in a manner known per se, by converting the active ingredient(s) to the desired administration form with the excipients customary in pharmaceutical formulation.

The excipients used may, for example, be carrier substances, fillers, disintegrants, binders, humectants, glidants, absorbents and adsorbents, diluents, solvents, cosolvents, emulsifiers, solubilizers, taste correctors, colourants, preservatives, stabilizers, wetting agents, salts for modifying osmotic pressure or buffers. Reference should be made to Remington's Pharmaceutical Science, 15th ed. Mack Publishing Company, East Pennsylvania (1980).

The pharmaceutical formulations may be in solid form, for example in the form of tablets, coated tablets, pills, suppositories, capsules, transdermal systems, or in semisolid form, for example in the form of ointments, creams, gels, suppositories, emulsions, or in liquid form, for example in the form of solutions, tinctures, suspensions or emulsions.

Excipients in the context of the invention may, for example, be salts, saccharides (mono-, di-, tri-, oligo- and/or polysaccharides), proteins, amino acids, peptides, fats, waxes, oils, hydrocarbons and derivatives thereof, and the excipients may be of natural origin or be obtained by synthetic or partially synthetic means.

Useful forms for oral or peroral administration are especially tablets, coated tablets, capsules, pills, powders, granules, pastilles, suspensions, emulsions or solutions.

Useful forms for parenteral administration are especially suspensions, emulsions, and particularly solutions.

The inventive compounds are suitable for prophylaxis and/or treatment of hyperproliferative disorders, for example psoriasis, keloids and other hyperplasias which affect the skin, benign prostate hyperplasias (BPH), solid tumours and haematological tumours.

Solid tumours that can be treated in accordance with the invention are, for example, tumours of the breast, the respiratory tract, the brain, the reproductive organs, the gastrointestinal tract, the urogenital tract, the eye, the liver, the skin, the head and the neck, the thyroid gland, the parathyroid gland, the bones, and the connective tissue and metastases of these tumours.

Haematological tumours that can be treated are, for example, multiple myeloma, lymphoma or leukaemia.

Breast tumours that can be treated are, for example, mammary carcinoma with positive hormone receptor status, mammary carcinoma with negative hormone receptor status, Her-2-positive mammary carcinoma, hormone receptor- and Her-2-negative mammary carcinoma, BRCA-associated mammary carcinoma and inflammatory mammary carcinoma.

Tumours of the respiratory tract that can be treated are, for example, non-small-cell bronchial carcinoma and small-cell bronchial carcinoma.

Brain tumours that can be treated are, for example, glioma, glioblastoma, astrocytoma, meningioma and medulloblastoma.

Tumours of the male reproductive organs that can be treated are, for example, prostate carcinoma, malignant epididymal tumours, malignant testicular tumours and penile carcinoma.

Tumours of the female reproductive organs that can be treated are, for example, endometrial carcinoma, cervical carcinoma, ovarian carcinoma, vaginal carcinoma and vulvar carcinoma.

Tumours of the gastrointestinal tract that can be treated are, for example, colorectal carcinoma, anal carcinoma, gastric carcinoma, pancreatic carcinoma, oesophageal carcinoma, gallbladder carcinoma, small-intestinal carcinoma, salivary gland carcinoma, neuroendocrine tumours and gastrointestinal stromal tumours.

Tumours of the urogenital tract that can be treated are, for example, urinary bladder carcinoma, renal cell carcinoma, and carcinoma of the renal pelvis and of the urinary tract.

Tumours of the eye that can be treated are, for example, retinoblastoma and intraocular melanoma.

Tumours of the liver that can be treated are, for example, hepatocellular carcinoma and cholangiocellular carcinoma.

Tumours of the skin that can be treated are, for example, malignant melanoma, basalioma, spinalioma, Kaposi's sarcoma and Merkel cell carcinoma.

Tumours of the head and neck that can be treated are, for example, laryngeal carcinoma and carcinoma of the pharynx and of the oral cavity.

Sarcomas that can be treated are, for example, soft tissue sarcoma and osteosarcoma.

Lymphomas that can be treated are, for example, non-Hodgkin's lymphoma, Hodgkin's lymphoma, cutaneous lymphoma, lymphoma of the central nervous system and AIDS-associated lymphoma.

Leukaemias that can be treated are, for example, acute myeloid leukaemia, chronic myeloid leukaemia, acute lymphatic leukaemia, chronic lymphatic leukaemia and hair cell leukaemia.

Advantageously, the inventive compounds can be used for prophylaxis and/or treatment of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

Particularly advantageously, the inventive compounds can be used for prophylaxis and/or treatment of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The inventive compounds are also suitable for prophylaxis and/or treatment of benign hyperproliferative diseases, for example endometriosis, leiomyoma and benign prostate hyperplasia.

The inventive compounds are also suitable for prophylaxis and/or treatment of systemic inflammatory diseases, especially LPS-induced endotoxic shock and/or bacteria-induced sepsis.

The inventive compounds are also suitable for prophylaxis and/or treatment of inflammatory or autoimmune disorders, for example:
pulmonary disorders associated with inflammatory, allergic and/or proliferative processes: chronic obstructive pulmonary disorders of any origin, particularly bronchial asthma; bronchitis of different origin; all forms of restrictive pulmonary disorders, particularly allergic alveolitis; all forms of pulmonary oedema, particularly toxic pulmonary oedema; sarcoidoses and granulomatoses, particularly Boeck's disease
rheumatic disorders/autoimmune disorders/joint disorders associated with inflammatory, allergic and/or proliferative processes: all forms of rheumatic disorders, especially rheumatoid arthritis, acute rheumatic fever, polymyalgia rheumatica; reactive arthritis; inflammatory soft-tissue disorders of other origin; arthritic symptoms in the case of degenerative joint disorders (arthroses); traumatic arthritides; collagenoses of any origin, e.g. systemic lupus erythematosus, scleroderma, polymyositis, dermatomyositis, Sjögren's syndrome, Still's syndrome, Felty's syndrome
allergies associated with inflammatory and/or proliferative processes: all forms of allergic reactions, e.g. angiooedema, hay fever, insect bites, allergic reactions to medicaments, blood derivatives, contrast agents, etc., anaphylactic shock, urticaria, contact dermatitis
vascular inflammation (vasculitis): panarteritis nodosa, temporal arteritis, erythema nodosum
dermatological disorders associated with inflammatory, allergic and/or proliferative processes: atopic dermatitis; psoriasis; pityriasis rubra pilaris; erythematous disorders triggered by different noxae, for example radiation, chemicals, burns, etc.; bullous dermatoses; lichenoid disorders; pruritus; seborrhoeic eczema; rosacea; pemphigus vulgaris; erythema exsudativum multiforme; balanitis; vulvitis; hair loss, such as alopecia areata; cutaneous T-cell lymphoma
renal disorders associated with inflammatory, allergic and/or proliferative processes: nephrotic syndrome; all nephritides
hepatic disorders associated with inflammatory, allergic and/or proliferative processes: acute hepatic disintegration; acute hepatitis of different origin, for example viral, toxic, medicament-induced; chronic aggressive and/or chronic intermittent hepatitis
gastrointestinal disorders associated with inflammatory, allergic and/or proliferative processes: regional enteritis (Crohn's disease); ulcerative colitis; gastritis; reflux oesophagitis; gastroenteritides of other origin, e.g. indigenous sprue
proctological disorders associated with inflammatory, allergic and/or proliferative processes: anal eczema; fissures; haemorrhoids; idiopathic proctitis
ocular disorders associated with inflammatory, allergic and/or proliferative processes: allergic keratitis, uveitis, iritis; conjunctivitis; blepharitis; optic neuritis; chlorioditis; sympathetic ophthalmia
disorders of the ear-nose-throat region associated with inflammatory, allergic and/or proliferative processes: allergic rhinitis, hay fever; otitis externa, for example caused by contact eczema, infection, etc.; otitis media
neurological disorders associated with inflammatory, allergic and/or proliferative processes: cerebral oedema, particularly tumour-related cerebral oedema; multiple sclerosis; acute encephalomyelitis; meningitis; various forms of seizure, for example West's syndrome
haematological disorders associated with inflammatory, allergic and/or proliferative processes: congenital haemolytic anaemia; idiopathic thrombocytopenia
neoplastic disorders associated with inflammatory, allergic and/or proliferative processes: acute lymphatic leukaemia; malignant lymphoma; lymphogranulomatoses; lymphosarcoma; extensive metastases, particularly in the case of mammary, bronchial and prostate carcinoma
endocrine disorders associated with inflammatory, allergic and/or proliferative processes: endocrine orbitopathy; thyrotoxic crisis; de Quervain's thyroiditis; Hashimoto's thyroiditis; Basedow's disease
organ and tissue transplants, graft-versus-host disease
severe states of shock, for example anaphylactic shock, systemic inflammatory response syndrome (SIRS)
substitution therapy in the case of: congenital primary renal insufficiency, for example congenital adrenogenital syndrome; acquired primary renal insufficiency, for example Addison's disease, autoimmune adrenalitis, postinfectious tumours, metastases, etc; congenital secondary renal insufficiency, for example congenital hypopituitarism; acquired secondary renal insufficiency, for example postinfectious, tumours, etc.
emesis associated with inflammatory, allergic and/or proliferative processes, for example in combination with a 5-HT3 antagonist in the case of cytostatic-induced vomiting
pain of inflammatory origin, for example lumbago

The inventive compounds are also suitable for the treatment of viral disorders, for example infections caused by papilloma viruses, herpes viruses, Epstein-Barr viruses, hepatitis B or C viruses, and human immunodeficiency viruses.

The inventive compounds are also suitable for the treatment of atherosclerosis, dyslipidaemia, hypercholesterolaemia, hypertriglyceridaemia, peripheral vascular disorders, cardiovascular disorders, angina pectoris, ischaemia, stroke, myocardial infarction, angioplastic restenosis, hypertension, thrombosis, obesity, endotoxaemia.

The inventive compounds are also suitable for the treatment of neurodegenerative diseases, for example multiple sclerosis, Alzheimer's disease and Parkinson's disease.

These disorders are well characterized in man, but also exist in other mammals.

The present application further provides the inventive compounds for use as medicaments, especially for prophylaxis and/or treatment of neoplastic disorders.

The present application further provides the inventive compounds for prophylaxis and/or treatment of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present application further provides the inventive compounds for prophylaxis and/or treatment of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The invention further provides for the use of the inventive compounds for production of a medicament.

The present application further provides for the use of the inventive compounds for production of a medicament for prophylaxis and/or treatment of neoplastic disorders.

The present application further provides for the use of the inventive compounds for production of a medicament for prophylaxis and/or treatment of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present application further provides for the use of the inventive compounds for production of a medicament for prophylaxis and/or treatment of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The present application further provides for the use of the inventive compounds for prophylaxis and/or treatment of neoplastic disorders.

The present application further provides for the use of the inventive compounds for prophylaxis and/or treatment of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present application further provides for the use of the inventive compounds for prophylaxis and/or treatment of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The present application further provides pharmaceutical formulations in the form of tablets comprising one of the inventive compounds for prophylaxis and/or treatment of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present application further provides pharmaceutical formulations in the form of tablets comprising one of the inventive compounds for prophylaxis and/or treatment of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The invention further provides for the use of the inventive compounds for treatment of disorders associated with proliferative processes.

The invention further provides for the use of the inventive compounds for treatment of benign hyperplasias, inflammation disorders, autoimmune disorders, sepsis, viral infections, vascular disorders and neurodegenerative disorders.

The inventive compounds can be used alone or, if required, in combination with one or more further pharmacologically active substances, provided that this combination does not lead to undesirable and unacceptable side effects. The present invention therefore further provides medicaments comprising an inventive compound and one or more further active ingredients, especially for prophylaxis and/or treatment of the aforementioned disorders.

For example, the inventive compounds can be combined with known antihyperproliferative, cytostatic or cytotoxic chemical and biological substances for treatment of cancer. The combination of the inventive compounds with other substances commonly used for cancer treatment, or else with radiotherapy, is particularly appropriate.

An illustrative but nonexhaustive list of suitable combination active ingredients is as follows:

abiraterone acetate, abraxane, acolbifene, Actimmune, actinomycin D (dactinomycin), afatinib, affinitak, Afinitor, aldesleukin, alendronic acid, alfaferone, alitretinoin, allopurinol, Aloprim, Aloxi, alpharadin, altretamine, aminoglutethimide, aminopterin, amifostine, amrubicin, amsacrine, anastrozole, anzmet, apatinib, Aranesp, arglabin, arsenic trioxide, Aromasin, arzoxifen, asoprisnil, L-asparaginase, atamestane, atrasentane, avastin, axitinib, 5-azacytidine, azathioprine, BCG or Tice BCG, bendamustine, bestatin, beta-methasone acetate, betamethasone sodium phosphate, bexarotene, bicalutamide, bleomycin sulphate, broxuridine, bortezomib, bosutinib, busulfan, cabazitaxel, calcitonin, campath, camptothecin, capecitabine, carboplatin, carfilzomib, carmustine, casodex, CCI-779, CDC-501, cediranib, cefesone, celebrex, celmoleukin, cerubidine, cediranib, chlorambucil, cisplatin, cladribine, clodronic acid, clofarabine, colaspase, copanlisib, corixa, crisnatol, crizotinib, cyclophosphamide, cyproterone acetate, cytarabine, dacarbazine, dactinomycin, dasatinib, daunorubicin, DaunoXome, Decadron, Decadron Phosphate, decitabine, degarelix, delestrogen, denileukin diftitox, depomedrol, deslorelin, dexrazoxane, diethylstilbestrol, diflucan, 2′,2′-difluorodeoxycytidine, DN-101, docetaxel, doxifluridine, doxorubicin (Adriamycin), dronabinol, dSLIM, dutasteride, DW-166HC, edotecarin, eflornithine, Eligard, Elitek, Ellence, Emend, enzalutamide, epirubicin, epoetin-alfa, Epogen, epothilone and derivatives thereof, eptaplatin, ergamisol, erlotinib, erythro-hydroxynonyladenine, estrace, oestradiol, oestramustine sodium phosphate, ethinyloestradiol, Ethyol, etidronic acid, etopophos, etoposide, everolimus, exatecan, exemestane, fadrozole, farston, fenretinide, filgrastim, finasteride, fligrastim, floxuridine, fluconazole, fludarabine, 5-fluorodeoxyuridine monophosphate, 5-fluorouracil (5-FU), fluoxymesterone, flutamide, folotin, formestane, fosteabine, fotemustine, fulvestrant, Gammagard, gefitinib, gemcitabine, gemtuzumab, Gleevec, Gliadel, goserelin, gossypol, granisetrone hydrochloride, hexamethylmelamine, histamine dihydrochloride, histrelin, holmium-166-DOTPM, hycamtin, hydrocortone, erythro-hydroxynonyladenine, hydroxyurea, hydroxyprogesterone caproate, ibandronic acid, ibritumomab tiuxetan, idarubicin, ifosfamide, imatinib, iniparib, interferon-alpha, interferon-alpha-2, interferon-alpha-2α, interferon-alpha-2β, interferon-alpha-n1, interferon-alpha-n3, interferon-beta, interferon-gamma-1α, interleukin-2, intron A, iressa, irinotecan, ixabepilone, keyhole limpet haemocyanin, kytril, lanreotide, lapatinib, lasofoxifene, lenalidomide, lentinan sulphate, lestaurtinib, letrozole, leucovorin, leuprolide, leuprolide acetate, levamisole, levofolic acid calcium salt, levothroid, levoxyl, Libra, liposomal MTP-PE, lomustine, lonafarnib, lonidamine, marinol, mechlorethamine, mecobalamine, medroxyprogesterone acetate, megestrol acetate, melphalan, Menest, 6-mercaptopurine, mesna, methotrexate, metvix, miltefosine, minocycline, minodronate, miproxifen, mitomycin C, mitotan, mitoxantrone, modrenal, MS-209, MX-6, myocet, nafarelin, nedaplatin, nelarabine, nemorubicin, neovastat, neratinib, neulasta, neumega, neupogen, nilotimib, nilutamide, nimustine, nolatrexed, nolvadex, NSC-631570, obatoclax, oblimersen, OCT-43, octreotide, olaparib, ondansetron hydrochloride, Onco-TCS, Orapred, Osidem, oxaliplatin, paclitaxel, pamidronate disodium, pazopanib, pediapred, pegaspargase, pegasys, pemetrexed, pentostatin, N-phosphonoacetyl-L-aspartate, picibanil, pilocarpine hydrochloride, pirarubicin, plerixafor, plicamycin, PN-401, porfimer sodium, prednimustine, prednisolone, prednisone, Premarin, procarbazine, Procrit, QS-21, quazepam, R-1589, raloxifene, raltitrexed, ranpirnas, RDEA119, Rebif, regorafenib, 13-cis-retinoic acid, rhenium-186 etidronate, rituximab, roferon-A, romidepsin, romurtide, ruxolitinib, salagen, salinomycin, sandostatin, sargramostim, satraplatin, semaxatinib, semustine, seocalcitol, sipuleucel-T, sizofiran, sobuzoxan, Solu-Medrol, sorafenib, streptozocin, strontium-89 chloride, sunitinib, Synthroid, T-138067, tamoxifen, tamsulosin, Tarceva, tasonermin, tastolactone, Taxoprexin, Taxoter, teceleukin, temozolomide, temsirolimus, teniposide, testosterone propionate, Testred, thalidomide, thymosin alpha-1, thioguanine, thiotepa, thyrotropin, tiazorufin, tiludronic acid, tipifarnib, tirapazamine, TLK-286, toceranib, topotecan, toremifen, tositumomab, tastuzumab, teosulfan, transMID-107R, tretinoin, Trexall, trimethylmelamine, trimetrexate, triptorelin acetate, triptorelin pamoate, trofosfamide, UFT, uridine, valrubicin, valspodar, vandetanib, vapreotide, vatalanib, vemurafinib, verte-porfin, vesnarinone, vinblastine, vincristine, vindesine, vinflumine, vinorelbine, virulizin, vismodegib, Xeloda, Z-100, Zinecard, zinostatin stimalamer, zofran, zoledronic acid.

More particularly, the inventive compounds can be combined with antibodies, for example aflibercept, alemtuzumab, bevacizumab, brentuximumab, catumaxomab, cetuximab, denosumab, edrecolomab, gemtuzumab, ibritumomab, ipilimumab, ofatumumab, panitumumab, pertuzumab, rituximab, tositumumab or trastuzumab, and also with recombinant proteins.

More particularly, the inventive compounds can be used in combination with treatments directed against angiogenesis, for example bevacizumab, axitinib, regorafenib, cediranib, sorafenib, sunitinib, lenalidomide, copanlisib or thalidomide.

Combinations with antihormones and steroidal metabolic enzyme inhibitors are particularly suitable because of their favourable profile of side effects.

Combinations with P-TEFb inhibitors and CDK9 inhibitors are likewise particularly suitable because of the possible synergistic effects.

Generally, the following aims can be pursued with the combination of the inventive compounds with other cytostatically or cytotoxically active agents:
improved efficacy in slowing the growth of a tumour, in reducing its size or even in completely eliminating it, compared with treatment with an individual active ingredient;
the possibility of using the chemotherapeutics used in a lower dosage than in the case of monotherapy;
the possibility of a more tolerable therapy with fewer side effects compared with individual administration;
the possibility of treatment of a broader spectrum of neoplastic disorders;
the attainment of a higher rate of response to the therapy;
a longer survival time of the patient compared with present standard therapy.

In addition, the inventive compounds can also be used in conjunction with radiotherapy and/or surgical intervention.

### Preparation of the Inventive Compounds:

### In the Present Description:

NMR signals are reported with their particular apparent multiplicities or combinations thereof. In this context, s=singlet, d=doublet, t=triplet, q=quartet, qi=quintet, sp=septet, m=multiplet, b=broad signal. Signals having combined multiplicities are reported, for example, as dd=doublet of doublets.

| | |
|---|---|
| CDCl₃ | deuterochloroform |
| dba | dibenzylideneacetone |
| DMA | N,N-dimethylacetamide |
| DMF | N,N-dimethylformamide |
| DMSO-d6 | deuterated dimethyl sulphoxide |
| DMSO | dimethyl sulphoxide |
| HATU | (7-aza-1H-benzotriazol-1-yl)-1,1,3,3-tetramethyluronium |
| | hexafluorophosphate |
| RP-HPLC | reverse-phase high-pressure liquid chromatography |
| RT | room temperature |
| THF | tetrahydrofuran |
| HBTU | O-benzotriazole-N,N,N′,N′-tetramethyluronium |
| | hexafluorophosphate |
| PyBOB | (benzotriazol-1-yl)oxytripyrrolidinophosphonium |
| | hexafluorophosphate |
| T3P | 2,4,6-tripropyl-1,3,5,2,4,6-trioxatriphosphorinane |
| | 2,4,6-trioxide |
| LCMS | liquid chromatography coupled with mass spectrometry |
| CHAPS | 3-{dimethyl[3-(4-{5,9,16-trihydroxy-2,15- |
| | dimethyltetracyclo-[8.7.0.0^{2,7}.0^{11,15}]heptadecan-14-yl} |
| | pentanamido)propyl]-azaniumyl}propane-1-sulphonate |
| (+)-BINAP | (R)-(+)-2,2′-bis(diphenylphosphino)-1,1′-binaphthyl |
| (±)-BINAP | 2,2′-bis(diphenylphosphino)-1,1′-binaphthyl (racemic) |
| TBTU | (benzotriazol-1-yloxy)bisdimethylaminomethylium |
| | fluoroborate |
| DCC | dicyclohexylcarbodiimide |
| Xanthphos | (9,9-dimethyl-9H-xanthene-4,5-diyl)bis(diphenylphosphine) |

### General Description of the Preparation of the Inventive Compounds of the General Formula I

The inventive compounds of the formulae (Ia), (Ib), (Ic) and (Id) shown in Scheme 1 can be prepared via synthesis routes described hereinafter. These formulae represent different portions of the general formula (I) in which A, R², R³, R⁴, R⁵, R⁶, R⁷, R⁸ and n are each as defined for the general formula (I). In compounds of the formula (Ia) of the dihydropyridopyrazinone type, —N— replaces X and a —C(═O)NR⁷R⁸ group replaces R¹; in compounds of the formula (Ib) of the dihydroquinoxalinone type, —CH— replaces X and a —C(═O)NR⁷R⁸ group replaces R¹; in compounds of the formula (Ic), X is as defined for the general formula (I) and a —S(═O)₂NR⁷R⁸ group replaces R¹, and in compounds (Id), finally, HetAr, which is 5-membered monocyclic heteroaryl- as defined in formula (I) for R¹, replaces R¹.

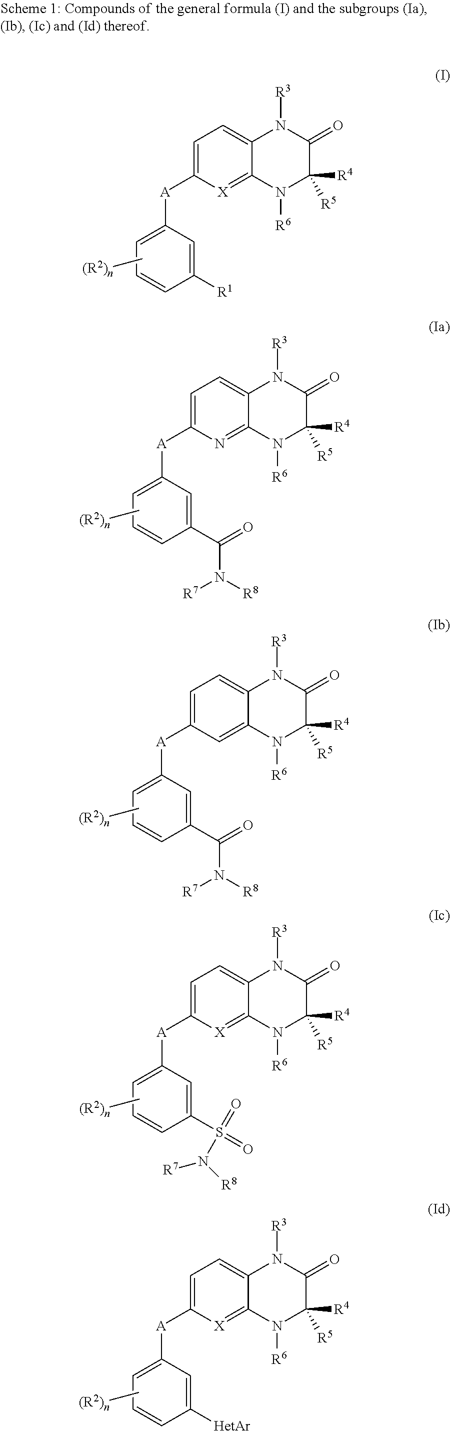

In addition to the synthesis sequences discussed hereinafter, it is also possible, in accordance with the general knowledge of the person skilled in the art in organic chemistry, to take other synthesis routes for the synthesis of inventive compounds of the general formula (I). The sequence of the synthesis steps shown in the schemes which follow is not binding, and synthesis steps from various of the schemes shown hereinafter may optionally be combined to form new sequences. In addition, interconversions of the substituents R², R³, R⁴, R⁵, R⁶, R⁷, R⁸ can be performed before or after the synthesis stages shown. Examples of such conversions are the introduction or elimination of protecting groups, reduction or oxidation of functional groups, halogenation, metallation, metal-catalysed coupling reactions, substitution reactions or further reactions known to the person skilled in the art. These reactions include conversions which introduce a functional group which enables the further conversion of substituents. Suitable protective groups and methods for their introduction and removal are known to the person skilled in the art (see, for example, T. W. Greene and P. G. M. Wuts in: Protective Groups in Organic Synthesis, 3rd Edition, Wiley 1999). In addition, it is possible to combine two or more reaction steps without intermediate workup in a manner known to the person skilled in the art (for example in what are called “one-pot” reactions).

Scheme 2 illustrates the formation of amides of the formula (V) from simple pyridine derivatives of the formula (II) in which the R^{Hal} groups may be the same or different and are each halogen, preferably fluorine or chlorine, for example 3-amino-2,6-dichloropyridine (CAS No. 62476-56-6) or 3-amino-2,6-difluoropyridine (CAS No. 108118-69-0). For the preparation of (III) from (II), it is possible to use a multitude of methods for preparing amides from the azidocarboxylic acids of the formula (IIa) in which R⁴ and R⁵ are each as defined for the general formula (I). Thus, it is possible to use coupling reagents known to the person skilled in the art, such as TBTU, HATU, T3P or DCC. Likewise suitable is the reaction of the azidocarboxylic acids used with an inorganic acid chloride such as thionyl chloride, phosphorus oxychloride or oxalyl chloride, followed by addition of the pyridineamine. The preparation of the azidocarboxylic acids required is described in the literature (Chem Eur J (2010), 16, p7572 ff, D. Tietze et al.; J Org Chem (2010), 75, p6532ff, Katritzky et al.). The azidocarboxylic acids have to be handled very carefully since they can decompose explosively. Storage of the reagents required for azide introduction should likewise be dispensed with. These aspects are discussed in Katritzky et al.

To reduce the azido group in (III), which leads to amines of the formula (IV), the reaction with trialkyl- or triarylphosphines can be conducted according to Staudinger (Tetrahedron (2012), 68, p697ff, Laschat et al.). An example of a suitable phosphine is trimethylphosphine. The amines (IV) can be isolated as the free base or, advantageously, in salt form, for instance as the hydrochloride. To this end, the crude amine of the formula (IV) is dissolved in a nonpolar solvent, for example diethyl ether, and precipitated as salt by addition of an acid, for example hydrogen chloride. The further conversion to compounds of the formula (V) with introduction of the R⁶ radical, which is as defined for the general formula (I), can preferably be conducted via the reductive amination known to the person skilled in the art (for representative procedures see, for example, US2010/105906 A1). This involves reacting the primary amine (IV), as the free base or in salt form, in situ with an aldehyde or ketone suitable for the introduction of R⁶ to give an imine, and then transforming the latter by addition of a suitable reducing agent such as sodium triacetoxyborohydride to give the secondary amine of the formula (V).

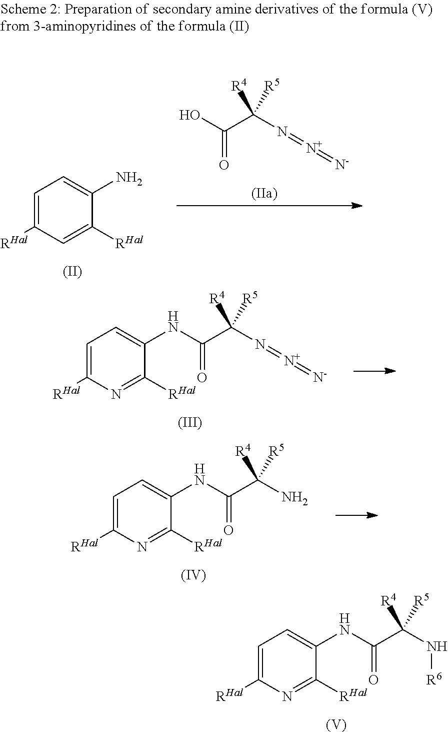

Alternatively, intermediates of the formula (IV), as the free base or in salt form, can be prepared by reaction of simple pyridine derivatives of the formula (II) in which the R^{Hal} groups may be the same or different and are each halogen, preferably fluorine or chlorine, for example 3-amino-2,6-dichloropyridine (CAS No. 62476-56-6) or 3-amino-2,6-difluoropyridine (CAS No. 108118-69-0), with an appropriate N-protected amino acid of the formula (IIb) in which R⁴ and R⁵ are each as defined for the general formula (I), and in which SG is a suitable protecting group SG, for example BOC, Fmoc or Cbz (Scheme 3). N-Protected amino acids are typically commercially available. It is possible to use coupling reagents known to the person skilled in the art, such as TBTU, HATU, T3P or DCC. Likewise suitable is the reaction of the N-protected amino acid of the formula (IIb) used with an inorganic acid chloride such as thionyl chloride, phosphorus oxychloride or oxalyl chloride, followed by addition of the pyridineamine. This gives compounds of the formula (VI), which can be converted by the methods known to those skilled in the art for detaching protecting groups to compounds of the formula (IV).

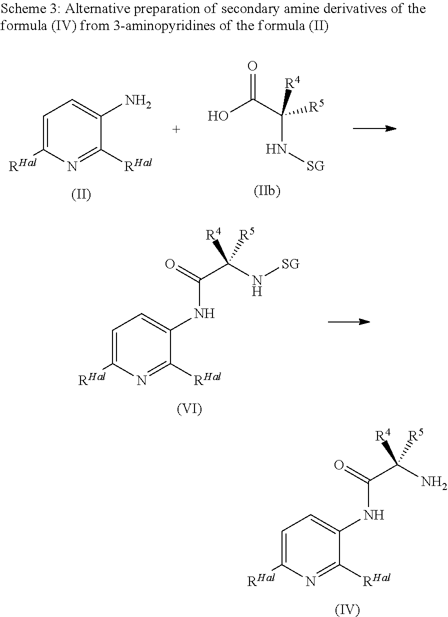

As shown in Scheme 4, the secondary amines of the formula (V) can be converted by cyclization to dihydropyridopyrazinones of the formula (VII). To this end, compounds of the formula (V) can be reacted in the presence of a suitable base at elevated temperature (see also WO2010/96426 A2, Example 16). The subsequent alkylation to give compounds (VIII) can be effected by reaction with R³-LG in which R³ is as defined in the general formula (I) and LG is a leaving group, preferably iodide, in the presence of a suitable base such as sodium hydride, under conditions known to the person skilled in the art. The further conversion of the resulting compounds of the formula (VIII) to the ester derivatives (IX) can be effected by reaction with compounds of the formula (VIIIa) in which A, R², and n are each as defined in the general formula 1, and in which R^{E} is C₁-C₆-alkyl, in a palladium-catalysed coupling reaction according to Buchwald and Hartwig (see, for example, J. Organomet. Chem. (1999), 576, p125ff). Examples of palladium sources suitable here are palladium acetate or palladium(dba) complexes, for example Pd₂(dba)₃ (CAS Nos. 51364-51-3 and 52409-22-0). The conversion depends significantly on the ligands used. The examples adduced in the experimental section were obtained in this way, for example through the use of (+)-BINAP (cf. also US2006/009457 A1).

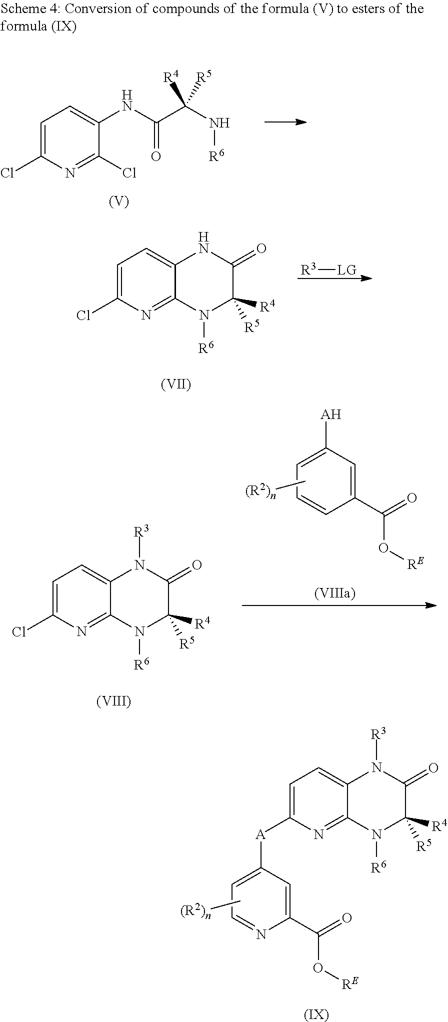

The preparation of carboxamides of the general formula (Ia) can be effected in accordance with Scheme 5 by means of hydrolysis of the respective esters of the formula (IX) to the corresponding carboxylic acids of the formula (X) by methods known to the person skilled in the art. These reactions can preferably be carried out using alkali metal hydroxides such as lithium hydroxide, sodium hydroxide or potassium hydroxide in aqueous alcoholic solutions.

The carboxylic acids (X) obtained in this way can be converted to the inventive carboxamides of the general formula (Ia) by reaction with the generally commercially available amines of the formula R⁷R⁸NH in which R⁷ and R⁸ are each as defined for the general formula (I), with additional activation by a method as commonly known to the person skilled in the art. Possible methods which should be mentioned here include the use of HATU, HBTU, PyBOB or T3P with the addition of a suitable base. The conversion of the carboxylic acids to their amides is described in general terms in reference books such as “Compendium of Organic Synthetic Methods”, volume I-VI (Wiley Interscience) or “The Practice of Peptide Synthesis”, Bodansky (Springer Verlag).

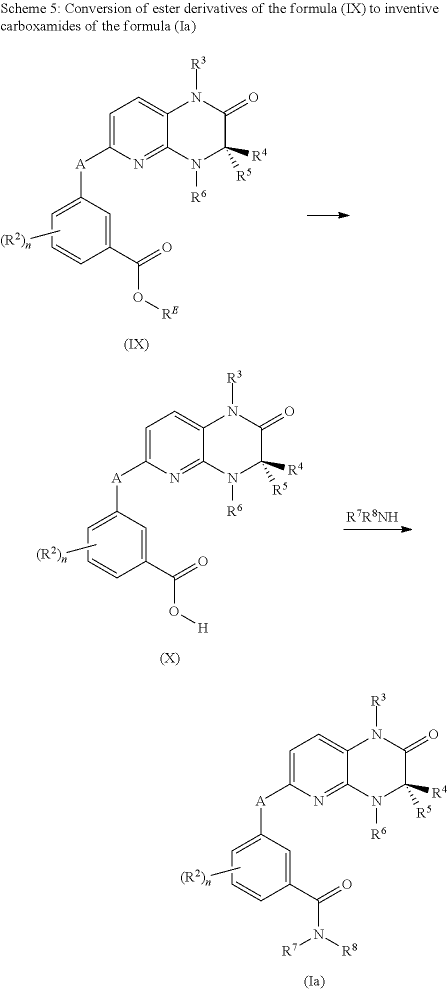

Dihydroquinoxalinones of the formula (Ib) can be obtained as described in Scheme 6. To this end, it is possible to react suitable ortho-fluoronitrobenzene derivatives, for example 4-bromo-2-fluoronitrobenzene ((XI); CAS No. 321-23-3), by nucleophilic ipso substitution with amino acids of the structure (XIa) in which R⁴ and R⁵ are each as defined for the general formula (I) to give compounds of the structure (XII). By selective reduction of the nitro group with a suitable reducing agent and subsequent workup in an acidic medium, the bicyclic compounds of the formula (XIII) are obtained directly. Suitable reducing agents that may be employed are, for example, alkali metal dithionites (J Heterocyclic Chem. (1992), 29, P1859-61, Shafiee et al.), or tin(II) chloride (J. Org. Chem. (1983), 48, p2515ff, Xing et al.). The entire reaction sequence of reduction and cyclization has likewise been described (WO2010/116270 A1, L.1.b). For preparation of the compounds (XIV) substituted on the basic nitrogen, in which R⁶ is as defined in the general formula (1), the compounds of the formula (XIII) can be reacted with aldehydes or ketones suitable for the introduction of R⁶ and a reducing agent by a reductive amination known to those skilled in the art. Here, for example, the use of an alkyl- or arylsilane, for example phenylsilane, optionally in combination with dibutyltin dichloride, as the reducing agent is a method which is known to those skilled in the art and gives the intermediates (XIV) in adequate yields (Bioorg. Med. Chem. Lett. (2009), 19, S. 688ff; D. V. Smil et al.).

Further conversion to the inventive compounds of the formula (Ib) via the intermediates (XV), (XVI) and (XVII) can be carried out under conditions comparable to those described in Schemes 4 and 5 for the conversion of intermediates of the formula (VII) to the inventive compounds of the formula (Ia) via the intermediates (VIII), (IX) and (X).

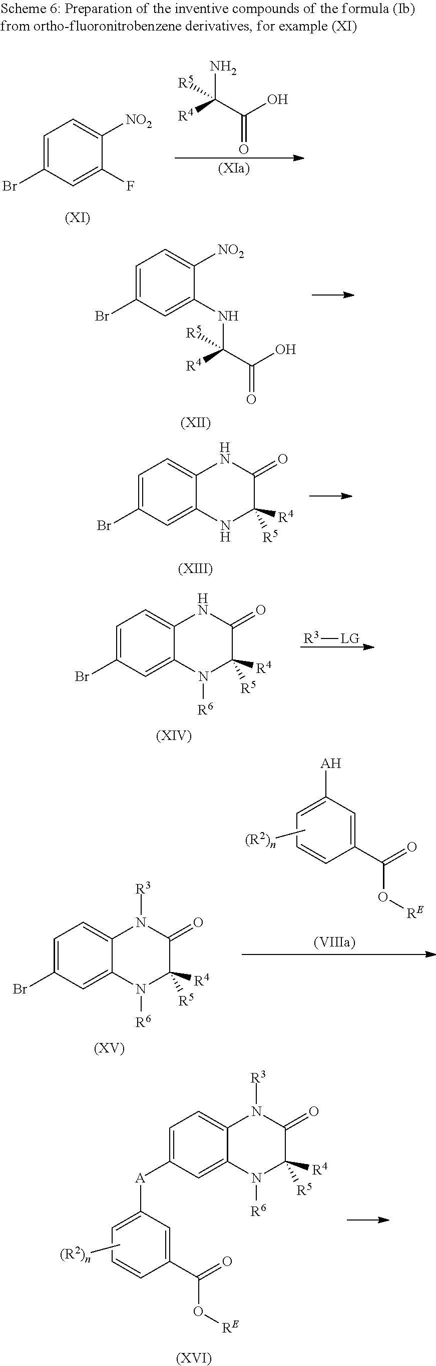

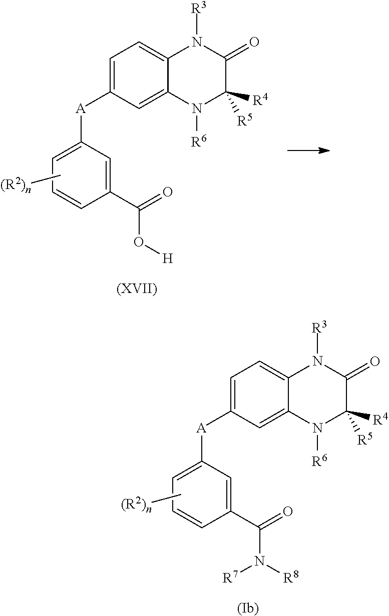

Alternatively, structures of the formula (XIV) can also be prepared as described in Scheme 7. In this case, the amino acid ester (XVIII) already bears the R⁶ radical as per formula (I). The amino acid ester (XVIII) is prepared by reacting the amino acid ester (XIb) unsubstituted on the nitrogen in situ with an aldehyde or ketone suitable for the introduction of R⁶ to give an imine, and then transforming the latter by addition of a suitable reducing agent such as sodium triacetoxyborohydride to give the secondary amine of the formula (XVIII). This reaction is effected under the conditions known to those skilled in the art for reductive amination (for representative methods see, for example, US2010/105906 A1).

Further reaction with suitable ortho-fluoronitrobenzene derivatives, for example 4-bromo-2-fluoronitrobenzene ((XI); CAS No. 321-23-3), through nucleophilic ipso substitution with the R⁶-substituted amino acid esters of the formula (XVIII) in which R⁴, R⁵ and R⁶ are each as defined for the general formula (I) leads to compounds of the structure (XIX) in which R^{E} is C₁-C₆-alkyl. By selective reduction of the nitro group with a suitable reducing agent and subsequent workup in an acidic medium, the bicyclic compounds of the formula (XIV) are obtained directly. Suitable reducing agents that may be employed are, for example, alkali metal dithionites (J Heterocyclic Chem. (1992), 29, P1859-61, Shafiee et al.), tin(II) chloride (J. Org. Chem. (1983), 48, p. 2515ff, Xing et al.), or iron powder in the presence of a suitable acid, for example hydrochloric acid, acetic acid or aqueous ammonium chloride solution. The entire reaction sequence of reduction and cyclization is effected analogously to a literature method (WO2010/116270 A1, L.1.b), optionally with replacement of the sodium dithionite, described as the reducing agent, in aqueous potassium carbonate solution with iron powder in a mixture of methanol and glacial acetic acid.

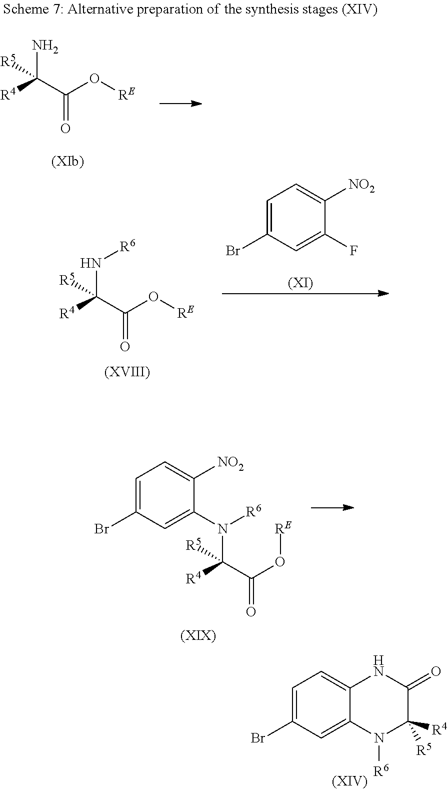

The preparation of intermediates of the formula (VIIa) in which R^{6′} is optionally substituted phenyl as per the definition of R⁶ in the general formula (1) is described in Scheme 8. 3-Amino-2,6-dichloropyridine ((IIc), CAS No. 62476-56-6) is reacted with compounds of the formula (XX) in which R⁴ and R⁵ are as defined for the general formula (I), and in which LG and LG′ are each independently of one another a leaving group, preferably chlorine or bromine, for example 2-bromopropionyl bromide (CAS 563-76-8). This is done by conversion, under conditions known to the person skilled in the art, with a suitable solvent such as dichloromethane or THF and with addition of a base such as triethylamine, diisopropylethylarnine or pyridine. The base can also be used as the solvent. This gives compounds of the formula (XXI). These intermediates (XXI) are reacted with anilines of the formula R^{6′}—NH₂ in which R^{6′} is optionally substituted phenyl as per the definition of R6 in the general formula (I) to give compounds of the formula (XXII). This reaction can be effected by reaction in various solvents such as toluene or acetonitrile and with addition of a base, for example potassium carbonate, di-iso-propylethylamine or triethylamine at elevated temperature (Org. Lett. (2008), 10, p. 2905ff, S. P. Marsden et al.). Dihydropyridopyrazinones of the formula (VIIa) in which R^{6′} is optionally substituted phenyl as per the definition of R⁶ in the general formula (I) are obtained by cyclizing the compounds of the formula (XXII) in the presence of a suitable base, for example triethylamine, diiso-propylethylamine or potassium carbonate, at elevated temperature in solvents, for example N,N-dimethylformamide, N,N-dimethylacetamide, N-methylpyrrolidone or else dimethyl sulphoxide (see also WO2010/96426 A2, Example 16). From these intermediates of the formula (VIIa), it is possible according to Schemes 4 and 5 to prepare the corresponding inventive compounds of the formula (I) in which X is —N— and R^{6′} is optionally substituted phenyl as per the definition of R⁶ in the general formula (I). This gives said compounds of the formula (I) as racemates if R⁴ and R⁵ are different from one another. These can optionally be separated into the enantiomers by separation methods familiar to the person skilled in the art, for example preparative HPLC on a chiral stationary phase.

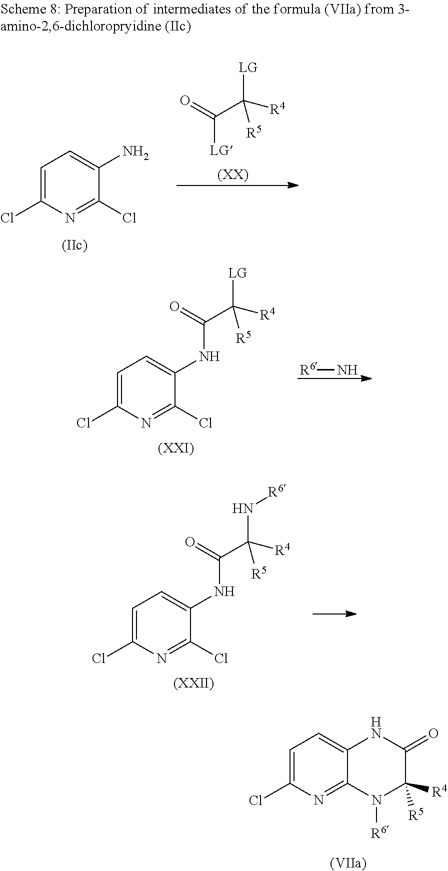

The inventive compounds of the formula (Ic) having a sulphonamide group in place of R¹ can be prepared according to Scheme 9. In this context, compounds of the formula (VIII) (for dihydropyridopyrazinone derivatives) or compounds of the formula (XV) (for dihydroquinoxalinone derivatives) can be reacted directly, in a manner analogous to that discussed in Scheme 4 for the conversion of (VIII) to (IX), with compounds of the formula (XXII) in which A, R², R⁷, R⁸ and n are each as defined in the general formula (I) in a palladium-catalysed coupling reaction according to Buchwald and Hartwig to give the inventive compounds of the formula (Ic). Compounds of the formula (XXII) are commercially available or can be prepared via methods known to those skilled in the art (e.g. J. Med. Chem. (1996), 39, p904ff., T. R. Jones et al.).

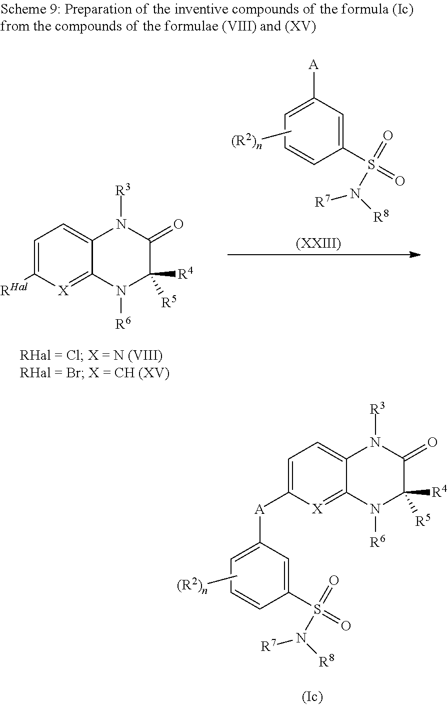

In an analogous manner, this method, as shown in Scheme 10, can also be used as an alternative method for the preparation of carboxamides of the general formulae (Ia) and (Ib), by replacing sulphonamide intermediates (XXIII) with the analogous carboxamides (XXIIIa) in which A, R², R⁷, R⁸ and n are each as defined in the general formula (I).

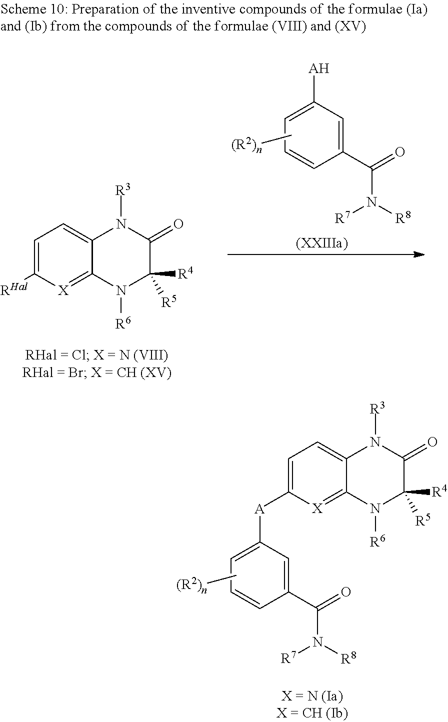

In addition, in a likewise analogous manner, the halogenated intermediates (VIII) and (XV), through reaction with compounds of the formula (XXIIIb) in which A, R² and n are each as defined in the general formula (I), and in which HetAr is 5-membered monocyclic heteroaryl-, as defined in formula (I) for R¹, can be used to obtain inventive compounds of the formula (Id), as shown in Scheme 11:

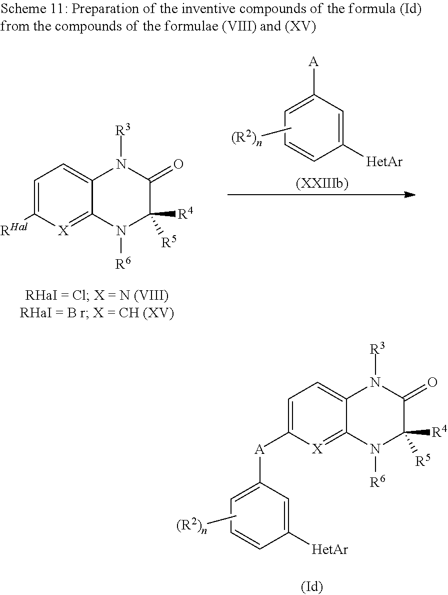

Compounds of the formula (XXIIIb) are in many cases commercially available or are known to those skilled in the art. Inventive compounds of the formula (Id) are additionally obtainable by, as shown in Scheme 12, reacting intermediates of the formula (XXIV), which can be prepared by the methods described above and in which A, X, R², R³, R⁴, R⁵, R⁶ and n are each as defined in the general formula (I), and in which R^{Hal} is a halogen, preferably bromine or iodine, in a Suzuki coupling familiar to those skilled in the art, with a heteroaromatic boronic acid or a corresponding boronic ester in which HetAr is 5-membered monocyclic heteroaryl-, as defined in formula (I) for R¹, and R is hydrogen or C₁-C₄-alkyl-, or —B(OR)₂ is a pinacolyl boronate, to give the inventive compounds of the formula (Id) (see also D. G. Hall, Boronic Acids, 2005 WILEY-VCH Verlag GmbH & Co. KGaA, Weinheim, ISBN 3-527-30991-8, and literature cited therein).

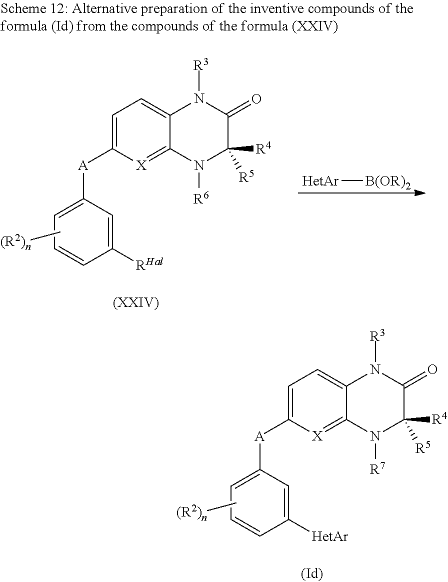

In addition, the inventive compounds of the formula (Id) can also be formed from the ester intermediates of the formulae (IX) and (XVI) shown in Schemes 5 and 6, and carboxylic acids of the formulae (X) and (XVII), in a manner known to those skilled in the art, for example via direct reaction of an ester with hydroxyamidines as described in the literature (Tetrahedron Lett. (2006), 47, p4271-4, W. Du et al.). By this process, it is possible to convert both aliphatically substituted hydroxyvamidines and aromatically substituted hydroxyamidines. Other heterocycles can also be prepared proceeding from carboxylic acids of the formulae (X) and (XVII), for example, which are first reacted with alkyl or aryl hydrazides using methods known to those skilled in the art (see also Scheme 5) to give bisacyl hydrazides and then using reagents for elimination of water which are known to those skilled in the art, for example phorphorus oxychloride, thionyl chloride, p-toluenesulphonyl chloride or the Burgess reagent. In this way, for example, 1,3,4-oxadiazoles (J. Med. Chem. (2005), 48, p4068ff Garcia et al.) are obtainable.

The reaction routes described allow, in the case of the use of an enantiomerically pure azidocarboxylic acid of the formula (IIa) or of enantiomerically pure amino acids of the formula (IIb) or (XIa), or the corresponding ester of the formula (XIb), at the start of the sequence, very substantial suppression of epimerization or racemization of the stereogenic centre at the carbon atom bonded to R⁴ and R⁵.

The present invention likewise provides the intermediate compounds of the general formulae (IX) and (XVI)

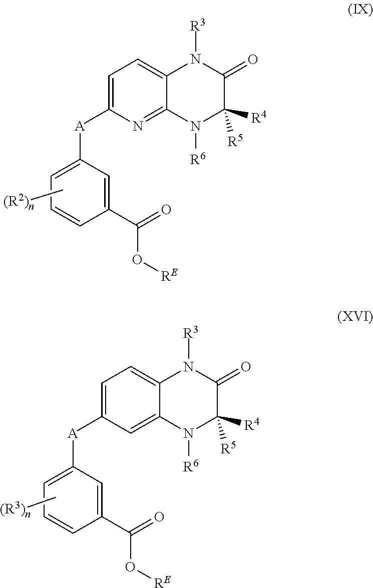

in which A, R², R³, R⁴, R⁵, R⁶ and n are each as defined in the general formula (I) and R^{E} is C₁-C₆-alkyl, which can be used preferentially for preparation of the inventive compounds of the general formula (I).

Preference is given to those intermediates of the general formulae (IX) and (XVI) in which R^{E} is methyl or ethyl.

The present invention also further provides the intermediate compounds of the general formulae (X) and (XVII)

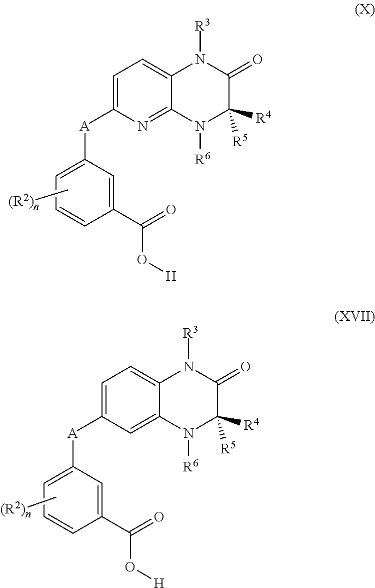

in which A, R², R³, R⁴, R⁵, R⁶ and n are each as defined in the general formula (I), which can be used preferentially for preparation of the inventive compounds of the general formula (I).

### WORKING EXAMPLES

The examples which follow illustrate the preparation of the inventive compounds, without restricting the invention to these examples.

Firstly, there is a description of the preparation of the intermediates which are ultimately used preferentially for preparation of the inventive compounds.

IUPAC names were created with the aid of the nomenclature software ACD Name batch, Version 12.01, from Advanced Chemical Development, Inc., and adapted if required, for example to German-language nomenclature.

### Stoichiometry of Salt Forms

If, in the synthesis intermediates and working examples of the invention described below, a compound is given in the form of a salt of the corresponding base or acid, the exact stoichiometric composition of such a salt as obtained by the respective preparation and/or purification process is generally not known. Unless specified in more detail, additions to names and structural formulae, such as “hydrochloride”, “trifluoroacetate”, “sodium salt” or “x HCl”, “x CF₃COOH”, “x Na⁺” are not to be understood stoichiometrically in the case of such salts, but have only descriptive character with regard to the salt-forming components comprised therein.

This applies correspondingly if the synthesis intermediates and working examples or salts thereof were obtained by the preparation and/or purification processes described in the form of solvates, for example hydrates, whose stoichiometric composition (if of a defined type) is not known.

### Preparation of the Intermediates

### Intermediate 1

### (2R)-2-Azido-N-(2,6-dichloropyridin-3-yl)propanamide

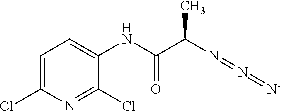 [SMILES: C[C@@H](N=[N+]=[N-])C(=O)Nc1ccc(Cl)nc1Cl]

To a solution of 6.6 g of (2R)-2-azidopropanoic acid (Chem. Eur. J. (2010), 16, p. 7572-7578) in 250 ml dimethylacetamide were added dropwise, at −10° C., 5.02 ml of thionyl chloride. The mixture was stirred at −10° C. for 30 minutes and then 10.6 g of 3-amino-2,6-dichloropyridine (commercially available; CAS No. 62476-56-6) were added. The mixture was gradually warmed up to RT and stirred for a further 3 hours. The reaction solution was admixed with water and extracted three times with ethyl acetate. The combined organic phases were washed with water and saturated sodium chloride solution, dried over sodium sulphate and concentrated under reduced pressure. The residue was purified by chromatography on silica gel (hexane/ethyl acetate gradient). This gave 10.6 g of (2R)-2-azido-N-(2,6-dichloropyridin-3-yl)propanamide.

¹H-NMR (400 MHz, DMSO-d6): δ=1.47 (d, 3H); 4.27 (q, 1H); 7.61 (d, 1H); 8.22 (d, 1H); 10.08 (bs, 1H).

### Intermediate 2

### N-(2,6-Dichloropyridin-3-yl)-D-alaninamide hydrochloride

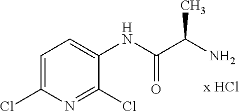

Under argon, a solution of 10.0 g of Intermediate 1 in 150 ml of THF was admixed gradually at RT with 50 ml of a solution of trimethylphosphine (1M in THF). The mixture was stirred at RT for 14 hours and then water was added. The mixture was concentrated fully under reduced pressure and the residue was taken up in water. The aqueous solution was extracted twice with dichloromethane and the combined organic phases were dried over sodium sulphate and concentrated fully under reduced pressure. The residue was taken up in diethyl ether and admixed with a solution of hydrogen chloride in diethyl ether (1M). The crystals which formed were filtered off with suction and dried in a drying cabinet under reduced pressure. This gave 11.4 g of N-(2,6-dichloropyridin-3-yl)-D-alaninamide hydrochloride. The product was converted further without further purification.

¹H NMR (400 MHz, DMSO-d6): δ=1.50 (d, 3H); 4.23 (bq, 1H); 7.63 (d, 1H); 8.15 (d, 1H); 8.42 bs, 3H); 10.58 (s, 1H).

### Intermediate 3

### N²-Cyclopentyl-N-(2,6-dichloropyridin-3-yl)-D-alaninamide

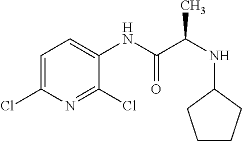 [SMILES: C[C@@H](NC1CCCC1)C(=O)Nc1ccc(Cl)nc1Cl]

Under argon, a solution of 10 g of Intermediate 2, 4.04 g of cyclopentanone and 6.06 g of sodium acetate in 400 ml of dichloromethane was admixed at 0° C. with 23.5 g of sodium triacetoxyborohydride. After 24 hours, the mixture was poured cautiously onto saturated sodium hydrogencarbonate solution, the phases were separated and the aqueous phase was extracted with dichloromethane. The combined organic phases were dried over sodium sulphate and concentrated under reduced pressure. The residue was purified by chromatography on silica gel (hexane/ethyl acetate gradient). This gave 8.4 g of N²-cyclopentyl-N-(2,6-dichloropyridin-3-yl)-D-alaninamide.

¹H-NMR (400 MHz, DMSO-d6): δ=1.27 (d, 3H); 1.31-1.41 (m, 2H); 1.42-1.55 (m, 2H); 1.59-1.73 (m, 3H); 1.73-1.83 (m, 1H); 3.06 (qi, 1H); 3.27 (q, 1H); 7.58 (d, 1H); 8.67 (d, 1H).

### Intermediate 4

### (3R)-6-Chloro-4-cyclopentyl-3-methyl-3,4-dihydropyrido[2,3-b]pyrazin-2(1H)-one

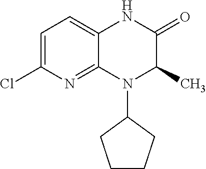 [SMILES: C[C@@H]1C(=O)Nc2ccc(Cl)nc2N1C1CCCC1]

A solution of 8.4 g of Intermediate 3 and 37.8 ml of N,N-diisopropylethylamine in 200 ml of DMF was stirred at bath temperature 170° C. for 96 hours. After cooling, the mixture was diluted with water and extracted three times with dichloromethane. The combined organic phases were concentrated under reduced pressure. Toluene was added, and the mixture was concentrated fully under reduced pressure once more. The residue was purified by chromatography on silica gel (hexane/ethyl acetate gradient). This gave 6.7 g of (3R)-6-chloro-4-cyclopentyl-3-methyl-3,4-dihydropyrido[2,3-b]pyrazin-2(1H)-one.

¹H-NMR (400 MHz, DMSO-d6): δ=1.15 (d, 3H); 1.47-1.83 (sm, 6H); 1.84-1.98 (m, 2H); 4.12 (q, 1H); 4.19 (qi, 1H); 6.67 (d, 1H); 7.00 (d, 1H); 10.61 (s, 1H).

### Intermediate 5

### (3R)-6-Chloro-4-cyclopentyl-1,3-dimethyl-3,4-dihydropyrido[2,3-b]pyrazin-2(1H)-one

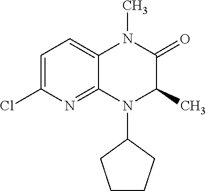 [SMILES: C[C@@H]1C(=O)N(C)c2ccc(Cl)nc2N1C1CCCC1]

A solution of 6.7 g of Intermediate 4 and 2.35 ml of methyl iodide in 180 ml of DMF was admixed at 0° C. with 1.51 g of sodium hydride (60% in white oil) in portions. After stirring at 0° C. for 1 hour, the mixture was poured onto ice-water and neutralized with saturated aqueous ammonium chloride solution. The mixture was extracted three times with ethyl acetate and the combined organic phases were washed with water, dried over sodium sulphate and concentrated fully under reduced pressure. The residue was purified by chromatography on silica gel (hexane/ethyl acetate 2:1). This gave 7.1 g of (3R)-6-chloro-4-cyclopentyl-1,3-dimethyl-3,4-dihydropyrido[2,3-b]pyrazin-2(1H)-one.

¹H-NMR (400 MHz, DMSO-d6): δ=1.11 (d, 3H); 1.48-1.62 (m, 2H); 1.63-1.82 (m, 4H); 1.87-1.98 (m, 2H); 3.23 (s, 3H); 4.21 (qi, 1H); 4.27 (q, 1H); 6.78 (d, 1H); 7.31 (d, 1H).

### Intermediate 6

### Methyl 3-{[(3R)-4-cyclopentyl-1,3-dimethyl-2-oxo-1,2,3,4-tetrahydropyrido[2,3-b]pyrazin-6-yl]amino}benzoate

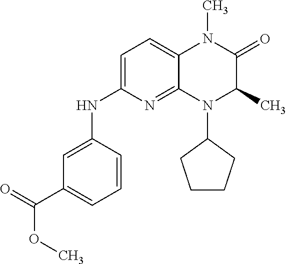 [SMILES: COC(=O)c1cccc(Nc2ccc3c(n2)N(C2CCCC2)[C@H](C)C(=O)N3C)c1]

A suspension of 900 mg of Intermediate 5, 923 mg of methyl 3-aminobenzoate (CAS 4518-10-9), 137 mg of palladium acetate, 4.98 g of caesium carbonate and 380 mg of (+)-BINAP in 68 ml of toluene was stirred at 110° C. under argon for 3.5 hours. The reaction solution was filtered, the residue was washed with ethyl acetate and the combined organic phases were concentrated fully under reduced pressure. The residue was purified by chromatography on silica gel (hexane/ethyl acetate gradient). This gave 850 mg of methyl 3-{[(3R)-4-cyclopentyl-1,3-dimethyl-2-oxo-1,2,3,4-tetrahydropyrido[2,3-b]pyrazin-6-yl]amino}benzoate.

¹H-NMR (400 MHz, CDCl₃): δ=1.22 (d, 3H); 1.57-1.85 (m, 6H); 1.99-2.14 (m, 2H); 3.31 (s, 3H); 3.92 (s, 3H); 4.29 (q, 1H); 4.51 (qi, 1H); 6.25 (d, 1H); 6.32 (s, 1H); 7.01 (d, 1H); 7.35 (t, 1H); 7.54 (d, 1H); 7.63 (d, 1H); 8.09 (s, 1H).

### Intermediate 7

### 3-{[(3R)-4-Cyclopentyl-1,3-dimethyl-2-oxo-1,2,3,4-tetrahydropyrido[2,3-b]pyrazin-6-yl]amino}benzoic acid

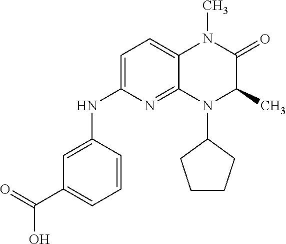 [SMILES: C[C@@H]1C(=O)N(C)c2ccc(Nc3cccc(C(=O)O)c3)nc2N1C1CCCC1]

A solution of 820 mg of Intermediate 6 in 6.5 ml of THF and 49 ml of methanol was admixed at RT with 21 ml of 1N lithium hydroxide solution and stirred at 60° C. for 5.5 hours. The mixture was added to water and extracted with ethyl acetate. The aqueous phase was adjusted to pH<3 with hydrochloric acid and extracted three times with ethyl acetate. The combined organic phases were dried over sodium sulphate and the solvent was removed completely under reduced pressure. This gave 820 mg of 3-{[(3R)-4-cyclopentyl-1,3-dimethyl-2-oxo-1,2,3,4-tetrahydropyrido[2,3-b]pyrazin-6-yl]amino}benzoic acid.

¹H NMR (400 MHz, DMSO-d6): δ=1.06 (d, 3H); 1.45-1.75 (m, 6H); 1.92-2.09 (m, 2H); 3.20 (s, 3H); 4.19 (q, 1H); 4.54 (qi, 1H); 6.24 (d, 1H); 7.25 (d, 1H); 7.31 (t, 1H); 7.39 (d, 1H); 7.64 (dd, 1H); 8.47 (s, 1H); 8.98 (s, 1H); 12.65 (bs, 1H).

### Intermediate 8

### N-(2,6-Dichloropyridin-3-yl)-N²-(tetrahydro-2H-pyran-4-yl)-D-alaninamide

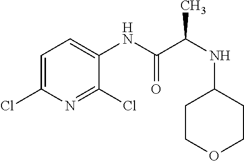 [SMILES: C[C@@H](NC1CCOCC1)C(=O)Nc1ccc(Cl)nc1Cl]

In analogy to the preparation of Intermediate 3, N-(2,6-dichloropyridin-3-yl)-N²-(tetrahydro-2H-pyran-4-yl)-D-alaninamide was prepared proceeding from 8 g of Intermediate 2, 3.85 mg of tetrahydro-2H-pyran-4-one, 4.81 g of sodium acetate and 18.8 g of sodium triacetoxyborohydride in 426 ml of dichloromethane at 0° C. This gave 8.7 g of N-(2,6-dichloropyridin-3-yl)-N²-(tetrahydro-2H-pyran-4-yl)-D-alaninamide. This was used as the crude product in the synthesis of Intermediate 9.

### Larger Reaction:

At 0° C., 12.1 g of sodium acetate and 47 g of sodium triacetoxyborohydride were added to a suspension of 20 g of Intermediate 2 and 9.6 g tetrahydro-4H-pyran-4-one in 1.07 l of dichloromethane. The mixture was stirred for 16 hours while warming to RT. The reaction was poured carefully into a saturated sodium bicarbonate solution and stirred. The phases were separated and the aqueous phase was extracted once with dichloromethane. The combined organic phases were dried over sodium sulphate and the solvent was removed completely under reduced pressure. The residue was purified by chromatography on silica gel (hexane/ethyl acetate gradient). This gave 15 g of N-(2,6-dichloropyridin-3-yl)-N²-(tetrahydro-2H-pyran-4-yl)-D-alaninamide.

¹H-NMR (400 MHz, CDCl₃): δ=1.37-1.55 (m+d, 5H); 1.81-1.89 (m, 1H); 1.91-1.99 (m, 1H); 2.67-2.76 (m, 1H); 3.38 (dt, 2H); 3.45 (q, 1H); 3.95-4.05 (m, 2H); 7.29 (d, 1H); 8.85 (d, 1H); 10.33 (s, 1H).

### Intermediate 9

### (3R)-6-Chloro-3-methyl-4-(tetrahydro-2H-pyran-4-yl)-3,4-dihydropyrido[2,3-b]pyrazin-2(1H)-one

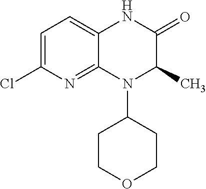 [SMILES: C[C@@H]1C(=O)Nc2ccc(Cl)nc2N1C1CCOCC1]

In analogy to the synthesis of Intermediate 4, proceeding from 5 g of Intermediate 8 and 40 ml of N,N-diisopropylethylamine in 242 ml of DMF, after 45 hours at bath temperature 170° C., (3R)-6-chloro-3-methyl-4-(tetrahydro-2H-pyran-4-yl)-3,4-dihydropyrido[2,3-b]pyrazin-2(1H)-one was prepared. This gave 2.33 g.

### Larger Reaction:

A solution of 7.8 g of Intermediate 8 and 31.7 ml of N,N-diisopropylethylamine in 170 ml of DMF was divided into 4 individual sealed pressure vessels and heated at a bath temperature of 175° C. for 10 hours. After cooling to RT, the solutions were re-combined, diluted with ethyl acetate and extracted three times with semisaturated sodium chloride solution. The organic phase was dried over sodium sulphate and the solvent was removed completely under reduced pressure. The residue was purified by chromatography on silica gel (dichloromethane/methanol gradient). This gave 4.1 g of (3R)-6-chloro-3-methyl-4-(tetrahydro-2H-pyran-4-yl)-3,4-dihydropyrido[2,3-b]pyrazin-2(1H)-one.

¹H-NMR (300 MHz, CDCl₃): δ=1.32 (d, 3H); 1.60-1.70 (m, 1H); 1.74-1.90 (m, 1H); 1.90-2.02 (m, 1H); 2.12-2.22 (m, 1H); 3.50-3.65 (m, 2H); 4.02-4.14 (m, 2H); 4.25 (q, 1H); 4.56 (tt, 1H); 6.65 (d, 1H); 6.91 (d, 1H); 8.68 (s, 1H).

### Intermediate 10

### (3R)-6-Chloro-1,3-dimethyl-4-(tetrahydro-2H-pyran-4-yl)-3,4-dihydropyrido[2,3-b]pyrazin-2(1H)-one

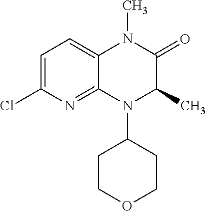 [SMILES: C[C@@H]1C(=O)N(C)c2ccc(Cl)nc2N1C1CCOCC1]

In analogy to the preparation of Intermediate 5, (3R)-6-chloro-1,3-dimethyl-4-(tetrahydro-2H-pyran-4-yl)-3,4-dihydropyrido[2,3-b]pyrazin-2(1H)-one was prepared proceeding from 2.3 g of Intermediate 9, 465 mg of sodium hydride (60% in white oil) and 0.73 ml of methyl iodide in 98 ml of DMF. Chromatography on silica gel (dichloromethane/methanol gradient) gave 2.3 g of (3R)-6-chloro-1,3-dimethyl-4-(tetrahydro-2H-pyran-4-yl)-3,4-dihydropyrido[2,3-b]pyrazin-2(1H)-one.

### Larger Reaction:

A solution of 3.2 g of Intermediate 9, 647 mg of sodium hydride (60% in white oil) and 1.01 ml of methyl iodide in 137 ml of DMF was stirred at RT for 16 hours. The reaction was poured into water and extracted three times with ethyl acetate. The combined organic phases were washed with saturated ammonium chloride solution and semisaturated sodium chloride solution and dried over sodium sulphate, and the solvent was removed completely under reduced pressure. This gave 2.8 g of (3R)-6-chloro-1,3-dimethyl-4-(tetrahydro-2H-pyran-4-yl)-3,4-dihydropyrido[2,3-b]pyrazin-2(1H)-one. This compound generally had an enantiomeric purity of >90% ee, but was purifiable further by chiral preparative HPLC.

¹H NMR (400 MHz, CDCl₃): δ=1.23 (d, 3H); 1.62-1.69 (m, 1H); 1.81 (dq, 1H); 1.96 (dq, 1H); 2.02-2.09 (m, 1H); 3.31 (s, 1H); 3.51-3.62 (m, 2H); 4.02-4.10 (m, 2H); 4.31 (q, 1H); 4.54 (tt, 1H); 6.70 (d, 1H); 7.00 (d, 1H).

Instrument: Agilent Prep 1200; column: Chiralpak IC 5 μm 250×30 mm; eluent: hexane/2-propanol 70:30 (v/v); flow rate 35 ml/min; temperature: 25° C.; detector: DAD 996 scan: 280 nm.

Rt=12.3-13.8 min

### Intermediate 11

### N-(5-Brom o-2-nitrophenyl)-D-alanine

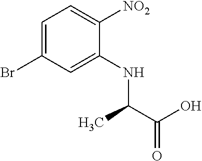 [SMILES: C[C@@H](Nc1cc(Br)ccc1[N+](=O)[O-])C(=O)O]

A solution of 13.57 g of 4-bromo-2-fluoronitrobenzene, 5.49 g of D-alanine and 10.66 g of potassium carbonate in 150 ml of ethanol and 60 ml of water was heated under reflux for 6 hours. After cooling to room temperature, the reaction mixture was acidified by addition of 1 M hydrochloric acid and the product formed was filtered off as a precipitate. This gave 17.36 g of N-(5-bromo-2-nitrophenyl)-D-alanine.

¹H-NMR (400 MHz, CDCl₃): δ=1.46 (d, 3H); 4.52-4.62 (m, 1H); 6.89 (dd, 1H); 7.22 (d, 1H); 8.01 (d, 1H); 8.38 (d, 1H).

### Intermediate 12

### (3R)-6-Bromo-3-methyl-3,4-dihydroquinoxalin-2(1H)-one

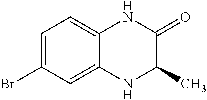

To a solution of 5.19 g of Intermediate 11 and 4.96 g of potassium carbonate in 150 ml of water was added dropwise a solution of 9.37 g of sodium dithionite in 50 ml of water at RT over the course of 30 minutes. After a further 30 minutes at RT, the reaction mixture was acidified by addition of 2 M hydrochloric acid and stirred briefly. The mixture was neutralized with potassium carbonate and extracted with dichloromethane. The organic phase was dried over sodium sulphate and concentrated fully under reduced pressure. This gave 1.88 g of (3R)-6-bromo-3-methyl-3,4-dihydroquinoxalin-2(1H)-one.

¹H-NMR (400 MHz, CDCl₃): δ=1.47 (d, 3H); 3.90 (bs, 1H); 4.03 (q, 1H); 6.62 (d, 1H); 6.82 (d, 1H); 6.87 (dd, 1H); 8.68 (bs, 1H).

### Intermediate 13

### (3R)-6-Bromo-3-methyl-4-(tetrahydro-2H-pyran-4-yl)-3,4-dihydroquinoxalin-2(1H)-one

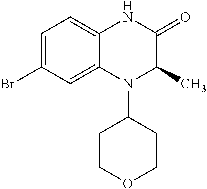 [SMILES: C[C@@H]1C(=O)Nc2ccc(Br)cc2N1C1CCOCC1]

A solution of 1.54 g of Intermediate 12, 1.9 g of tetrahydro-4H-pyran-4-one, 2.1 g of phenylsilane and 1.94 g of dibutyltin dichloride in 40 ml of THF was stirred at RT for 96 hours. The solution was concentrated fully under reduced pressure. The residue was purified by chromatography on silica gel (hexane/ethyl acetate gradient). This gave 1.97 g of (3R)-6-bromo-3-methyl-4-(tetrahydro-2H-pyran-4-yl)-3,4-dihydroquinoxalin-2(1H)-one.

¹H-NMR (400 MHz, CDCl₃): δ=1.18 (d, 3H); 1.62-1.71 (m, 1H); 1.78-2.00 (m, 3H); 3.41-3.56 (m, 2H); 3.62 (tt, 1H); 4.00-4.17 (m, 3H); 6.71 (d, 1H); 6.94 (dd, 1H); 6.98 (d, 1H); 9.50 (s, 1H).

### Intermediate 14

### (3R)-6-Bromo-1,3-dimethyl-4-(tetrahydro-2H-pyran-4-yl)-3,4-dihydroquinoxalin-2(1H)-one

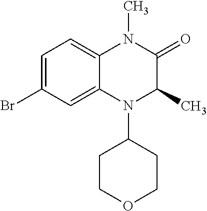 [SMILES: C[C@@H]1C(=O)N(C)c2ccc(Br)cc2N1C1CCOCC1]

In analogy to the preparation of Intermediate 5, (3R)-6-bromo-1,3-dimethyl-4-(tetrahydro-2H-pyran-4-yl)-3,4-dihydroquinoxalin-2(1H)-one was prepared proceeding from 1.97 g of Intermediate 13, 363 mg of sodium hydride (60% in white oil) and 0.57 ml of methyl iodide in 35 ml of DMF. Chromatography on silica gel (hexane/ethyl acetate gradient) gave 1.54 g of (3R)-6-bromo-1,3-dimethyl-4-(tetrahydro-2H-pyran-4-yl)-3,4-dihydroquinoxalin-2(1H)-one.

¹H-NMR (400 MHz, CDCl₃): δ=1.10 (d, 3H); 1.62-1.73 (m, 1H); 1.74-2.00 (m, 3H); 3.35 (s, 3H); 3.41-3.57 (m, 2H); 3.61 (tt, 1H); 4.00-4.20 (m, 3H); 6.81 (d, 1H); 6.97 (d, 1H); 7.01 (dd, 1H).

### Intermediate 15

### (3R)-6-Bromo-4-(4-methoxybenzyl)-3-methyl-3,4-dihydroquinoxalin-2(1H)-one

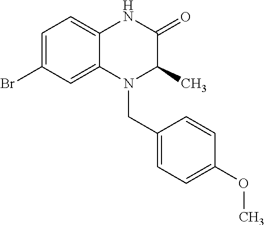 [SMILES: COc1ccc(CN2c3cc(Br)ccc3NC(=O)[C@H]2C)cc1]

A solution of 1.54 g of Intermediate 12, 2.59 g of 4-methoxybenzaldehyde, 2.06 g of phenylsilane and 1.93 g of dibutyltin dichloride in 40 ml of THF was stirred at RT for 96 hours. The solution was concentrated fully under reduced pressure. The residue was purified by chromatography on silica gel (hexane/ethyl acetate gradient). This gave 2.06 g of (3R)-6-bromo-4-(4-methoxybenzyl)-3-methyl-3,4-dihydroquinoxalin-2(1H)-one.

¹H-NMR (400 MHz, CDCl₃): δ=1.18 (d, 3H); 3.82 (s, 3H); 3.90 (q, 1H); 4.09 (d, 1H); 4.51 (d, 1H); 6.65 (d, 1H); 6.85-6.95 (m, 4H); 7.24 (d, 2H); 9.00 (bs, 1H).

### Intermediate 16

### (3R)-6-Bromo-4-(4-methoxybenzyl)-1,3-dimethyl-3,4-dihydroquinoxalin-2(1H)-one

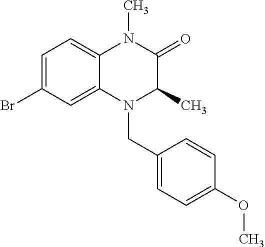 [SMILES: COc1ccc(CN2c3cc(Br)ccc3N(C)C(=O)[C@H]2C)cc1]

In analogy to the preparation of Intermediate 5, (3R)-6-bromo-4-(4-methoxybenzyl)-1,3-dimethyl-3,4-dihydroquinoxalin-2(1H)-one was prepared proceeding from 2.03 g of Intermediate 15, 337 mg of sodium hydride (60% in white oil) and 0.52 ml of methyl iodide in 35 ml of DMF. Chromatography on silica gel (hexane/ethyl acetate gradient) gave 1.34 g of (3R)-6-bromo-4-(4-methoxybenzyl)-1,3-dimethyl-3,4-dihydroquinoxalin-2(1H)-one.

¹H-NMR (400 MHz, CDCl₃): δ=0.99 (d, 3H); 3.26 (s, 3H); 3.74 (s, 3H); 3.90 (q, 1H); 4.15 (d, 1H); 4.50 (d, 1H); 6.88 (bs, 1H); 6.91 (d, 2H); 6.96-7.01 (m, 2H); 7.27 (d, 2H).

### Intermediate 17

### Methyl 3-{[(3R)-1,3-dimethyl-2-oxo-4-(tetrahydro-2H-pyran-4-yl)-1,2,3,4-tetrahydropyrido[2,3-b]pyrazin-6-yl]amino}benzoate

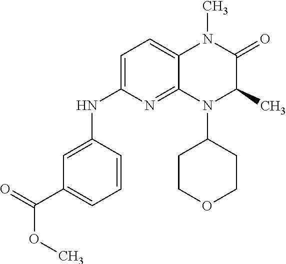 [SMILES: COC(=O)c1cccc(Nc2ccc3c(n2)N(C2CCOCC2)[C@H](C)C(=O)N3C)c1]

In analogy to the preparation of Intermediate 6, methyl 3-{[(3R)-1,3-dimethyl-2-oxo-4-(tetrahydro-2H-pyran-4-yl)-1,2,3,4-tetrahydropyrido[2,3-b]pyrazin-6-yl]amino}benzoate was prepared proceeding from 1 g of Intermediate 10 and 971 mg of methyl 3-aminobenzoate. Chromatography on silica gel (hexane/ethyl acetate gradient) gave 280 mg of methyl 3-{[(3R)-1,3-dimethyl-2-oxo-4-(tetrahydro-2H-pyran-4-yl)-1,2,3,4-tetrahydropyrido[2,3-b]pyrazin-6-yl]amino}benzoate. This reaction was conducted twice.

¹H-NMR (400 MHz, CDCl₃): δ=1.25 (d, 3H); 1.68 (bd, 1H); 1.82 (dq, 1H); 1.99 (dq, 1H); 2.05-2.15 (m, 1H); 3.32 (s, 3H); 3.50-3.65 (m, 2H); 3.92 (s, 3H); 4.01-4.13 (m, 2H); 4.31 (q, 1H); 4.59 (bs, 1H); 6.26 (d, 1H); 6.32 (bs, 1H); 7.06 (bd, 1H); 7.37 (t, 1H); 7.58 (d, 1H); 7.66 (bs, 1H); 8.05 (s, 1H).

### Intermediate 18

### 3-{[(3R)-1,3-Dimethyl-2-oxo-4-(tetrahydro-2H-pyran-4-yl)-1,2,3,4-tetrahydropyrido[2,3-b]pyrazin-6-yl]amino}benzoic acid

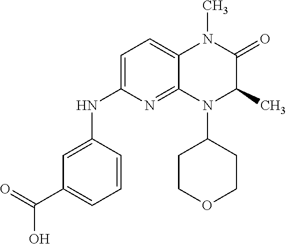 [SMILES: C[C@@H]1C(=O)N(C)c2ccc(Nc3cccc(C(=O)O)c3)nc2N1C1CCOCC1]

In analogy to the preparation of Intermediate 7, 3-{[(3R)-1,3-dimethyl-2-oxo-4-(tetrahydro-2H-pyran-4-yl)-1,2,3,4-tetrahydropyrido[2,3-b]pyrazin-6-yl]amino}benzoic acid was prepared proceeding from 500 mg of methyl 3-{[(3R)-1,3-dimethyl-2-oxo-4-(tetrahydro-2H-pyran-4-yl)-1,2,3,4-tetrahydropyrido[2,3-b]pyrazin-6-yl]amino}benzoate (prepared as described for Intermediate 17) and 277 mg of lithium hydroxide. This gave 370 mg of 3-{[(3R)-1,3-dimethyl-2-oxo-4-(tetrahydro-2H-pyran-4-yl)-1,2,3,4-tetrahydropyrido[2,3-b]pyrazin-6-yl]amino}benzoic acid.

¹H-NMR (400 MHz, DMSO-d6): δ=1.08 (d, 3H); 1.53-1.63 (m, 1H); 1.72 (dq, 1H); 1.87 (dq, 1H); 1.89-1.99 (m, 1H); 3.21 (s, 3H); 3.44 (dt, 1H); 3.52 (dt, 1H); 3.86-3.97 (m, 2H); 4.23 (q, 1H); 4.51 (tt, 1H); 6.25 (d, 1H); 7.27 (d, 1H); 7.33 (t, 1H); 7.41 (d, 1H); 7.74 (bd, 1H); 8.29 (t, 1H); 9.01 (s, 1H); 12.79 (bs, 1H).

### Intermediate 19

### Methyl 3-{[(3R)-1,3-dimethyl-2-oxo-4-(tetrahydro-2H-pyran-4-yl)-1,2,3,4-tetrahydropyrido[2,3-b]pyrazin-6-yl]amino}-4-methoxybenzoate

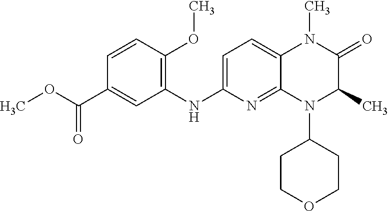

In analogy to the preparation of Intermediate 6, methyl 3-{[(3R)-1,3-dimethyl-2-oxo-4-(tetrahydro-2H-pyran-4-yl)-1,2,3,4-tetrahydropyrido[2,3-b]pyrazin-6-yl]amino}-4-methoxybenzoate was prepared proceeding from 1 g of Intermediate 10 and 1.16 g of methyl 3-amino-4-methoxybenzoate (CAS 24812-90-6). Chromatography on silica gel (hexane/ethyl acetate gradient) gave 950 mg of methyl 3-{[(3R)-1,3-dimethyl-2-oxo-4-(tetrahydro-2H-pyran-4-yl)-1,2,3,4-tetrahydropyrido[2,3-b]pyrazin-6-yl]amino}-4-methoxybenzoate.

¹H-NMR (400 MHz, CDCl₃): δ=1.23 (d, 3H); 1.62-1.72 (m, 1H); 1.79 (dq, 1H); 1.94 (dq, 1H); 2.07-2.14 (m, 1H); 3.32 (s, 3H); 3.57 (dt, 1H); 3.63 (dt, 1H); 3.89 (s, 3H); 3.97 (s, 3H); 3.97-4.06 (m, 2H); 4.31 (q, 1H); 4.71 (tt, 1H); 6.26 (d, 1H); 6.76 (s, 1H); 6.90 (d, 1H); 7.06 (d, 1H); 7.63 (dd, 1H); 8.74 (d, 1H).

### Intermediate 20

### 3-{[(3R)-1,3-Dimethyl-2-oxo-4-(tetrahydro-2H-pyran-4-yl)-1,2,3,4-tetrahydropyrido[2,3-b]pyrazin-6-yl]amino}-4-methoxybenzoic acid

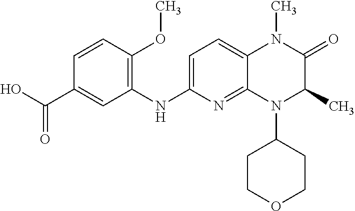 [SMILES: COc1ccc(C(=O)O)cc1Nc1ccc2c(n1)N(C1CCOCC1)[C@H](C)C(=O)N2C]

In analogy to the preparation of Intermediate 7, 3-{[(3R)-1,3-dimethyl-2-oxo-4-(tetrahydro-2H-pyran-4-yl)-1,2,3,4-tetrahydropyrido[2,3-b]pyrazin-6-yl]amino}-4-methoxybenzoic acid was prepared proceeding from 930 mg of Intermediate 19 and 480 mg of lithium hydroxide. This gave 520 mg of 3-{[(3R)-1,3-dimethyl-2-oxo-4-(tetrahydro-2H-pyran-4-yl)-1,2,3,4-tetrahydropyrido[2,3-b]pyrazin-6-yl]amino}-4-methoxybenzoic acid.

¹H-NMR (400 MHz, CDCl₃): δ=1.31 (d, 3H); 1.64-1.72 (m, 1H); 1.84 (dq, 1H); 1.98 (dq, 1H); 2.25-2.33 (m, 1H); 3.34 (s, 3H); 3.69 (dt, 1H); 3.88 (dt, 1H); 3.99 (s, 3H); 3.99-4.14 (m, 2H); 4.36 (q, 1H); 4.94-5.05 (m, 1H); 6.33 (d, 1H); 7.01 (d, 1H); 7.24 (d, 1H); 7.92 (d, 1H); 8.26 (bs, 1H).

### Intermediate 21

### N-(2,6-Difluorobenzyl)alanine methyl ester

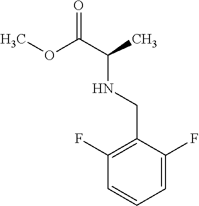 [SMILES: COC(=O)[C@@H](C)NCc1c(F)cccc1F]

A solution of 2.9 g of 2,6-difluorobenzaldehyde, 3.35 g of D-alanine methyl ester hydrochloride and 3.3 ml of triethylamine in 100 ml of dichloromethane was admixed at RT with 8.5 g of sodium triacetoxyborohydride. The mixture was stirred for 30 minutes and then 2.3 ml of glacial acetic acid were added gradually. The mixture was stirred overnight and then sodium hydrogencarbonate solution was added. The organic phase was removed and dried over sodium sulphate, and the solvent was removed under reduced pressure. This gave 4.7 g of N-(2,6-difluorobenzyl)alanine methyl ester.

UPLC-MS: Instrument: Waters Acquity UPLC-MS SQD; column: Acquity UPLC BEH C18 1.7 50×2.1 mm; eluent A: water+0.1% by vol. of formic acid (99%), eluent B: acetonitrile; gradient: 0-1.6 min 1-99% B, 1.6-2.0 min 99% B; flow rate 0.8 ml/min; temperature: 60° C.; injection: 2 μl;

DAD scan: 210-400 nm.

Rt=0.53 min.

### Intermediate 22

### 6-Bromo-4-(2,6-difluorobenzyl)-3-methyl-3,4-dihydroquinoxalin-2(1H)-one

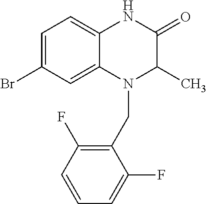 [SMILES: CC1C(=O)Nc2ccc(Br)cc2N1Cc1c(F)cccc1F]

A mixture of 2.3 g of N-(2,6-difluorobenzyl)alanine methyl ester (Intermediate 21), 2 g of 4-bromo-2-fluoronitrobenzene and 1.53 g of potassium carbonate in 20 ml of ethanol and 8 ml of water was stirred at 100° C. for 6 hours. The mixture was stirred at RT for a further 72 hours and then diluted with water. 1 N hydrochloric acid was added until the pH of the mixture was <7. The precipitate formed was filtered off with suction. The reaction was repeated on the same scale and a total of 4.7 g of N-(5-bromo-2-nitrophenyl)-N-(2,6-difluorobenzyl)alanine were obtained. Of this, 2.2 g in 12 ml of methanol and 12 ml of glacial acetic acid were admixed with 1.04 g of iron powder and stirred at 105° C. for 2 hours. This reaction was repeated with a further 2.4 g of N-(5-bromo-2-nitrophenyl)-N-(2,6-difluorobenzyl)alanine and 1.13 g of iron powder. On completion of reaction, the two batches were combined. The mixture was filtered, saturated sodium hydrogencarbonate solution was added to the filtrate and the filtrate was extracted with dichloromethane. The organic phase was concentrated under reduced pressure and the residue was purified by chromatography on silica gel (dichloromethane/methanol gradient). This gave 970 mg of 6-bromo-4-(2,6-difluorobenzyl)-3-methyl-3,4-dihydroquinoxalin-2(1H)-one.

¹H NMR (300 MHz, DMSO-d₆) δ=1.07 (d, 3H); 3.73 (d, 1H); 4.31 (s, 1H); 4.26 (s, 1H); 4.69 (s, 1H); 4.64 (s, 1H); 6.72 (d, 1H); 6.88 (dd, 1H); 7.03 (d, 1H); 7.09-7.22 (m, 2H); 7.36-7.52 (m, 1H); 10.51 (s, 1H).

### Intermediate 23

### 6-Bromo-4-(2,6-difluorobenzyl)-1,3-dimethyl-3,4-dihydroquinoxalin-2(1H)-one

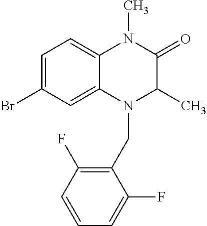 [SMILES: CC1C(=O)N(C)c2ccc(Br)cc2N1Cc1c(F)cccc1F]

In analogy to the preparation of Intermediate 5, 6-bromo-4-(2,6-difluorobenzyl)-1,3-dimethyl-3,4-dihydroquinoxalin-2(1H)-one was prepared proceeding from 970 mg of Intermediate 22, 170 mg of sodium hydride (60% in white oil) and 0.24 ml of methyl iodide in 15 ml of DMF. After extractive workup, 1.15 g of (3R)-6-bromo-4-(4-methoxybenzyl)-1,3-dimethyl-3,4-dihydroquinoxalin-2(1H)-one were obtained as crude product.

UPLC-MS: Instrument: Waters Acquity UPLC-MS SQD; column: Acquity UPLC BEH C18 1.7 50×2.1 mm; eluent A: water+0.1% by vol. of formic acid (99%), eluent B: acetonitrile; gradient: 0-1.6 min 1-99% B, 1.6-2.0 min 99% B; flow rate 0.8 ml/min; temperature: 60° C.; injection: 2 μl;

DAD scan: 210-400 nm.

Rt=1.36 min.

### Intermediate 24

### Ethyl 3-{[4-(2,6-difluorobenzyl)-1,3-dimethyl-2-oxo-1,2,3,4-tetrahydroquinoxalin-6-yl]amino}benzoate

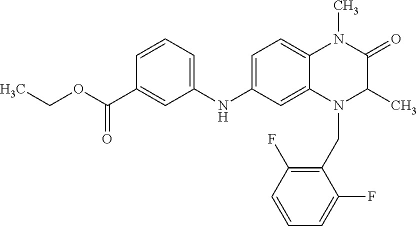 [SMILES: CCOC(=O)c1cccc(Nc2ccc3c(c2)N(Cc2c(F)cccc2F)C(C)C(=O)N3C)c1]

In analogy to the preparation of Intermediate 6, ethyl 3-{[4-(2,6-difluorobenzyl)-1,3-dimethyl-2-oxo-1,2,3,4-tetrahydroquinoxalin-6-yl]amino}benzoate was prepared proceeding from 182 mg of Intermediate 23 and 148 mg of ethyl 3-aminobenzoate. Chromatography on silica gel (hexane/ethyl acetate gradient) gave 60 mg of ethyl 3-{[4-(2,6-difluorobenzyl)-1,3-dimethyl-2-oxo-1,2,3,4-tetrahydroquinoxalin-6-yl]amino}benzoate.

UPLC-MS: Instrument: Waters Acquity UPLC-MS SQD; column: Acquity UPLC BEH C18 1.7 50×2.1 mm; eluent A: water+0.1% by vol. of formic acid (99%), eluent B: acetonitrile; gradient: 0-1.6 min 1-99% B, 1.6-2.0 min 99% B; flow rate 0.8 ml/min; temperature: 60° C.; injection: 2 μl;

DAD scan: 210-400 nm.

Rt=1.38 min.

### Intermediate 25

### 3-{[4-(2,6-Difluorobenzyl)-1,3-dimethyl-2-oxo-1,2,3,4-tetrahydroquinoxalin-6-yl]amino}benzoic acid

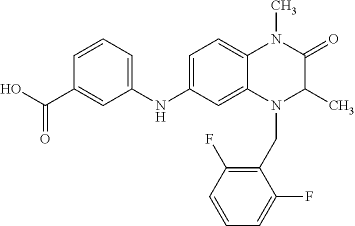 [SMILES: CC1C(=O)N(C)c2ccc(Nc3cccc(C(=O)O)c3)cc2N1Cc1c(F)cccc1F]

In analogy to the preparation of Intermediate 7, 3-{[4-(2,6-difluorobenzyl)-1,3-dimethyl-2-oxo-1,2,3,4-tetrahydroquinoxalin-6-yl]amino}benzoic acid was prepared proceeding from 60 mg of Intermediate 24 and 26 mg of sodium hydroxide. This gave 40 mg of 3-{[4-(2,6-difluorobenzyl)-1,3-dimethyl-2-oxo-1,2,3,4-tetrahydroquinoxalin-6-yl]amino}benzoic acid.

UPLC-MS: Instrument: Waters Acquity UPLC-MS SQD; column: Acquity UPLC BEH C18 1.7 50×2.1 mm; eluent A: water+0.1% by vol. of formic acid (99%), eluent B: acetonitrile; gradient: 0-1.6 min 1-99% B, 1.6-2.0 min 99% B; flow rate 0.8 ml/min; temperature: 60° C.; injection: 2 μl;

DAD scan: 210-400 nm.

Rt=1.12 min.

### Intermediate 26

### N²-Cyclohexyl-N-(2,6-dichloropyridin-3-yl)-D-alaninamide

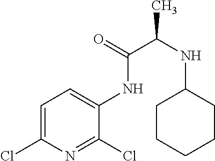 [SMILES: C[C@@H](NC1CCCCC1)C(=O)Nc1ccc(Cl)nc1Cl]

Analogously to the preparation of Intermediate 3, N²-cyclohexyl-N-(2,6-dichloropyridin-3-yl)-D-alaninamide was prepared from 1.5 g of Intermediate 2, 707 mg of cyclohexanone, 909 mg of sodium acetate and 3.5 g of sodium triacetoxyborohydride in 80 ml of dichloromethane at 0° C. This gave 1.3 g of N²-cyclohexyl-N-(2,6-dichloropyridin-3-yl)-D-alaninamide as a crude product which could be used without further purification for the next step.

UPLC-MS: Instrument: Waters Acquity UPLC-MS SQD; column: Acquity UPLC BEH C18 1.7 50×2.1 mm; eluent A: water+0.1% by vol. of formic acid (99%), eluent B: acetonitrile; gradient: 0-1.6 min 1-99% B, 1.6-2.0 min 99% B; flow rate 0.8 ml/min; temperature: 60° C.; injection: 2 μl;

DAD scan: 210-400 nm.

Rt=1.49 min (M⁺+1=316, 318, 320)

### Intermediate 27

### (3R)-6-Chloro-4-cyclohexyl-3-methyl-3,4-dihydropyrido[2,3-b]pyrazin-2(1H)-one

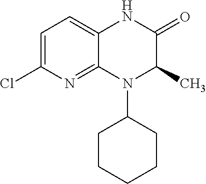 [SMILES: C[C@@H]1C(=O)Nc2ccc(Cl)nc2N1C1CCCCC1]

Analogously to the synthesis of Intermediate 4, (3R)-6-chloro-4-cyclohexyl-3-methyl-3,4-dihydropyrido[2,3-b]pyrazin-2(1H)-one was prepared from 1.3 g of Intermediate 26 and 5.59 ml of N,N-diisopropylethylamine in 100 ml of DMF by heating for 120 hours at a bath temperature of 170° C. This gave 1.08 g of (3R)-6-chloro-4-cycloxyl-3-methyl-3,4-dihydropyrido[2,3-b]pyrazin-2(1H)-one.

¹H-NMR (300 MHz, DMSO-d6): δ=1.14 (d, 3H); 1.15-1.97 (5 m, 10H); 4.03-4.13 (m, 1H); 4.15 (q, 1H); 6.65 (d, 1H); 7.00 (d, 1H); 10.58 (s, 1H).

### Intermediate 28

### (3R)-6-Chloro-4-cyclohexyl-1,3-dimethyl-3,4-dihydropyrido[2,3-b]pyrazin-2(1H)-one

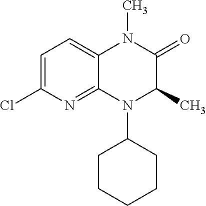 [SMILES: C[C@@H]1C(=O)N(C)c2ccc(Cl)nc2N1C1CCCCC1]

Analogously to the preparation of Intermediate 5, (3R)-6-chloro-4-cyclohexyl-1,3-dimethyl-3,4-dihydropyrido[2,3-b]pyrazin-2(1H)-one was prepared from 1.08 g of Intermediate 27, 232 mg of sodium hydride (60% in white oil) and 0.36 ml of methyl iodide in 50 ml of DMF. Purification by chromatography on silica gel (hexane/ethyl acetate 3:1) gave 1.06 g of (3R)-6-chloro-4-cyclohexyl-1,3-dimethyl-3,4-dihydropyrido[2,3-b]pyrazin-2(1H)-one.

¹H-NMR (400 MHz, DMSO-d6): δ=1.11 (d, 3H); 1.48-1.62 (m, 2H); 1.63-1.82 (m, 4H); 1.94-1.98 (m, 2H); 3.23 (s, 3H); 4.21 (qi, 1H); 4.27 (q, 1H); 6.76 (d, 1H); 7.31 (d, 1H).

### Intermediate 29

### Methyl 3-{[(3R)-4-cyclohexyl-1,3-dimethyl-2-oxo-1,2,3,4-tetrahydropyrido[2,3-b]pyrazin-6-yl]amino}benzoate

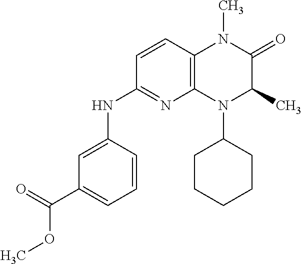 [SMILES: COC(=O)c1cccc(Nc2ccc3c(n2)N(C2CCCCC2)[C@H](C)C(=O)N3C)c1]

In analogy to the preparation of Intermediate 6, methyl 3-{[(3R)-4-cyclohexyl-1,3-dimethyl-2-oxo-1,2,3,4-tetrahydropyrido[2,3-b]pyrazin-6-yl]amino}benzoate was prepared proceeding from 1.5 g of Intermediate 28 and 1.57 g of methyl 3-aminobenzoate. Chromatography on silica gel (hexane/ethyl acetate gradient up to 50% ethyl acetate content) gave 2 g of methyl 3-{[(3R)-4-cyclohexyl-1,3-dimethyl-2-oxo-1,2,3,4-tetrahydropyrido[2,3-b]pyrazin-6-yl]amino}benzoate.

UPLC-MS: Instrument: Waters Acquity UPLC-MS SQD; column: Acquity UPLC BEH C18 1.7 50×2.1 mm; eluent A: water+0.1% by vol. of formic acid (99%), eluent B: acetonitrile; gradient: 0-1.6 min 1-99% B, 1.6-2.0 min 99% B; flow rate 0.8 ml/min; temperature: 60° C.; injection: 2 μl;

DAD scan: 210-400 nm.

Rt=1.40 min.

### Intermediate 30

### 3-{[(3R)-4-Cyclohexyl-1,3-dimethyl-2-oxo-1,2,3,4-tetrahydropyrido[2,3-b]pyrazin-6-yl]amino}benzoic acid

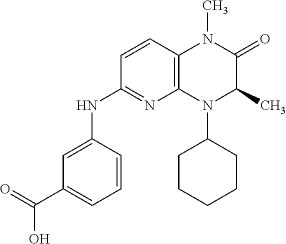 [SMILES: C[C@@H]1C(=O)N(C)c2ccc(Nc3cccc(C(=O)O)c3)nc2N1C1CCCCC1]

In analogy to the preparation of Intermediate 7, 3-{[(3R)-4-cyclohexyl-1,3-dimethyl-2-oxo-1,2,3,4-tetrahydropyrido[2,3-b]pyrazin-6-yl]amino}benzoic acid was prepared proceeding from 2.0 g of Intermediate 29 and 0.98 g of sodium hydroxide. This gave 1.78 g of 3-{[(3R)-4-cyclohexyl-1,3-dimethyl-2-oxo-1,2,3,4-tetrahydropyrido[2,3-b]pyrazin-6-yl]amino}benzoic acid.

UPLC-MS: Instrument: Waters Acquity UPLC-MS SQD; column: Acquity UPLC BEH C18 1.7 50×2.1 mm; eluent A: water+0.1% by vol. of formic acid (99%), eluent B: acetonitrile; gradient: 0-1.6 min 1-99% B, 1.6-2.0 min 99% B; flow rate 0.8 ml/min; temperature: 60° C.; injection: 2 μl;

DAD scan: 210-400 nm.

Rt=1.18 min.

### Intermediate 31

### N²-(1-Methylethyl)-N-(2,6-dichloropyridin-3-yl)-D-alaninamide

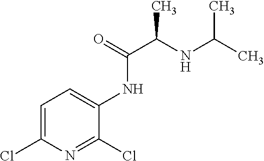 [SMILES: CC(C)N[C@H](C)C(=O)Nc1ccc(Cl)nc1Cl]

Analogously to the preparation of Intermediate 3, N2-(1-methylethyl)-N1-(2,6-dichloropyridin-3-yl)-D-alaninamide was prepared from 0.5 g of Intermediate 2, 0.27 ml of acetone, 303 mg of sodium acetate and 1.18 g of sodium triacetoxyborohydride in 40 ml of dichloromethane at 0° C. This gave 420 mg of N²-(1-methylethyl)-N-(2,6-dichloropyridin-3-yl)-D-alaninamide. This was used directly in the synthesis of the next stage.

¹H-NMR (400 MHz, DMSO-d6): δ=1.02 (d, 3H); 1.05 (d, 3H); 1.27 (d, 3H); 2.77 (sp, 1H); 3.30 (q, 1H); 7.58 (d, 1H); 8.67 (d, 1H).

### Intermediate 32

### (3R)-6-Chloro-3-methyl-4-(propan-2-yl)-3,4-dihydropyrido[2,3-b]pyrazin-2(1H)-one

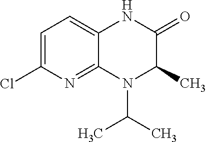 [SMILES: CC(C)N1c2nc(Cl)ccc2NC(=O)[C@H]1C]

Analogously to the synthesis of Intermediate 4, (3R)-6-chloro-3-methyl-4-(propan-2-yl)-3,4-dihydropyrido[2,3-b]pyrazin-2(1H)-one was prepared from 420 mg of Intermediate 31 and 2.1 ml of N,N-diisopropylethylamine in 40 ml of DMF by heating for 72 hours at a bath temperature of 170° C. This gave 320 mg of (3R)-6-chloro-3-methyl-4-(propan-2-yl)-3,4-dihydropyrido[2,3-b]pyrazin-2(1H)-one.

¹H-NMR (300 MHz, DMSO-d6): δ=1.16 (d, 3H); 1.24 (d, 3H); 1.27 (d, 3H); 4.16 (q, 1H); 4.43 (sp, 1H); 6.65 (d, 1H); 7.00 (d, 1H); 10.56 (s, 1H).

### Intermediate 33

### (3R)-6-Chloro-1,3-dimethyl-4-(propan-2-yl)-3,4-dihydropyrido[2,3-b]pyrazin-2(1H)-one

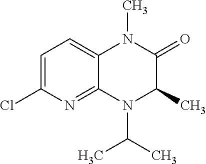 [SMILES: CC(C)N1c2nc(Cl)ccc2N(C)C(=O)[C@H]1C]

Analogously to the preparation of Intermediate 5, (3R)-6-chloro-1,3-dimethyl-4-(propan-2-yl)-3,4-dihydropyrido[2,3-b]pyrazin-2(1H)-one was prepared from 320 mg of Intermediate 32, 80 mg of sodium hydride (60% in white oil) and 0.13 ml of methyl iodide in 20 ml of DMF. Purification by chromatography on silica gel (hexane/ethyl acetate 2:1) gave 280 mg of (3R)-6-chloro-1,3-dimethyl-4-(propan-2-yl)-3,4-dihydropyrido[2,3-b]pyrazin-2(1H)-one.

¹H-NMR (400 MHz, DMSO-d6): δ=1.12 (d, 3H); 1.23 (d, 3H); 1.27 (d, 3H); 3.22 (s, 3H); 4.32 (q, 1H); 4.47 (sp, 1H); 6.76 (d, 1H); 7.31 (d, 1H).

### Intermediate 34

### Methyl 3-{[(3R)-1,3-dimethyl-2-oxo-4-(propan-2-yl)-1,2,3,4-tetrahydropyrido[2,3-b]pyrazin-6-yl]amino}benzoate

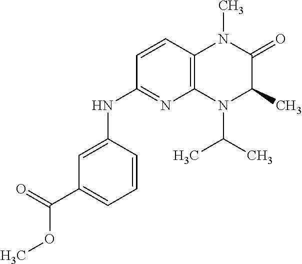 [SMILES: COC(=O)c1cccc(Nc2ccc3c(n2)N(C(C)C)[C@H](C)C(=O)N3C)c1]

In analogy to the preparation of Intermediate 6, methyl 3-{[(3R)-1,3-dimethyl-2-oxo-4-(propan-2-yl)-1,2,3,4-tetrahydropyrido[2,3-b]pyrazin-6-yl]amino}benzoate was prepared proceeding from 1.1 g of Intermediate 33 and 1.24 g of methyl 3-aminobenzoate. Chromatography on silica gel (hexane/ethyl acetate gradient up to 50% ethyl acetate content) gave 1.1 g of methyl 3-{[(3R)-1,3-dimethyl-2-oxo-4-(propan-2-yl)-1,2,3,4-tetrahydropyrido[2,3-b]pyrazin-6-yl]amino}benzoate.

¹H-NMR (400 MHz, CDCl₃): δ=1.25 (d, 3H); 1.30 (d, 3H); 1.36 (d, 3H); 3.31 (s, 3H); 3.92 (s, 3H); 4.32 (q, 1H); 4.77 (sept, 1H); 6.22 (d, 1H); 6.33 (bs, 1H); 7.02 (d, 1H); 7.35 (t, 1H); 7.50-7.56 (m, 1H); 7.64 (bd, 1H); 8.17 (bs, 1H).

### Intermediate 35

### 3-{[(3R)-1,3-Dimethyl-2-oxo-4-(propan-2-yl)-1,2,3,4-tetrahydropyrido[2,3-b]pyrazin-6-yl]amino}benzoic acid

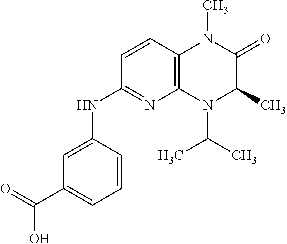 [SMILES: CC(C)N1c2nc(Nc3cccc(C(=O)O)c3)ccc2N(C)C(=O)[C@H]1C]

A solution of 1.1 g of Intermediate 34 in 9 ml of THF and 67 ml of methanol was admixed at RT with 281 ml of 1N lithium hydroxide solution and stirred at 60° C. for 4 hours. The mixture was adjusted to pH=7 using 1N hydrochloric acid and extracted twice with ethyl acetate. The combined organic phases were dried over sodium sulphate and the solvent was removed completely under reduced pressure. This gave 1.78 g of 3-{[(3R)-1,3-dimethyl-2-oxo-4-(propan-2-yl)-1,2,3,4-tetrahydropyrido[2,3-b]pyrazin-6-yl]amino}benzoic acid.

¹H-NMR (400 MHz, DMSO-d6): δ=1.09 (d, 3H); 1.23 (d, 3H); 1.30 (d, 3H); 3.20 (s, 3H); 4.24 (q, 1H)M; 4.75 (sept, 1H); 6.22 (d, 1H); 7.24 (d, 1H); 7.31 8t, 1H); 7.38 (bd, 1H); 7.64 (bd, 1H); 8.58 (t, 1H); 8.99 (s, 1H); 12.73 (bs, 1H).

### Intermediate 36

### N²-Cycloheptyl-N-(2,6-dichloropyridin-3-yl)-D-alaninamide

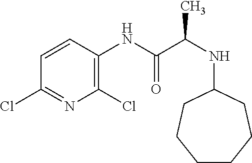 [SMILES: C[C@@H](NC1CCCCCC1)C(=O)Nc1ccc(Cl)nc1Cl]

Analogously to the preparation of Intermediate 3, N²-cycloheptyl-N-(2,6-dichloropyridin-3-yl)-D-alaninamide was prepared from 1.5 g of Intermediate 2, 809 mg of cycloheptanone, 909 mg of sodium acetate and 3.5 g of sodium triacetoxyborohydride in 80 ml of dichloromethane at 0° C. This gave 1.4 g of N²-cycloheptyl-N-(2,6-dichloropyridin-3-yl)-D-alaninamide.

¹H-NMR (400 MHz, DMSO-d6): δ=1.26 (d, 3H); 1.29-1.42 (m, 4H); 1.42-1.55 (m, 4H); 1.55-1.69 (m, 3H); 1.75-1.88 (m, 2H); 2.56-2.67 (m, 1H); 3.30 (m, 1H); 7.58 (d, 1H); 8.68 (d, 1H).

### Intermediate 37

### (3R)-6-Chloro-4-cycloheptyl-3-methyl-3,4-dihydropyrido[2,3-b]pyrazin-2(1H)-one

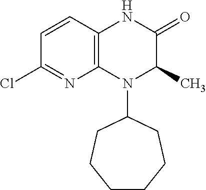 [SMILES: C[C@@H]1C(=O)Nc2ccc(Cl)nc2N1C1CCCCCC1]

Analogously to the synthesis of Intermediate 4, (3R)-6-chloro-4-cycloheptyl-3-methyl-3,4-dihydropyrido[2,3-b]pyrazin-2(1H)-one was prepared from 1.4 g of Intermediate 36 and 5.77 ml of N,N-diisopropylethylamine in 70 ml of DMF by heating for 72 hours at a bath temperature of 170° C. This gave 1.18 g of (3R)-6-chloro-4-cycloheptyl-3-methyl-3,4-dihydropyrido[2,3-b]pyrazin-2(1H)-one.

¹H-NMR (300 MHz, DMSO-d6): δ=1.16 (d, 3H); 1.37-1.63 (m, 6H); 1.63-2.00 (m, 6H); 3.96-4.09 (m, 1H); 4.17 (q, 1H); 6.64 (d, 1H); 6.98 (d, 1H); 10.57 (s, 1H).

### Intermediate 38

### (3R)-6-Chloro-4-cycloheptyl-1,3-dimethyl-3,4-dihydropyrido[2,3-b]pyrazin-2(1H)-one

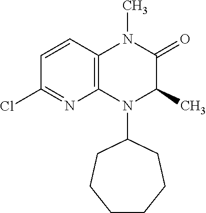 [SMILES: C[C@@H]1C(=O)N(C)c2ccc(Cl)nc2N1C1CCCCCC1]

Analogously to the preparation of Intermediate 5, (3R)-6-chloro-4-cycloheptyl-1,3-dimethyl-3,4-dihydropyrido[2,3-b]pyrazin-2(1H)-one was prepared from 1.18 g of Intermediate 37, 241 mg of sodium hydride (60% in white oil) and 0.38 ml of methyl iodide in 50 ml of DMF. Purification by chromatography on silica gel (hexane/ethyl acetate 3:1) gave 1.11 g of (3R)-6-chloro-4-cycloheptyl-1,3-dimethyl-3,4-dihydropyrido[2,3-b]pyrazin-2(1H)-one.

¹H-NMR (300 MHz, DMSO-d6): δ=1.13 (d, 3H); 1.38-1.63 (m, 6H); 1.63-1.84 (m, 4H); 1.83-2.03 (m, 2H); 3.21 (s, 3H); 4.00-4.14 (m, 1H); 4.32 (q, 1H); 6.75 (d, 1H); 7.29 (d, 1H).

### Intermediate 39

### N²-Benzyl-N-(2,6-dichloropyridin-3-yl)-D-alaninamide

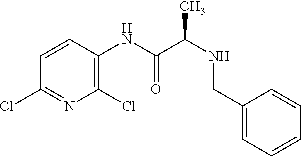 [SMILES: C[C@@H](NCc1ccccc1)C(=O)Nc1ccc(Cl)nc1Cl]

Analogously to the preparation of Intermediate 3, N²-benzyl-N-(2,6-dichloropyridin-3-yl)-D-alaninamide was prepared from 1.5 g of Intermediate 2, 765 mg of benzaldehyde, 909 mg of sodium acetate and 3.5 g of sodium triacetoxyborohydride in 80 ml of dichloromethane at 0° C. This gave 1.5 g of N²-benzyl-N-(2,6-dichloropyridin-3-yl)-D-alaninamide.

¹H NMR (400 MHz, DMSO-d6): δ=1.29 (d, 3H); 3.29 (q, 1H); 3.76 (s, 2H); 7.23 (t, 1H); 7.32 (t, 2H); 7.39 (d, 2H); 7.58 (d, 1H); 8.59 (d, 1H).

### Intermediate 40

### (3R)-4-Benzyl-6-chloro-3-methyl-3,4-dihydropyrido[2,3-b]pyrazin-2(1H)-one

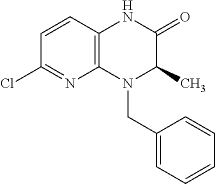 [SMILES: C[C@@H]1C(=O)Nc2ccc(Cl)nc2N1Cc1ccccc1]

Analogously to the synthesis of Intermediate 4, (3R)-4-benzyl-6-chloro-3-methyl-3,4-dihydropyrido[2,3-b]pyrazin-2(1H)-one was prepared from 1.4 g of Intermediate 39 and 5.88 ml of N,N-diisopropylethylamine in 100 ml of DMF by heating for 72 hours at a bath temperature of 170° C. This gave 1.14 g of (3R)-4-benzyl-6-chloro-3-methyl-3,4-dihydropyrido[2,3-b]pyrazin-2(1H)-one.

¹H-NMR (300 MHz, DMSO-d6): δ=1.18 (d, 3H); 3.95 (q, 1H); 4.29 (d, 1H); 5.10 (d, 1H); 6.71 (d, 1H); 7.04 (d, 1H); 7.23-7.33 (m, 1H); 7.33-7.41 (m, 4H); 10.70 (s, 1H).

### Intermediate 41

### (3R)-4-Benzyl-6-chloro-1,3-dimethyl-3,4-dihydropyrido[2,3-b]pyrazin-2(1H)-one

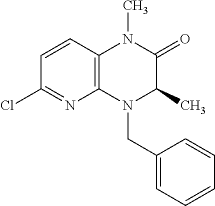 [SMILES: C[C@@H]1C(=O)N(C)c2ccc(Cl)nc2N1Cc1ccccc1]

In analogy to the preparation of Intermediate 5, (3R)-4-benzyl-6-chloro-1,3-dimethyl-3,4-dihydropyrido[2,3-b]pyrazin-2(1H)-one was prepared proceeding from 1.14 g of Intermediate 40, 238 mg of sodium hydride (60% in white oil) and 0.37 ml of methyl iodide in 50 ml of DMF. Purification by chromatography on silica gel (hexane/ethyl acetate 3:1) gave 1.15 g of (3R)-4-benzyl-6-chloro-1,3-dimethyl-3,4-dihydropyrido[2,3-b]pyrazin-2(1H)-one.

1H-NMR (300 MHz, DMSO-d6): δ=1.15 (d, 3H); 3.24 (s, 3H); 4.08 (q, 1H); 4.28 (d, 1H); 5.11 (d, 1H); 6.82 (d, 1H); 7.22-7.42 (m, 6H).

### Intermediate 42

### Methyl 3-{[(3R)-4-benzyl-1,3-dimethyl-2-oxo-1,2,3,4-tetrahydropyrido[2,3-b]pyrazin-6-yl]amino}benzoate

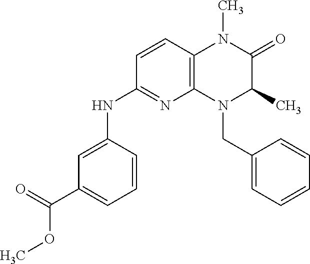 [SMILES: COC(=O)c1cccc(Nc2ccc3c(n2)N(Cc2ccccc2)[C@H](C)C(=O)N3C)c1]

A suspension of 900 mg of Intermediate 41, 857 mg of methyl 3-aminobenzoate (CAS 4518-10-9), 64 mg of palladium(II) acetate, 2.77 g of caesium carbonate and 176 mg of (+)-BINAP in 63.4 ml of toluene was stirred at 120° C. under an argon atmosphere for 14 hours. The reaction solution was added to water and extracted twice with ethyl acetate, the combined organic phases were dried over sodium sulphate, and the solvent was removed under reduced pressure. The residue was purified by chromatography on silica gel (dichloromethane/methanol gradient up to 1% methanol content). This gave 920 mg of methyl 3-{[(3R)-4-benzyl-1,3-dimethyl-2-oxo-1,2,3,4-tetrahydropyrido[2,3-b]pyrazin-6-yl]amino}benzoate.

¹H NMR: (300 MHz, 25° C., DMSO-d6): δ=1.12 (d, 3H); 3.23 (s, 3H); 3.67 (s, 3H); 3.95 (q, 1H); 4.28 (d, 1H); 5.33 (d, 1H); 6.27 (d, 1H); 7.22-7.42 (m, 8H); 7.63 (bd, 1H); 8.50 (t, 1H); 9.09 (s, 1H).

### Intermediate 43

### 3-{[(3R)-4-Benzyl-1,3-dimethyl-2-oxo-1,2,3,4-tetrahydropyrido[2,3-b]pyrazin-6-yl]amino}benzoic acid

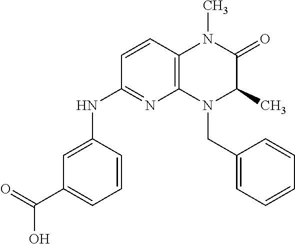 [SMILES: C[C@@H]1C(=O)N(C)c2ccc(Nc3cccc(C(=O)O)c3)nc2N1Cc1ccccc1]

A solution of 900 mg of Intermediate 42 in 6.8 ml of THF and 51 ml of methanol was admixed at RT with 22 ml of 1N lithium hydroxide solution and stirred at 60° C. for 2 hours. The mixture was adjusted to pH=7 using 1N hydrochloric acid and extracted twice with ethyl acetate. The combined organic phases were dried over sodium sulphate and the solvent was removed completely under reduced pressure. This gave 770 mg of 3-{[(3R)-4-benzyl-1,3-dimethyl-2-oxo-1,2,3,4-tetrahydropyrido[2,3-b]pyrazin-6-yl]amino}benzoic acid.

¹H-NMR (400 MHz, DMSO-d6): δ=1.12 (d, 3H); 3.23 (s, 3H); 3.98 (q, 1H); 4.27 (d, 1H); 5.31 (d, 1H); 6.27 (d, 1H); 7.18-7.40 (m, 8H); 7.67 (bd, 1H); 8.34 (t, 1H); 9.01 (s, 1H); 12.50 (bs, 1H).

### Intermediate 44

### N-(2,6-Dichloropyridin-3-yl)-2-oxopropanamide

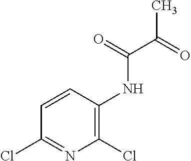

At 0° C., 14.6 ml of thionyl chloride were added slowly to a solution of 17.6 g of pyruvic acid in 150 ml of DMF. The mixture was stirred for 15 minutes, and 16.3 g of 2,6-dichloropyridine-3-amine (CAS 62476-56-6) were then added. The mixture was left stirring at RT for 16 hours and poured into 300 ml of ice-water. The precipitate was filtered off and washed with water. This gave 9.8 g of N-(2,6-dichloropyridin-3-yl)-2-oxopropanamide.

¹H-NMR (300 MHz, DMSO-d6): δ=2.44 (s, 3H); 7.65 (d, 1H); 8.28 (d, 1H); 10.03 (bs, 1H).

### Intermediate 45

### N-(2,6-Dichloropyridin-3-yl)-N²-(2-methoxyethyl)alaninamide

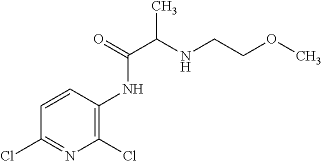 [SMILES: COCCNC(C)C(=O)Nc1ccc(Cl)nc1Cl]

At RT, 2.16 g of sodium triacetoxyborohydride were added to a solution of 1.7 g of Intermediate 44 and 603 mg of 2-methoxyethylamine in 52 ml of 1,2-dichloroethane and 0.42 ml of acetic acid. The mixture was stirred for 16 hours. The reaction was stirred into water and extracted with dichloromethane. The organic phase was washed with sodium bicarbonate solution and water and dried over sodium sulphate, and the solvent was removed under reduced pressure. This gave 2.13 g of N-(2,6-dichloropyridin-3-yl)-N²-(2-methoxyethyl)alaninamide.

UPLC-MS: Instrument: Waters Acquity UPLC-MS SQD; column: Acquity UPLC BEH C18 1.7 50×2.1 mm; eluent A: water+0.1% by vol. of formic acid (99%), eluent B: acetonitrile; gradient: 0-1.6 min 1-99% B, 1.6-2.0 min 99% B; flow rate 0.8 ml/min; temperature: 60° C.; injection: 2 μl;

DAD scan: 210-400 nm.

Rt=0.62 min (M⁺+1=292/294/296)

### Intermediate 46

### 6-Chloro-4-(2-methoxyethyl)-3-methyl-3,4-dihydropyrido[2,3-b]pyrazin-2(1H)-one

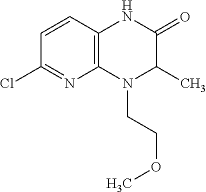 [SMILES: COCCN1c2nc(Cl)ccc2NC(=O)C1C]

Analogously to the synthesis of Intermediate 4, 6-chloro-4-(2-methoxyethyl)-3-methyl-3,4-dihydropyrido[2,3-b]pyrazin-2(1H)-one was prepared from 2.9 g of Intermediate 45 and 13.8 ml of N,N-diisopropylethylamine in 5 ml of DMF by heating for 72 hours at a bath temperature of 170° C. This gave 1.0 g of 6-chloro-4-(2-methoxyethyl)-3-methyl-3,4-dihydropyrido[2,3-b]pyrazin-2(1H)-one.

¹H-NMR (300 MHz, DMSO-d6): δ=1.21 (d, 3H); 3.19-3.31 (m+s, 4H); 3.45-3.59 (m, 2H); 3.99 (dt, 1H); 4.14 (q, 1H); 6.65 (d, 1H); 6.97 (d, 1H); 10.62 (bs, 1H).

### Intermediate 47

### 6-Chloro-4-(2-methoxyethyl)-1,3-dimethyl-3,4-dihydropyrido[2,3-b]pyrazin-2(1H)-one

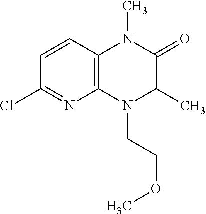 [SMILES: COCCN1c2nc(Cl)ccc2N(C)C(=O)C1C]

Analogously to the preparation of Intermediate 5, 6-chloro-4-(2-methoxyethyl)-1,3-dimethyl-3,4-dihydropyrido[2,3-b]pyrazin-2(1H)-one was prepared from 1.0 g of Intermediate 46, 256 mg of sodium hydride (60% in white oil) and 0.37 ml of methyl iodide in 9 ml of DMF. Purification by chromatography on silica gel (hexane/ethyl acetate gradient) gave 730 mg of 6-chloro-4-(2-methoxyethyl)-1,3-dimethyl-3,4-dihydropyrido[2,3-b]pyrazin-2(1H)-one.

¹H-NMR (300 MHz, DMSO-d6): δ=1.17 (d, 3H); 3.19-3.31 (m+2s, 7H); 3.45-3.60 (m, 2H); 4.02 (dt, 1H); 4.28 (q, 1H); 6.77 (d, 1H); 7.29 (d, 1H).

### Intermediate 48

### Methyl 3-{[4-(2-methoxyethyl)-1,3-dimethyl-2-oxo-1,2,3,4-tetrahydropyrido[2,3-b]pyrazin-6-yl]amino}benzoate

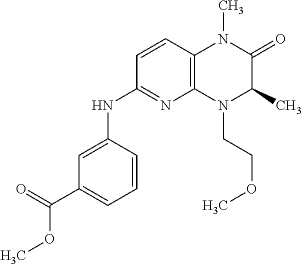 [SMILES: COCCN1c2nc(Nc3cccc(C(=O)OC)c3)ccc2N(C)C(=O)[C@H]1C]

A suspension of 1.6 g of Intermediate 47, 1.7 g of methyl 3-aminobenzoate (CAS 4518-10-9), 127 mg of palladium(II) acetate, 5.5 g of caesium carbonate and 351 mg of (+)-BINAP in 126 ml of toluene was stirred at 120° C. under an argon atmosphere for 14 hours. The reaction solution was added to water and extracted twice with ethyl acetate, the combined organic phases were dried over sodium sulphate, and the solvent was removed under reduced pressure. The residue was purified by chromatography on silica gel (hexane/ethyl acetate gradient up to 100% ethyl acetate content). This gave 1.5 g of methyl 3-{[4-(2-methoxyethyl)-1,3-dimethyl-2-oxo-1,2,3,4-tetrahydropyrido[2,3-b]pyrazin-6-yl]amino}benzoate.

¹H NMR: (300 MHz, 25° C., DMSO-d6): δ=1.13 (d, 3H); 3.21 (s, 3H); 3.24 (s, 3H); 3.59 (t, 2H); 3.84 (s, 3H); 4.11-4.24 (m, 2H); 6.22 (d, 1H); 7.23 (d, 1H); 7.34 8t, 1H); 7.40 (d, 1H); 7.71 (d, 1H); 8.46 (t, 1H); 9.05 (s, 1H).

### Intermediate 49

### 3-{[4-(2-Methoxyethyl)-1,3-dimethyl-2-oxo-1,2,3,4-tetrahydropyrido[2,3-b]pyrazin-6-yl]amino}benzoic acid

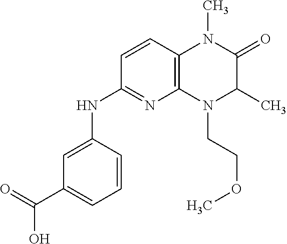 [SMILES: COCCN1c2nc(Nc3cccc(C(=O)O)c3)ccc2N(C)C(=O)C1C]

A solution of 1.5 g of Intermediate 48 in 12 ml of THF and 92 ml of methanol was admixed at RT with 39 ml of 1N lithium hydroxide solution and stirred at 60° C. for 2 hours. The mixture was adjusted to pH=7 using 1N hydrochloric acid and extracted twice with ethyl acetate. The combined organic phases were dried over sodium sulphate and the solvent was removed completely under reduced pressure. This gave 1.3 mg of 3-{[4-(2-methoxyethyl)-1,3-dimethyl-2-oxo-1,2,3,4-tetrahydropyrido[2,3-b]pyrazin-6-yl]amino}benzoic acid.

¹H-NMR (300 MHz, DMSO-d6): δ=1.13 (d, 3H); 3.21 (s, 3H); 3.24 (s, 3H); 3.24-3.31 (m, 1H); 3.54-3.64 (m, 2H); 4.11-4.22 (m, 2H); 6.21 (d, 1H); 7.23 (d, 1H); 7.31 (t, 1H); 7.39 (d, 1H); 7.71 (d, 1H); 8.38 (t, 1H); 8.99 (s, 1H); 12.61 (bs, 1H).

### Intermediate 50

### tert-Butyl 4-({(2R)-1-[(2,6-dichloropyridin-3-yl)amino]-1-oxopropan-2-yl}amino)piperidine-1-carbonate

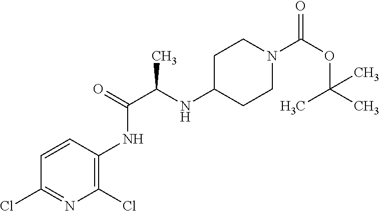 [SMILES: C[C@@H](NC1CCN(C(=O)OC(C)(C)C)CC1)C(=O)Nc1ccc(Cl)nc1Cl]

Analogously to the preparation of Intermediate 3, tert-butyl 4-({(2R)-1-[(2,6-dichloropyridin-3-yl)amino]-1-oxopropan-2-yl}amino)piperidine-1-carbonate was prepared from 2 g of Intermediate 2, 2.02 g of 1-Boc-4-piperidin-1-one (CAS 79099-07-3), 1.21 g of sodium acetate and 4.7 g of sodium triacetoxyborohydride in 60 ml of dichloromethane at 0° C. This gave 4.1 g of tert-butyl 4-({(2R)-1-[(2,6-dichloropyridin-3-yl)amino]-1-oxopropan-2-yl}amino)piperidine-1-carbonate as a crude product which was used without further purification for the next step.

¹H-NMR (400 MHz, DMSO-d6): δ=1.10.1.25 (m, 2H); 1.27 (d, 3H); 1.38 (s, 9H); 1.74 (bd, 1H); 1.89 (bd, 1H); 2.67-2.83 (bs, 2H); 3.39 (q, 1H); 3.80-3.90 (m, 2H); 7.58 (d, 1H); 8.66 (d, 1H).

### Intermediate 51

### tert-Butyl 4-[(3R)-6-chloro-3-methyl-2-oxo-2,3-dihydropyrido[2,3-b]pyrazin-4(1H)-yl]piperidine-1-carbonate

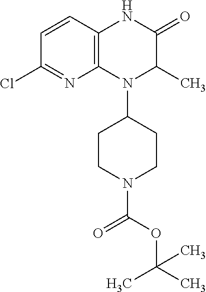 [SMILES: CC1C(=O)Nc2ccc(Cl)nc2N1C1CCN(C(=O)OC(C)(C)C)CC1]

Analogously to the synthesis of Intermediate 4, tert-butyl 4-[(3R)-6-chloro-3-methyl-2-oxo-2,3-dihydropyrido[2,3-b]pyrazin-4(1H)-yl]piperidine-1-carbonate was prepared from 1.02 g of Intermediate 50 and 3.4 ml of N,N-diisopropylethylamine in 5 ml of DMF by heating for 18 hours at a bath temperature of 170° C. This gave 577 mg of tert-butyl 4-[(3R)-6-chloro-3-methyl-2-oxo-2,3-dihydropyrido[2,3-b]pyrazin-4(1H)-yl]piperidine-1-carbonate.

¹H-NMR (300 MHz, DMSO-d6): δ=1.14 (d, 3H); 1.41 (s, 9H); 1.53-1.62 (m, 1H); 1.65-1.77 (m, 1H); 1.82-1.93 (m, 2H); 2.68-2.90 (bs, 2H); 3.98-4.10 (m, 2H); 4.10-4.20 (m, 2H); 6.69 (d, 1H); 7.02 (d, 1H); 10.58 (s, 1H).

### Intermediate 52

### tert-Butyl 4-[(3R)-6-chloro-1,3-dimethyl-2-oxo-2,3-dihydropyrido[2,3-b]pyrazin-4(1H)-yl]piperidine-1-carbonate

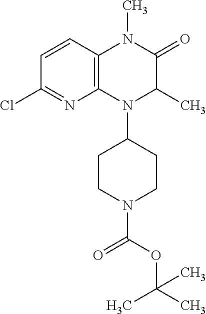 [SMILES: CC1C(=O)N(C)c2ccc(Cl)nc2N1C1CCN(C(=O)OC(C)(C)C)CC1]

Analogously to the preparation of Intermediate 5, tert-butyl 4-[(3R)-6-chloro-1,3-dimethyl-2-oxo-2,3-dihydropyrido[2,3-b]pyrazin-4(1H)-yl]piperidine-1-carbonate was prepared from 573 mg of Intermediate 51, 98 mg of sodium hydride (60% in white oil) and 0.14 ml of methyl iodide in 6.6 ml of DMF. Purification by chromatography on silica gel (hexane/ethyl acetate gradient) gave 460 mg of tert-butyl 4-[(3R)-6-chloro-1,3-dimethyl-2-oxo-2,3-dihydropyrido[2,3-b]pyrazin-4(1H)-yl]piperidine-1-carbonate.

¹H-NMR (300 MHz, DMSO-d6): δ=1.11 (d, 3H); 1.41 (s, 9H); 1.55-1.63 (m, 1H); 1.70 (qd, 1H); 1.81-1.93 (m, 2H); 2.71-2.91 (bs, 2H); 3.22 (s, 3H); 3.99-4.11 (m, 2H); 4.19 (tt, 1H); 4.30 (q, 1H); 6.80 (d, 1H); 7.33 (d, 1H).

### Intermediate 53

### tert-Butyl 4-[(3R)-6-{[3-(ethoxycarbonyl)phenyl]amino}-1,3-dimethyl-2-oxo-2,3-dihydropyrido[2,3-b]pyrazin-4(1H)-yl]piperidine-1-carboxylate

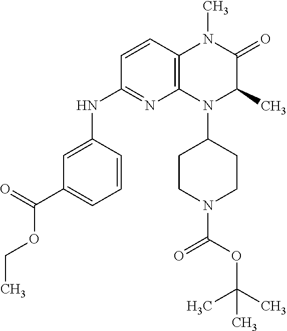 [SMILES: CCOC(=O)c1cccc(Nc2ccc3c(n2)N(C2CCN(C(=O)OC(C)(C)C)CC2)[C@H](C)C(=O)N3C)c1]

A suspension of 209 mg of Intermediate 52, 255 g of ethyl 3-aminobenzoate (CAS 582-33-2), 33 mg of palladium(II) acetate, 1.2 g of caesium carbonate and 91 mg of (+)-BINAP in 6.5 ml of toluene was stirred at 120° C. under an argon atmosphere for 2 hours and at RT for 14 hours. The reaction solution was added to water and extracted twice with ethyl acetate, the combined organic phases were dried over sodium sulphate, and the solvent was removed under reduced pressure. The residue was purified by chromatography on silica gel (hexane/ethyl acetate gradient up to 60% ethyl acetate content). This gave 338 mg of tert-butyl 4-[(3R)-6-{[3-(ethoxycarbonyl)phenyl]amino}-1,3-dimethyl-2-oxo-2,3-dihydropyrido[2,3-b]pyrazin-4(1H)-yl]piperidine-1-carboxylate.

¹H NMR: (300 MHz, 25° C., DMSO-d6): δ=1.07 (d, 3H); 1.31 (t, 3H); 1.41 (s, 9H); 1.56 (qd, 1H); 1.62 (bd, 1H); 1.74 (qd, 1H); 2.00 (bd, 1H); 2.68-2.93 (m, 2H); 3.20 (s, 3H); 3.99-4.10 (m, 2H); 4.21 (q, 1H); 4.29 (q, 1H); 4.36 (tt, 1H); 6.25/d, 1H); 7.28 (d, 1H); 7.36 (t, 1H); 7.41 (d, 1H); 7.85 (d, 1H); 8.18 (t, 1H); 9.04 (s, 1H).

### Intermediate 54

### 3-({(3R)-4-[1-(tert-Butoxycarbonyl)piperidin-4-yl]-1,3-dimethyl-2-oxo-1,2,3,4-tetrahydropyrido[2,3-b]pyrazin-6-yl}amino)benzoic acid

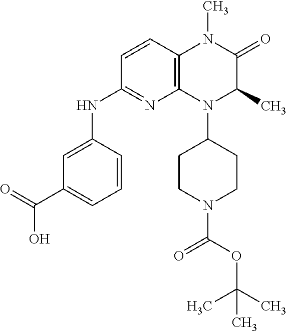 [SMILES: C[C@@H]1C(=O)N(C)c2ccc(Nc3cccc(C(=O)O)c3)nc2N1C1CCN(C(=O)OC(C)(C)C)CC1]

A solution of 334 mg of Intermediate 53 in 5 ml of methanol was admixed at RT with 128 mg of sodium hydroxide and stirred at 50° C. for 2 hours. The mixture was adjusted to pH=7 using 1N hydrochloric acid and extracted twice with ethyl acetate. The combined organic phases were dried over sodium sulphate and the solvent was removed completely under reduced pressure. This gave 270 mg of 3-({(3R)-4-[1-(tert-butoxycarbonyl)piperidin-4-yl]-1,3-dimethyl-2-oxo-1,2,3,4-tetrahydropyrido[2,3-b]pyrazin-6-yl}amino)benzoic acid.

¹H-NMR (400 MHz, DMSO-d6): δ=1.06 (d, 3H); 1.41 (s, 9H); 1.48-1.78 (m, 3H); 1.97 (bd, 1H); 2.73-3.00 (m, 2H); 3.20 (s, 3H); 4.05 (bs, 2H); 4.20 (q, 1H); 4.46 (tt, 1H); 6.26 (d, 1H); 7.27 (d, 1H); 7.33 (t, 1H); 7.41 (d, 1H); 7.68 (d, 1H); 8.35 (bs, 1H); 9.00 (s, 1H); 12.83 (bs, 1H).

### Intermediate 55

### N-(2,6-Dichloropyridin-3-yl)-N²-(4,4-dimethylcyclohexyl)-D-alaninamide

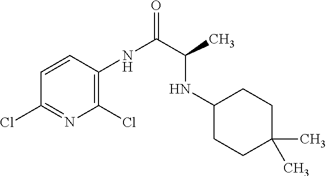 [SMILES: C[C@@H](NC1CCC(C)(C)CC1)C(=O)Nc1ccc(Cl)nc1Cl]

In analogy to the preparation of Intermediate 3, N-(2,6-dichloropyridin-3-yl)-N²-(4,4-dimethylcyclohexyl)-D-alaninamide was prepared proceeding from 2.85 g of Intermediate 2, 1.76 g of 4,4-dimethylcyclohexanone (CAS 4255-62-3), 1.73 g of sodium acetate and 6.7 g of sodium triacetoxyborohydride in 100 ml of dichloromethane at 0° C. This gave 4.0 g of N-(2,6-dichloropyridin-3-yl)-N²-(4,4-dimethylcyclohexyl)-D-alaninamide.

¹H-NMR (400 MHz, DMSO-d6): δ=0.82-0.89 (m, 8H); 1.09-1.18 (m, 3H); 1.20-1.39 (m, 9H); 1.52-1.63 (m, 2H); 1.69-1.78 (m, 1H); 2.30-2.41 (m, 1H); 3.33 (q, 1H); 7.57 (d, 1H); 8.68 (d, 1H).

### Intermediate 56

### (3R)-6-Chloro-4-(4,4-dimethylcyclohexyl)-3-methyl-3,4-dihydropyrido[2,3-b]pyrazin-2(1H)-one

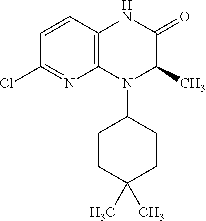 [SMILES: C[C@@H]1C(=O)Nc2ccc(Cl)nc2N1C1CCC(C)(C)CC1]

In analogy to the synthesis of Intermediate 4, (3R)-6-chloro-4-(4,4-dimethylcyclohexyl)-3-methyl-3,4-dihydropyrido[2,3-b]pyrazin-2(1H)-one was prepared proceeding from 3.96 g of Intermediate 55 and 16 ml of N,N-diisopropylethylamine in 20 ml of DMF by heating at bath temperature 170° C. for 16 hours. This gave 2.49 mg of (3R)-6-chloro-4-(4,4-dimethylcyclohexyl)-3-methyl-3,4-dihydropyrido[2,3-b]pyrazin-2(1H)-one.

¹H-NMR (400 MHz, DMSO-d6): δ=0.86 (d, 1H); 0.91 (s, 3H); 0.98 (s, 3H); 1.16 (d, 3H); 1.24-1.35 (m, 3H); 1.36-1.47 (m, 3H); 1.83 (dd, 1H); 1.97-2.11 (m, 1H); 3.81-3.93 (m, 1H); 6.63 (d, 1H); 6.98 (d, 1H); 10.54 (s, 1H).

### Intermediate 57

### (3R)-6-Chloro-4-(4,4-dimethylcyclohexyl)-1,3-dimethyl-3,4-dihydropyrido[2,3-b]pyrazin-2(1H)-one

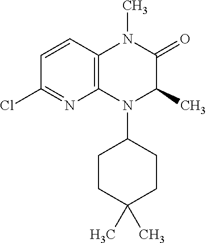 [SMILES: C[C@@H]1C(=O)N(C)c2ccc(Cl)nc2N1C1CCC(C)(C)CC1]

In analogy to the preparation of Intermediate 5, (3^{R}-6-chloro-4-(4,4-dimethylcyclohexyl)-1,3-dimethyl-3,4-dihydropyrido[2,3-b]pyrazin-2(1H)-one was prepared proceeding from 2.49 g of Intermediate 56, 529 mg of sodium hydride (60% in white oil) and 0.76 ml of methyl iodide in 36 ml of DMF. Purification by chromatography on silica gel (hexane/ethyl acetate gradient up to 30% ethyl acetate content) gave 1.3 g of (3R)-6-chloro-4-(4,4-dimethylcyclohexyl)-1,3-dimethyl-3,4-dihydropyrido[2,3-b]pyrazin-2(1H)-one.

¹H-NMR (300 MHz, DMSO-d6): δ=0.87-0.94 (m, 3H); 0.98 (s, 3H); 1.12 (d, 3H); 1.20-1.50 (m, 6H); 1.64-1.73 (m, 1H); 1.79 (td, 1H); 3.21 (s, 3H); 3.85-3.97 (m, 1H); 4.34 (q, 1H); 6.74 (d, 1H); 7.29 (d, 1H).

### Intermediate 58

### N-(2,6-Dichloropyridin-3-yl)-N²-(2-methylpropyl)-D-alaninamide

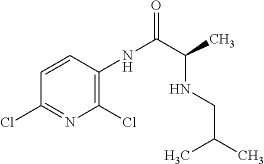 [SMILES: CC(C)CN[C@H](C)C(=O)Nc1ccc(Cl)nc1Cl]

In analogy to the preparation of Intermediate 3, N-(2,6-dichloropyridin-3-yl)-N²-(2-methylpropyl)-D-alaninamide was prepared proceeding from 2.92 g of Intermediate 2, 1.24 g of isobutyraldehyde, 0.67 ml of acetic acid and 7.3 g of sodium triacetoxyborohydride in 34 ml of dichloromethane at 0° C. This gave 1.22 g of N-(2,6-dichloropyridin-3-yl)-N²-(2-methylpropyl)-D-alaninamide.

¹H-NMR (400 MHz, DMSO-d6): δ=0.83-0.98 (m, 6H); 1.27 (d, 3H); 1.63-1.80 (m, 1H); 2.26 (dd, 1H); 2.46 (dd, 1H); 3.23 (q, 1H); 7.59 (d, 1H); 8.66 (d, 1H).

### Intermediate 59

### (3R)-6-Chloro-3-methyl-4-(2-methylpropyl)-3,4-dihydropyrido[2,3-b]pyrazin-2(1H)-one

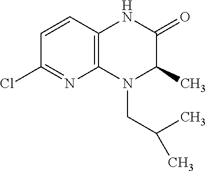 [SMILES: CC(C)CN1c2nc(Cl)ccc2NC(=O)[C@H]1C]

In analogy to the synthesis of Intermediate 4, (3R)-6-chloro-3-methyl-4-(2-methylpropyl)-3,4-dihydropyrido[2,3-b]pyrazin-2(1H)-one was prepared proceeding from 800 mg of Intermediate 58 and 3.8 ml of N,N-diisopropylethylamine in 10 ml of DMA by heating for 14 hours at bath temperature 165° C. This gave 1.05 g of (3R)-6-chloro-3-methyl-4-(2-methylpropyl)-3,4-dihydropyrido[2,3-b]pyrazin-2(1H)-one as crude product, which was used without further purification in the next stage.

UPLC-MS: Instrument: Waters Acquity UPLC-MS SQD; column: Acquity UPLC BEH C18 1.7 50×2.1 mm; eluent A: water+0.1% by vol. of formic acid (99%), eluent B: acetonitrile; gradient: 0-1.6 min 1-99% B, 1.6-2.0 min 99% B; flow rate 0.8 ml/min; temperature: 60° C.; injection: 2 μl;

DAD scan: 210-400 nm.

Rt=1.19 min.

### Intermediate 60

### (3R)-6-Chloro-1,3-dimethyl-4-(2-methylpropyl)-3,4-dihydropyrido[2,3-b]pyrazin-2(1H)-one

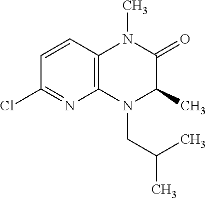 [SMILES: CC(C)CN1c2nc(Cl)ccc2N(C)C(=O)[C@H]1C]

In analogy to the preparation of Intermediate 5, (3R)-6-chloro-1,3-dimethyl-4-(2-methylpropyl)-3,4-dihydropyrido[2,3-b]pyrazin-2(1H)-one was prepared proceeding from 1.05 g of Intermediate 59 (crude product), 181 mg of sodium hydride (60% in white oil) and 0.26 ml of methyl iodide in 10 ml of DMF. Purification by chromatography on silica gel (dichloromethane/methanol gradient up to 5% methanol content) gave 390 mg of (3R)-6-chloro-1,3-dimethyl-4-(2-methylpropyl)-3,4-dihydropyrido[2,3-b]pyrazin-2(1H)-one.

¹H-NMR (300 MHz, DMSO-d6): δ=0.82 (d, 3H); 0.88 (d, 3H); 1.12 (d, 3H); 1.89-2.05 (m, 1H); 2.66-2.77 (m, 1H); 3.23 (s, 3H); 3.79 (dd, 1H); 4.15 (q, 1H); 6.74 (d, 1H); 7.28 (d, 1H).

### Intermediate 61

### N²-(1-Benzylpiperidin-4-yl)-N-(2,6-dichloropyridin-3-yl)-D-alaninamide

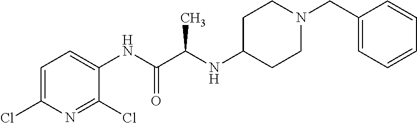 [SMILES: C[C@@H](NC1CCN(Cc2ccccc2)CC1)C(=O)Nc1ccc(Cl)nc1Cl]

A solution of 10 g of Intermediate 2 and 8.89 g of 1-benzylpiperidone (CAS 3612-20-2) in 100 ml of dichloromethane was admixed at RT with 18.2 g of sodium triacetoxyborohydride. After 16 hours, the mixture was poured cautiously onto saturated sodium hydrogencarbonate solution, the phases were separated and the aqueous phase was extracted with dichloromethane. The combined organic phases were dried over sodium sulphate and concentrated under reduced pressure. The residue was purified by chromatography on silica gel (heptane/ethyl acetate gradient). This gave 15.1 g of N²-(1-benzylpiperidin-4-yl)-N-(2,6-dichloropyridin-3-yl)-D-alaninamide.

¹H NMR (400 MHz, 25° C., CDCl₃): δ=1.17 (bs, 1H), 1.37-1.52 (m, 5H), 1.86 (d, 1H), 1.91-2.04 (m, 3H), 2.48 (bs, 1H), 2.83-2.88 (m, 2H), 3.38 (q, 1H), 3.51 (s, 2H), 7.22-7.33 (m, 6H), 8.82 (d, 1H), 10.4 (bs, 1H).

### Intermediate 62

### (3R)-4-(1-Benzylpiperidin-4-yl)-6-chloro-3-methyl-3,4-dihydropyrido[2,3-b]pyrazin-2(1H)-one

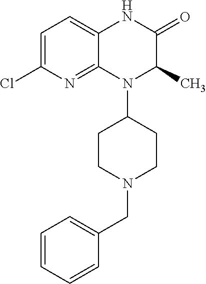 [SMILES: C[C@@H]1C(=O)Nc2ccc(Cl)nc2N1C1CCN(Cc2ccccc2)CC1]

A solution of 15.1 g of Intermediate 61 and 32.3 ml of N,N-diisopropylethylamine in 277 ml of DMA was stirred in a tightly sealed vessel at bath temperature 170° C. for 48 hours. After cooling, the mixture was diluted with water and extracted three times with ethyl acetate. The combined organic phases were concentrated under reduced pressure. Toluene was added, and the mixture was concentrated fully under reduced pressure once more. The residue was stirred in a heptane/water mixture, and the precipitate was filtered off with suction and then dried by distillation with toluene. This gave 13.8 g of (3R)-4-(1-benzylpiperidin-4-yl)-6-chloro-3-methyl-3,4-dihydropyrido[2,3-b]pyrazin-2(1H)-one.

¹H NMR (400 MHz, 25° C., CDCl₃): δ=1.27 (d, 3H), 1.54-1.81 (m, 3H), 1.86-2.26 (m, 3H), 2.90-3.05 (m, 2H), 3.54 (s, 2H), 4.22-4.39 (m, 2H), 6.60 (d, 1H), 6.87 (d, 1H), 7.25-7.32 (m, 5H), 8.72 (bs, 1H).

### Intermediate 63

### (R)-4-(1-Benzylpiperidin-4-yl)-6-chloro-1,3-dimethyl-3,4-dihydropyrido[2,3-b]pyrazin-2(1H)-one

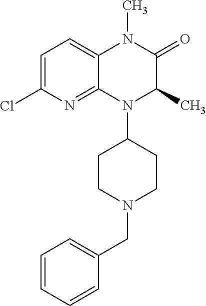 [SMILES: C[C@@H]1C(=O)N(C)c2ccc(Cl)nc2N1C1CCN(Cc2ccccc2)CC1]

A solution of 13.1 g of Intermediate 62 in 131 ml of DMF was admixed at 0° C. with 2.08 mg of sodium hydride (60% in white oil) in portions. The mixture was stirred at RT for another 30 min, then cooled again to 0° C., and 2.28 ml of methyl iodide were added. After about 10 min, the mixture was added rapidly to ice-water under an argon atmosphere, and the precipitate was filtered off with suction and washed with heptane. This gave 12.7 g of (R)-4-(1-benzylpiperidin-4-yl)-6-chloro-1,3-dimethyl-3,4-dihydropyrido[2,3-b]pyrazin-2(1H)-one.

¹H NMR (400 MHz, 25° C., CDCl₃): δ=1.19 (d, 3H), 1.57-1.79 (m, 2H+H₂O), 1.92 (bq, 1H), 2.04-2.22 (m, 3H), 2.96 (bs, 2H), 3.28 (s, 3H), 3.54 (s, 2H), 4.30-4.35 (m, 2H), 6.65 (d, 1H), 6.96 (d, 1H), 7.31-7.37 (m, 5H).

### Intermediate 64

### (3R)-6-Chloro-1,3-dimethyl-4-(piperidin-4-yl)-3,4-dihydropyrido[2,3-b]pyrazin-2(1H)-one hydrochloride

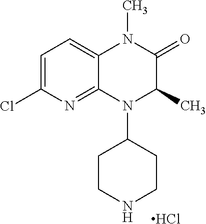 [SMILES: C[C@@H]1C(=O)N(C)c2ccc(Cl)nc2N1C1CCNCC1 ; Cl]

A solution of 12.2 g of Intermediate 63 and 4.46 ml of 1-chlorethyl carbonochloridate (CAS 50893-53-3) in 131 ml of 1,2-dichloroethane was heated under reflux for 4 hours. The mixture was concentrated fully and taken up in ethyl acetate/heptane (1:1). This solution was filtered through silica gel and washed first with heptane, then with ethyl acetate. The eluted residue was heated in methanol and then concentrated again. This gave 8.2 g of (3R)-6-chloro-1,3-dimethyl-4-(piperidin-4-yl)-3,4-dihydropyrido[2,3-b]pyrazin-2(1H)-one hydrochloride.

¹H NMR (400 MHz, 25° C., DMSO-d6₃): δ=1.22 (d, 3H), 1.94-2.01 (m, 1H), 2.13 (dq, 1H), 2.23-2.37 (m, 2H), 3.16 (tt, 2H), 3.30 (s, 3H), 3.43-3.53 (m, 2H), 4.28 (q, 1H), 4.39 (tt, 1H), 6.80 (d, 1H), 7.07-7.21 (m, 1H), 7.32 (d, 1H).

### Intermediate 65

### (3R)-6-Chloro-1,3-dimethyl-4-(1-methylpiperidin-4-yl)-3,4-dihydropyrido[2,3-b]pyrazin-2(1H)-one

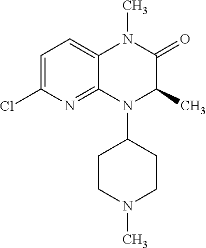 [SMILES: C[C@@H]1C(=O)N(C)c2ccc(Cl)nc2N1C1CCN(C)CC1]

A solution of 8.2 g of Intermediate 64 in 77.1 ml of methanol was admixed at RT first with 77.1 ml of formaldehyde solution (37% in water) and then with 2.19 g of sodium cyanoborohydride and 3.49 g of acetic acid. The mixture was stirred for 16 hours, and then 2 N sodium hydroxide solution was added. The reaction solution was extracted with ethyl acetate, the organic phase was dried over sodium sulphate and the solvent was removed under reduced pressure. The residue was purified by chromatography on silica gel (start with heptane/ethyl acetate, 1:1 gradient to 92:5:3 ethyl acetate/triethylamine/methanol). This gave 6.7 g of (3R)-6-chloro-1,3-dimethyl-4-(1-methylpiperidin-4-yl)-3,4-dihydropyrido[2,3-b]pyrazin-2(1H)-one.

¹H NMR (400 MHz, 25° C., CDCl₃): δ=1.20 (d, 3H), 1.62-1.68 (m, 1H), 1.75 (dq, 1H), 1.95 (dq, 1H), 2.07-2.21 (m, 3H), 2.31 (s, 3H), 2.94 (d, 2H), 3.29 (s, 3H), 4.25-4.35 (m, 2H), 6.66 (d, 1H), 6.97 (d, 1H).

### Intermediate 66

### 2-Bromo-N-(2,6-dichloropyridin-3-yl)propanamide

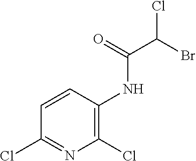 [SMILES: O=C(Nc1ccc(Cl)nc1Cl)C(Cl)Br]

At RT, 20.3 g of 2-bromopropionyl bromide (CAS 563-76-8) were added slowly to a solution of 8.5 g of 3-amino-2,6-dichloropyridine (CAS 62476-59-9) in 200 ml of THF and 12.7 ml of pyridine. The mixture was left stirring at RT for 72 hours. Water was then added, and the mixture was extracted with ethyl acetate. The organic phase was dried over sodium sulphate and concentrated fully under reduced pressure. The residue was purified by chromatography on silica gel (dichloromethane). This gave 8.2 g of 2-bromo-N-(2,6-dichloropyridin-3-yl)propanamide.

¹H-NMR (300 MHz, DMSO-d6): δ=1.76 (d, 3H); 4.94 (q, 1H); 7.60 (d, 1H); 8.22 (d, 1H); 10.17 (s, 1H).

### Intermediate 67

### N-(2,6-Dichloropyridin-3-yl)-N²-phenylalaninamide

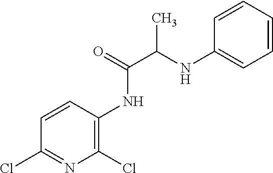 [SMILES: CC(Nc1ccccc1)C(=O)Nc1ccc(Cl)nc1Cl]

A solution of 2.7 g of Intermediate 66 and 759 mg of aniline in 27 ml of toluene and 2.7 ml of diisopropylethylamine was stirred at 140° C. for 3 hours. After cooling to RT, water was added and the mixture was extracted with ethyl acetate. The organic phase was dried over sodium sulphate and concentrated fully under reduced pressure. The residue was purified by chromatography on silica gel (dichloromethane). This gave 3.1 g of N-(2,6-dichloropyridin-3-yl)-N2-phenylalaninamide which was sufficiently pure for further reactions.

¹H-NMR (300 MHz, DMSO-d6): δ=1.44 (d, 3H); 4.12 (qi, 1H); 6.11 (d, 1H); 6.64 (d, 2H); 6.99 (t, 1H); 7.10 (t, 2H); 7.56 (d, 1H); 8.29 (d, 1H); 9.79 (s, 1H).

### Intermediate 68

### 6-Chloro-3-methyl-4-phenyl-3,4-dihydropyrido[2,3-b]pyrazin-2(1H)-one

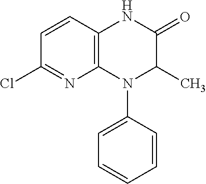 [SMILES: CC1C(=O)Nc2ccc(Cl)nc2N1c1ccccc1]

Analogously to the synthesis of Intermediate 4, 6-chloro-3-methyl-4-phenyl-3,4-dihydropyrido[2,3-b]pyrazin-2(1H)-one was prepared from 1.8 g of Intermediate 67 and 12.3 ml of N,N-dicyclohexylmethylamine in 10 ml of DMF by heating for 18 hours at a bath temperature of 170° C. This gave 350 mg of 6-chloro-3-methyl-4-phenyl-3,4-dihydropyrido[2,3-b]pyrazin-2(1H)-one.

¹H-NMR (300 MHz, DMSO-d6): δ=1.29 (d, 3H); 4.48 (q, 1H); 6.84 (d, 1H); 7.17 (d, 1H); 7.22 (t, 1H); 7.33 (d, 2H); 7.41 (t, 2H); 10.82 (s, 1H).

### Intermediate 69

### 6-Chloro-1,3-dimethyl-4-phenyl-3,4-dihydropyrido[2,3-b]pyrazin-2(1H)-one

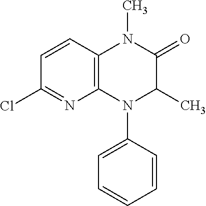 [SMILES: CC1C(=O)N(C)c2ccc(Cl)nc2N1c1ccccc1]

Analogously to the preparation of Intermediate 5, 6-chloro-1,3-dimethyl-4-phenyl-3,4-dihydropyrido[2,3-b]pyrazin-2(1H)-one was prepared from 500 mg of Intermediate 68 (obtained from 2 reactions), 120 mg of sodium hydride (60% in white oil) and 0.171 ml of methyl iodide in 9 ml of DMF. Chromatography on silica gel (hexane/ethyl acetate gradient) gave 380 mg of 6-chloro-1,3-dimethyl-4-phenyl-3,4-dihydropyrido[2,3-b]pyrazin-2(1H)-one.

¹H-NMR (300 MHz, DMSO-d6): δ=1.29 (d, 3H); 3.32 (s, 3H); 4.60 (q, 1H); 6.96 (d, 1H); 7.21 (t, 1H); 7.33 (d, 2H); 7.41 I/t, 2H); 7.50 (d, 1H).

### Intermediate 70

### N-(2,6-Dichloropyridin-3-yl)-N²-(4-fluorophenyl)alaninamide

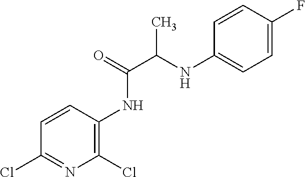 [SMILES: CC(Nc1ccc(F)cc1)C(=O)Nc1ccc(Cl)nc1Cl]

A solution of 2.0 g of Intermediate 66 and 746 mg of 4-fluoroaniline in 25 ml of toluene and 2.0 ml of di-iso-propylethylamine was stirred at 130° C. for 5 hours. After cooling to RT, water was added and the mixture was extracted with ethyl acetate. The organic phase was dried over sodium sulphate and concentrated fully under reduced pressure. The residue was purified by chromatography on silica gel (hexane/ethyl acetate gradient up to 50% ethyl acetate content). This gave 1.8 g of N-(2,6-dichloropyridin-3-yl)-N²-(4-fluorophenyl)alaninamide.

¹H-NMR (300 MHz, DMSO-d6): δ=1.43 (d, 3H); 4.04-4.12 (m, 1H); 6.08 (d, 1H); 6.63 (dd, 2H); 6.95 (t, 2H); 7.57 (d, 1H); 8.28 (d, 1H); 9.80 (bs, 1H).

### Intermediate 71

### 6-Chloro-4-(4-fluorophenyl)-3-methyl-3,4-dihydropyrido[2,3-b]pyrazin-2(1H)-one

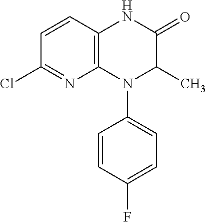 [SMILES: CC1C(=O)Nc2ccc(Cl)nc2N1c1ccc(F)cc1]

Analogously to the synthesis of Intermediate 4, 6-chloro-4-(4-fluorophenyl)-3-methyl-3,4-dihydropyrido[2,3-b]pyrazin-2(1H)-one was prepared proceeding from 1.8 g of Intermediate 70 and 7.6 ml of N,N-di-iso-propylethylamine in 18 ml of DMA by heating at bath temperature 175° C. for 48 hours. This gave 1.0 mg of 6-chloro-4-(4-fluorophenyl)-3-methyl-3,4-dihydropyrido[2,3-b]pyrazin-2(1H)-one.

¹H-NMR (300 MHz, DMSO-d6): δ=1.27 (d, 3H); 4.45 (q, 1H); 6.81 (d, 1H); 7.14 (d, 1H); 7.25/t, 2H); 7.38 (dd, 2H); 10.8 (bs, 1H).

### Intermediate 72

### 6-Chloro-4-(4-fluorophenyl)-1,3-dimethyl-3,4-dihydropyrido[2,3-b]pyrazin-2(1H)-one

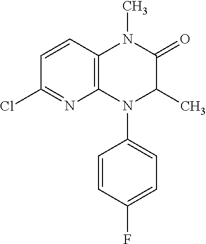 [SMILES: CC1C(=O)N(C)c2ccc(Cl)nc2N1c1ccc(F)cc1]

Analogously to the preparation of Intermediate 5, 6-chloro-4-(4-fluorophenyl)-1,3-dimethyl-3,4-dihydropyrido[2,3-b]pyrazin-2(1H)-one was prepared from 1.0 mg of Intermediate 71, 224 mg of sodium hydride (60% in white oil) and 0.32 ml of methyl iodide in 20 ml of DMF. Chromatography on silica gel (dichloromethane/methanol gradient up to 1% methanol content) gave 870 mg of 6-chloro-4-(4-fluorophenyl)-1,3-dimethyl-3,4-dihydropyrido[2,3-b]pyrazin-2(1H)-one.

¹H-NMR (300 MHz, DMSO-d6): δ=1.27 (d, 3H); 3.31 (s, 3H); 4.56 (q, 1H); 6.93 (d, 1H); 7.25 (t, 2H); 7.39 (dd, 1H); 7.47 (d, 1H).

### Intermediate 73

### Methyl 3-{[4-(4-fluorophenyl)-1,3-dimethyl-2-oxo-1,2,3,4-tetrahydropyrido[2,3-b]pyrazin-6-yl]amino}benzoate

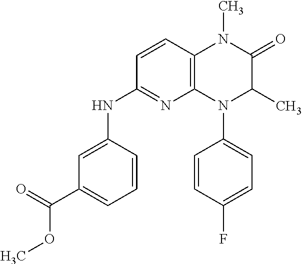 [SMILES: COC(=O)c1cccc(Nc2ccc3c(n2)N(c2ccc(F)cc2)C(C)C(=O)N3C)c1]

A suspension of 225 mg of Intermediate 73, 227 g of methyl 3-aminobenzoate (CAS 4518-10-9), 33 mg of palladium(II) acetate, 1.2 g of caesium carbonate and 92 mg of (+)-BINAP in 3 ml of toluene was stirred at 120° C. under an argon atmosphere for 3 hours and at RT for 56 hours. The reaction solution was added to water and extracted twice with ethyl acetate, the combined organic phases were dried over sodium sulphate, and the solvent was removed under reduced pressure. The residue was purified by RP-HPLC (Waters SQD autopurification system; column: Waters XBridge C18 5μ 100×30 mm; eluent A: water+0.1% by vol. of formic acid (99%), eluent B: acetonitrile; gradient: 0-8.0 min 1-100% B, 8.0-10.0 min 100% B; flow rate 50.0 ml/min; temperature: RT; injection: 2500 μl; DAD scan: 210-400 nm). This gave 163 mg of methyl 3-{[4-(4-fluorophenyl)-1,3-dimethyl-2-oxo-1,2,3,4-tetrahydropyrido[2,3-b]pyrazin-6-yl]amino}benzoate.

UPLC-MS: Instrument: Waters Acquity UPLC-MS SQD; column: Acquity UPLC BEH C18 1.7 50×2.1 mm; eluent A: water+0.1% by vol. of formic acid (99%), eluent B: acetonitrile; gradient: 0-1.6 min 1-99% B, 1.6-2.0 min 99% B; flow rate 0.8 ml/min; temperature: 60° C.; injection: 2 μl;

DAD scan: 210-400 nm.

Rt=1.27 min.

### Intermediate 74

### 3-{[4-(4-Fluorophenyl)-1,3-dimethyl-2-oxo-1,2,3,4-tetrahydropyrido[2,3-b]pyrazin-6-yl]amino}benzoic acid

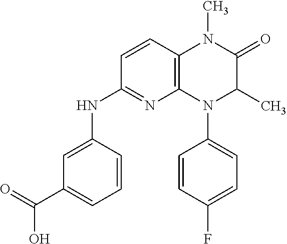 [SMILES: CC1C(=O)N(C)c2ccc(Nc3cccc(C(=O)O)c3)nc2N1c1ccc(F)cc1]

A solution of 150 mg of Intermediate 73 in 4 ml of THF was admixed at RT with 0.89 ml of sodium hydroxide solution (2 N) and stirred at 50° C. for 3 hours and at 90° C. for 30 min. The THF was removed completely under reduced pressure and the solution was acidified with water and 1 N hydrochloric acid. The precipitate formed was filtered off and dried under reduced pressure. This gave 135 mg of 3-{[4-(4-fluorophenyl)-1,3-dimethyl-2-oxo-1,2,3,4-tetrahydropyrido[2,3-b]pyrazin-6-yl]amino}benzoic acid.

¹H-NMR (300 MHz, DMSO-d6): δ=1.27 (d, 3H); 3.28 (s, 3H); 4.50 (q, 1H); 6.35 (d, 1H); 6.94 (t, 1H); 7.21-7.30 (m, 3H); 7.34-7.43 (m, 4H); 7.57-7.63 (m, 1H); 7.72 (dd, 1H); 9.01 (s, 1H).

### Intermediate 75

### N-(2,6-Dichloropyridin-3-yl)-N²-(3-methylphenyl)-D-alaninamide

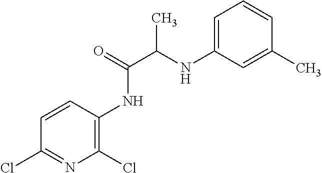 [SMILES: Cc1cccc(NC(C)C(=O)Nc2ccc(Cl)nc2Cl)c1]

A solution of 1.5 g of Intermediate 66 and 539 mg of 3-methylaniline in 20 ml of toluene and 1.5 ml of di-iso-propylethylamine was stirred at 130° C. for 5 hours. After cooling to RT, water was added and the mixture was extracted with ethyl acetate. The organic phase was dried over sodium sulphate and concentrated fully under reduced pressure. The residue was purified by chromatography on silica gel (hexane/ethyl acetate gradient up to 50% ethyl acetate content). This gave 1.6 g of N-(2,6-dichloropyridin-3-yl)-N²-(3-methylphenyl)-D-alaninamide.

¹H-NMR (300 MHz, DMSO-d6): δ=1.43 (d, 3H); 2.18 (s, 3H); 4.03-4.16 (m, 1H); 6.01 (d, 1H); 6.40-6.50 (m, 3H); 6.98 (t, 1H); 7.57 (d, 1H); 8.28 (d, 1H), 9.80 (bs, 1H).

### Intermediate 76

### 6-Chloro-3-methyl-4-(3-methylphenyl)-3,4-dihydropyrido[2,3-b]pyrazin-2(1H)-one

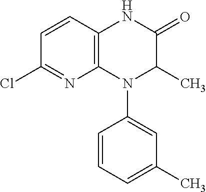 [SMILES: Cc1cccc(N2c3nc(Cl)ccc3NC(=O)C2C)c1]

A solution of 1.6 g of Intermediate 75 and 5.5 ml of N,N-diisopropylethylamine in 16 ml of DMA was stirred at bath temperature 175° C. for 72 hours. After cooling, the mixture was diluted with water and extracted with ethyl acetate. The combined organic phases were concentrated under reduced pressure. The residue was stirred in dichloromethane and filtered off with suction. This gave 830 mg of 6-chloro-3-methyl-4-(3-methylphenyl)-3,4-dihydropyrido[2,3-b]pyrazin-2(1H)-one.

¹H-NMR (400 MHz, DMSO-d6): δ=1.29 (d, 3H); 2.32 (s, 3H); 4.44 (q, 1H); 6.83 (d, 1H); 7.04 (d, 1H); 7.10-7.18 (m, 3H); 7.28 (t, 1H); 10.8 (bs, 1H).

### Intermediate 77

### 6-Chloro-1,3-dimethyl-4-(3-methylphenyl)-3,4-dihydropyrido[2,3-b]pyrazin-2(1H)-one

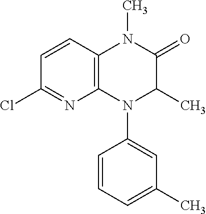 [SMILES: Cc1cccc(N2c3nc(Cl)ccc3N(C)C(=O)C2C)c1]

A solution of 830 mg of Intermediate 76 and 0.27 ml of methyl iodide in 20 ml of DMF was admixed at 0° C. with 0.17 g of sodium hydride (60% in white oil). After stirring at 0° C. for one hour and at RT for 14 hours, the mixture was diluted with dichloromethane and washed three times with semisaturated aqueous sodium chloride solution. The combined organic phases were dried over sodium sulphate and concentrated fully under reduced pressure. The residue was purified by chromatography on silica gel (dichloromethane/methanol gradient up to 2% methanol content). This gave 850 mg of 6-chloro-1,3-dimethyl-4-(3-methylphenyl)-3,4-dihydropyrido[2,3-b]pyrazin-2(1H)-one.

¹H-NMR (400 MHz, DMSO-d6): δ=1.28 (d, 3H); 2.32 (s, 3H); 3.31 (s, 3H); 4.56 (q, 1H); 6.95 (d, 1H); 7.03 (bd, 1H); 7.10-7.17 (m, 2H); 7.28 (t, 1H); 7.49 (d, 1H).

### Intermediate 78

### Methyl 5-{[(3R)-1,3-dimethyl-2-oxo-4-(tetrahydro-2H-pyran-4-yl)-1,2,3,4-tetrahydropyrido[2,3-b]pyrazin-6-yl]amino}-2-methoxybenzoate

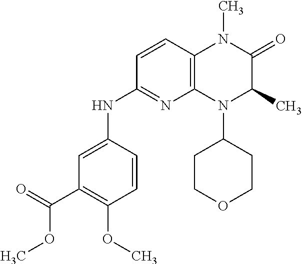 [SMILES: COC(=O)c1cc(Nc2ccc3c(n2)N(C2CCOCC2)[C@H](C)C(=O)N3C)ccc1OC]

A suspension of 800 mg of Intermediate 10, 931 mg of methyl 5-amino-2-methoxybenzoate (CAS 22802-67-1), 115 mg of palladium(II) acetate, 4.19 g of caesium carbonate and 320 mg of (+)-BINAP in 57.5 ml of toluene was stirred at 120° C. under an argon atmosphere for 5 hours. The reaction solution was added to water and extracted twice with ethyl acetate, the combined organic phases were dried over sodium sulphate, and the solvent was removed under reduced pressure. The residue was purified by chromatography on silica gel (hexane/ethyl acetate gradient up to 100% ethyl acetate content). This gave 810 mg of methyl 5-{[(3R)-1,3-dimethyl-2-oxo-4-(tetrahydro-2H-pyran-4-yl)-1,2,3,4-tetrahydropyrido[2,3-b]pyrazin-6-yl]amino}-2-methoxybenzoate.

1H NMR: (300 MHz, 25° C., DMSO-d6): δ=1.07 (d, 3H); 1.57 (bd, 1H); 1.72 (qd, 1H); 1.79-1.96 (m, 2H); 3.19 (s, 3H); 3.31-3.49 (m, 2H); 3.77 (s, 3H); 3.78 (s, 3H); 3.88-3.99 (m, 2H); 4.20 (q, 1H); 4.38 (tt, 1H); 6.16 (d, 1H); 7.04 (d, 1H); 7.23 (d, 1H); 7.69 (dd, 1H); 7.87 (d, 1H); 8.72 (s, 1H).

### Intermediate 79

### 5-{[(3R)-1,3-Dimethyl-2-oxo-4-(tetrahydro-2H-pyran-4-yl)-1,2,3,4-tetrahydropyrido[2,3-b]pyrazin-6-yl]amino}-2-methoxybenzoic acid

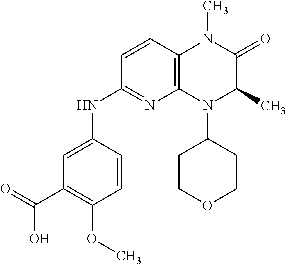 [SMILES: COc1ccc(Nc2ccc3c(n2)N(C2CCOCC2)[C@H](C)C(=O)N3C)cc1C(=O)O]

A solution of 780 mg of Intermediate 78 in 6 ml of THF and 40 ml of methanol was admixed at RT with 17.7 ml of 1N lithium hydroxide solution and stirred at 60° C. for 7 hours. The mixture was adjusted to pH=7 using 1N hydrochloric acid and extracted twice with ethyl acetate. The combined organic phases were dried over sodium sulphate and the solvent was removed completely under reduced pressure. This gave 680 mg of 5-{[(3R)-1,3-dimethyl-2-oxo-4-(tetrahydro-2H-pyran-4-yl)-1,2,3,4-tetrahydropyrido[2,3-b]pyrazin-6-yl]amino}-2-methoxybenzoic acid.

¹H NMR: (400 MHz, 25° C., DMSO-d6): δ=1.06 (d, 3H); 1.55 (bd, 1H); 1.71 (qd, 1H); 1.79-1.95 (m, 2H); 3.19 (s, 3H); 3.38-3.52 (m, 2H); 3.47 (s, 3H); 3.87-3.97 (m, 2H); 4.20 (q, 1H); 4.44 (tt, 1H); 6.17 (d, 1H); 7.02 (d, 1H); 7.23 (d, 1H); 7.60 (dd, 1H); 7.96 (d, 1H); 8.70 (s, 1H); 12.29 (bs, 1H).

### Intermediate 80

### Methyl 3-{[(3R)-1,3-dimethyl-2-oxo-4-(tetrahydro-2H-pyran-4-yl)-1,2,3,4-tetrahydropyrido[2,3-b]pyrazin-6-yl]amino}-2-methoxybenzoate

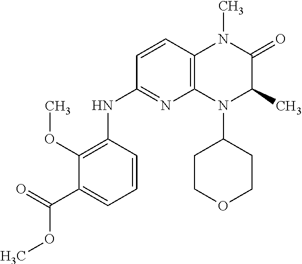 [SMILES: COC(=O)c1cccc(Nc2ccc3c(n2)N(C2CCOCC2)[C@H](C)C(=O)N3C)c1OC]

A suspension of 600 mg of Intermediate 10, 698 mg of methyl 3-amino-2-methoxybenzoate (CAS 5129-25-9), 87 mg of palladium(II) acetate, 2.5 g of caesium carbonate and 240 mg of (+)-BINAP in 43 ml of toluene was stirred at 120° C. under an argon atmosphere for 7.5 hours. The reaction solution was added to water and extracted twice with ethyl acetate, the combined organic phases were dried over sodium sulphate, and the solvent was removed under reduced pressure. The residue was purified by chromatography on silica gel (hexane/ethyl acetate gradient up to 100% ethyl acetate content). This gave 325 mg of methyl 3-{[(3R)-1,3-dimethyl-2-oxo-4-(tetrahydro-2H-pyran-4-yl)-1,2,3,4-tetrahydropyrido[2,3-b]pyrazin-6-yl]amino}-2-methoxybenzoate.

1H NMR: (400 MHz, 25° C., CDCl₃): δ=1.25 (d, 3H); 1.71 (bd, 1H); 1.85 (qd, 1H); 2.01 (qd, 1H); 2.12 (bd, 1H); 3.32 (s, 3H); 3.57 (t, 2H); 3.91 (s, 3H); 3.94 (s, 3H); 4.07-4.18 (m, 2H); 4.32 (q, 1H); 4.56 (tt, 1H); 6.25 (d, 1H); 6.95 (bs, 1H); 7.06 (d, 1H); 7.09 (t, 1H); 7.38 (dd, 1H); 8.43 (dd, 1H).

### Intermediate 81

### 3-{[(3R)-1,3-Dimethyl-2-oxo-4-(tetrahydro-2H-pyran-4-yl)-1,2,3,4-tetrahydropyrido[2,3-b]pyrazin-6-yl]amino}-2-methoxybenzoic acid

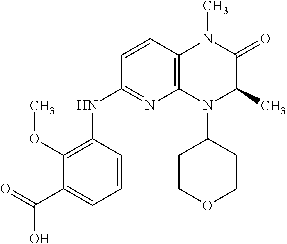 [SMILES: COc1c(Nc2ccc3c(n2)N(C2CCOCC2)[C@H](C)C(=O)N3C)cccc1C(=O)O]

A solution of 300 mg of Intermediate 80 in 2 ml of THF and 16 ml of methanol was admixed at RT with 6.8 ml of 1N lithium hydroxide solution and stirred at 60° C. for 6 hours. The mixture was adjusted to pH=7 using 1N hydrochloric acid and extracted twice with ethyl acetate. The combined organic phases were dried over sodium sulphate and the solvent was removed completely under reduced pressure. This gave 275 mg of 5-{[(3R)-1,3-dimethyl-2-oxo-4-(tetrahydro-2H-pyran-4-yl)-1,2,3,4-tetrahydropyrido[2,3-b]pyrazin-6-yl]amino}-2-methoxybenzoic acid.

¹H NMR: (400 MHz, 25° C., DMSO-d6): δ=1.09 (d, 3H); 1.60 (bd, 1H); 1.76 (qd, 1H); 1.86-2.00 (m, 2H); 3.21 (s, 3H); 3.34-3.48 (m, 2H); 3.76 (s, 3H); 3.92-4.03 (m, 2H); 4.23 (q, 1H); 4.35 (tt, 1H); 6.59 (d, 1H); 7.08 (t, 1H); 7.18 (dd, 1H); 7.27 (d, 1H); 8.14 (s, 1H); 8.49 (dd, 1H); 12.51 (bs, 1H).

### Intermediate 82

### tert-Butyl 4-[4-({1-[(2,6-dichloropyridin-3-yl)amino]-1-oxopropan-2-yl}amino)phenyl]piperazine-1-carboxylate

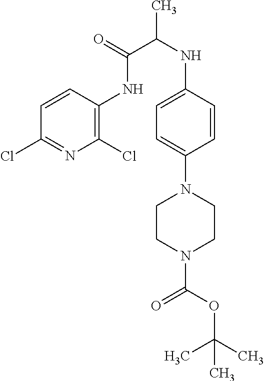 [SMILES: CC(Nc1ccc(N2CCN(C(=O)OC(C)(C)C)CC2)cc1)C(=O)Nc1ccc(Cl)nc1Cl]

A solution of 5.1 g of Intermediate 66 and 4.75 g of tert-butyl 4-(4-aminophenyl)piperazine-1-carboxylate (CAS 170911-92-9) in 63.75 ml of toluene and 5.96 ml of di-iso-propylethylamine was stirred at 140° C. for 14 hours. After cooling to RT, water was added and the mixture was extracted with ethyl acetate. The organic phase was dried over sodium sulphate and concentrated fully under reduced pressure. The residue was purified by chromatography on silica gel (dichloromethane/methanol gradient up to 2% methanol content). This gave 7.7 g of tert-butyl 4-[4-({1-[(2,6-dichloropyridin-3-yl)amino]-1-oxopropan-2-yl}amino)phenyl]piperazine-1-carboxylate.

1H NMR: (400 MHz, 25° C., DMSO-d6): δ=1.37-1.45 (m, 12H); 2.82-2.90 (m, 4H); 3.37-3.46 (m, 4H); 3.95-4.05 (m, 1H); 5.75 (d, 1H); 6.59 (d, 2H); 6.79 (d, 2H); 7.56 (d, 1H); 8.35 (d, 1H); 9.76 (s, 1H).

### Intermediate 83

### tert-Butyl 4-{4-[6-chloro-3-methyl-2-oxo-2,3-dihydropyrido[2,3-b]pyrazin-4(1H)-yl]phenyl}piperazine-1-carboxylate

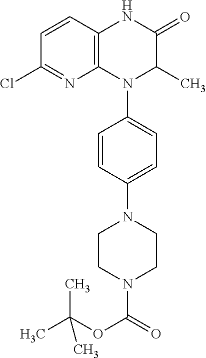 [SMILES: CC1C(=O)Nc2ccc(Cl)nc2N1c1ccc(N2CCN(C(=O)OC(C)(C)C)CC2)cc1]

A solution of 7.7 g of Intermediate 82 and 5.3 ml of N,N-diisopropylethylamine in 40 ml of DMA, divided between 4 tightly sealed glass vessels, was stirred at bath temperature 165° C. for 48 hours. After cooling, the combined solutions were diluted with water and extracted with ethyl acetate. The combined organic phases were concentrated under reduced pressure. The residue was purified by chromatography on silica gel (dichloromethane/methanol gradient up to 3% methanol content) and a further chromatography procedure on silica gel (hexane/ethyl acetate gradient up to 25% ethyl acetate content). This gave 5.2 g of tert-butyl 4-{4-[6-chloro-3-methyl-2-oxo-2,3-dihydropyrido[2,3-b]pyrazin-4(1H)-yl]phenyl}piperazine-1-carboxylate.

¹H-NMR (400 MHz, 25° C., DMSO-d6): δ=1.25 (d, 3H); 1.42 (s, 9H); 3.08-3.17 (m, 4H); 3.41-3.51 (m, 4H); 4.35 (d, 1H); 6.72 (d, 1H); 6.98 (d, 2H); 7.08 (d, 1H); 7.18 (d, 2H); 10.73 (s, 1H).

### Intermediate 84

### tert-Butyl 4-[4-(6-chloro-1,3-dimethyl-2-oxo-2,3-dihydropyrido[2,3-b]pyrazin-4(1H)-yl)phenyl]piperazine-1-carboxylate

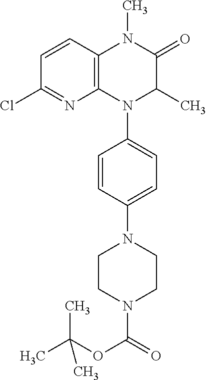 [SMILES: CC1C(=O)N(C)c2ccc(Cl)nc2N1c1ccc(N2CCN(C(=O)OC(C)(C)C)CC2)cc1]

A solution of 750 mg of Intermediate 83 and 0,145 ml of methyl iodide in 30 ml of DMF was admixed at 0° C. with 0.93 g of sodium hydride (60% in white oil). After stirring at 0° C. for one hour and at RT for 14 hours, the mixture was diluted with ethyl acetate and washed three times with semisaturated aqueous sodium chloride solution. The combined organic phases were dried over sodium sulphate and concentrated fully under reduced pressure. The residue was purified by chromatography on silica gel (dichloromethane/methanol gradient up to 2% methanol content). This gave 630 mg of tert-butyl 4-[4-(6-chloro-1,3-dimethyl-2-oxo-2,3-dihydropyrido[2,3-b]pyrazin-4(1H)-yl)phenyl]piperazine-1-carboxylate.

¹H-NMR (400 MHz, 25° C., DMSO-d6): δ=1.25 (d, 3H); 1.43 (s, 9H); 3.10-3.16 (m, 4H); 3.30 (s, 3H); 3.43-3.50 (m, 4H); 4.45 (q, 1H); 6.85 (d, 1H); 6.99 (d, 2H); 7.19 (d, 2H); 7.41 (d, 1H).

### Intermediate 85

### (3R)-4-Benzyl-6-bromo-3-methyl-3,4-dihydroquinoxalin-2(1H)-one

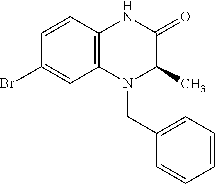 [SMILES: C[C@@H]1C(=O)Nc2ccc(Br)cc2N1Cc1ccccc1]

A solution of 5 g of Intermediate 12, 6.6 g of benzaldehyde, 6.7 g of phenylsilane and 6.3 g of dibutyltin dichloride in 70 ml of THF was stirred at RT for 85 hours. The solution was concentrated fully under reduced pressure. The residue was purified by chromatography on silica gel (hexane/ethyl acetate gradient up to 30% ethyl acetate content). This gave 6.14 g of (3R)-4-benzyl-6-bromo-3-methyl-3,4-dihydroquinoxalin-2(1H)-one.

¹H-NMR (400 MHz, 25° C., CDCl₃): δ=1.25 (d, 3H); 3.97 (q, 1H); 4.21 (d, 1H); 4.62 (d, 1H); 6.70 (d, 1H); 6.89 (d, 1H); 6.94 (dd, 1H); 7.32-7.44 (m, 5H); 8.98 (bs, 1H).

### Intermediate 86

### (3R)-4-Benzyl-6-bromo-1,3-dimethyl-3,4-dihydroquinoxalin-2(1H)-one

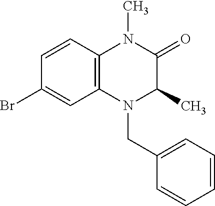 [SMILES: C[C@@H]1C(=O)N(C)c2ccc(Br)cc2N1Cc1ccccc1]

A solution of 6.14 g of Intermediate 85 and 1.73 ml of methyl iodide in 80 ml of DMF was admixed at 0° C. with 1.11 g of sodium hydride (60% in white oil). After stirring at 0° C. for one hour and at RT for 30 minutes, the mixture was admixed with saturated ammonium chloride solution and extracted with ethyl acetate. The combined organic phases were dried over sodium sulphate and concentrated fully under reduced pressure. The residue was purified by chromatography on silica gel (hexane/ethyl acetate gradient up to 35% ethyl acetate content). This gave 5.9 g of (3R)-4-benzyl-6-bromo-1,3-dimethyl-3,4-dihydroquinoxalin-2(1H)-one.

¹H-NMR (400 MHz, 25° C., CDCl₃): δ=1.11 (d, 3H); 3.37 (s, 3H); 3.96 (q, 1H); 4.12 (d, 1H); 4.54 (d, 1H); 6.81 (d, 1H); 6.85 (d, 1H); 6.99 (dd, 1H); 7.28-7.40 (m, 5H).

### Intermediate 87

### tert-Butyl 4-[(2R)-7-bromo-2-methyl-3-oxo-3,4-dihydroquinoxalin-1(2H)-yl]piperidine-1-carboxylate

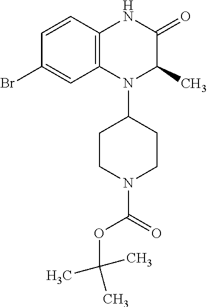 [SMILES: C[C@@H]1C(=O)Nc2ccc(Br)cc2N1C1CCN(C(=O)OC(C)(C)C)CC1]

A solution of 3 g of Intermediate 12, 7.4 g of tert-butyl 4-oxopiperidine-1-carboxylate (CAS 79099-07-3), 4.2 g of phenylsilane and 3.78 g of dibutyltin dichloride in 100 ml of THF was stirred at RT for 76 hours. After addition of diatomaceous earth, the mixture was concentrated fully under reduced pressure. The residue was purified by chromatography on silica gel (hexane/ethyl acetate gradient up to 100% ethyl acetate content) and RP-HPLC (Waters SQD autopurification system; column: Waters XBridge C18 5μ 100×30 mm; eluent A: water+0.2% by vol. of ammonia (32%), eluent B: acetonitrile; gradient: 0-8.0 min 1-100% B, 8.0-10.0 min 100% B; flow rate 50.0 ml/min; temperature: 60° C.; injection: 2500 μl; DAD scan: 210-400 nm). This gave 2.4 g of tert-butyl 4-[(2R)-7-bromo-2-methyl-3-oxo-3,4-dihydroquinoxalin-1(2H)-yl]piperidine-1-carboxylate.

¹H-NMR (400 MHz, DMSO-d6): δ=0.97 (d, 3H); 1.41 (s, 9H); 1.46-1.65 (m, 3H); 1.88 (bd, 1H); 2.70-2.98 (m, 2H); 3.67 (tt, 1H); 3.91 (q, 1H); 3.95-4.08 (m, 2H); 6.74 (d, 1H); 6.89 (dd, 1H); 7.07 (d, 1H); 10.43 (s, 1H).

### Intermediate 88

### tert-Butyl 4-[(2R)-7-bromo-2,4-dimethyl-3-oxo-3,4-dihydroquinoxalin-1(2H)-yl]piperidine-1-carboxylate

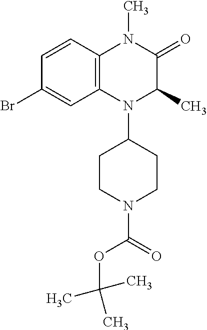 [SMILES: C[C@@H]1C(=O)N(C)c2ccc(Br)cc2N1C1CCN(C(=O)OC(C)(C)C)CC1]

A solution of 2.4 g of Intermediate 87 and 0.52 ml of methyl iodide in 40 ml of DMF was admixed at 0° C. with 520 mg of sodium hydride (60% in white oil). After stirring at 0° C. for one hour, the mixture was admixed with semisaturated sodium hydrogencarbonate solution and extracted three times with dichloromethane. The combined organic phases were dried over sodium sulphate and concentrated fully under reduced pressure. The residue was purified by chromatography on modified silica gel (column: Biotage KP-NH, hexane/ethyl acetate gradient up to 30% ethyl acetate content). This gave 2.47 g of tert-butyl 4-[(2R)-7-bromo-2,4-dimethyl-3-oxo-3,4-dihydroquinoxalin-1(2H)-yl]piperidine-1-carboxylate.

¹H-NMR (400 MHz, CDCl₃): δ=1.11 (d, 3H); 1.48 (s, 9H); 1.57-1.74 (m, 3H); 1.98 (bd, 1H); 2.70-2.97 (m, 2H); 3.35 (s, 3H); 3.46-3.56 (m, 1H); 4.10 (q, 1H); 4.15-4.34 (m, 2H); 6.83 (d, 1H); 7.01-7.08 (m, 2H).

### Intermediate 89

### Methyl 3-{[(3R)-1,3-dimethyl-2-oxo-4-(tetrahydro-2H-pyran-4-yl)-1,2,3,4-tetrahydroquinoxalin-6-yl]amino}benzoate

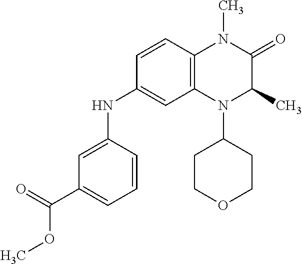 [SMILES: COC(=O)c1cccc(Nc2ccc3c(c2)N(C2CCOCC2)[C@H](C)C(=O)N3C)c1]

A suspension of 2.0 g of Intermediate 14, 1.69 g of methyl 3-amino-2-methoxybenzoate (CAS 5129-25-9), 126 mg of palladium(II) acetate, 5.48 g of caesium carbonate and 349 mg of (+)-BINAP in 125 ml of toluene was stirred at 120° C. under an argon atmosphere for 14 hours. The reaction solution was filtered through kieselguhr and the solvent was removed under reduced pressure. The residue was purified by chromatography on silica gel (hexane/ethyl acetate gradient up to 100% ethyl acetate content). This gave 1.5 g of methyl 3-{[(3R)-1,3-dimethyl-2-oxo-4-(tetrahydro-2H-pyran-4-yl)-1,2,3,4-tetrahydroquinoxalin-6-yl]amino}methoxybenzoate.

1H NMR: (400 MHz, 25° C., CDCl₃): δ=1.13 (d, 3H); 1.68-1.97 (m, 4H); 3.37 (s, 3H); 3.43 (dt, 2H); 3.56 (tt, 1H); 3.90 (s, 3H); 3.99-4.10 (m, 2H); 4.10-4.19 (m, 1H); 5.74 (bs, 1H); 6.63-6.71 (m, 2H); 6.90 (d, 1H); 7.16 (dd, 1H); 7.32/t, 1H); 7.55 (d, 1H); 7.73 (bs, 1H).

### Intermediate 90

### 3-{[(3R)-1,3-Dimethyl-2-oxo-4-(tetrahydro-2H-pyran-4-yl)-1,2,3,4-tetrahydroquinoxalin-6-yl]amino}benzoic acid

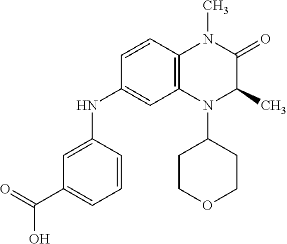 [SMILES: C[C@@H]1C(=O)N(C)c2ccc(Nc3cccc(C(=O)O)c3)cc2N1C1CCOCC1]

A solution of 300 mg of Intermediate 89 in 2 ml of THF and 16 ml of methanol was admixed at RT with 6.9 ml of 1N lithium hydroxide solution and stirred at 60° C. for 3 hours. The mixture was adjusted to pH=7 using 1N hydrochloric acid and extracted twice with ethyl acetate. The combined organic phases were dried over sodium sulphate and the solvent was removed completely under reduced pressure. This gave 270 mg of 3-{[(3R)-1,3-dimethyl-2-oxo-4-(tetrahydro-2H-pyran-4-yl)-1,2,3,4-tetrahydroquinoxalin-6-yl]amino}benzoic acid.

¹H NMR: (300 MHz, 25° C., DMSO-d6): δ=0.98 (d, 3H); 1.58-1.93 (m, 4H); 3.25 (s, 3H); 3.36-3.45 (m, 2H); 3.59 (tt, 1H); 3.85-3.98 (m, 2H); 4.06 (q, 1H); 6.64 (dd, 1H); 6.72 (d, 1H); 6.99 (d, 1H); 7.15-7.23 (m, 1H); 7.27-7.36 (m, 2H); 7.67 (bs, 1H); 8.25 (s, 1H); 12.70 (bs, 1H).

### Intermediate 91

### N-(1-Methylpiperidin-4-yl)-3-nitrobenzenesulphonamide

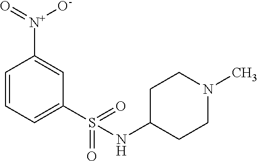 [SMILES: CN1CCC(NS(=O)(=O)c2cccc([N+](=O)[O-])c2)CC1]

A solution of 3.0 g of 3-nitrobenzenesulphonyl chloride (CAS 121-51-7) and 1.62 g of 1-methylpiperidine-4-amine (CAS 41838-46-4) in 75 ml of dichloromethane was admixed at 0° C. with 7.2 ml of triethylamine and stirred for 18 hours, in the course of which the temperature was raised gradually to RT. The reaction was diluted with dichloromethane and washed with water and saturated sodium chloride solution, dried over sodium sulphate and concentrated fully under reduced pressure. The residue that remained was purified by chromatography on silica gel (dichloromethane/methanol gradient). This gave 3.13 g of the title compound.

¹H-NMR (400 MHz, 25° C., CDCl₃): δ=1.55-1.67 (m, 2H); 1.86 (dd, 2H); 2.11 (t, 2H); 2.30 (s, 3H); 2.79 (d, 2H); 3.25-3.34 (m, 1H); 7.77 (t, 1H); 8.25 (dt, 1H); 8.46 (ddd, 1H); 8.76 (t, 1H).

### Intermediate 92

### 3-Amino-N-(1-methylpiperidin-4-yl)benzenesulphonamide

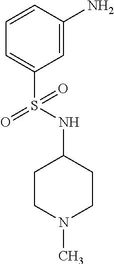 [SMILES: CN1CCC(NS(=O)(=O)c2cccc(N)c2)CC1]

A suspension of 3.5 g of Intermediate 91 and 350 mg of palladium (10% on activated carbon) in 100 ml of methanol was shaken under a hydrogen atmosphere at RT for 8 hours. The mixture was filtered through kieselguhr and the solution was concentrated fully under reduced pressure. This gave 2.8 g of the title compound.

¹H-NMR (300 MHz, 25° C., DMSO-d6): δ=1.26-1.42 (m, 2H); 1.45-1.57 (m, 2H); 1.70-1.83 (m, 2H); 2.05 (s, 3H); 2.58 (bd, 2H); 2.75-2.89 (m, 1H); 5.54 (bs, 2H); 6.71 (bdd, 1H); 6.88 (bd, 1H); 6.97 (t, 1H); 7.16 (t, 1H); 7.45 (d, 1H).

### Intermediate 93

### N-[2-(Dimethylamino)ethyl]-3-nitrobenzenesulphonamide

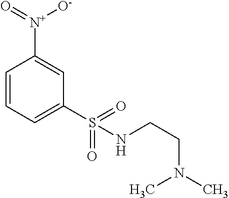 [SMILES: CN(C)CCNS(=O)(=O)c1cccc([N+](=O)[O-])c1]

A solution of 3.0 g of 3-nitrobenzenesulphonyl chloride (CAS 121-51-7) and 1.245 g of N,N-dimethylethane-1,2-diamine (CAS 108-00-9) in 75 ml of dichloromethane was admixed at 0° C. with 7.2 ml of triethylamine and stirred for 19 hours, in the course of which the temperature was raised gradually to RT. The reaction was diluted with dichloromethane and washed with water and saturated sodium chloride solution, dried over sodium sulphate and concentrated fully under reduced pressure. The residue that remained was purified by chromatography on silica gel (dichloromethane/methanol gradient). This gave 3.30 g of the title compound.

¹H-NMR (300 MHz, 25° C., CDCl₃): δ=2.15 (s, 6H); 2.38-2.46 (m, 2H); 3.04-3.11 (m, 2H); 7.77 (t, 1H); 8.25 (d, 1H); 8.46 (ddd, 1H); 8.75 (bt, 1H).

### Intermediate 94

### 3-Amino-N-[2-(dimethylamino)ethyl]benzenesulphonamide

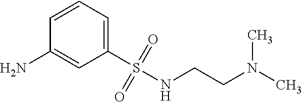 [SMILES: CN(C)CCNS(=O)(=O)c1cccc(N)c1]

A suspension of 3.3 g of Intermediate 93 and 330 mg of palladium (10% on activated carbon) in 200 ml of methanol was shaken under a hydrogen atmosphere at RT for 7 hours. The mixture was filtered through kieselguhr and the solution was concentrated fully under reduced pressure. This gave 2.8 g of the title compound as a yellow foam.

¹H-NMR (400 MHz, 25° C., DMSO-d6): δ=2.05 (s, 6H); 2.22 (t, 2H); 2.78 (t, 2H); 5.53 (bs, 2H); 6.73 (ddd, 1H); 6.86 (dd, 1H); 6.97 (t, 1H); 7.17 (t, 1H); 7.20-7.27 (m, 1H).

### Intermediate 95

### 4-[(4-Chloro-3-nitrophenyl)sulphonyl]morpholine

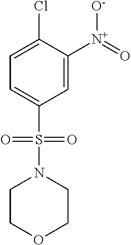 [SMILES: O=[N+]([O-])c1cc(S(=O)(=O)N2CCOCC2)ccc1Cl]

To a solution of 22 g of 4-chloro-3-nitrobenzenesulphonyl chloride (CAS 97-08-5) in 330 ml of dichloromethane were added dropwise, at −40° C., 18 ml of triethylamine and 7.49 g of morpholine (CAS 110-91-8), the mixture was stirred at −40° C. for 1 hour, and the temperature was raised gradually to RT. The mixture was washed with water and saturated sodium chloride solution, dried over magnesium sulphate and concentrated fully under reduced pressure. This gave 24 g of the title compound as a yellow solid.

¹H NMR (400 MHz, 25° C., CDCl₃): δ=3.05-3.07 (m, 4H); 3.76-3.78 (m, 4H); 7.77 (d, 1H); 7.87 (dd, 1H); 8.23 (d, 1H).

### Intermediate 96

### 4-[(4-Methoxy-3-nitrophenyl)sulphonyl]morpholine

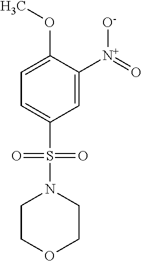 [SMILES: COc1ccc(S(=O)(=O)N2CCOCC2)cc1[N+](=O)[O-]]

A solution of 28 g of 4-[(4-chloro-3-nitrophenyl)sulphonyl]morpholine (Intermediate 95) in 250 ml of methanol was admixed at 25° C. with 84 ml of sodium methoxide (30% solution in methanol), and the mixture was stirred for 2 hours. The mixture was admixed with ice, thawed and then filtered at RT, washed with water and dried. This gave 26 g of the title compound as a yellow solid.

¹H NMR (400 MHz, 25° C., CDCl₃): δ=3.02 (dd, 4H); 3.76 (dd, 4H); 4.06 (s, 3H); 7.23-7.25 (m, 1H); 7.91 (dd, 1H); 8.21 (d, 1H).

### Intermediate 97

### 2-Methoxy-5-(morpholin-4-ylsulphonyl)aniline

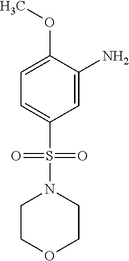 [SMILES: COc1ccc(S(=O)(=O)N2CCOCC2)cc1N]

A suspension of 1.0 g of Intermediate 96 in 45.8 ml of ethanol was stirred under reflux together with 3.73 g of tin(II) chloride dihydrate for 1.5 hours. Then the pH was adjusted to 8 with saturated sodium carbonate solution, and the mixture was stirred for 1 hour, extracted three times with ethyl acetate, washed twice with saturated sodium chloride solution, dried over magnesium sulphate and concentrated fully under reduced pressure. This gave 790 mg of the title compound as a beige solid.

¹H NMR (400 MHz, 25° C., DMSO-d6): δ=2.78-2.80 (m, 4H); 3.60-3.63 (m, 4H); 3.85 (s, 3H); 5.27 (s, 2H); 6.88 (dd, 1H); 6.96-7.00 (m, 2H).

### Intermediate 98

### 3,3-Difluoro-1-[(3-nitrophenyl)sulphonyl]azetidine

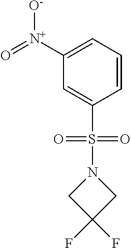 [SMILES: O=[N+]([O-])c1cccc(S(=O)(=O)N2CC(F)(F)C2)c1]

A solution of 10.27 g of 3-nitrobenzenesulphonyl chloride (CAS 121-51-7) and 4.0 g of 3,3-difluoroazetidine hydrochloride (CAS 288315-03-7) in 120 ml of dichloromethane was admixed at 0° C. with 17.2 ml of triethylamine and stirred for 1 hour; the temperature was raised gradually to RT. The mixture was washed with water and saturated sodium chloride solution, dried over magnesium sulphate, concentrated fully under reduced pressure and crystallized from warm ethyl acetate. This gave 7.28 g of the title compound in solid form.

¹H NMR (400 MHz, 25° C., CDCl3): δ=4.27 (t, 4H); 7.83 (t, 1H); 8.19 (dt, 1H); 8.53 (ddd, 1H); 8.70 (d, 1H).

### Intermediate 99

### 3-[(3,3-Difluoroazetidin-1-yl)sulphonyl]aniline

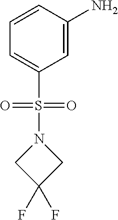 [SMILES: Nc1cccc(S(=O)(=O)N2CC(F)(F)C2)c1]

A suspension of 114 mg of Intermediate 98 and 14 mg of palladium (5% on activated carbon) in 1 ml of ethyl acetate was stirred under a hydrogen atmosphere at RT for 2 hours. The mixture was filtered through Celite and the solution was concentrated fully under reduced pressure. This gave 97 mg of crude title compound, which was stirred with diethyl ether to give a beige solid.

¹H NMR (400 MHz, 25° C., DMSO-d6): δ=4.15 (t, 4H), 5.72 (s, 2H), 6.88 (bs, 2H), 6.97 (s, 1H), 7.28 (t, 1H).

### Intermediate 100

### 6-[(4-Methoxy-3-nitrophenyl)sulphonyl]-2-oxa-6-azaspiro[3.3]heptane

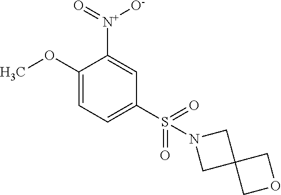 [SMILES: COc1ccc(S(=O)(=O)N2CC3(COC3)C2)cc1[N+](=O)[O-]]

A solution of 3.0 g 4-methoxy-3-nitrobenzenesulphonyl chloride (CAS 22117-79-9) in 60 ml of dichloromethane was admixed with 2.11 g of 2-oxa-6-azaspiro[3.3]heptane oxalate (1:2) (CAS 1045709-32-7, 1159599-99-1), 6.45 ml of triethylamine were added thereto at bath temperature 0° C., and the mixture was stirred for 18 hours, in the course of which the temperature was raised gradually to RT. The reaction was diluted with dichloromethane and washed with water and saturated sodium chloride solution, dried over sodium sulphate and concentrated fully under reduced pressure. The remaining residue was purified by chromatography on silica gel (dichloromethane/methanol gradient up to 1% methanol content). This gave 1.5 g of 6-[(4-methoxy-3-nitrophenyl)sulphonyl]-2-oxa-6-azaspiro[3.3]heptane.

¹H NMR (400 MHz, 25° C., DMSO-d6): δ=3.92 (s, 4H); 4.03 (s, 3H); 4.45 (s, 4H); 7.59 (d, 1H); 8.05 (dd, 1H); 8.25 (d, 1H).

### Intermediate 101

### 2-Methoxy-5-(2-oxa-6-azaspiro[3.3]hept-6-ylsulphonyl)aniline

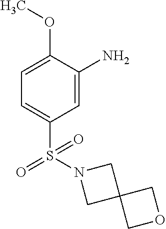 [SMILES: COc1ccc(S(=O)(=O)N2CC3(COC3)C2)cc1N]

A suspension of 1.50 g of Intermediate 100 and 0.15 g of palladium (10% on activated carbon) in 300 ml of methanol was shaken under a hydrogen atmosphere at RT for 8 hours. The mixture was filtered through kieselguhr and the solution was concentrated fully under reduced pressure. This gave 1.20 g of 2-methoxy-5-(2-oxa-6-azaspiro[3.3]hept-6-ylsulphonyl)aniline as a beige solid.

¹H NMR (400 MHz, 25° C., DMSO-d6): δ=3.81 (s, 4H); 3.86 (s, 3H); 4.46 (s, 4H); 5.27 (bs, 2H); 6.94 (dd, 1H); 7.00 (d, 1H); 7.04 (d, 1H).

### Intermediate 102

### 2-[(3-Nitrophenyl)sulphonyl]-2-azaspiro[3.3]heptane

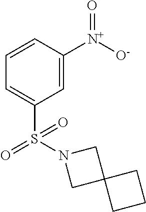 [SMILES: O=[N+]([O-])c1cccc(S(=O)(=O)N2CC3(CCC3)C2)c1]

A solution of 0.27 g of 3-nitrobenzenesulphonyl chloride (CAS 121-51-7) and 0.17 g of 2-azaspiro[3.3]heptane hydrochloride (1:1) (CAS 665-04-3) in 27 ml of dichloromethane was admixed at bath temperature 0° C. with 0.65 ml of triethylamine and stirred for 18 hours, in the course of which the temperature was raised gradually to RT. The reaction was diluted with dichloromethane and washed with water and saturated sodium chloride solution, dried over sodium sulphate and concentrated fully under reduced pressure. This gave 310 mg of 2-[(3-nitrophenyl)sulphonyl]-2-azaspiro[3.3]heptane.

¹H NMR (400 MHz, 25° C., DMSO-d6): δ=1.58-1.67 (m, 2H); 1.85-1.90 (m, 4H); 3.73 (s, 4H); 7.97 (t, 1H); 8.23 (dt, 1H); 8.40 (t, 1H); 8.57 (ddd, 1H).

### Intermediate 103

### 3-(2-Azaspiro[3.3]hept-2-ylsulphonyl)aniline

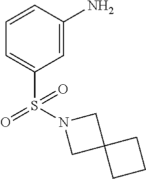 [SMILES: Nc1cccc(S(=O)(=O)N2CC3(CCC3)C2)c1]

A suspension of 0.30 g of Intermediate 102 and 37.5 mg of palladium (10% on activated carbon) in 24 ml of methanol was shaken under a hydrogen atmosphere at RT for 7 hours. The mixture was filtered through kieselguhr and the solution was concentrated fully under reduced pressure. This gave 0.23 g of 3-(2-azaspiro[3.3]hept-2-ylsulphonyl)aniline as a yellow oil.

¹H NMR (400 MHz, 25° C., DMSO-d6): δ=1.59-1.70 (m, 2H); 1.88 (t, 4H); 3.61 (s, 4H); 5.63 (bs, 2H); 6.83 (t, 2H); 6.95 (bs, 1H); 7.26 (t, 1H).

### Intermediate 104

### N-{4-[4-(Cyclopropylmethyl)piperazin-1-yl]cyclohexyl}-3-nitrobenzenesulphonamide

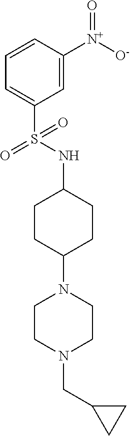 [SMILES: O=[N+]([O-])c1cccc(S(=O)(=O)NC2CCC(N3CCN(CC4CC4)CC3)CC2)c1]

A solution of 3.0 g of 3-nitrobenzenesulphonyl chloride (CAS 121-51-7) and 3.36 g of 4-[4-(cyclopropylmethyl)piperazin-1-yl]cyclohexanamine (CAS 876461-31-3, prepared analogously to WO2012049153) in 75 ml of dichloromethane was admixed at 0° C. with 7.17 ml of triethylamine and stirred for 16 hours, in the course of which the temperature was raised gradually to RT. The reaction was diluted with dichloromethane and washed with water and saturated sodium chloride solution, dried over sodium sulphate and concentrated fully under reduced pressure. The remaining residue was purified by chromatography on silica gel (dichloromethane/methanol gradient up to 1% methanol content). This gave 1.3 mg of N-{4-[4-(cyclopropylmethyl)piperazin-1-yl]cyclohexyl}-3-nitrobenzenesulphonamide.

¹H NMR (300 MHz, 25° C., DMSO-d6): δ=0.01 (q, 2H); 0.35-0.46 (m, 2H); 0.75 (t, 1H); 1.13 (t, 4H); 1.65 (d, 4H); 2.05-2.13 (m, 4H); 2.38 (bs, 8H); 7.88 (t, 1H); 8.03 (d, 1H); 8.22 (bd, 1H); 8.45 (ddd, 1H); 8.53 (t, 1H).

### Intermediate 105

### 3-Amino-N-{cis-4-[4-(cyclopropylmethyl)piperazin-1-yl]cyclohexyl}benzenesulphonamide

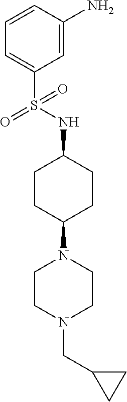 [SMILES: Nc1cccc(S(=O)(=O)N[C@H]2CC[C@@H](N3CCN(CC4CC4)CC3)CC2)c1]

A suspension of 1.30 g of Intermediate 104 and 0.19 g of palladium (10% on activated carbon) in 100 ml of methanol was shaken under a hydrogen atmosphere at RT for 4 hours. The mixture was filtered through kieselguhr, and the solution was concentrated fully under reduced pressure and purified by chromatography. This gave 1.1 g of 3-amino-N-{4-[4-(cyclopropylmethyl)piperazin-1-yl]cyclohexyl}benzenesulphonamide. The latter was separated into the cis/trans isomers by RP-HPLC (Waters SQD autopurification system; column: Waters XBridge C18 5 μm 100×30 mm; eluent A: water+0.1% by vol. of formic acid (99%), eluent B: acetonitrile; gradient: 0-8.0 min 1-100% B, 8.0-10.0 min 100% B; flow rate 50.0 ml/min; temperature: 60° C.; injection: 2500 μl; DAD scan: 210-400 nm). This gave 255 mg of 3-amino-N-{cis-4-[4-(cyclopropylmethyl)piperazin-1-yl]cyclohexyl}benzenesulphonamide.

¹H NMR (500 MHz, 25° C., DMSO-d6): δ=0.00-0.04 (m, 2H); 0.39-0.44 (m, 2H); 0.73-0.80 (m, 1H); 1.06-1.18 (m, 6H); 1.68 (d, 4H); 2.03-2.11 (m, 3H); 2.32-2.44 (m, 6H); 2.76-2.87 (m, 1H); 5.51 (s, 2H); 6.72 (ddd, 1H); 6.89 (bd, 1H); 6.99 (t, 1H); 7.16 (t, 1H); 7.39 (d, 1H).

UPLC-MS: Instrument: Waters Acquity UPLC-MS SQD; column: Acquity UPLC BEH C18 1.7 50×2.1 mm; eluent A: water+0.2% by vol. of ammonia (32%), eluent B: acetonitrile; gradient: 0-1.6 min 1-99% B, 1.6-2.0 min 99% B; flow rate 0.8 ml/min; temperature: 60° C.; injection: 2 μl; DAD scan: 210-400 nm

Rt=0.95 min.

### Intermediate 106

### 3-Amino-N-{trans-4-[4-(cyclopropylmethyl)piperazin-1-yl]cyclohexyl}benzenesulphonamide

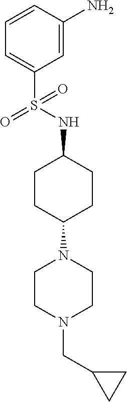 [SMILES: Nc1cccc(S(=O)(=O)N[C@H]2CC[C@H](N3CCN(CC4CC4)CC3)CC2)c1]

A suspension of 1.30 g of Intermediate 104 and 0.19 g of palladium (10% on activated carbon) in 100 ml of methanol was shaken under a hydrogen atmosphere at RT for 4 hours. The mixture was filtered through kieselguhr, and the solution was concentrated fully under reduced pressure and purified by chromatography. This gave 1.1 g of 3-amino-N-{4-[4-(cyclopropylmethyl)piperazin-1-yl]cyclohexyl}benzenesulphonamide. The latter was separated into the cis/trans isomers by RP-HPLC (Waters SQD autopurification system; column: Waters XBridge C18 5 μm 100×30 mm; eluent A: water+0.1% by vol. of formic acid (99%), eluent B: acetonitrile; gradient: 0-8.0 min 1-100% B, 8.0-10.0 min 100% B; flow rate 50.0 ml/min; temperature: 60° C.; injection: 2500 μl; DAD scan: 210-400 nm). This gave 40 mg of 3-amino-N-{trans-4-[4-(cyclopropylmethyl)piperazin-1-yl]cyclohexyl}benzenesulphonamide.

¹H NMR (500 MHz, 25° C., DMSO-d6): δ=0.00-0.04 (m, 2H); 0.39-0.44 (m, 2H); 0.73-0.80 (m, 1H); 1.06-1.18 (m, 6H); 1.68 (d, 4H); 2.03-2.11 (m, 3H); 2.32-2.44 (m, 6H); 2.76-2.87 (m, 1H); 5.51 (s, 2H); 6.72 (ddd, 1H); 6.89 (bd, 1H); 6.99 (t, 1H); 7.16 (t, 1H); 7.39 (d, 1H).

UPLC-MS: Instrument: Waters Acquity UPLC-MS SQD; column: Acquity UPLC BEH C18 1.7 50×2.1 mm; eluent A: water+0.2% by vol. of ammonia (32%), eluent B: acetonitrile; gradient: 0-1.6 min 1-99% B, 1.6-2.0 min 99% B; flow rate 0.8 ml/min; temperature: 60° C.; injection: 2 μl; DAD scan: 210-400 nm

Rt=0.92 min.

### Intermediate 107

### 1-[(3-Nitrophenyl)sulphonyl]-4-(2,2,2-trifluorethyl)piperazine

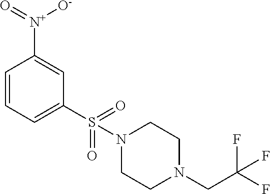 [SMILES: O=[N+]([O-])c1cccc(S(=O)(=O)N2CCN(CC(F)(F)F)CC2)c1]

A solution of 2.0 g of 3-nitrobenzenesulphonyl chloride (CAS 121-51-7) and 1.59 g of 1-(2,2,2-trifluoroethyl)piperazine (CAS 13349-90-1) in 50 ml of dichloromethane was admixed at 0° C. with 4.78 ml of triethylamine and stirred for 18 hours, in the course of which the temperature was raised gradually to RT. The reaction was diluted with dichloromethane and washed with water and saturated sodium chloride solution, dried over sodium sulphate and concentrated fully under reduced pressure. This gave 3.1 g of 1-[(3-nitrophenyl)sulphonyl]-4-(2,2,2-trifluorethyl)piperazine as a yellow solid.

¹H NMR (400 MHz, 25° C., DMSO-d6): δ=2.70 (bt, 4H); 2.96-3.03 (m, 4H); 3.20 (q, 2H); 7.97 (t, 1H); 8.18 (dt, 1H); 8.37 (t, 1H); 8.57 (ddd, 1H).

### Intermediate 108

### 3-{[4-(2,2,2-Trifluoroethyl)piperazin-1-yl]sulphonyl}aniline

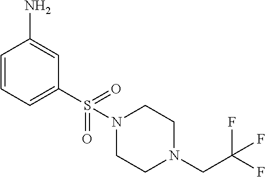 [SMILES: Nc1cccc(S(=O)(=O)N2CCN(CC(F)(F)F)CC2)c1]

A suspension of 3.1 g of Intermediate 107 and 0.31 g of palladium (10% on activated carbon) in 200 ml of methanol was shaken under a hydrogen atmosphere at RT for 7 hours. The mixture was filtered through kieselguhr and the solution was concentrated fully under reduced pressure. This gave 2.8 g of 3-{[4-(2,2,2-trifluoroethyl)piperazin-1-yl]sulphonyl}aniline as a yellow solid.

¹H NMR (400 MHz, 25° C., DMSO-d6): δ=2.64-2.69 (m, 4H); 2.82-2.88 (m, 4H); 3.17 (q, 2H); 5.63 (s, 2H); 6.77 (ddd, 1H); 6.83 (ddd, 1H); 6.88 (t, 1H); 7.24 (t, 1H).

### Intermediate 109

### 1-Methyl-4-[(3-nitrophenyl)sulphonyl]piperazine

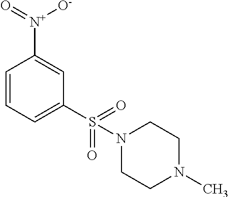 [SMILES: CN1CCN(S(=O)(=O)c2cccc([N+](=O)[O-])c2)CC1]

A solution of 3.0 g of 3-nitrobenzenesulphonyl chloride (CAS 121-51-7) and 1.42 g of 1-methylpiperazine (CAS 109-01-3) in 75 ml of dichloromethane was admixed at 0° C. with 7.2 ml of triethylamine and stirred for 19 hours, in the course of which the temperature was raised gradually to RT. The reaction was diluted with dichloromethane and washed with water and saturated sodium chloride solution, dried over sodium sulphate and concentrated fully under reduced pressure. The remaining residue was purified by chromatography on silica gel (dichloromethane/methanol gradient up to 1% methanol content). This gave 3.55 g of 1-methyl-4-[(3-nitrophenyl)sulphonyl]piperazine as a beige solid.

¹H NMR (400 MHz, 25° C., DMSO-d6): δ=2.13 (s, 3H); 2.32-2.38 (m, 4H); 2.94-3.00 (m, 4H); 7.95 (t, 1H); 8.17 (bd, 1H); 8.36 (t, 1H); 8.54 (ddd, 1H).

### Intermediate 110

### 3-[(4-Methylpiperazin-1-yl)sulphonyl]aniline

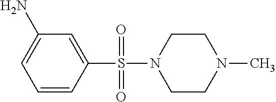 [SMILES: CN1CCN(S(=O)(=O)c2cccc(N)c2)CC1]

A suspension of 3.55 g of Intermediate 109 and 0.35 g of palladium (10% on activated carbon) in 100 ml of methanol was shaken under a hydrogen atmosphere at RT for 7 hours. The mixture was filtered through kieselguhr and the solution was concentrated fully under reduced pressure. This gave 3.18 g of 3-[(4-methylpiperazin-1-yl)sulphonyl]aniline as a beige solid.

¹H NMR (300 MHz, 25° C., DMSO-d6): δ=2.12 (s, 3H); 2.33 (t, 4H); 2.84 (bt, 4H); 5.63 (s, 2H); 6.76 (ddd, 1H); 6.81 (ddd, 1H); 6.88 (bt, 1H); 7.23 (t, 1H).

### Intermediate 111

### N-[(1-Methylpiperidin-4-yl)methyl]-3-nitrobenzenesulphonamide

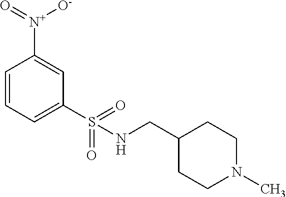 [SMILES: CN1CCC(CNS(=O)(=O)c2cccc([N+](=O)[O-])c2)CC1]

A solution of 1.50 g of 3-nitrobenzenesulphonyl chloride (CAS 121-51-7) and 0.91 g of 1-(1-methylpiperidin-4-yl)methanamine (CAS 7149-42-0) in 37.5 ml of dichloromethane was admixed at 0° C. with 3.59 ml of triethylamine and stirred for 19 hours, in the course of which the temperature was raised gradually to RT. The reaction was diluted with dichloromethane and washed with water and saturated sodium chloride solution, dried over sodium sulphate and concentrated fully under reduced pressure. The residue that remained was purified by chromatography on silica gel (dichloromethane/methanol gradient). This gave 1.6 g of N-[(1-methylpiperidin-4-yl)methyl]-3-nitrobenzenesulphonamide as a yellow foam.

¹H NMR (300 MHz, 25° C., DMSO-d6): δ=0.96-1.11 (m, 2H); 1.18-1.32 (m, 1H); 1.55 (bd, 2H); 1.72 (td, 2H); 2.09 (s, 3H); 2.66 (d, 4H); 7.89 (t, 1H); 7.98 (bs, 1H); 8.20 (dt, 1H); 8.47 (ddd, 1H); 8.49-8.53 (m, 1H).

### Intermediate 112

### 3-Amino-N-[(1-methylpiperidin-4-yl)methyl]benzenesulphonamide

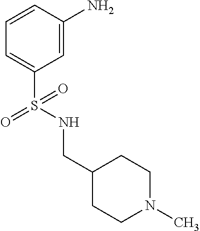 [SMILES: CN1CCC(CNS(=O)(=O)c2cccc(N)c2)CC1]

A suspension of 1.60 g of Intermediate 111 and 0.16 g of palladium (10% on activated carbon) in 50 ml of methanol was shaken under a hydrogen atmosphere at RT for 7 hours. The mixture was filtered through kieselguhr and the solution was concentrated fully under reduced pressure. This gave 1.40 g of 3-amino-N-[(1-methylpiperidin-4-yl)methyl]benzenesulphonamide as a beige foam.

¹H NMR (400 MHz, 25° C., DMSO-d6): δ=0.96-1.09 (m, 2H); 1.26 (td, 1H); 1.56 (d, 2H); 1.68-1.77 (m, 2H); 2.09 (s, 3H); 2.57 (t, 2H); 2.63-2.72 (m, 2H); 5.52 (s, 2H); 6.72 (ddd, 1H); 6.83-6.86 (m, 1H); 6.95 (t, 1H); 7.16 (t, 1H); 7.36 (t, 1H).

### Intermediate 113

### (3S)-6-Chloro-1,3-dimethyl-4-(tetrahydro-2H-pyran-4-yl)-3,4-dihydropyrido[2,3-b]pyrazin-2(1H)-one

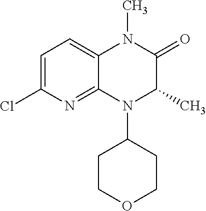 [SMILES: C[C@H]1C(=O)N(C)c2ccc(Cl)nc2N1C1CCOCC1]

In the enantiomer separation of Intermediate 10, smaller amounts of (3S)-6-chloro-1,3-dimethyl-4-(tetrahydro-2H-pyran-4-yl)-3,4-dihydropyrido[2,3-b]pyrazin-2(1H)-one were isolated.

Instrument: Waters Alliance 2695; column: Chiralpak IC 5 m 250×30 mm; eluent: hexane/2-propanol 70:30 (v/v); flow rate 35 ml/min; temperature: 25° C.; DAD 996 scan: 280 nm.

Rt=15.1-16.6 min

¹H NMR (400 MHz, CDCl₃): δ=1.23 (d, 3H); 1.62-1.69 (m, 1H); 1.81 (dq, 1H); 1.96 (dq, 1H); 2.02-2.09 (m, 1H); 3.31 (s, 1H); 3.51-3.62 (m, 2H); 4.02-4.10 (m, 2H); 4.31 (q, 1H); 4.54 (tt, 1H); 6.70 (d, 1H); 7.00 (d, 1H).

### Intermediate 114

### 1-[(4-Chloro-3-nitrophenyl)sulphonyl]-4-methylpiperazine

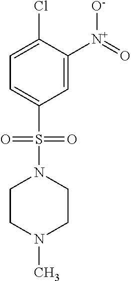 [SMILES: CN1CCN(S(=O)(=O)c2ccc(Cl)c([N+](=O)[O-])c2)CC1]

To a solution of 24 g of 4-chloro-3-nitrobenzenesulphonyl chloride (CAS 109-01-3) in 360 ml of dichloromethane were added dropwise, at −40° C., 19.6 ml of triethylamine and 10.4 ml of 1-methylpiperazine (CAS 110-91-8), the mixture was stirred at −40° C. for 1 hour, and the temperature was raised gradually to RT. The mixture was washed with water and saturated sodium chloride solution, dried over magnesium sulphate and concentrated fully under reduced pressure. This gave 28 g of 1-[(4-chloro-3-nitrophenyl)sulphonyl]-4-methylpiperazine as a yellow solid.

¹H NMR (400 MHz, 25° C., CDCl₃) δ=2.27 (s, 3H); 2.49 (t, 4H); 3.08 (bs, 4H); 7.73 (d, 1H); 7.86 (dd, 1H); 8.21 (d, 1H).

### Intermediate 115

### 1-[(4-Methoxy-3-nitrophenyl)sulphonyl]-4-methylpiperazine

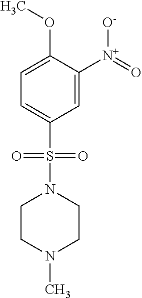 [SMILES: COc1ccc(S(=O)(=O)N2CCN(C)CC2)cc1[N+](=O)[O-]]

A solution of 28 g of 1-[(4-chloro-3-nitrophenyl)sulphonyl]-4-methylpiperazine (Intermediate 114) in 250 ml of methanol was admixed at 25° C. with 84 ml of sodium methoxide (30% in methanol), and the mixture was stirred for 2 hours. The mixture was admixed with ice, thawed and then filtered at RT, washed with water and dried. This gave 26 g of 1-[(4-methoxy-3-nitrophenyl)sulphonyl]-4-methylpiperazine as a yellow solid.

¹H NMR (400 MHz, 25° C., CDCl₃) δ=2.27 (s, 3H); 2.46-2.50 (bs, 4H); 3.05 (bs, 4H); 4.04 (s, 3H); 7.21 (d, 1H); 7.91 (dd, 1H); 8.21 (d, 1H).

### Intermediate 116

### 2-Methoxy-5-[(4-methylpiperazin-1-yl)sulphonyl]aniline

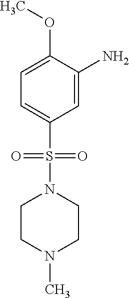 [SMILES: COc1ccc(S(=O)(=O)N2CCN(C)CC2)cc1N]

A suspension of 12.0 g of 1-[(4-methoxy-3-nitrophenyl)sulphonyl]-4-methylpiperazine (Intermediate 115) in 100 ml of ethanol and 24 ml of saturated aqueous ammonium chloride solution was admixed with 10.6 g of iron powder and stirred under reflux for 2 hours. Then a further 12.6 ml of saturated aqueous ammonium chloride solution were added and the mixture was stirred under reflux for a further 4 hours. The reaction solution was filtered through kieselguhr and washed through with ethanol and ethyl acetate. The solution was concentrated under reduced pressure and the residue was suspended with water and extracted three times with ethyl acetate. The combined organic phases were dried over magnesium sulphate and concentrated fully under reduced pressure. The residue was stirred with methanol. This gave 4.7 g of 2-methoxy-5-[(4-methylpiperazin-1-yl)sulphonyl]aniline as a yellow solid.

¹H NMR (400 MHz, 25° C., DMSO-d6) δ=2.14 (s, 3H); 2.36 (bs, 4H); 2.81 (bs, 4H); 3.84 (s, 3H); 5.25 (bs, 2H); 6.87 (dd, 1H); 6.95-6.98 (m, 2H).

### Intermediate 117

### N-{4-[(1-Methylpiperidin-4-yl)amino]cyclohexyl}acetamide, cisitrans isomer mixture

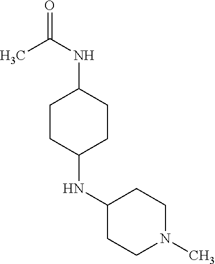 [SMILES: CC(=O)NC1CCC(NC2CCN(C)CC2)CC1]

To a solution of 772 mg of 4-amino-1-methylpiperine (CAS 41838-46-4) and 1 g of N-(4-oxocyclohexyl)acetamide (CAS 27514-08-5) in 10 ml of 1,2-dichloroethane and 0.37 ml of acetic acid were added, in portions at RT, 2.05 g of sodium triacetoxyborohydride. The mixture was stirred for 14 hours and then added to 1 N sodium hydroxide solution. The mixture was extracted twice with ethyl acetate, the combined organic phases were washed with saturated sodium chloride solution and dried over sodium sulphate, and the solvent was removed fully under reduced pressure. The residue was taken up again in ethyl acetate and the precipitate formed was filtered off with suction. This gave 3.8 g of N-{4-[(1-methylpiperidin-4-yl)amino]cyclohexyl}acetamide cis/trans isomer mixture as a crude product, which was used without further purification in the subsequent reaction.

UPLC-MS: Instrument: Waters Acquity UPLC-MS SQD; column: Acquity UPLC BEH C18 1.7 50×2.1 mm; eluent A: water+0.2% by vol. of ammonia (32%), eluent B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

Rt=0.66 min (M⁺+1=254)

### Intermediate 118

### N-(1-Methylpiperidin-4-yl)cyclohexane-1,4-diamine hydrochloride, cis/trans isomer mixture

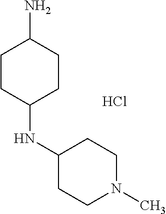

3.8 g of Intermediate 117 in 50 ml of hydrochloric acid (24% in water) were stirred at 115° C. for 16 hours. The mixture was then concentrated fully under reduced pressure and the residue was taken up in 2-propanol. This solution was heated and cooled again gradually. The precipitate formed was filtered off with suction. This gave 1.9 g of N-(1-methylpiperidin-4-yl)cyclohexane-1,4-diamine hydrochloride, cis/trans isomer mixture as a crude product, which was used without further purification in the subsequent reaction.

UPLC-MS: Instrument: Waters Acquity UPLC-MS SQD; column: Acquity UPLC BEH C18 1.7 50×2.1 mm; eluent A: water+0.2% by vol. of ammonia (32%), eluent B: acetonitrile; gradient: 0-1.6 min 1-99% B, 1.6-2.0 min 99% B; flow rate 0.8 ml/min; temperature: 60° C.; injection: 2 μl; DAD scan: 210-400 nm

Rt=0.66 and 0.77 min (M⁺+1=212): cis and trans isomer

### Intermediate 119

### tert-Butyl [4-(4,4-difluoropiperidin-1-yl)cyclohexyl]carbamate, cis/trans isomer mixture

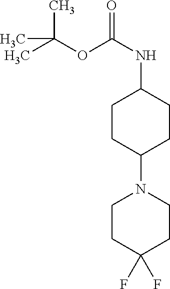 [SMILES: CC(C)(C)OC(=O)NC1CCC(N2CCC(F)(F)CC2)CC1]

To a solution of 2.26 g of 4,4-difluoropiperidine hydrochloride (CAS 144230-52-4) and 2 g of tert-butyl (4-oxocyclohexyl)carbamate (CAS 179321-49-4) in 50 ml of dichloromethane and 1.77 ml of triethylamine were added, in portions at RT, 4.48 g of sodium triacetoxyborohydride and a little acetic acid. The mixture was stirred for 14 hours, and 50 ml of methanol were then added. The mixture was stirred for 1 hour and diluted with dichloromethane. The reaction was washed with 1 N aqueous sodium hydroxide solution, water and saturated sodium chloride solution and dried over sodium sulphate, and the solvent was removed completely under reduced pressure. This gave 3.1 g of tert-butyl [4-(4,4-difluoropiperidin-1-yl)cyclohexyl]carbamate as a cis/trans isomer mixture.

UPLC-MS: Instrument: Waters Acquity UPLC-MS SQD; column: Acquity UPLC BEH C18 1.7 50×2.1 mm; eluent A: water+0.1% by vol. of formic acid (99%), eluent B: acetonitrile; gradient: 0-1.6 min 1-99% B, 1.6-2.0 min 99% B; flow rate 0.8 ml/min; temperature: 60° C.; injection: 2 μl;

DAD scan: 210-400 nm

Rt=0.68 min (M+1=319)

### Intermediate 120

### 4-(4,4-Difluoropiperidin-1-yl)cyclohexanamine, cis/trans isomer mixture

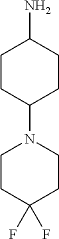 [SMILES: NC1CCC(N2CCC(F)(F)CC2)CC1]

11.3 ml of trifluoroacetic acid were added to 3.1 g of Intermediate 119 in 90 ml of dichloromethane, and the mixture was stirred at boiling point for 5 hours. The mixture was then concentrated fully under reduced pressure and the residue was taken up in ethyl acetate. The mixture was extracted with saturated sodium bicarbonate solution. The aqueous phase was then extracted three times with dichloromethane. The combined dichloromethane phases were dried over sodium sulphate and the solvent was removed completely under reduced pressure. This gave 920 mg of 4-(4,4-difluoropiperidin-1-yl)cyclohexanamine as a cis/trans isomer mixture.

UPLC-MS: Instrument: Waters Acquity UPLC-MS SQD; column: Acquity UPLC BEH C18 1.7 50×2.1 mm; eluent A: water+0.2% by vol. of ammonia (32%), eluent B: acetonitrile; gradient: 0-1.6 min 1-99% B, 1.6-2.0 min 99% B; flow rate 0.8 ml/min; temperature: 60° C.; injection: 2 μl; DAD scan: 210-400 nm

Rt=0.91+0.87 min (M⁺+1=219): cis and trans isomers

### Intermediate 121

### N-[4-(4-Cyclopropylpiperazin-1-yl)cyclohexyl]acetamide, cis/trans isomer mixture

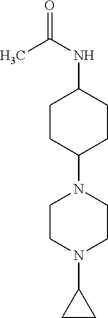 [SMILES: CC(=O)NC1CCC(N2CCN(C3CC3)CC2)CC1]

To a solution of 1.25 g of 1-cyclopropylpiperazine dihydrochloride (CAS 139256-79-4) and 928 mg of N-(4-oxocyclohexyl)acetamide (CAS 27514-08-5) in 9.3 ml of 1,2-dichloroethane and 0.34 ml of acetic acid were added, in portions at RT, 1.9 g of sodium triacetoxyborohydride. The mixture was stirred for 14 hours and then added to saturated sodium hydrogencarbonate solution. The mixture was extracted twice with ethyl acetate, the combined organic phases were washed with saturated sodium chloride solution and dried over sodium sulphate, and the solvent was removed fully under reduced pressure. This gave 600 g of N-[4-(4-cyclopropylpiperazin-1-yl)cyclohexyl]acetamide, cis/trans isomer mixture as a crude product, which was used without further purification in the subsequent reaction.

UPLC-MS: Instrument: Waters Acquity UPLC-MS SQD; column: Acquity UPLC BEH C18 1.7 50×2.1 mm; eluent A: water+0.2% by vol. of ammonia (32%), eluent B: acetonitrile; gradient: 0-1.6 min 1-99% B, 1.6-2.0 min 99% B; flow rate 0.8 ml/min; temperature: 60° C.; injection: 2 μl; DAD scan: 210-400 nm

Rt=0.71 and 0.74 min (M⁺+1=266)

### Intermediate 122

### 4-(4-Cyclopropylpiperazin-1-yl)cyclohexanamine hydrochloride, cis/trans isomer mixture

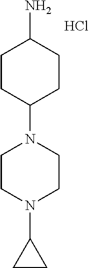 [SMILES: NC1CCC(N2CCN(C3CC3)CC2)CC1 ; Cl]

600 mg of Intermediate 121 in 10 ml of hydrochloric acid (24% in water) were stirred at 115° C. for 13 hours. The mixture was then concentrated fully under reduced pressure and the residue was taken up in 2-propanol. This solution was heated and cooled again gradually. The precipitate formed was filtered off with suction. This gave 170 mg of 4-(4-cyclopropylpiperazin-1-yl)cyclohexanamine hydrochloride, cis/trans isomer mixture as a crude product, which was used without further purification in the subsequent reaction.

UPLC-MS: Instrument: Waters Acquity UPLC-MS SQD; column: Acquity UPLC BEH C18 1.7 50×2.1 mm; eluent A: water+0.2% by vol. of ammonia (32%), eluent B: acetonitrile; gradient: 0-1.6 min 1-99% B, 1.6-2.0 min 99% B; flow rate 0.8 ml/min; temperature: 60° C.; injection: 2 μl; DAD scan: 210-400 nm

Rt=0.78 min (M⁺+1=224): cis and trans isomer

### Intermediate 123

### N-(1-Methylpiperidin-4-yl)-3-nitrobenzamide

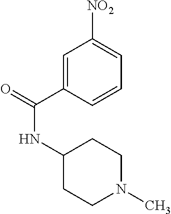 [SMILES: CN1CCC(NC(=O)c2cccc([N+](=O)[O-])c2)CC1]

A solution of 16.25 g of 3-nitrobenzoyl chloride (CAS 121-90-4) in 175 ml of pyridine was admixed at RT with 10 g of 4-amino-1-methylpiperine (CAS 41838-46-4). The mixture was stirred for 1 hour, then poured onto ice-water and stirred at RT for 14 hours. The reaction solution was extracted with ethyl acetate, and the organic phase was washed with 2 M sodium hydroxide solution and water, dried over sodium sulphate and concentrated under reduced pressure. The residue was taken up again in ethyl acetate and stirred. The residue was filtered off with suction. This gave 19.5 g of N-(1-methylpiperidin-4-yl)-3-nitrobenzamide.

UPLC-MS: Instrument: Waters Acquity UPLC-MS SQD; column: Acquity UPLC BEH C18 1.7 50×2.1 mm; eluent A: water+0.2% by vol. of ammonia (32%), eluent B: acetonitrile; gradient: 0-1.6 min 1-99% B, 1.6-2.0 min 99% B; flow rate 0.8 ml/min; temperature: 60° C.; injection: 2 μl; DAD scan: 210-400 nm

Rt=0.84 min (M+1=264)

### Intermediate 124

### 3-Amino-N-(1-methylpiperidin-4-yl)benzamide

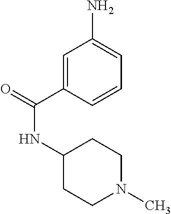 [SMILES: CN1CCC(NC(=O)c2cccc(N)c2)CC1]

A suspension of 19.5 g of Intermediate 123 and 1.85 g of palladium (10% on activated carbon) in 740 ml of ethanol was stirred under a hydrogen atmosphere at RT for 6 hours. The mixture was filtered through kieselguhr and the solution was concentrated fully under reduced pressure. This gave 15.5 g of 3-amino-N-(1-methylpiperidin-4-yl)benzamide.

¹H NMR (400 MHz, 25° C., DMSO-d6) δ=1.55 (dq, 2H); 1.71 (bd, 2H); 1.91 (dt, 2H); 2.15 (s, 3); 2.74 (bd, 2H); 3.61-3.74 (m, 1H); 5.18 (bs, 2H); 6.66 (ddd, 1H); 6.93 (bd, 1H); 7.00 (t, 1H); 7.05 (t, 1H); 7.97 (d, 1H).

The working examples which follow were prepared by additionally using the amines shown in Table 1 below, which are either commercially available or can be prepared by or analogously to the procedures cited.

TABLE 1

| Amine | Name | CAS No | Structure | Preparation described: |
|---|---|---|---|---|
| 1 | 3-(pyrrolidin-1- ylsulphonyl)- aniline | 91619-38-4 | 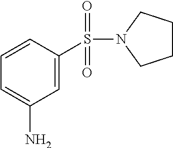 [SMILES: Nc1cccc(S(=O)(=O)N2CCCC2)c1] | |
| 2 | 3-amino-N,N- dimethylbenzene- sulphonamide | 6274-18-6 | 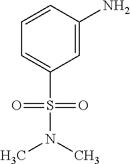 | |
| 3 | N,N-dimethyl-3- (methylamino)- benzenesulphon- amide | 86317-09-1 | 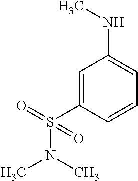 | |
| 4 | 2,3-dihydro-1,2- benzothiazol-6- amine 1,1-dioxide | 916438-46-5 | 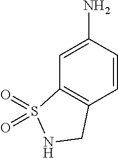 [SMILES: Nc1ccc2c(c1)S(=O)(=O)NC2] | |
| 5 | 3-aminobenzene- sulphonamide | 98-18-0 | 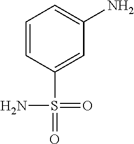 [SMILES: Nc1cccc(S(N)(=O)=O)c1] | |
| 6 | 3-(morpholin-4- ylsulphonyl)aniline | 22184-97-0 | 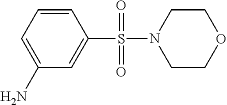 [SMILES: Nc1cccc(S(=O)(=O)N2CCOCC2)c1] | |
| 7 | 3-amino-N- ethylbenzene- sulphonamide | 56445-08-0 | 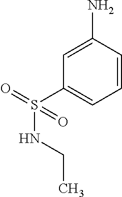 | |
| 8 | 3-amino-N- cyclopropyl- benzenesulphon- amide | 459434-39-0 | 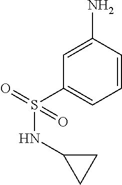 [SMILES: Nc1cccc(S(=O)(=O)NC2CC2)c1] | |
| 9 | 3-amino-N,N- diethylbenzene- sulphonamide | 10372-41-5 | 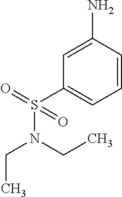 | |
| 10 | 3-amino-N- methylbenzene sulphonamide | 459434-40-3 | 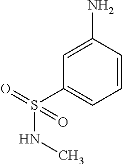 | |
| 11 | 3-amino-N- isopropylbenzene- sulphonamide | 118837-66-4 | 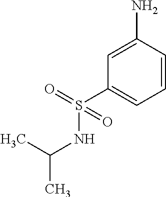 | |
| 12 | 5-amino-2- methylbenzene sulphonamide | 07/09/6973 | 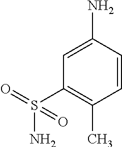 | |
| 13 | trans-4-[4- (cyclopropyl- methyl)piperazin- 1-yl]- cyclohexanamine | 876461-31-3 | 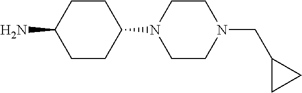 [SMILES: N[C@H]1CC[C@H](N2CCN(CC3CC3)CC2)CC1] | analogously to WO2012049153 |
| 14 | 4-amino-1- methylpiperidine | 41838-46-4 | 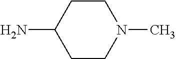 | |
| 15 | 2-(4-methyl- piperazin-1-yl)- ethanamine | 934-98-5 | 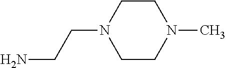 | |
| 16 | 1-methylpiperazine | 109-01-3 | 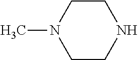 | |
| 17 | 1-isopropyl- piperazine | 4318-42-7 | 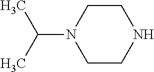 | |
| 18 | 1-cyclopropyl- piperazine | 20327-23-5 | 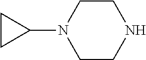 | |
| 19 | N,N- dimethylethane- 1,2-diamine | 108-00-9 | 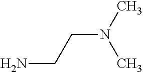 | |
| 20 | 1-methylazetidin- 3-amine dihydrochloride | 1139634-75-5 | 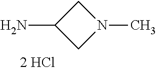 | |
| 21 | 3-amino-N,N- dimethylbenzamide | 33322-60-0 | 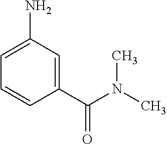 | |
| 22 | 1-(cyclopropyl- methyl)piperazine | 57184-25-5 | 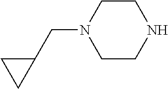 | |

Intermediates of the general formulae (IX) and (XVI) which are preferably employed for preparation of the compounds of the general formula (I) are, for example:
methyl 3-{[(3R)-4-cyclopentyl-1,3-dimethyl-2-oxo-1,2,3,4-tetrahydropyrido[2,3-b]pyrazin-6-yl]amino}benzoate;
methyl 3-{[(3R)-1,3-dimethyl-2-oxo-4-(tetrahydro-2H-pyran-4-yl)-1,2,3,4-tetrahydropyrido[2,3-b]pyrazin-6-yl]amino}benzoate;
methyl 3-{[(3R)-1,3-dimethyl-2-oxo-4-(tetrahydro-2H-pyran-4-yl)-1,2,3,4-tetrahydropyrido[2,3-b]pyrazin-6-yl]amino}-4-methoxybenzoate;
ethyl 3-{[4-(2,6-difluorobenzyl)-1,3-dimethyl-2-oxo-1,2,3,4-tetrahydroquinoxalin-6-yl]amino}benzoate;
methyl 3-{[(3R)-4-cyclohexyl-1,3-dimethyl-2-oxo-1,2,3,4-tetrahydropyrido[2,3-b]pyrazin-6-yl]amino}benzoate;
methyl 3-{[(3R)-1,3-dimethyl-2-oxo-4-(propan-2-yl)-1,2,3,4-tetrahydropyrido[2,3-b]pyrazin-6-yl]amino}benzoate;
methyl 3-{[(3R)-4-benzyl-1,3-dimethyl-2-oxo-1,2,3,4-tetrahydropyrido[2,3-b]pyrazin-6-yl]amino}benzoate;
methyl 3-{[4-(2-methoxyethyl)-1,3-dimethyl-2-oxo-1,2,3,4-tetrahydropyrido[2,3-b]pyrazin-6-yl]amino}benzoate;
tert-butyl 4-[(3R)-6-{[3-(ethoxycarbonyl)phenyl]amino}-1,3-dimethyl-2-oxo-2,3-dihydropyrido[2,3-b]pyrazin-4(1H)-yl]piperidine-1-carboxylate;
methyl 3-{[4-(4-fluorophenyl)-1,3-dimethyl-2-oxo-1,2,3,4-tetrahydropyrido[2,3-b]pyrazin-6-yl]amino}benzoate;
methyl 5-{[(3R)-1,3-dimethyl-2-oxo-4-(tetrahydro-2H-pyran-4-yl)-1,2,3,4-tetrahydropyrido[2,3-b]pyrazin-6-yl]amino}-2-methoxybenzoate;
Methyl 3-{[(3R)-1,3-dimethyl-2-oxo-4-(tetrahydro-2H-pyran-4-yl)-1,2,3,4-tetrahydropyrido[2,3-b]pyrazin-6-yl]amino}-2-methoxybenzoate
   and
methyl 3-{[(3R)-1,3-dimethyl-2-oxo-4-(tetrahydro-2H-pyran-4-yl)-1,2,3,4-tetrahydroquinoxalin-6-yl]amino}benzoate.

These intermediates of the general formulae (IX) and (XVI) likewise form part of the subject-matter of the present invention.

Intermediates of the general formulae (X) and (XVII) which are preferably employed for preparation of the compounds of the general formula (I) are, for example:
3-{[(3R)-4-cyclopentyl-1,3-dimethyl-2-oxo-1,2,3,4-tetrahydropyrido[2,3-b]pyrazin-6-yl]amino}benzoic acid;
3-{[(3R)-1,3-dimethyl-2-oxo-4-(tetrahydro-2H-pyran-4-yl)-1,2,34-tetrahydropyrido[2,3-b]pyrazin-6-yl]amino}benzoic acid;
3-{[(3R)-1,3-dimethyl-2-oxo-4-(tetrahydro-2H-pyran-4-yl)-1,2,34-tetrahydropyrido[2,3-b]pyrazin-6-yl]amino}-4-methoxybenzoic acid;
3-{[4-(2,6-difluorobenzyl)-1,3-dimethyl-2-oxo-1,2,3,4-tetrahydroquinoxalin-6-yl]amino}benzoic acid;
3-{[(3R)-4-cyclohexyl-1,3-dimethyl-2-oxo-1,2,3,4-tetrahydropyrido[2,3-b]pyrazin-6-yl]amino}benzoic acid;
3-{[(3R)-1,3-dimethyl-2-oxo-4-(propan-2-yl)-1,2,3,4-tetrahydropyrido[2,3-b]pyrazin-6-yl]amino}benzoic acid;
3-{[(3R)-4-benzyl-1,3-dimethyl-2-oxo-1,2,3,4-tetrahydropyrido[2,3-b]pyrazin-6-yl]amino}benzoic acid;
3-{[4-(2-methoxyethyl)-1,3-dimethyl-2-oxo-1,2,3,4-tetrahydropyrido[2,3-b]pyrazin-6-yl]amino}benzoic acid;
3-({(3R)-4-[1-(tert-butoxycarbonyl)piperidin-4-yl]-1,3-dimethyl-2-oxo-1,2,3,4-tetrahydropyrido[2,3-b]pyrazin-6-yl}amino)benzoic acid;
3-{[4-(4-fluorophenyl)-1,3-dimethyl-2-oxo-1,2,3,4-tetrahydropyrido[2,3-b]pyrazin-6-yl]amino}benzoic acid;
5-{[(3R)-1,3-dimethyl-2-oxo-4-(tetrahydro-2H-pyran-4-yl)-1,2,3,4-tetrahydropyrido[2,3-b]pyrazin-6-yl]amino}-2-methoxybenzoic acid;
3-{[(3R)-1,3-Dimethyl-2-oxo-4-(tetrahydro-2H-pyran-4-yl)-1,2,3,4-tetrahydropyrido[2,3-b]pyrazin-6-yl]amino}-2-methoxybenzoic acid
   and
3-{[(3R)-1,3-dimethyl-2-oxo-4-(tetrahydro-2H-pyran-4-yl)-1,2,3,4-tetrahydroquinoxalin-6-yl]amino}benzoic acid.

These intermediates of the general formulae (X) and (XVII) likewise form part of the subject-matter of the present invention.

### Preparation of the Inventive Compounds

### Example 1, General Synthesis Method A

### (3R)-4-Cyclopentyl-1,3-dimethyl-6-({3-[(4-methylpiperazin-1-yl)carbonyl]phenyl}amino)-3,4-dihydropyrido[2,3-b]pyrazin-2(1H)-one

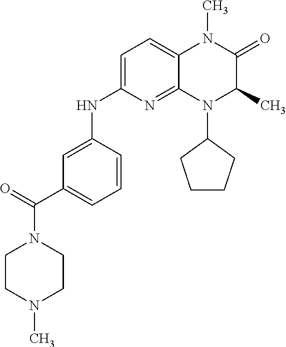 [SMILES: C[C@@H]1C(=O)N(C)c2ccc(Nc3cccc(C(=O)N4CCN(C)CC4)c3)nc2N1C1CCCC1]

A solution of 150 mg of Intermediate 7, 79 mg of N-methylpiperazine (Amine 16, Table 1), 0.22 ml of triethylamine and 224 mg of HATU in 16 ml of DMF was stirred at RT for 16 hours. The mixture was added to semisaturated sodium chloride solution and extracted three times with ethyl acetate. The combined organic phases were washed with saturated sodium chloride solution and dried over sodium sulphate, and the solvent was removed under reduced pressure. The residue was purified by RP-HPLC (Waters SQD autopurification system; column: Waters XBridge C18 5 μm 100×30 mm; eluent A: water+0.1% by vol. of formic acid (99%), eluent B: acetonitrile; gradient: 0-8.0 min 1-100% B, 8.0-10.0 min 100% B; flow rate 50.0 ml/min; temperature: RT; injection: 2500 μl; DAD scan: 210-400 nm). This gave 70 mg of (3R)-4-cyclopentyl-1,3-dimethyl-6-({3-[(4-methylpiperazin-1-yl)carbonyl]phenyl}amino)-3,4-dihydropyrido[2,3-b]pyrazin-2(1H)-one.

¹H NMR (300 MHz, DMSO-d6, selected signals): δ=1.06 (d, 3H); 1.48-1.77 (m, 6H); 1.89-2.02 (m, 2H); 2.19 (s, 3H); 2.22-2.42 (m, 4H); 3.20 (s, 3H); 4.29 (q, 1H); 4.42 (qi, 1H); 6.25 (d, 1H); 6.76 (d, 1H); 7.21-7.29 (m, 2H); 7.44 (d, 1H); 7.94 (s, 1H); 8.93 (s, 1H).

### Example 2

### 3-{[(3R)-4-Cyclopentyl-1,3-dimethyl-2-oxo-1,2,3,4-tetrahydropyrido[2,3-b]pyrazin-6-yl]amino}-N-(1-methylpiperidin-4-yl)benzamide

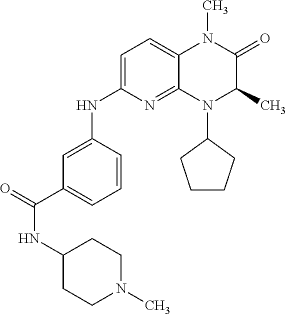 [SMILES: C[C@@H]1C(=O)N(C)c2ccc(Nc3cccc(C(=O)NC4CCN(C)CC4)c3)nc2N1C1CCCC1]

In analogy to Example 1, 3-{[(3R)-4-cyclopentyl-1,3-dimethyl-2-oxo-1,2,3,4-tetrahydropyrido[2,3-b]pyrazin-6-yl]amino}-N-(1-methylpiperidin-4-yl)benzamide was prepared proceeding from 150 mg of Intermediate 7, 90 mg of 1-methylpiperidine-4-amine (Amine 14, Table 1), 0.22 ml of triethylamine and 224 mg of HATU in 16 ml of DMF. Purification by RP-HPLC (Waters SQD autopurification system; column: Waters XBridge C18 5 μm 100×30 mm; eluent A: water+0.1% by vol. of formic acid (99%), eluent B: acetonitrile; gradient: 0-8.0 min 1-100% B, 8.0-10.0 min 100% B; flow rate 50.0 ml/min; temperature: RT; injection: 2500 μl; DAD scan: 210-400 nm) gave 100 mg of 3-{[(3R)-4-cyclopentyl-1,3-dimethyl-2-oxo-1,2,3,4-tetrahydropyrido[2,3-b]pyrazin-6-yl]amino}-N-(1-methylpiperidine-4-yl)benzamide.

¹H NMR (400 MHz, DMSO-d6): δ=1.07 (d, 3H); 1.47-1.72 (m, 8H); 1.72-1.85 (m, 2H); 1.88-2.05 (m, 2H); 2.13 (t, 2H); 2.25 (s, 3H); 2.86 (d, 2H); 3.20 (s, 3H); 3.67-3.83 (m, 1H); 4.18 (q, 1H); 4.34-4.48 (m, 1H); 6.24 (m, 1H); 7.20-7.32 (m, 3H); 7.62-7.73 (m, 1H); 8.06 (s, 1H); 8.13 (d, 1H); 8.87 (s, 1H).

### Example 3

### 3-{[(3R)-4-Cyclopentyl-1,3-dimethyl-2-oxo-1,2,3,4-tetrahydropyrido[2,3-b]pyrazin-6-yl]amino}-N-(1-methylazetidin-3-yl)benzamide

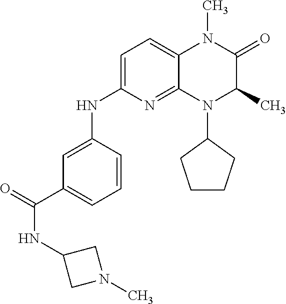 [SMILES: C[C@@H]1C(=O)N(C)c2ccc(Nc3cccc(C(=O)NC4CN(C)C4)c3)nc2N1C1CCCC1]

In analogy to Example 1, 3-{[(3R)-4-cyclopentyl-1,3-dimethyl-2-oxo-1,2,3,4-tetrahydropyrido[2,3-b]pyrazin-6-yl]amino}-N-(1-methylazetidin-3-yl)benzamide was prepared proceeding from 150 mg of Intermediate 7, 125 mg of 1-methylazetidin-3-amine dihydrochloride (Amine 20, Table 1), 0.22 ml of triethylamine and 224 mg of HATU in 16 ml of DMF. Purification by RP-HPLC (Waters SQD autopurification system; column: Waters XBridge C18 5 μm 100×30 mm; eluent A: water+0.1% by vol. of formic acid (99%), eluent B: acetonitrile; gradient: 0-8.0 min 1-100% B, 8.0-10.0 min 100% B; flow rate 50.0 ml/min; temperature: RT; injection: 2500 μl; DAD scan: 210-400 nm) gave 40 mg of 3-{[(3R)-4-cyclopentyl-1,3-dimethyl-2-oxo-1,2,3,4-tetrahydropyrido[2,3-b]pyrazin-6-yl]amino}-N-(1-methylazetidin-3-yl)benzamide.

¹H NMR (400 MHz, DMSO-d6): δ=1.07 (d, 3H); 1.47-1.73 (m, 6H); 1.88-2.92 (m, 2H); 2.29 (s, 3H); 3.04 (t, 2H); 3.61 (t, 2H); 4.18 (q, 1H); 4.36-4.51 (m, 2H); 6.24 (d, 1H); 7.20-7.33 (m, 3H); 7.57-7.66 (m, 1H); 8.16 (s, 1H); 8.65 (d, 1H); 8.89 (s, 1H).

### Example 4

### 3-{[(3R)-4-Cyclopentyl-1,3-dimethyl-2-oxo-1,2,3,4-tetrahydropyrido[2,3-b]pyrazin-6-yl]amino}-N-[2-(dimethylamino)ethyl]benzamide

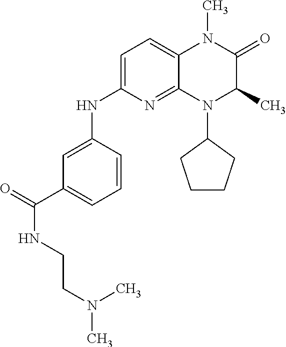 [SMILES: C[C@@H]1C(=O)N(C)c2ccc(Nc3cccc(C(=O)NCCN(C)C)c3)nc2N1C1CCCC1]

In analogy to Example 1, 3-{[(3R)-4-cyclopentyl-1,3-dimethyl-2-oxo-1,2,3,4-tetrahydropyrido[2,3-b]pyrazin-6-yl]amino}-N-[2-(dimethylamino)ethyl]benzamide was prepared proceeding from 150 mg of Intermediate 7, 69 mg of N,N-dimethylethane-1,2-diamine (Amine 19, Table 1), 0.22 ml of triethylamine and 224 mg of HATU in 16 ml of DMF. Purification by RP-HPLC (Waters SQD autopurification system; column: Waters XBridge C18 5 μm 100×30 mm; eluent A: water+0.1% by vol. of formic acid (99%), eluent B: acetonitrile; gradient: 0-8.0 min 1-100% B, 8.0-10.0 min 100% B; flow rate 50.0 ml/min; temperature: RT; injection: 2500 μl; DAD scan: 210-400 nm) gave 40 mg of 3-{[(3R)-4-cyclopentyl-1,3-dimethyl-2-oxo-1,2,3,4-tetrahydropyrido[2,3-b]pyrazin-6-yl]amino}-N-[2-(dimethylamino)ethyl]benzamide.

¹H NMR (400 MHz, DMSO-d6, selected signals): δ=1.07 (d, 3H); 1.47-1.73 (m, 6H); 1.89-2.06 (m, 2H); 2.23 (s, 6H); 3.2 (s, 3H); 3.35 (q, 2H); 4.18 (q, 1H); 4.46 (qi, 1H); 6.25 (d, 1H); 7.20-7.31 (m, 3H); 7.64 (d, 1H); 8.14 (s, 1H); 8.24 (t, 1H); 8.88 (s, 1H).

### Example 5

### 3-{[(3R)-4-Cyclopentyl-1,3-dimethyl-2-oxo-1,2,3,4-tetrahydropyrido[2,3-b]pyrazin-6-yl]amino}-N-{trans-4-[4-(cyclopropylmethyl)piperazin-1-yl]cyclohexyl}benzamide

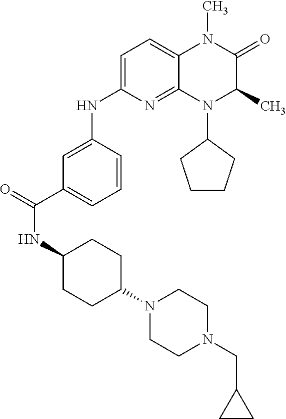

In analogy to Example 1, 3-{[(3R)-4-cyclopentyl-1,3-dimethyl-2-oxo-1,2,3,4-tetrahydropyrido[2,3-b]pyrazin-6-yl]amino}-N-{trans-4-[4-(cyclopropylmethyl)piperazin-1-yl]cyclohexyl}benzamide was prepared proceeding from 150 mg of Intermediate 7, 187 mg of trans-4-[4-(cyclopropylmethyl)piperazin-1-yl]cyclohexanamine (Amine 13, Table 1), 0.22 ml of triethylamine and 224 mg of HATU in 16 ml of DMF. Purification by RP-HPLC (Waters SQD autopurification system; column: Waters XBridge C18 5 μm 100×30 mm; eluent A: water+0.1% by vol. of formic acid (99%), eluent B: acetonitrile; gradient: 0-8.0 min 1-100% B, 8.0-10.0 min 100% B; flow rate 50.0 ml/min; temperature: RT; injection: 2500 μl; DAD scan: 210-400 nm) gave 95 mg of 3-{[(3R)-4-cyclopentyl-1,3-dimethyl-2-oxo-1,2,3,4-tetrahydropyrido[2,3-b]pyrazin-6-yl]amino}-N-{trans-4-[4-(cyclopropylmethyl)piperazin-1-yl]cyclohexyl}benzamide.

¹H NMR (400 MHz, CDCl₃): δ=0.02-0.10 (m, 2H); 0.40-0.49 (m, 2H); 0.73-0.88 (m, 1H); 1.06 (d, 3H); 1.19-1.43 (m, 4H); 1.46-1.73 (m, 6H); 1.76-2.04 (m, 6H); 2.12-2.30 (d+m, 3H); 4.18 (q, 1H); 4.32-4.48 (m, 1H); 6.24 (d, 1H); 7.18-7.30 (m, 3H); 7.67 (d, 1H); 8.04 (s, 1H); 8.08 (d, 1H); 8.89 (s, 1H).

### Example 6

### 3-{[(3R)-1,3-Dimethyl-2-oxo-4-(tetrahydro-2H-pyran-4-yl)-1,2,3,4-tetrahydropyrido[2,3-b]pyrazin-6-yl]amino}-N,N-dimethylbenzenesulphonamide

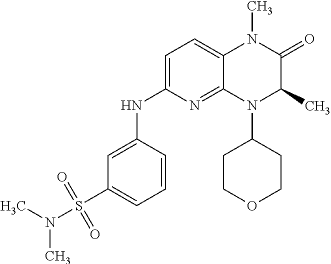

A suspension of 150 mg of Intermediate 10, 192 mg of 3-amino-N,N-dimethylbenzenesulphonamide (Amine 2, Table 1), 21 mg of palladium acetate, 784 mg of caesium carbonate and 60 mg of (+)-BINAP in 10.8 ml of toluene was stirred at bath temperature 120° C. under argon for 5 hours. The reaction solution was filtered, the residue was washed with ethyl acetate and the combined organic phases were concentrated fully under reduced pressure. The residue was purified by RP-HPLC (Waters SQD autopurification system; column: Waters XBridge C18 5 μm 100×30 mm; eluent A: water+0.1% by vol. of formic acid (99%), eluent B: acetonitrile; gradient: 0-8.0 min 1-100% B, 8.0-10.0 min 100% B; flow rate 50.0 ml/min; temperature: RT; injection: 2500 μl; DAD scan: 210-400 nm). This gave 110 mg of 3-{[(3R)-1,3-dimethyl-2-oxo-4-(tetrahydro-2H-pyran-4-yl)-1,2,3,4-tetrahydropyrido[2,3-b]pyrazin-6-yl]amino}-N,N-dimethylbenzenesulphonamide.

¹H NMR (400 MHz, DMSO-d6): δ=1.08 (d, 3H); 1.59 (d, 1H); 1.77 (dq, 1H); 1.83-1.98 (m, 2H); 2.61 (s, 6H); 3.21 (s, 3H); 3.51 (t, 2H); 3.94 (t, 2H); 4.24 (q, 1H); 4.41-4.52 (m, 1H); 6.28 (d, 1H); 7.15 (d, 1H); 7.30 (d, 1H); 7.48 (t, 1H); 7.81 (t, 1H); 8.10 (d, 1H); 9.21 (s, 1H).

### Example 7

### 3-{[(3R)-1,3-Dimethyl-2-oxo-4-(tetrahydro-2H-pyran-4-yl)-1,2,3,4-tetrahydroguinoxalin-6-yl]amino}-N,N-dimethylbenzenesulphonamide

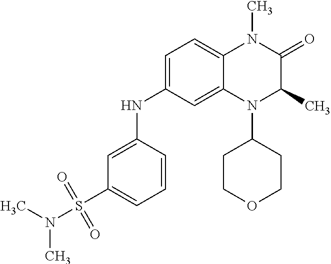

In analogy to the preparation of Example 6, 3-{[(3R)-1,3-dimethyl-2-oxo-4-(tetrahydro-2H-pyran-4-yl)-1,2,3,4-tetrahydroquinoxalin-6-yl]amino}-N,N-dimethylbenzenesulphonamide (Amine 2, Table 1) was prepared proceeding from 47 mg of Intermediate 14 and 55 mg of 3-amino-N,N-dimethylbenzenesulphonamide. This gave 37 mg of the title compound.

¹H NMR (400 MHz, CDCl₃): δ=1.13 (d, 3H); 1.70 (d, 1H); 1.75-1.96 (m, 3H); 2.74 (s, 6H); 3.38 (s, 3H); 3.46 (ddt, 2H); 3.58 (tt, 1H); 4.05 (dt, 2H); 4.15 (q, 1H); 5.85 (s, 1H); 6.67-6.73 (m, 2H); 6.91 (d, 1H); 7.14 (dd, 1H); 7.23 (dd, 1H); 7.39 (t, 1H); 7.42 (t, 1H).

### Example 8

### (3R)-1,3-Dimethyl-6-{[3-(morpholin-4-ylsulphonyl)phenyl]amino}-4-(tetrahydro-2H-pyran-4-yl)-3,4-dihydroquinoxalin-2(1H)-one

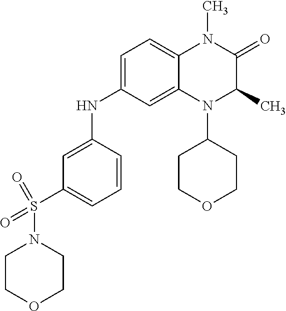 [SMILES: C[C@@H]1C(=O)N(C)c2ccc(Nc3cccc(S(=O)(=O)N4CCOCC4)c3)cc2N1C1CCOCC1]

In analogy to the preparation of Example 6, (3R)-1,3-dimethyl-6-{[3-(morpholin-4-ylsulphonyl)phenyl]amino}-4-(tetrahydro-2H-pyran-4-yl)-3,4-dihydroquinoxalin-2(1H)-one was prepared proceeding from 47 mg of Intermediate 14 and 67 mg of 3-(morpholin-4-ylsulphonyl)aniline (Amine 6, Table 1). This gave 37 mg of the title compound.

¹H NMR (400 MHz, CDCl₃): δ=1.14 (d, 3H); 1.67-1.75 (m, 1H); 1.76-1.97 (m, 3H); 3.00-3.10 (m, 4H); 3.38 (s, 3H); 3.46 (ddt, 2H); 3.58 (tt, 1H); 3.72-3.81 (m, 4H); 4.05 (dt, 2H); 4.15 (q, 1H); 5.85 (s, 1H); 6.67-6.74 (m, 2H); 6.92 (d, 1H); 7.15 (dd, 1H); 7.20 (d, 1H); 7.36-7.44 (m, 2H).

### Example 9

### (3R)-1,3-Dimethyl-6-{[3-(morpholin-4-ylsulphonyl)phenyl]amino}-4-(tetrahydro-2H-pyran-4-yl)-3,4-dihydropyrido[2,3-b]pyrazin-2(1H)-one

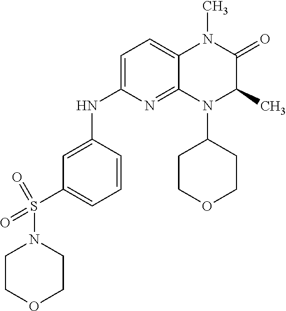 [SMILES: C[C@@H]1C(=O)N(C)c2ccc(Nc3cccc(S(=O)(=O)N4CCOCC4)c3)nc2N1C1CCOCC1]

In analogy to the preparation of Example 6, (3R)-1,3-dimethyl-6-{[3-(morpholin-4-ylsulphonyl)phenyl]amino}-4-(tetrahydro-2H-pyran-4-yl)-3,4-dihydropyrido[2,3-b]pyrazin-2(1H)-one was prepared proceeding from 150 mg of Intermediate 10 and 233 mg of 3-(morpholin-4-ylsulphonyl)aniline (Amine 6, Table 1). This gave 24 mg of the title compound.

¹H NMR (400 MHz, DMSO-d6): δ=1.08 (t, 3H); 1.60 (bd, 1H); 1.71-1.83 (m, 1H); 1.83-1.98 (m, 2H); 2.81-2.91 (m, 4H); 3.21 (s, 3H); 3.46-3.57 (m, 2H); 3.60-3.67 (m, 4H); 3.89-3.99 (m, 2H); 4.24 (q, 1H); 4.46 (tt, 1H); 6.28 (d, 1H); 7.14 (d, 1H); 7.30 (d, 1H); 7.50 (t, 1H); 7.80 (t, 1H); 8.11 (d, 1H); 9.24 (s, 1H).

### Example 10

### 3-{[(3R)-4-(4-Methoxybenzyl)-1,3-dimethyl-2-oxo-1,2,3,4-tetrahydroquinoxalin-6-yl]amino}-N,N-dimethylbenzenesulphonamide

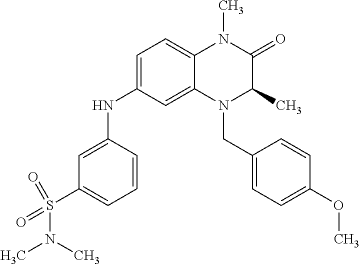 [SMILES: COc1ccc(CN2c3cc(Nc4cccc(S(=O)(=O)N(C)C)c4)ccc3N(C)C(=O)[C@H]2C)cc1]

In analogy to the preparation of Example 6, 3-{[(3R)-4-(4-methoxybenzyl)-1,3-dimethyl-2-oxo-1,2,3,4-tetrahydroquinoxalin-6-yl]amino}-N,N-dimethylbenzenesulphonamide was prepared proceeding from 50 mg of Intermediate 16 and 53 mg of 3-amino-N,N-dimethylbenzenesulphonamide (Amine 2, Table 1). This gave 22 mg of the title compound.

¹H NMR (400 MHz, CDCl₃): δ=1.15 (d, 3H); 2.73 (s, 6H); 3.41 (s, 3H); 3.82 (s, 3H); 4.01 (q, 1H); 4.09 (d, 1H); 4.43 (d, 1H); 5.95 (s, 1H); 6.48 (d, 1H); 6.64 (dd, 1H); 6.86-6.94 (m, 3H); 6.97 (dd, 1H); 7.19 (d, 1H); 7.22-7.28 (m, 3H); 7.32 (t, 1H).

### Example 11

### (3R)-4-(4-Methoxybenzyl)-1,3-dimethyl-6-{[3-(morpholin-4-ylsulphonyl)phenyl]amino}-3,4-dihydroquinoxalin-2(1H)-one

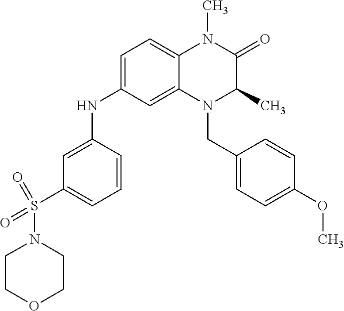 [SMILES: COc1ccc(CN2c3cc(Nc4cccc(S(=O)(=O)N5CCOCC5)c4)ccc3N(C)C(=O)[C@H]2C)cc1]

In analogy to the preparation of Example 6, (3R)-4-(4-methoxybenzyl)-1,3-dimethyl-6-{[3-(morpholin-4-ylsulphonyl)phenyl]amino}-3,4-dihydroquinoxalin-2(1H)-one was prepared proceeding from 50 mg of Intermediate 16 and 65 mg of 3-(morpholin-4-ylsulphonyl)aniline (Amine 6, Table 1). This gave 38 mg of the title compound.

¹H NMR (400 MHz, DMSO-d6): δ=1.00 (d, 3H); 2.83-2.90 (m, 4H); 3.28 (s, 3H); 3.59-3.65 (m, 4H); 3.75 (s, 3H); 3.91 (q, 1H); 4.17 (d, 1H); 4.39 (d, 1H); 6.50 (d, 1H); 6.59 (dd, 1H); 6.92 (d, 2H); 6.98-7.03 (m, 3H); 7.23-7.28 (m, 3H); 7.30 (t, 1H); 8.44 (s, 1H).

### Example 12

### (3R)-1,3-Dimethyl-6-({3-[(4-methylpiperazin-1-yl)sulphonyl]phenyl}amino)-4-(tetrahydro-2H-pyran-4-yl)-3,4-dihydropyrido[2,3-b]pyrazin-2(1H)-one

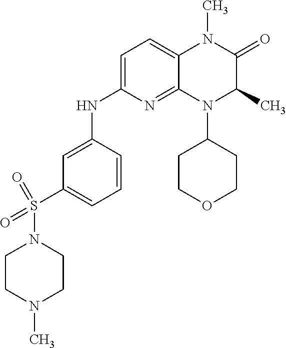 [SMILES: C[C@@H]1C(=O)N(C)c2ccc(Nc3cccc(S(=O)(=O)N4CCN(C)CC4)c3)nc2N1C1CCOCC1]

In analogy to the preparation of Example 6, (3R)-1,3-dimethyl-6-({3-[(4-methylpiperazin-1-yl)sulphonyl]phenyl}amino)-4-(tetrahydro-2H-pyran-4-yl)-3,4-dihydropyrido[2,3-b]pyrazin-2(1H)-one was prepared proceeding from 150 mg of Intermediate 10 and 246 mg of 3-[(4-methylpiperazin-1-yl)sulphonyl]aniline (Intermediate 110, US20030225106). This gave 95 mg of the title compound.

¹H NMR (400 MHz, DMSO-d6): δ=1.08 (d, 3H); 2.59 (bd, 1H); 1.76 (dq, 1H); 1.89 (dq, 1H); 1.93 (bd, 1H); 2.13 (s, 3H); 2.30-2.40 (m, 4H); 2.82-2.94 (m, 4H); 3.21 (s, 1H); 3.45-3.56 (m, 2H); 3.88-3.99 (m, 2H); 4.24 (q, 1H); 4.45 (tt, 1H); 6.28 (d, 1H); 7.13 (dd, 1H); 7.30 (d, 1H); 7.49 (t, 1H); 7.80 (t, 1H); 8.09 (dd, 1H); 9.22 (s, 1H).

### Example 13

### 3-{[(3R)-1,3-Dimethyl-2-oxo-4-(tetrahydro-2H-pyran-4-yl)-1,2,3,4-tetrahydropyrido[2,3-b]pyrazin-6-yl]amino}-N-(1-methylpiperidin-4-yl)benzamide

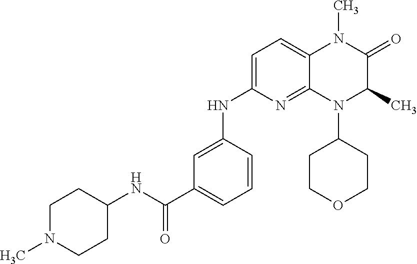

In analogy to the preparation of Example 1, 3-{[(3R)-1,3-dimethyl-2-oxo-4-(tetrahydro-2H-pyran-4-yl)-1,2,3,4-tetrahydropyrido[2,3-b]pyrazin-6-yl]amino}-N-(1-methylpiperidin-4-yl)benzamide was prepared proceeding from 115 mg of Intermediate 18 and 63 mg of 1-methylpiperidine-4-amine (Amine 14, Table 1). This gave 80 mg of the title compound.

¹H NMR (400 MHz, DMSO-d6): δ=1.08 (d, 3H); 1.53-1.80 (m, 6H); 1.89 (dq, 1H); 1.94 (bd, 1H); 2.04 (t, 2H); 2.21 (s, 3H); 2.77-2.86 (m, 2H); 3.20 (s, 3H); 3.40 (dt, 1H); 3.48 (dt, 1H); 3.67-3.81 (m, 2H); 3.87-3.95 (m, 2H); 4.22 (q, 1H); 4.44 (tt, 1H); 6.24 (d, 1H); 7.23-7.31 (m, 3H); 7.71-7.78 (m, 1H); 7.99 (bs, 1H); 8.14 (d, 1H); 8.92 (s, 1H).

### Example 14

### N-{trans-4-[4-(Cyclopropylmethyl)piperazin-1-yl]cyclohexyl}-3-{[(3R)-1,3-dimethyl-2-oxo-4-(tetrahydro-2H-pyran-4-yl)-1,2,3,4-tetrahydropyrido[2,3-b]pyrazin-6-yl]amino}benzamide

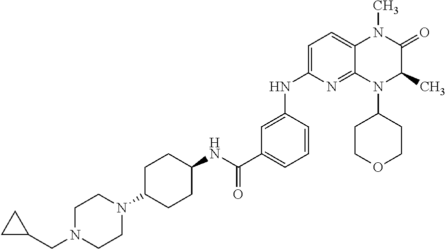

In analogy to the preparation of Example 1, N-{trans-4-[4-(cyclopropylmethyl)piperazin-1-yl]cyclohexyl}-3-{[(3R)-1,3-dimethyl-2-oxo-4-(tetrahydro-2H-pyran-4-yl)-1,2,3,4-tetrahydropyrido[2,3-b]pyrazin-6-yl]amino}benzamide was prepared proceeding from 120 mg of Intermediate 18 and 136 mg of trans-4-[4-(cyclopropylmethyl)piperazin-1-yl]cyclohexanamine (Amine 13, Table 1). This gave 65 mg of the title compound.

¹H NMR (400 MHz, DMSO-d6): δ=0.03-0.09 (m, 2H); 0.41-0.48 (m, 2H); 0.75-0.86 (m, 1H); 1.08 (d, 3H); 1.22-1.42 (m, 4H); 1.54-1.63 (m, 1H); 1.71 (dq, 1H); 1.79-1.99 (m, 6H); 2.16 (d, 2H); 2.17-2.27 (m, 1H); 3.20 (s, 3H); 3.40 (dt, 1H); 3.48 (dt, 1H); 3.60-3.76 (m); 3.87-3.96 (m, 2H); 4.22 (q, 1H); 4.44 (tt, 1H); 6.24 (d, 1H); 7.22-7.31 (m, 3H); 7.75 (dt, 1H); 7.97 (bs, 1H); 8.08 (d, 1H); 8.92 (s, 1H).

### Example 15

### (3R)-1,3-Dimethyl-6-({3-[(4-methylpiperazin-1-yl)carbonyl]phenyl}amino)-4-(tetrahydro-2H-pyran-4-yl)-3,4-dihydropyrido[2,3-b]pyrazin-2(1H)-one

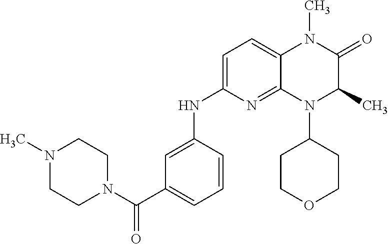

In analogy to the preparation of Example 1, (3R)-1,3-dimethyl-6-({3-[(4-methylpiperazin-1-yl)carbonyl]phenyl}amino)-4-(tetrahydro-2H-pyran-4-yl)-3,4-dihydropyrido[2,3-b]pyrazin-2(1H)-one was prepared proceeding from 120 mg of Intermediate 18 and 57 mg of 1-methylpiperazine (Amine 16, Table 1). This gave 53 mg of the title compound.

¹H NMR (400 MHz, DMSO-d6): δ=1.08 (d, 3H); 1.53-1.62 (m, 1H); 1.76 (dq, 1H); 1.82-1.96 (m, 2H); 2.20 (s, 3H); 2.23-2.44 (m, 4H); 3.20 (s, 3H); 3.34-3.49 (m, 2H); 3.89-3.99 (m, 2H); 4.23 (q, 1H); 4.43 (tt, 1H); 6.26 (d, 1H); 6.78 (d, 1H); 7.22-7.30 (m, 3H); 7.53 (d, 1H); 7.84 (s, 1H); 8.97 (s, 1H).

### Example 16

3-{[(3R)-1,3-Dimethyl-2-oxo-4-(tetrahydro-2H-pyran-4-yl)-1,2,3,4-tetrahydropyrido[2,3-b]pyrazin-6-yl]amino}-4-methoxy-N-(1-methylpiperidin-4-yl)benzamide

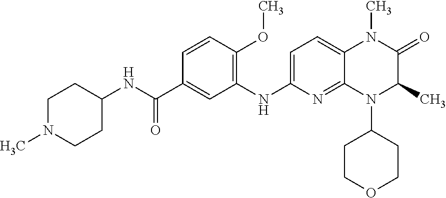

In analogy to the preparation of Example 1, 3-{[(3R)-1,3-dimethyl-2-oxo-4-(tetrahydro-2H-pyran-4-yl)-1,2,3,4-tetrahydropyrido[2,3-b]pyrazin-6-yl]amino}-4-methoxy-N-(1-methylpiperidin-4-yl)benzamide was prepared proceeding from 120 mg of Intermediate 20 and 61 mg of 4-amino-1-methylpiperidine (Amine 14, Table 1). This gave 68 mg of the title compound.

¹H NMR (400 MHz, DMSO-d6): δ=1.06 (d, 3H); 1.46-1.68 (m, 4H); 1.68-1.92 (m, 4H); 2.01 (t, 2H); 2.19 (s, 3H); 2.75-2.86 (m, 2H); 3.25 (dt, 1H); 3.45 (dt, 1H); 3.67-3.87 (m); 3.89 (s, 3H); 4.29 (q, 1H); 4.44 (tt, 1H); 6.45 (d, 1H); 6.98 (d, 3H); 7.24 (d, 1H); 7.38 (dd, 1H); 7.91 (s, 1H); 8.03 (d, 1H); 8.54 (d, 1H).

### Example 17

(3R)-6-({2-Methoxy-5-[(4-methylpiperazin-1-yl)carbonyl]phenyl}amino)-1,3-dimethyl-4-(tetrahydro-2H-pyran-4-yl)-3,4-dihydropyrido[2,3-b]pyrazin-2(1H)-one

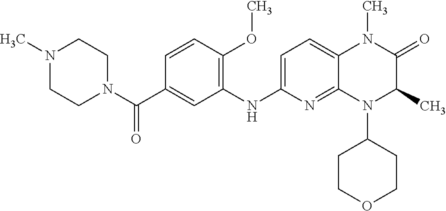

In analogy to the preparation of Example 1, (3R)-6-({2-methoxy-5-[(4-methylpiperazin-1-yl)carbonyl]phenyl}amino)-1,3-dimethyl-4-(tetrahydro-2H-pyran-4-yl)-3,4-dihydropyrido[2,3-b]pyrazin-2(1H)-one was prepared proceeding from 120 mg of Intermediate 20 and 54 mg of 1-methylpiperazine (Amine 16, Table 1). This gave 59 mg of the title compound.

¹H NMR (400 MHz, DMSO-d6): δ=1.07 (d, 3H); 1.87-1.58 (m, 1H); 1.63-1.90 (m, 3H); 2.18 (s, 3H); 2.23-2.39 (m, 4H); 3.20 (s, 3H); 3.28-3.47 (m, 2H); 3.82-3.95 (m+s, 5H); 4.22 (q, 1H); 4.43 (tt, 1H); 6.55 (d, 1H); 6.85 (dd, 1H); 6.88 (d, 1H); 7.26 (d, 1H); 7.97 (s, 1H); 8.44 (d, 1H).

### Example 18

### N-{trans-4-[4-(Cyclopropylmethyl)piperazin-1-yl]cyclohexyl}-3-{[(3R)-1,3-dimethyl-2-oxo-4-(tetrahydro-2H-pyran-4-yl)-1,2,3,4-tetrahydropyrido[2,3-b]pyrazin-6-yl]amino}-4-methoxybenzamide

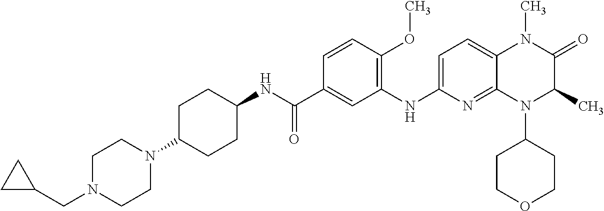

In analogy to the preparation of Example 1, N-{trans-4-[4-(cyclopropylmethyl)piperazin-1-yl]cyclohexyl}-3-{[(3R)-1,3-dimethyl-2-oxo-4-(tetrahydro-2H-pyran-4-yl)-1,2,3,4-tetrahydropyrido[2,3-b]pyrazin-6-yl]amino}-4-methoxybenzamide was prepared proceeding from 120 mg of Intermediate 20 and 127 mg of trans-4-[4-(cyclopropylmethyl)piperazin-1-yl]cyclohexanamine (Amine 13, Table 1). This gave 66 mg of the title compound.

¹H NMR (400 MHz, DMSO-d6): δ=0.02-0.10 (m, 2H); 0.40-0.49 (m, 2H); 0.74-0.88 (m, 1H); 1.06 (d, 3H); 1.19-1.43 (m, 4H); 1.46-1.68 (m, 2H); 1.71-1.94 (m, 6H); 2.12-2.29 (m+d, 3H); 3.20 (s, 3H); 3.25 (r, 1H); 3.45 (t, 1H); 3.89 (s, 3H); 4.19 (q, 1H); 4.43 (tt, 1H); 6.44 (d, 1H); 6.98 (d, 1H); 7.24 (d, 1H); 7.37 (dd, 1H); 7.90 (s, 1H); 7.98 (d, 1H); 8.53 (d, 1H).

### Example 19

### N-2-(Dimethylamino)ethyl-3-{[(3R)-1,3-dimethyl-2-oxo-4-(tetrahydro-2H-pyran-4-yl)-1,2,3,4-tetrahydropyrido[2,3-b]pyrazin-6-yl]amino}-4-methoxybenzamide

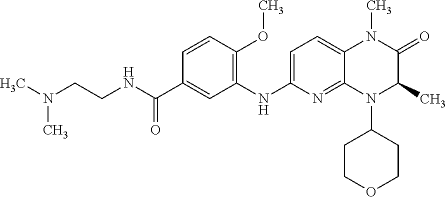

In analogy to the preparation of Example 1, N-[2-(dimethylamino)ethyl]-3-{[(3R)-1,3-dimethyl-2-oxo-4-(tetrahydro-2H-pyran-4-yl)-1,2,3,4-tetrahydropyrido[2,3-b]pyrazin-6-yl]amino}-4-methoxybenzamide was prepared proceeding from 120 mg of Intermediate 20 and 47 mg of N,N-dimethylethane-1,2-diamine (Amine 19, Table 1). This gave 50 mg of the title compound.

¹H NMR (400 MHz, CD₃OH): δ=1.16 (d, 3H); 1.66 (bd, 1H); 1.76 (dq, 1H); 1.89 (dq, 1H); 2.02 (bd, 1H); 2.59 (s, 6H); 2.80 (t, 2H); 3.30 (s, 3H); 3.50-3.61 (m, 3H); 3.65 (dt, 1H); 3.92 (dd, 1H); 3.96 (s, 3H); 4.27 (q, 1H); 4.67 (tt, 1H); 6.38 (d, 1H); 7.00 (d, 1H); 7.25 (d, 1H); 7.37 (dd, 1H); 8.73 (d, 1H).

### Example 20

### 3-{[(3R)-1,3-Dimethyl-2-oxo-4-(tetrahydro-2H-pyran-4-yl)-1,2,3,4-tetrahydropyrido[2,3-b]pyrazin-6-yl]amino}-N-(1-methylpiperidin-4-yl)benzenesulphonamide

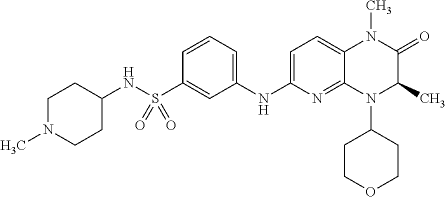

In analogy to the preparation of Example 6, 3-{[(3R)-1,3-dimethyl-2-oxo-4-(tetrahydro-2H-pyran-4-yl)-1,2,3,4-tetrahydropyrido[2,3-b]pyrazin-6-yl]amino}-N-(1-methylpiperidin-4-yl)benzenesulphonamide was prepared proceeding from 200 mg of Intermediate 10 and 346 mg of 3-amino-N-(1-methylpiperidin-4-yl)benzenesulphonamide (Intermediate 92). This gave 75 mg of the title compound.

¹H NMR (400 MHz, DMSO-d6): δ=1.08 (d, 3H); 1.34-1.47 (m, 2H); 1.51-1.62 (m, 3H); 1.75 (dq, 1H); 1.82-1.92 (m, 4H); 2.12 (s, 3H); 2.62-2.70 (m, 2H); 2.87-2.98 (m, 1H); 3.21 (s, 3H); 3.87-3.97 (m, 2H); 4.23 (q, 1H); 4.47 (tt, 1H); 6.28 (d, 1H); 7.24 (d, 1H); 7.29 (d, 1H); 7.42 (t, 1H); 7.62 (d, 1H); 7.88 (dd, 1H); 7.98 (t, 1H); 9.15 (s, 1H).

### Example 21

### N-[2-(Dimethylamino)ethyl]-3-{(3R)-1,3-dimethyl-2-oxo-4-(tetrahydro-2H-pyran-4-yl)-1,2,3,4-tetrahydropyrido[2,3-b]pyrazin-6-yl]amino}benzenesulphonamide

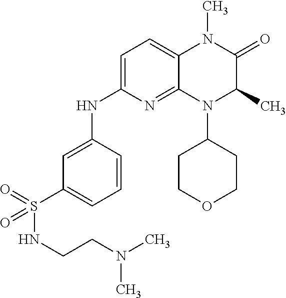 [SMILES: C[C@@H]1C(=O)N(C)c2ccc(Nc3cccc(S(=O)(=O)NCCN(C)C)c3)nc2N1C1CCOCC1]

In analogy to the preparation of Example 6, N-[2-(dimethylamino)ethyl]-3-{[(3R)-1,3-dimethyl-2-oxo-4-(tetrahydro-2H-pyran-4-yl)-1,2,3,4-tetrahydropyrido[2,3-b]pyrazin-6-yl]amino}benzenesulphonamide was prepared proceeding from 200 mg of Intermediate 10 and 312 mg of 3-amino-N-[2-(dimethylamino)ethyl]benzenesulphonamide (Intermediate 94). This gave 20 mg of the title compound.

¹H NMR (400 MHz, DMSO-d6): δ=1.08 (d, 3H); 1.52-1.63 (m, 1H); 1.75 (dq, 1H); 1.81-1.99 (m, 2H); 2.00-2.09 (m+s, 7H); 2.20-2.30 (m, 2H); 2.82 (t, 2H); 3.52 (t); 3.86-3.98 (m, 2H); 4.24 (q, 1H); 4.48 (tt, 1H); 6.27 (d, 1H); 7.22 (d, 1H); 7.29 (d, 1H); 7.43 (t, 1H); 7.91 (dd, 1H); 7.96 (t, 1H); 9.17 (s, 1H).

### Example 22

### N-{trans-4-[4-(Cyclopropylmethyl)piperazin-1-yl]cyclohexyl}-3-{[4-(2,6-difluorobenzyl)-1,3-dimethyl-2-oxo-1,2,3,4-tetrahydroquinoxalin-6-yl]amino}benzamide

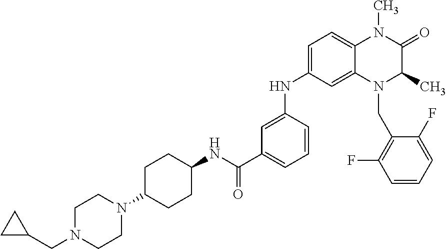

In analogy to the preparation of Example 1, N-{trans-4-[4-(cyclopropylmethyl)piperazin-1-yl]cyclohexyl}-3-{[4-(2,6-difluorobenzyl)-1,3-dimethyl-2-oxo-1,2,3,4-tetrahydroquinoxalin-6-yl]amino}benzamide was prepared proceeding from 40 mg of Intermediate 25 and 44 mg of trans-4-[4-(cyclopropylmethyl)piperazin-1-yl]cyclohexanamine (Amine 13, Table 1). This gave 33 mg of the title compound.

¹H NMR (400 MHz, DMSO-d6): δ=0.01-0.09 (m, 2H); 0.40-0.48 (m, 2H); 0.72-0.87 (m, 1H); 1.03 (d, 3H); 1.18-1.40 (m, 5H); 1.73-1.90 (m, 4H); 2.08-2.20 (m+d, 3H); 3.22 (s, 3H); 3.60-3.74 (m, 1H); 3.79 (q, 1H); 4.24 (d, 1H); 4.55 (d, 1H); 6.60 (dd, 1H); 6.71 (d, 1H); 6.97 (d, 1H); 7.05 (bd, 1H); 7.08-7.28 (m, 4H); 7.39-7.52 (m, 2H); 8.09 (d, 1H); 8.17 (s, 1H).

### Example 23, General Synthesis Method B

### 3-{[(3R)-1,3-dimethyl-2-oxo-4-(tetrahydro-2H-pyran-4-yl)-1,2,3,4-tetrahydropyrido[2,3-b]pyrazin-6-yl]amino}-N-ethylbenzenesulphonamide

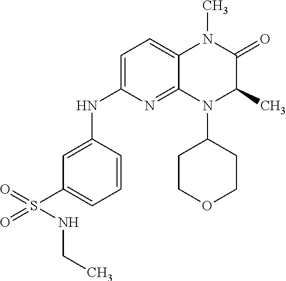 [SMILES: CCNS(=O)(=O)c1cccc(Nc2ccc3c(n2)N(C2CCOCC2)[C@H](C)C(=O)N3C)c1]

A mixture of 150 mg of Intermediate 10, 144 mg of 3-amino-N-ethylbenzenesulphonamide (Amine 7, Table 1), 6.6 mg of tris(dibenzylideneacetone)dipalladium(0) (CAS 51364-51-3), 235 mg of caesium carbonate and 12.3 mg of Xanthphos (CAS 161265-03-8) in 15 ml of dioxane was stirred under an argon atmosphere at 120° C. for 20 hours. The mixture was added to water and extracted twice with ethyl acetate. The organic phase was washed with saturated sodium chloride solution and dried over sodium sulphate, and the solvent was removed under reduced pressure. The residue was purified by chromatography on silica gel (dichloromethane/methanol gradient up to 3% methanol content). This gave 50 mg of 3-{[(3R)-1,3-dimethyl-2-oxo-4-(tetrahydro-2H-pyran-4-yl)-1,2,3,4-tetrahydropyrido[2,3-b]pyrazin-6-yl]amino}-N-ethylbenzenesulphonamide.

¹H NMR (300 MHz, 25° C., DMSO-d6): δ=0.97 (t, 3H); 1.07 (d, 3H); 1.57 (br. d, 1H); 1.90 (bs, 3H); 2.77 (ddd, 2H); 3.20 (s, 3H); 3.51 (t, 2H); 3.85-3.98 (m, 2H); 4.23 (q, 1H); 4.47 (tt, 1H); 6.27 (d, 1H); 7.20 (br. d, 1H); 7.28 (d, 1H); 7.38-7.49 (m, 2H); 7.88-7.97 (m, 2H); 9.16 (s, 1H).

### Example 24

### (3R)-1,3-Dimethyl-4-(1-methylpiperidin-4-yl)-6-{[3-(pyrrolidin-1-ylsulphonyl)phenyl]amino}-3,4-dihydropyrido[2,3-b]pyrazin-2(1H)-one

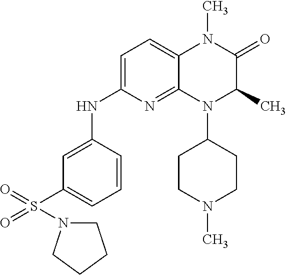 [SMILES: C[C@@H]1C(=O)N(C)c2ccc(Nc3cccc(S(=O)(=O)N4CCCC4)c3)nc2N1C1CCN(C)CC1]

A mixture of 200 mg of Intermediate 65, 146 mg of 3-(pyrrolidin-1-ylsulphonyl)aniline (Amine 1, Table 1), 10.6 mg of 2-dicyclohexylphosphino-2′,6′-diisopropoxybiphenyl (CAS: 787618-22-8), 17.6 mg of chloro(2-dicyclohexylphosphino-2′,6′-diisopropoxy-1,1′-biphenyl)[2-(2′-amino-1,1′-biphenyl)]palladium(II) (CAS: 1375325-68-0) and 253 mg of caesium carbonate in 2 ml of dioxane was stirred at 130° C. under an argon atmosphere for 2 hours. The mixture was diluted with water and dichloromethane and filtered through a phase separation cartridge (Biotage Isolute® phase separator, part number 120-1903-B). The organic phase was concentrated under reduced pressure. The residue was purified by chromatography on silica gel (dichloromethane/methanol gradient up to 10% methanol content). This gave 180 mg of (3R)-1,3-dimethyl-4-(1-methylpiperidin-4-yl)-6-{[3-(pyrrolidin-1-ylsulphonyl)phenyl]amino}-3,4-dihydropyrido[2,3-b]pyrazin-2(1H)-one.

¹H NMR (300 MHz, 25° C., CDCl₃): δ=1.22 (d, 3H); 1.67-1.88 (m, 6H); 2.01-2.21 (m, 4H); 2.33 (s, 3H); 2.97 (d, 2H); 3.25-3.30 (m, 7H); 4.23-4.36 (m, 2H); 6.25 (d, 1H); 6.45 (s, 1H); 7.04 (d, 1H); 7.33-7.45 (m, 2H); 7.67 (s, 1H); 7.80 (d, 1H).

### Example 25, General Synthesis Method C

### (3R)-6-{[2-Methoxy-5-(morpholin-4-ylsulphonyl)phenyl]amino}-1,3-dimethyl-4-(1-methylpiperidin-4-yl)-3,4-dihydropyrido[2,3-b]pyrazin-2(1H)-one

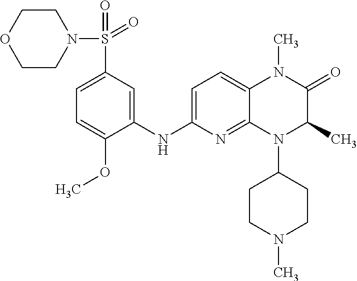 [SMILES: COc1ccc(S(=O)(=O)N2CCOCC2)cc1Nc1ccc2c(n1)N(C1CCN(C)CC1)[C@H](C)C(=O)N2C]

A mixture of 200 mg of Intermediate 65, 176 mg of Intermediate 97, 10.6 mg of 2-dicyclohexylphosphino-2′,6′-diisopropoxybiphenyl (CAS: 787618-22-8), 17.6 mg of chloro(2-dicyclohexylphosphino-2′,6′-diisopropoxy-1,1′-biphenyl)[2-(2′-amino-1,1′-biphenyl)]palladium(II) (CAS: 1375325-68-0) and 253 mg of caesium carbonate in 2 ml of dioxane was stirred at 130° C. under an argon atmosphere for 3 hours. The mixture was diluted with water and dichloromethane and filtered through a phase separation cartridge (Biotage Isolute® phase separator, part number 120-1903-B). The organic phase was concentrated under reduced pressure. The residue was purified by chromatography on silica gel (dichloromethane/methanol gradient up to 10% methanol content). This gave 85 mg of (3R)-6-{[2-methoxy-5-(morpholin-4-ylsulphonyl)phenyl]amino}-1,3-dimethyl-4-(1-methylpiperidin-4-yl)-3,4-dihydropyrido[2,3-b]pyrazin-2(1H)-one.

¹H NMR (400 MHz, 25° C., CDCl₃): δ=1.19 (d, 3H); 1.60-1.79 (m, 3H); 1.93 (dq, 1H); 2.08 (d, 1H); 2.26-2.34 (m, 4H); 2.86-3.00 (m, 6H); 3.30 (s, 3H); 3.70-3.73 (m, 4H); 3.99 (s, 3H); 4.31 (q, 1H); 4.40 (tt, 1H); 6.28 (d, 1H); 6.81 (s, 1H); 6.96 (d, 1H); 7.04 (d, 1H); 7.24-7.28 (m, 1H); 8.32 (d, 1H).

### Example 26

N-Cyclopropyl-3-{[(3R)-1,3-dimethyl-2-oxo-4-(tetrahydro-2H-pyran-4-yl)-1,2,3,4-tetrahydropyrido[2,3-b]pyrazin-6-yl]amino}benzenesulphonamide

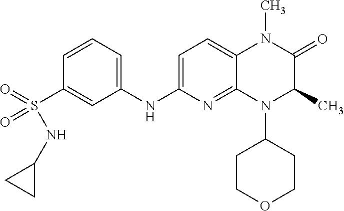 [SMILES: C[C@@H]1C(=O)N(C)c2ccc(Nc3cccc(S(=O)(=O)NC4CC4)c3)nc2N1C1CCOCC1]

A mixture of 150 mg of Intermediate 10, 153 mg of 3-amino-N-cyclopropylbenzenesulphonamide (Amine 8, Table 1), 6.6 mg of tris(dibenzylideneacetone)dipalladium(0) (CAS 51364-51-3), 235 mg of caesium carbonate and 12.3 mg of Xanthphos (CAS 161265-03-8) in 15 ml of dioxane was stirred under an argon atmosphere at 120° C. for 20 hours. The mixture was added to water and extracted twice with ethyl acetate. The organic phase was washed with saturated sodium chloride solution and dried over sodium sulphate, and the solvent was removed under reduced pressure. The residue was purified by chromatography on silica gel (dichloromethane/methanol gradient up to 2% methanol content). This gave 20 mg of N-cyclopropyl-3-{[(3R)-1,3-dimethyl-2-oxo-4-(tetrahydro-2H-pyran-4-yl)-1,2,3,4-tetrahydropyrido[2,3-b]pyrazin-6-yl]amino}benzenesulphonamide.

¹H NMR (400 MHz, 25° C.; DMSO-d6): δ=0.40 (d, 2H); 0.43-0.50 (m, 2H); 1.08 (d, 3H); 1.54-1.61 (m, 1H); 1.68-1.80 (m, 1H); 1.81-1.90 (m, 1H); 1.90-1.98 (m, 1H); 2.06-2.13 (m, 1H); 3.20 (s, 3H); 3.46-3.57 (m, 2H); 3.87-3.95 (m, 2H); 4.23 (q, 1H); 4.47 (tt, 1H); 6.27 (d, 1H); 7.23 (dt, 1H); 7.28 (d, 1H); 7.41-7.47 (m, 1H); 7.80 (d, 1H); 7.96 (d, 2H); 9.16 (s, 1H).

### Example 27

### (3R)-1,3-Dimethyl-6-{[3-(pyrrolidin-1-ylsulphonyl)phenyl]amino}-4-(tetrahydro-2H-pyran-4-yl)-3,4-dihydropyrido[2,3-b]pyrazin-2(1H)-one

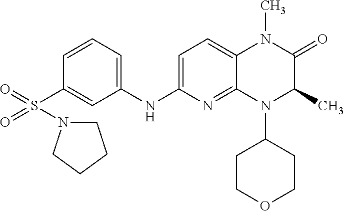 [SMILES: C[C@@H]1C(=O)N(C)c2ccc(Nc3cccc(S(=O)(=O)N4CCCC4)c3)nc2N1C1CCOCC1]

A mixture of 150 mg of Intermediate 10, 163 mg of 3-(pyrrolidin-1-ylsulphonyl)aniline (Amine 1, Table 1), 6.6 mg of tris(dibenzylideneacetone)dipalladium(0) (CAS 51364-51-3), 235 mg of caesium carbonate and 12.3 mg of Xanthphos (CAS 161265-03-8) in 15 ml of dioxane was stirred under an argon atmosphere at 120° C. for 20 hours. The mixture was added to water and extracted twice with ethyl acetate. The organic phase was washed with saturated sodium chloride solution and dried over sodium sulphate, and the solvent was removed under reduced pressure. The residue was purified by chromatography on silica gel (dichloromethane/methanol gradient up to 2% methanol content). This gave 150 mg of (3R)-1,3-Dimethyl-6-{[3-(pyrrolidin-1-ylsulphonyl)phenyl]amino}-4-(tetrahydro-2H-pyran-4-yl)-3,4-dihydropyrido[2,3-b]pyrazin-2(1H)-one.

¹H NMR (400 MHz, 25° C.; DMSO-d6): δ=1.08 (d, 3H); 1.58 (bd, 1H); 1.62-1.69 (m, 4H); 1.77 (qd, 1H); 1.83-1.98 (m, 2H); 3.10-3.18 (m, 4H); 3.21 (s, 3H); 3.47-3.57 (m, 2H); 3.89-3.98 (m, 2H); 4.24 (q, 1H); 4.47 (tt, 1H); 6.28 (d, 1H); 7.20 (bd, 1H); 7.30 (d, 1H); 7.47 (t, 1H); 7.89 (t, 1H); 8.08 (dd, 1H); 9.21 (s, 1H).

### Example 28

### (3R)-6-{[2-Methoxy-5-(morpholin-4-ylsulphonyl)phenyl]amino}-1,3-dimethyl-4-(tetrahydro-2H-pyran-4-yl)-3,4-dihydropyrido[2,3-b]pyrazin-2(1H)-one

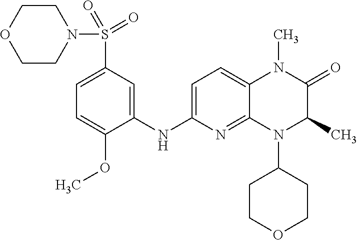 [SMILES: COc1ccc(S(=O)(=O)N2CCOCC2)cc1Nc1ccc2c(n1)N(C1CCOCC1)[C@H](C)C(=O)N2C]

A mixture of 200 mg of Intermediate 10, 202 mg of Intermediate 97, 11 mg of 2-dicyclohexylphosphino-2′,6′-diisopropoxybiphenyl (CAS: 787618-22-8), 18.3 mg of chloro(2-dicyclohexylphosphino-2′,6′-diisopropoxy-1,1′-biphenyl) [2-(2′-amino-1,1′-biphenyl)]palladium(II) (CAS: 1375325-68-0) and 264 mg of caesium carbonate in 2.3 ml of dioxane was stirred at 130° C. under an argon atmosphere for 1.5 hours. The reaction was diluted with water and extracted three times with dichloromethane. The combined organic phases were dried over magnesium sulphate and the solvent was removed under reduced pressure. The residue was purified by chromatography on silica gel (dichloromethane/methanol gradient up to 2% methanol content). This gave 40 mg of (3R)-6-{[2-methoxy-5-(morpholin-4-ylsulphonyl)phenyl]amino}-1,3-dimethyl-4-(tetrahydro-2H-pyran-4-yl)-3,4-dihydropyrido[2,3-b]pyrazin-2(1H)-one.

¹H NMR (400 MHz, CDCl₃): δ=1.21 (d, 3H); 1.60-1.67 (m, 1H); 1.77 (dq, 1H); 1.91 (dq, 1H); 1.99-2.06 (m, 1H); 2.94-2.97 (m, 4H); 3.31 (s, 3H); 3.63-3.75 (m, 6H); 3.96-4.02 (m, 5H); 4.30 (q, 1H); 4.65-4.73 (m, 1H); 6.29 (d, 1H); 6.85 (s, 1H); 6.97 (d, 1H); 7.07 (d, 1H); 8.42 (s, 1H).

### Example 29

(3R)-6-({3-[(3,3-Difluoroazetidin-1-yl)sulphonyl]phenyl}amino)-1,3-dimethyl-4-(1-methylpiperidin-4-yl)-3,4-dihydropyrido[2,3-b]pyrazin-2(1H)-one

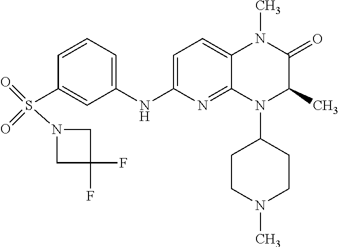 [SMILES: C[C@@H]1C(=O)N(C)c2ccc(Nc3cccc(S(=O)(=O)N4CC(F)(F)C4)c3)nc2N1C1CCN(C)CC1]

A mixture of 200 mg of Intermediate 65, 161 mg of 3-[(3,3-difluoroazetidin-1-yl)sulphonyl]aniline (see Intermediate 99), 10.6 mg of 2-dicyclohexylphosphino-2′,6′-diisopropoxybiphenyl (CAS: 787618-22-8), 17.6 mg of chloro(2-dicyclohexylphosphino-2′,6′-diisopropoxy-1,1′-biphenyl)[2-(2′-amino-1,1′-biphenyl)]palladium(II) (CAS: 1375325-68-0) and 253 mg of caesium carbonate in 2 ml of dioxane was stirred at 130° C. under an argon atmosphere for 2 hours. The mixture was diluted with water and dichloromethane and filtered through a phase separation cartridge (Biotage Isolute® phase separator, part number 120-1903-B). The organic phase was concentrated under reduced pressure. The residue was purified by chromatography on silica gel (dichloromethane/methanol gradient up to 10% methanol content). This gave 115 mg of (3R)-6-({3-[(3,3-difluoroazetidin-1-yl)sulphonyl]phenyl}amino)-1,3-dimethyl-4-(1-methylpiperidin-4-yl)-3,4-dihydropyrido[2,3-b]pyrazin-2(1H)-one.

¹H NMR (300 MHz, CDCl₃): δ=1.23 (d, 3H); 1.58-1.90 (m, 2H); 2.01-2.23 (m, 4H); 2.32 (s, 3H); 2.96 (d, 2H); 3.31 (s, 3H); 4.14-4.21 (t, 4H); 4.24-4.35 (m, 2H); 6.24 (d, 1H); 6.48 (s, 1H); 7.04 (d, 1H); 7.37 (d, 1H); 7.49 (t, 1H); 7.75 (s, 1H); 7.82 (d, 1H).

### Example 30

### (3R)-6-{[2-Methoxy-5-(2-oxa-6-azaspiro[3.3]hept-6-ylsulphonyl)phenyl]amino}-1,3-dimethyl-4-(tetrahydro-2H-pyran-4-yl)-3,4-dihydropyrido[2,3-b]pyrazin-2(1H)-one

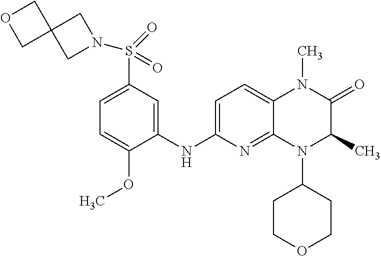 [SMILES: COc1ccc(S(=O)(=O)N2CC3(COC3)C2)cc1Nc1ccc2c(n1)N(C1CCOCC1)[C@H](C)C(=O)N2C]

A mixture of 150 mg of Intermediate 10, 205 mg of Intermediate 101, 22 mg of tris(dibenzylideneacetone)dipalladium(0) (CAS 51364-51-3), 235 mg of caesium carbonate and 28 mg of Xanthphos (CAS 161265-03-8) in 15 ml of dioxane was stirred under an argon atmosphere at 120° C. for 6 hours. Then another 22 mg of tris(dibenzylideneacetone)dipalladium(0) (CAS 51364-51-3) and 28 mg of Xanthphos (CAS 161265-03-8) were added and the mixture was stirred at 120° C. for 8 hours. The mixture was added to water and extracted twice with ethyl acetate. The organic phase was washed with saturated sodium chloride solution and dried over sodium sulphate, and the solvent was removed under reduced pressure. The residue was purified by RP-HPLC (Waters SQD autopurification system; column: Waters XBridge C18 5 μm 100×30 mm; eluent A: water+0.1% by vol. of formic acid (99%), eluent B: acetonitrile; gradient: 0-8.0 min 1-100% B, 8.0-10.0 min 100% B; flow rate 50.0 ml/min; temperature: RT; injection: 2500 μl; DAD scan: 210-400 nm). This gave 105 mg of (3R)-6-{[2-methoxy-5-(2-oxa-6-azaspiro[3.3]hept-6-ylsulphonyl)phenyl]amino}-1,3-dimethyl-4-(tetrahydro-2H-pyran-4-yl)-3,4-dihydropyrido[2,3-b]pyrazin-2(1H)-one.

¹H NMR (400 MHz, DMSO-d6): δ=1.06 (d, 3H); 1.51 (bd, 1H); 1.68 (qd, 1H); 1.76 (qd, 1); 1.84 (bd, 1H); 3.21 (s, 3H); 3.47-3.59 (m, 2H); 3.77-3.87 (m, 6H); 3.97 (s, 3H); 4.21 (q, 1H); 4.44 (s, 4H); 4.53 (tt, 1H); 6.58 (d, 1H); 7.21 (d, 1H); 7.27-7.33 (m, 2H); 8.14 (s, 1H); 8.56 (d, 1H).

### Example 31

### (3R)-6-({3-[(3,3-Difluoroazetidin-1-yl)sulphonyl]phenyl}amino)-1,3-dimethyl-4-(tetrahydro-2H-pyran-4-yl)-3,4-dihydropyrido[2,3-b]pyrazin-2(1H)-one

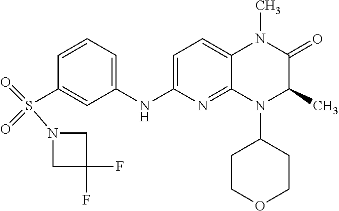 [SMILES: C[C@@H]1C(=O)N(C)c2ccc(Nc3cccc(S(=O)(=O)N4CC(F)(F)C4)c3)nc2N1C1CCOCC1]

A mixture of 100 mg of Intermediate 10, 119 mg of Intermediate 99, 14.7 mg of tris(dibenzylideneacetone)dipalladium(0) (CAS 51364-51-3), 157 mg of caesium carbonate and 18.6 mg of Xanthphos (CAS 161265-03-8) in 15 ml of dioxane was stirred under an argon atmosphere at 120° C. for 8 hours. Then another 14.7 mg of tris(dibenzylideneacetone)dipalladium(0) (CAS 51364-51-3) and 18.6 mg of Xanthphos (CAS 161265-03-8) were added and the mixture was stirred at 120° C. for 7 hours. The mixture was added to water and extracted twice with ethyl acetate. The organic phase was washed with saturated aqueous sodium chloride solution and dried over sodium sulphate, and the solvent was removed under reduced pressure. The residue was purified by RP-HPLC (Waters SQD autopurification system; column: Waters XBridge C18 5 μm 100×30 mm; eluent A: water+0.1% by vol. of formic acid (99%), eluent B: acetonitrile; gradient: 0-8.0 min 1-100% B, 8.0-10.0 min 100% B; flow rate 50.0 ml/min; temperature: RT; injection: 2500 μl; DAD scan: 210-400 nm). This gave 19 mg of (3R)-6-({3-[(3,3-difluoroazetidin-1-yl)sulphonyl]phenyl}amino)-1,3-dimethyl-4-(tetrahydro-2H-pyran-4-yl)-3,4-dihydropyrido[2,3-b]pyrazin-2(1H)-one.

¹H NMR (400 MHz, DMSO-d6): δ=1.08 (d, 3H); 1.58 (bd, 1H); 1.69-1.99 (m, 3H); 3.21 (s, 3H); 3.43-3.58 (m, 2H); 3.86-4.00 (m, 2H); 4.17-4.29 (m, 5H); 4.46 (tt, 1H); 6.29 (d, 1H); 7.26-7.35 (m, 2H); 7.56 (t, 1H); 7.99 (t, 1H); 8.10 (dd, 1H); 9.30 (s, 1H).

TABLE 2
The following examples were prepared in accordance with the named general synthesis
method from respective intermediates/amines (from Table 1):

| | | | Intermediate/ | |
|---|---|---|---|---|
| Ex. | Structure | Name | Amine | Analytical data ¹H NMR |
| 32 | 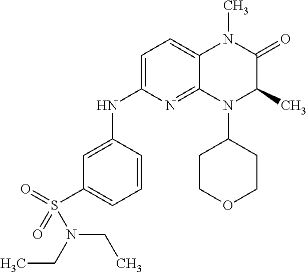 [SMILES: CCN(CC)S(=O)(=O)c1cccc(Nc2ccc3c(n2)N(C2CCOCC2)[C@H](C)C(=O)N3C)c1] | 3-{[(3R)-1,3-dimethyl-2- oxo-4-(tetrahydro-2H- pyran-4-yl)-1,2,3,4- tetrahydropyrido[2,3- b]pyrazin-6-yl]amino}- N,N-diethylbenzene- sulphonamide | Intermediate 10, Intermediate 9 General Synthesis Method (B) | (400 MHz, DMSO-d6): δ = 0.99-1.12 (m, 9H); 1.58 (bd, 1H); 1.68-2.00 (m, 3H); 3.15 (q, 4H); 3.21 (s, 3H); 3.46- 3.57 (m, 2H); 3.87-3.99 (m, 2H); 4.24 (q, 1H); 4.47 (tt, 1H); 6.27 (d, 1H); 7.17 (bd, 1H); 7.29 (d, 1H); 7.43 (t, 1H); 7.89 (t, 1H); 8.03 (bd, 1H); 9.19 (s, 1H). |
| 33 | 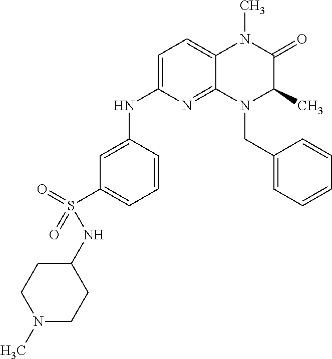 [SMILES: C[C@@H]1C(=O)N(C)c2ccc(Nc3cccc(S(=O)(=O)NC4CCN(C)CC4)c3)nc2N1Cc1ccccc1] | 3-{[(3R)-4-benzyl-1,3- dimethyl-2-oxo-1,2,3,4- tetrahydropyrido[2,3- b]pyrazin-6-yl]amino}-N- (1-methylpiperidin-4- yl)benzenesulphonamide | Intermediate 41, Intermediate 92, General Synthesis Method B | (400 MHz, DMSO-d6): δ = 1.11 (d, 3H); 1.24-1.37 (m, 2H); 1.45 (d, 2H); 1.64 (d, 2H); 2.00 (s, 3H); 2.50-2.56 (m, 2H); 2.82 (d, 1H); 3.23 (s, 3H); 3.95 (q, 1H); 4.24 (d, 1H); 5.30 (d, 1H); 6.28 (d, 1H); 7.18 (d, 1H); 7.23- 7.37 (m, 7H); 7.48 (d, 1H); 7.60 (dd, 1H); 8.23 (t, 1H); 9.15 (s, 1H). |
| 34 | 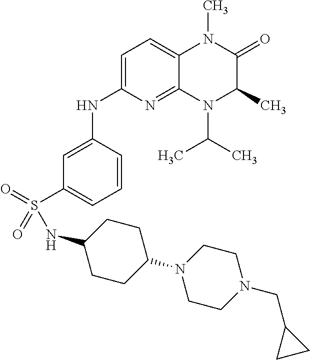 | N-{trans-4-[4- (cyclopropylmethyl) piperazin-1-yl] cyclohexyl}-3- {[(3R)-4-isopropyl-1,3- dimethyl-2-oxo-1,2,3,4- tetrahydropyrido[2,3- b]pyrazin-6-yl]amino}- benzenesulphonamide | Intermediate 33, Intermediate 106, General Synthesis Method B | (300 MHz, DMSO-d6): δ = 0.02 (q, 2H); 0.33-0.46 (m, 2H); 0.69-0.82 (m, 1H); 0.97- 1.06 (m, 1H); 1.09 (d, 3H); 1.11-1.19 (m, 3H); 1.23 (d, 3H); 1.31 (d, 3H); 1.67 (d, 4H); 2.03-2.12 (m, 1H); 2.15 (d, 2H); 2.42 (bs, 8H); 2.87 (bs, 1H); 3.20 (s, 3H): 4.24 (q, 1H); 4.72 (spt, 1H); 6.24 (d, 1H); 7.21 (d, 1H); 7.25 (d, 1H); 7.39 (t, 1H); 7.47 (d, 1H); 7.66 (br. d, 1H); 8.31 (br. s, 1H); 9.12 (s, 1H). |
| 35 | 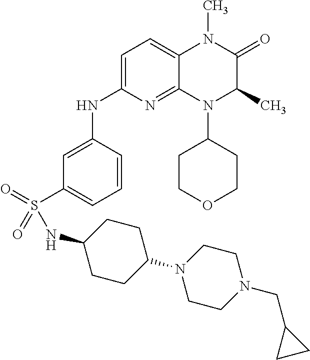 | N-{trans-4-[4- (cyclopropylmethyl)- piperazin-1- yl]cyclohexyl}-3-{[(3R)- 1,3-dimethyl-2-oxo-4- (tetrahydro-2H-pyran-4- yl)-1,2,3,4- tetrahydropyrido[2,3- b]pyrazin-6-yl]amino}- benzenesulphonamide | Intermediate 10, Intermediate 106, General Synthesis Method B | (400 MHz, DMSO-d6): δ = 0.01-0.07 (m, 2H); 0.39- 0.45 (m, 2H); 0.77 (bs, 1H); 1.03-1.20 (m, 7H); 1.57 (d, 1H); 1.63-1.79 (m, 5H); 1.81-1.98 (m, 2H); 2.08- 2.19 (m, 3H); 2.43 (bs, 6H); 2.52-2.54 (m, 1H); 2.80- 2.91 (m, 1H); 3.20 (s, 3H); 3.51 (ddd, 2H); 3.91 (s, 2H); 4.23 (q, 1H); 4.47 (tt, 1H); 6.27 (d, 1H); 7.23 (br. d, 1H); 7.28 (d, 1H); 7.41 (t, 1H); 7.54 (d, 1H); 7.87 (dd, 1H); 7.97 (t, 1H); 8.19 (s, 1H); 9.13 (s, 1H). |
| 36 | 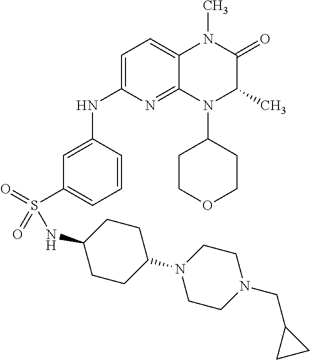 | N-{trans-4-[4- (cyclopropylmethyl)- piperazin-1- yl]cyclohexyl}-3-{[(3S)- 1,3-dimethyl-2-oxo-4- (tetrahydro-2H-pyran-4- yl)-1,2,3,4- tetrahydropyrido[2,3- b]pyrazin-6-yl]amino}- benzenesulphonamide | Intermediate 113, Intermediate 106, General Synthesis Method B | (400 MHz, DMSO-d6): δ = 0.00-0.06 (m, 2H); 0.39- 0.45 (m, 2H); 0.72-0.82 (m, 1H); 1.03-1.20 (m, 7H); 1.58 (bd, 1H); 1.63-1.79 (m, 5H); 1.81-1.98 (m, 2H); 2.08-2.19 (m, 3H); 2.42 (bs, 6H); 2.52-2.54 (m, 1H); 2.80-2.91 (m, 1H); 3.21 (s, 3H); 3.51 (ddd, 2H); 3.91 (s, 2H); 4.24 (q, 1H); 4.48 (tt, 1H); 6.28 (d, 1H); 7.24 (br. d, 1H); 7.29 (d, 1H); 7.42 (t, 1H); 7.54 (d, 1H); 7.88 (dd, 1H); 7.97 (t, 1H); 8.18 (s, 1H); 9.14 (s, 1H). |
| 37 | 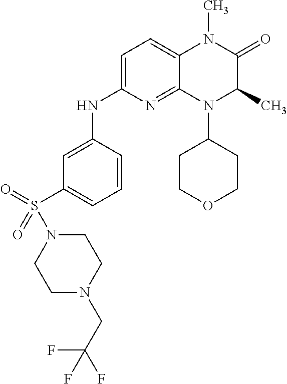 [SMILES: C[C@@H]1C(=O)N(C)c2ccc(Nc3cccc(S(=O)(=O)N4CCN(CC(F)(F)F)CC4)c3)nc2N1C1CCOCC1] | (3R)-1,3-dimethyl-4- (tetrahydro-2H-pyran-4- yl)-6-[(3-{[4-(2,2,2- trifluoroethyl)piperazin-1- yl]sulphonyl}phenyl)- amino]-3,4-dihydropyrido- [2,3-b]pyrazin-2(1H)-one | Intermediate 10. Intermediate 108, General Synthesis Method B | (300 MHz, DMSO-d6): δ = 0.96-1.13 (m, 3H); 1.58 (br. d, 1H); 1.91 (bs, 3H); 2.67 (bs, 5H); 2.87 (bs, 4H); 3.10-3.26 (m, 5H); 3.50 (br. t, 2H); 3.92 (bs, 2H); 4.23 (q, 1H); 4.44 (br. t, 1H); 6.27 (d, 1H); 7.12 (br. d, 1H); 7.29 (d, 1H); 7.49 (t, 1H); 7.78 (br. s, 1H); 8.10 (br. d, 1H); 9.24 (s, 1H). |
| 38 | 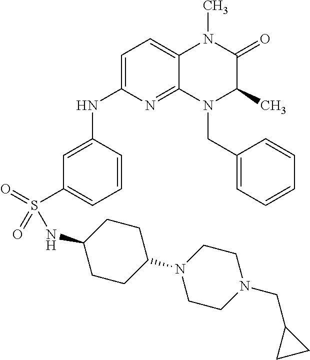 | 3-{[(3R)-4-benzyl-1,3- dimethyl-2-oxo-1,2,3,4- tetrahydropyrido[2,3- b]pyrazin-6-yl]amino}-N- {trans-4-[4-(cyclopropyl- methyl)piperazin-1- yl]cyclohexyl}- benzenesulphonamide | Intermediate 41, Intermediate 106, General Synthesis Method B | (400 MHz, DMSO-d6): δ = −0.02-0.04 (m, 2H); 0.36- 0.43 (m, 2H); 0.75 (bs, 1H); 0.88-1.09 (m, 4H); 1.11 (d, 3H); 1.61 (d, 4H); 2.01 (bs, 1H); 2.07 (d, 2H); 2.34 (bs, 8H); 2.73-2.85 (m, 1H); 3.23 (s, 3H); 3.96 (q, 1H); 4.25 (d, 1H); 5.30 (d, 1H); 6.28 (d, 1H); 7.17 (d, 1H); 7.22-7.37 (m, 7H); 7.43 (d, 1H); 7.61 (dd, 1H); 8.20 (t, 1H); 9.14 (s, 1H). |
| 39 | 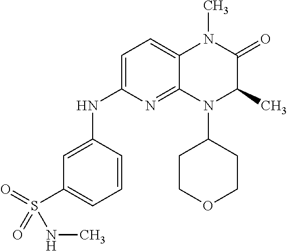 [SMILES: CNS(=O)(=O)c1cccc(Nc2ccc3c(n2)N(C2CCOCC2)[C@H](C)C(=O)N3C)c1] | 3-{[(3R)-1,3-dimethyl-2- oxo-4-(tetrahydro-2H- pyran-4-yl)-1,2,3,4- tetrahydropyrido[2,3- b]pyrazin-6-yl]amino}-N- methylbenzenesulphon- amide | Intermediate 10, Amine 10, General Synthesis Method B | (400 MHz, DMSO-d6): δ = 1.07 (d, 3H); 1.53-1.60 (m 1H); 1.68-1.80 (m, 1H); 1.80-1.90 (m, 1H); 1.90- 1.97 (m, 1H); 2.41 (d, 3H); 3.20 (s, 3H); 3.48-3.56 (m, 2H); 3.86-3.96 (m, 2H); 4.23 (q, 1H); 4.47 (tt, 1H); 6.27 (d, 1H); 7.19 (br. d, 1H); 7.26-7.33 (m, 2H); 7.43 (t, 1H); 7.90-7.95 (m, 2H); 9.16 (s, 1H). |
| 40 | 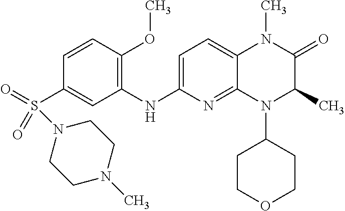 [SMILES: COc1ccc(S(=O)(=O)N2CCN(C)CC2)cc1Nc1ccc2c(n1)N(C1CCOCC1)[C@H](C)C(=O)N2C] | (3R)-6-({2-methoxy-5-[(4- methylpiperazin-1- yl)sulphonyl]phenyl}- amino)-1,3-dimethyl-4- (tetrahydro-2H-pyran-4- yl)-3,4-dihydropyrido[2,3- b]pyrazin-2(1H)-one | Intermediate 10, Intermediate 116, General Synthesis Method B | (400 MHz, CDCl3): δ = 1.20 (d, 3H); 1.62 (d, 1H); 1.77 (dq, 1H); 1.90 (dq, 1H); 2.02 (d, 1H); 2.25 (bs, 3H); 2.45 (bs, 4H); 2.99 (bs, 4H); 3.31 (s, 3H); 3.64-3.75 (m, 2H); 3.97-4.02 (m, 5H); 4.30 (q, 1H); 4.69 (tt, 1H); 6.28 (d, 1H); 6.82 (s, 1H); 6.93 (d, 1H); 7.06 (d, 1H); 7.25-7.27 (m, 1H); 8.41 (d, 1H). |
| 41 | 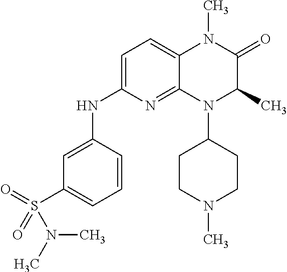 [SMILES: C[C@@H]1C(=O)N(C)c2ccc(Nc3cccc(S(=O)(=O)N(C)C)c3)nc2N1C1CCN(C)CC1] | 3-{[(3R)-1,3-dimethyl-4- (1-methylpiperidin-4-yl)- 2-oxo-1,2,3,4- tetrahydropyrido[2,3- b]pyrazin-6-yl]amino}- N,N-dimethylbenzene- sulphonamide | Intermediate 65, Amine 2, General Synthesis Method C | (400 MHz, CDCl3): δ = 1.22 (d, 3H); 1.65-1.70 (m, 1H); 1.84 (q, 1H); 2.00-2.18 (m, 4H); 2.32 (s, 3H); 2.72 (s, 6H); 2.97 (d, 2H); 3.30 (s, 3H); 4.21-4.33 (m, 2H); 6.25 (d, 1H); 6.51 (s, 1H); 7.03 (d, 1H); 7.79 (ddd, 1H); 7.43 (t, 1H); 7.59 (s, 1H); 7.81 (d, 1H). |
| 42 | 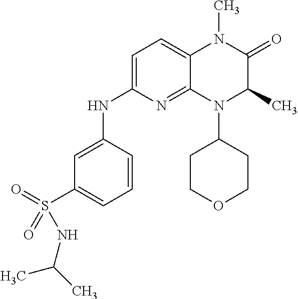 [SMILES: CC(C)NS(=O)(=O)c1cccc(Nc2ccc3c(n2)N(C2CCOCC2)[C@H](C)C(=O)N3C)c1] | 3-{[(3R)-1,3-dimethyl-2- oxo-4-(tetrahydro-2H- pyran-4-yl)-1,2,3,4- tetrahydropyrido[2,3- b]pyrazin-6-yl]amino}-N- isopropylbenzene- sulphonamide | Intermediate 10, Amine 11, General Synthesis Method B | (400 MHz, CDCl3): δ = 0.95 (d, 6H); 1.08 (d, 3H); 1.57 (bd, 1H); 1.66-2.00 (m, 3H); 3.16-3.29 (m+s, 4H); 3.45- 3.59 (m, 2H); 3.85-3.99 (m, 2H); 4.24 (q, 1H); 4.47 8tt, 1H); 6.27 (d, 1H); 7.23 (d, 1H); 7.29 (d, 1H); 7.43 (t, 1H); 7.48 (d, 1H); 7.91 (d, 1H); 7.95 (t, 1H); 9.16 (s, 1H). |
| 43 | 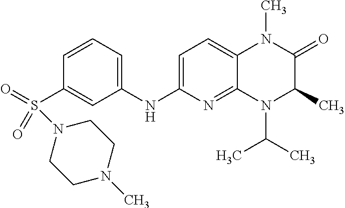 [SMILES: CC(C)N1c2nc(Nc3cccc(S(=O)(=O)N4CCN(C)CC4)c3)ccc2N(C)C(=O)[C@H]1C] | (3R)-4-isopropyl-1,3- dimethyl-6-({3-[(4- methylpiperazin-1- yl)sulphonyl]phenyl}- amino)-3,4-dihydropyrido- [2,3-b]pyrazin-2(1H)-one | Intermediate 33, Intermediate 110, General Synthesis Method B | (400 MHz, DMSO-d6): δ = 1.08 (d, 3H); 1.21 (d, 3H); 1.30 (d, 3H); 1.99-2.23 (m, 3H); 2.34 (t, 4H); 2.87 (bs, 4H); 3.19 (s, 3H); 4.24 (q, 1H); 4.74 (spt, 1H); 6.25 (d, 1H); 7.11 (d, 1H); 7.27 (d, 1H); 7.45 (dd, 1H); 7.71 (dd, 1H); 8.30 (br. t, 1H); 9.21 (s, 1H). |
| 44 | 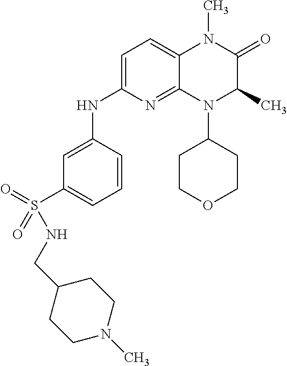 [SMILES: C[C@@H]1C(=O)N(C)c2ccc(Nc3cccc(S(=O)(=O)NCC4CCN(C)CC4)c3)nc2N1C1CCOCC1] | 3-{[(3R)-1,3-dimethyl-2- oxo-4-(tetrahydro-2H- pyran-4-yl)-1,2,3,4- tetrahydropyrido[2,3- b]pyrazin-6-yl]amino}-N- [(1-methylpiperidin-4- yl)methyl]benzene- sulphonamide | Intermediate 10, Intermediate 112, General Synthesis Method B | (400 MHz, DMSO-d6): δ = 0.96-1.05 (m, 2H); 1.07 (d, 3H); 1.21-1.35 (m, 1H); 1.53-1.61 (m, 3H); 1.70- 1.82 (m, 3H); 1.82-1.97 (m, 2H); 2.11 (s, 3H); 2.60 (s, 2H); 2.65-2.74 (m, 2H); 3.20 (s, 3H); 3.46-3.56 (m, 3H); 3.86-3.95 (m, 2H); 4.19-4.27 (m, 1H); 4.41- 4.51 (m, 1H); 6.26 (d, 1H); 7.19 (d, 1H); 7.28 (d, 1H); 7.41 (s, 1H); 7.52 (t, 1H); 7.89 (br. d, 1H); 7.93 (s, 1H); 9.16 (s, 1H). |
| 45 | 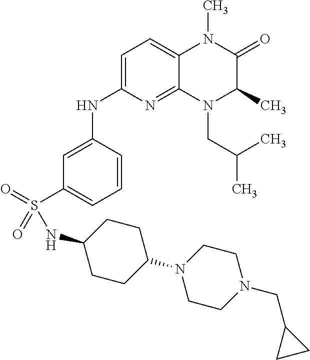 | N-{trans-4-[4- (cyclopropylmethyl)- piperazin-1- yl]cyclohexyl}-3-{[(3R)-4- isobulyl-1,3-dimethyl-2- oxo-1,2,3,4- tetrahydropyrido[2,3- b]pyrazin-6-yl]amino}- benzenesulphonamide | Intermediate 60, Intermediate 106, General Synthesis Method B | (400 MHz. DMSO-d6): δ = −0.03-0.05 (m, 2H); 0.37- 0.43 (m, 2H); 0.69-0.79 (m, 1H); 0.87 (d, 3H); 0.91 (d, 3H); 1.02-1.19 (m, 7H); 1.67 (d, 4H); 2.07 (d, 4H); 2.27-2.43 (m, 7H); 2.66 (s, 2H); 2.80-2.92 (m, 1H); 3.18-3.27 (m, 3H); 3.93- 4.01 (m, 1H); 4.07 (q, 1H); 6.21 (d, 1H); 7.19-7.25 (m, 2H); 7.38 (t, 1H); 7.50 (d, 1H); 7.89 (dd, 1H); 8.02 (t, 1H); 9.10 (s, 1H). |
| 46 | 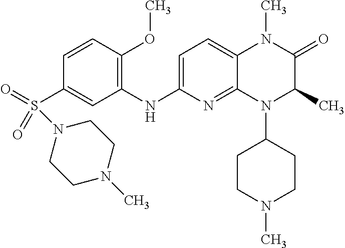 [SMILES: COc1ccc(S(=O)(=O)N2CCN(C)CC2)cc1Nc1ccc2c(n1)N(C1CCN(C)CC1)[C@H](C)C(=O)N2C] | (3R)-6-({2-methoxy-5-[(4- methylpiperazin-1- yl)sulphonyl]phenyl}- amino)-1,3-dimethyl-4-(1- methylpiperidin-4-yl)-3,4- dihydropyrido[2,3- b]pyrazin-2(1H)-one | Intermediate 65, Intermediate 116, General Synthesis Method C | (400 MHz, CDCl3): δ = 1.20 (d, 3H); 1.63-1.80 (m, 3H); 1.94 (q, 1H); 2.08 (d, 1H); 2.21-2.49 (m, 11H); 2.41- 3.09 (m, 6H); 3.29 (s, 3H); 3.97 (s, 3H); 4.30 (q, 1H); 4.41 (tt, 1H); 6.28 (d, 1H); 6.78 (s, 1H); 6.93 (s, 1H); 7.03 (d, 1H); 7.24-7.28 (m, 1H); 8.32 (s, 1H). |
| 47 | 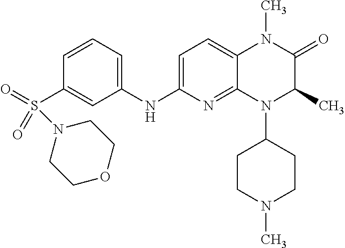 [SMILES: C[C@@H]1C(=O)N(C)c2ccc(Nc3cccc(S(=O)(=O)N4CCOCC4)c3)nc2N1C1CCN(C)CC1] | (3R)-1,3-dimethyl-4-(1- methylpiperidin-4-yl)-6- {[3-(morpholin-4- ylsulphonyl)phenyl]- amino}-3-4-dihydropyrido- [2,3-b]pyrazin-2(1H)-one | Intermediate 65, Intermediate 97, General Synthesis Method C | (300 MHz, CDCl3): δ = 1.23 (d, 3H); 1.71 (d, 1H); 1.91 (br. s, 1H); 2.06-2.29 (m, 4H); 2.36 (s, 3H); 2.97-3.04 (m, 6H); 3.30 (s, 3H); 3.73- 3.76 (m, 4H); 4.20-4.34 (m, 2H); 6.25 (d, 1H); 6.61 (br. s, 1H); 7.03 (d, 1H); 7.25-7.28 (m, 1H); 7.44 (t, 1H); 7.63 (s, 1H); 7.80 (d, 1H). |
| 48 | 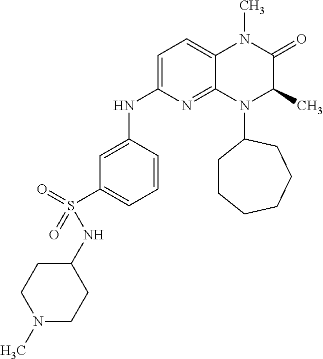 [SMILES: C[C@@H]1C(=O)N(C)c2ccc(Nc3cccc(S(=O)(=O)NC4CCN(C)CC4)c3)nc2N1C1CCCCCC1] | 3-{[(3R)-4-cycloheptyl- 1,3-dimethyl-2-oxo- 1,2,3,4-tetrahydropyrido- [2,3-b]pyrazin-6- yl]amino}-N-(1- methylpiperidin-4- yl)benzenesulphonamide | Intermediate 38, Intermediate 92, General Synthesis Method B | (400 MHz, DMSO-d6): δ = 1.07 (d, 3H); 1.30-1.45 (m, 2H); 1.48-1.73 (m, 12H); 1.84 (t, 3H); 2.03 (d, 1H); 2.06-2.11 (m, 3H); 2.62 (d, 2H); 2.83-2.97 (m, 1H); 3.19 (s, 3H); 4.23 (q, 1H); 4.33 (br. t, 1H); 6.23 (d, 1H): 7.19-7.28 (m, 2H); 7.38 (t, 1H); 7.61 (d, 1H); 7.77 (t, 1H); 8.09 (dd, 1H); 9.12 (s, 1H). |
| 49 | 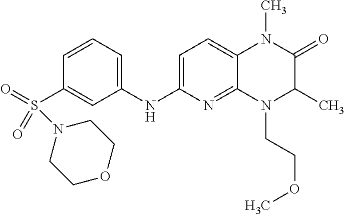 [SMILES: COCCN1c2nc(Nc3cccc(S(=O)(=O)N4CCOCC4)c3)ccc2N(C)C(=O)C1C] | 4-(2-methoxyethyl)-1,3- dimethyl-6-{[3- (morpholin-4-ylsulphonyl) phenyl]amino}-3,4- dihydropyrido[2,3- b]pyrazin-2(1H)-one | Intermediate 47, Amine 6, General Synthesis Method B | (400 MHz, DMSO-d6): δ = 1.12 (d, 3H); 2.81-2.93 (m, 4H); 3.21 (s, 3H); 3.24 (s, 3H); 3.50-3.69 (m, 6H); 4.13-4.26 (m, 2H); 6.24 (d, 1H); 7.12 (d, 1H); 7.26 (d, 1H); 7.48 8t, 1H); 7.77 (dd, 1H); 8.23 (t, 1H); 9.26 (s, 1H). |
| 50 | 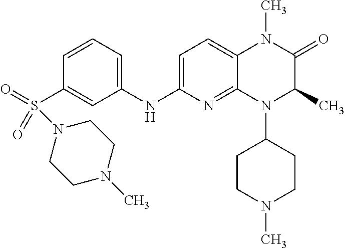 [SMILES: C[C@@H]1C(=O)N(C)c2ccc(Nc3cccc(S(=O)(=O)N4CCN(C)CC4)c3)nc2N1C1CCN(C)CC1] | (3R)-1,3-dimethyl-6-({3- [(4-methylpiperazin-1- yl)sulphonyl]phenyl}- amino)-4-(1-methyl- piperidin-4-yl)-3,4- dihydropyrido[2,3- b]pyrazin-2(1H)-one | Intermediate 65, Intermediate 110, General Synthesis Method C | (300 MHz, CDCl3): δ = 1.21 (d, 3H); 1.70 (d, 1H); 1.88 (s, 1H); 2.06-2.32 (m, 7H); 2.36 (s, 3H); 2.46-2.49 (m, 4H); 2.95-3.07 (m, 6H); 3.30 (s, 3H); 4.20-4.34 (m, 2H); 6.23 (d, 1H); 6.52 (s, 1H); 7.02 (d, 1H); 7.26-7.28 (m, 1H); 7.41 (t, 1H); 7.62 (s, 1H); 7.72 (s, 1H). |
| 51 | 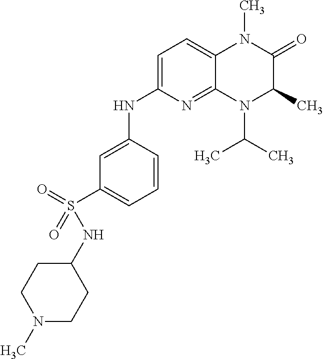 [SMILES: CC(C)N1c2nc(Nc3cccc(S(=O)(=O)NC4CCN(C)CC4)c3)ccc2N(C)C(=O)[C@H]1C] | 3-{[(3R)-4-isopropyl-1,3- dimethyl-2-oxo-1,2,3,4- tetrahydropyrido[2,3- b]pyrazin-6-yl]amino}-N- (1-methylpiperidin-4- yl)benzenesulphonamide | Intermediate 33, Intermediate 92, General Synthesis Method B | (300 MHz, DMSO-d6): δ = 1.08 (d, 3H); 1.22 (d, 3H); 1.30 (d, 3H); 1.33-1.44 (m, 2H); 1.45-1.58 (m, 2H); 1.68-1.80 (m, 2H); 2.04 (s, 3H); 2.54-2.62 (m, 2H); 2.80-2.94 (m, 1H); 3.20 (s, 3H); 4.24 (q, 1H); 4.65- 4.78 (m, 1H); 6.24 (d, 1H); 7.21 (br. d, 1H); 7.26 (d, 1H); 7.39 (t, 1H); 7.52 (d, 1H); 7.65 (br. d, 1H); 8.33 (br. s, 1H); 9.13 (s, 1H). |
| 52 | 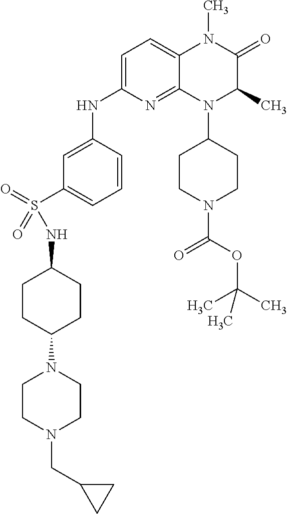 [SMILES: C[C@@H]1C(=O)N(C)c2ccc(Nc3cccc(S(=O)(=O)N[C@H]4CC[C@H](N5CCN(CC6CC6)CC5)CC4)c3)nc2N1C1CCN(C(=O)OC(C)(C)C)CC1] | tert-butyl 4-[(3R)-6-{[3- ({trans-4-[4-(cyclo- propylmethyl)piperazin-1- yl]cyclohexyl}- sulphamoyl)phenyl]amino}- 1,3-dimethyl-2-oxo-2,3- dihydropyrido[2,3- b]pyrazin-4(1H)- yl]piperidine-1- carboxylate | Intermediate 52, Intermediate 106, General Synthesis Method B | (400 MHz, DMSO-d6): δ = −0.02-0.05 (m, 2H); 0.37- 0.46 (m, 2H); 0.71-0.82 (m, 1H); 1.06 (d, 3H); 1.08- 1.21 (m, 5H); 1.42 (s, 9H); 1.61-1.74 (m, 6H); 1.93- 2.06 (m, 2H); 2.09 (d, 3H); 2.39 (bs, 6H); 2.78-3.06 (m, 4H); 3.21 (s, 3H); 4.05 (br. s, 2H); 4.22 (q, 1H); 4.43 (br. t, 1H); 6.28 (d, 1H); 7.24 (br. d, 1H); 7.30 (d, 1H); 7.43 (t, 1H); 7.57 (d, 1H); 7.78 (dd, 1H); 8.07 (br. t, 1H); 9.16 (s, 1H). |
| 53 | 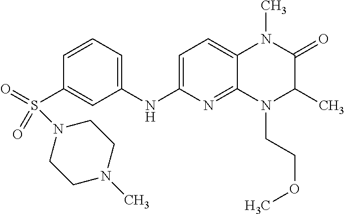 [SMILES: COCCN1c2nc(Nc3cccc(S(=O)(=O)N4CCN(C)CC4)c3)ccc2N(C)C(=O)C1C] | 4-(2-methoxyethyl)-1,3- dimethyl-6-({3-[(4- methylpiperazin-1- yl)sulphonyl]phenyl}- amino)-3,4-dihydropyrido- [2,3-b]pyrazin-2(1H)-one | Intermediate 47, Intermediate 110, General Synthesis Method B | (400 MHz, DMSO-d6): δ = 1.10 (s, 3H); 2.12 (s, 3H); 2.32-2.38 (m, 4H); 2.85- 2.92 (m, 4H); 3.20 (s, 3H); 3.22-3.25 (m, 3H); 3.50- 3.64 (m, 2H); 4.13-4.24 (m, 3H); 6.24 (d, 1H); 7.11 (d, 1H); 7.25 (d, 1H); 7.45 (t, 1H); 7.75 (br. d, 1H); 8.20 (br. s, 1H); 9.21 (s, 1H). |
| 54 | 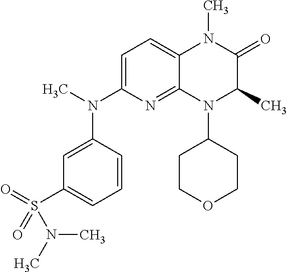 [SMILES: C[C@@H]1C(=O)N(C)c2ccc(N(C)c3cccc(S(=O)(=O)N(C)C)c3)nc2N1C1CCOCC1] | 3-{[(3R)-1,3-dimethyl-2- oxo-4-(tetrahydro-2H- pyran-4-yl)-1,2,3,4- tetrahydropyrido[2,3- b]pyrazin-6-yl](methyl)- amino}-N,N-dimethyl- benzenesulphonamide | Intermediate 10, Amine 3, General Synthesis Method C | (400 MHz, CDCl3): δ = 1.26 (d, 3H); 1.51-1.74 (m, 2H); 1.80 (dd, 1H); 1.91-2.08 (m, 2H); 2.75 (s, 5H); 3.26- 3.43 (m, 4H); 3.44-3.68 (m, 4H); 3.94-4.13 (m, 2H); 4.24-4.41 (m, 2H); 6.18 (d, 1H); 6.98 (d, 1H); 7.37- 7.55 (m, 3H); 7.66 (s, 1H). |
| 55 | 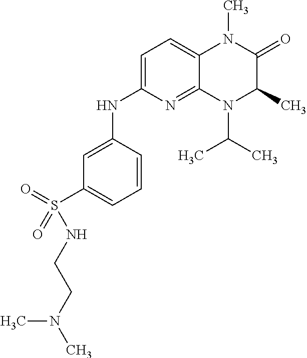 | N-[2-(dimethylamino)- ethyl]-3-{[(3R)-4- isopropyl-1,3-dimethyl-2- oxo-1,2,3,4-tetrahydro- pyrido[2,3-b]pyrazin-6- yl]amino}benzene- sulphonamide | Intermediate 33, Intermediate 94, General Synthesis Method B | (300 MHz, DMSO-d6): δ = 1.08 (d, 3H); 1.22 (d, 3H); 1.30 (d, 3H); 2.08 (s, 6H); 2.28 (t, 2H); 2.83 (t, 2H); 3.20 (s, 3H); 4.24 (q, 1H); 4.72 (spt, 1H); 6.24 (d, 1H); 7.20 (d, 1H); 7.25 (d, 1H); 7.40 (t, 1H); 7.65 (br. d, 1H); 8.16 (s, 1H); 8.34 (br. s, 1H); 9.13 (s, 1H). |
| 56 | 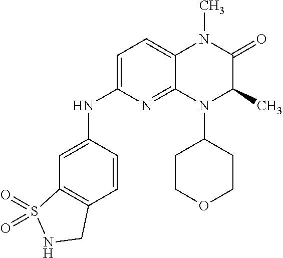 [SMILES: C[C@@H]1C(=O)N(C)c2ccc(Nc3ccc4c(c3)S(=O)(=O)NC4)nc2N1C1CCOCC1] | (3R)-6-[(1,1-dioxido-2,3- dihydro-1,2-benzothiazol- 6-yl)amino]-1,3-dimethyl- 4-(tetrahydro-2H-pyran-4- yl)-3,4-dihydropyrido[2,3- b]pyrazin-2(1H)-one | Intermediate 10, Amine 4, General Svnthcsis Method B | (400 MHz, DMSO-d6): δ = 1.10 (d, 3H); 1.58 (br. d, 1H); 1.71-1.96 (m, 3H); 3.20-3.24 (m, 3H); 3.47- 3.56 (m, 1H); 3.60-3.68 (m, 1H); 3.89-3.96 (m, 2H); 4.26 (q, 1H); 4.30 (d, 2H); 4.52 (tt, 1H); 6.30 (d, 1H); 7.32 (d, 1H); 7.39 (d, 1H); 7.57 (dd, 1H); 7.70 (br. t, 1H); 8.34 (d, 1H); 9.29 (s, 1H). |
| 57 | 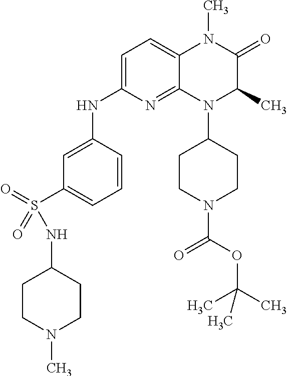 [SMILES: C[C@@H]1C(=O)N(C)c2ccc(Nc3cccc(S(=O)(=O)NC4CCN(C)CC4)c3)nc2N1C1CCN(C(=O)OC(C)(C)C)CC1] | tert-butyl 4-[(3R)-1,3- dimethyl-6-({3-[(1- methylpiperidin-4- yl)sulphamoyl]phenyl}- amino)-2-oxo-2,3-dihydro- pyrido[2,3-b]pyrazin- 4(1H)-yl]piperidine-1- carboxylate | Intermediate 52, Intermediate 92, General Synthesis Method B | (400 MHz, DMSO-d6): δ = 1.05 (d, 3H); 1.38 (d, 2H); 1.41 (s, 9H); 1.51 (d, 3H); 1.55-1.64 (m, 2H); 1.75 (bs, 4H); 1.93-2.01 (m, 1H); 2.04 (s, 3H); 2.57 (d, 2H); 2.80-2.98 (m, 3H); 3.20 (s, 3H); 3.96-4.10 (m, 2H); 4.20 (d, 1H); 4.41 (tt, 1H); 6.27 (d, 1H); 7.23 (br, d, 1H); 7.28 (d, 1H); 7.42 (t, 1H); 7.59 (d, 1H); 7.79 (dd, 1H); 8.04 (t, 1H); 9.13 (s, 1H). |
| 58 | 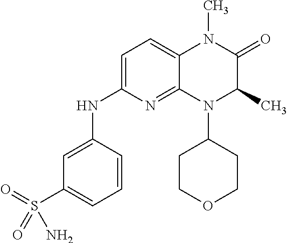 [SMILES: C[C@@H]1C(=O)N(C)c2ccc(Nc3cccc(S(N)(=O)=O)c3)nc2N1C1CCOCC1] | 3-{[(3R)-1,3-dimethyl-2- oxo-4-(tetrahydro-2H- pyran-4-yl)-1,2,3,4- tetrahydropyrido[2,3- b]pyrazin-6-yl]amino}- benzenesulphonamide | Intermediate 10, Amine 5, General Synthesis Method B | (400 MHz, DMSO-d6): δ = 1.07 (d, 3H); 1.53-1.60 (m, 1H); 1.67-1.79 (m, 1H); 1.80-1.97 (m, 2H); 3.20 (s, 3H); 3.48-3.56 (m, 2H); 3.86-3.94 (m, 2H); 4.22 (q, 1H); 4.47 (tt, 1H); 6.26 (d, 1H); 7.23-7.30 (m, 4H); 7.37-7.43 (m, 1H); 7.86 (dd, 1H); 7.98 (t, 1H); 9.15 (s, 1H). |
| 59 | 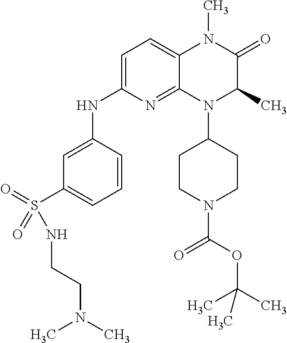 [SMILES: C[C@@H]1C(=O)N(C)c2ccc(Nc3cccc(S(=O)(=O)NCCN(C)C)c3)nc2N1C1CCN(C(=O)OC(C)(C)C)CC1] | tert-butyl 4-[(3R)-6-[(3- {[2- (dimethylamino)ethyl]- sulphamoyl}phenyl)amino]- 1,3-dimethyl-2-oxo-2,3- dihydropyrido[2,3- b]pyrazin-4(1H)- yl]piperidine-1- carboxylate | Intermediate 52, Intermediate 94, General Synthesis Method B | (400 MHz, DMSO-d6): δ = 1.05 (d, 3H); 1.21-1.32 (m, 1H); 1.41 (s, 9H); 1.47-1.63 (m, 2H); 1.63-1.75 (m, 1H); 1.93-2.00 (m, 1H); 2.04 (s, 7H); 2.22 (t, 2H); 2.77-2.84 (m, 2H); 3.20 (s, 3H); 3.97- 4.10 (m, 2H); 4.20 (q, 1H); 4.42 (tt, 1H); 6.27 (d, 1H); 7.21 (d, 1H); 7.28 (d, 1H); 7.34-7.40 (m, 1H); 7.43 (t, 1H); 7.81 (br. d, 1H); 8.02 (br. s, 1H); 9.15 (s, 1H). |
| 60 | 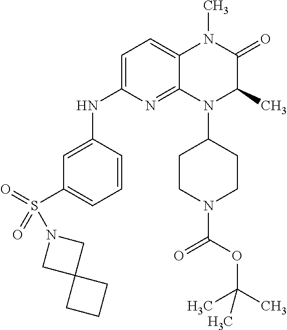 [SMILES: C[C@@H]1C(=O)N(C)c2ccc(Nc3cccc(S(=O)(=O)N4CC5(CCC5)C4)c3)nc2N1C1CCN(C(=O)OC(C)(C)C)CC1] | tert-butyl 4-[(3R)-6-{[3-(2- azaspiro[3.3]hept-2- ylsulphonyl)phenyl]- amino}-1,3-dimethyl-2- oxo-2,3- dihydropyrido[2,3- b]pyrazin-4(1H)- yl]piperidine-1- carboxylate | Intermediate 52, Intermediate 103, General Synthesis Method B | (300 MHz, DMSO-d6): δ = 1.05 (d, 3H); 1.41 (s, 9H); 1.51-1.73 (m, 5H); 1.82- 1.90 (m, 4H); 1.91-2.00 (m, 1H); 2.80-3.00 (m, 2H); 3.20 (s, 3H); 3.64 (s, 4H); 3.96-4.11 (m, 2H); 4.20 (q, 1H); 4.40 (br. t, 1H); 6.28 (d, 1H); 7.18 (br. d, 1H); 7.30 (d, 1H); 7.51 (t, 1H); 7.94- 8.00 (m, 2H); 9.22-9.27 (m, 1H). |
| 61 | 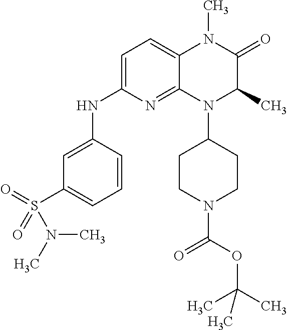 [SMILES: C[C@@H]1C(=O)N(C)c2ccc(Nc3cccc(S(=O)(=O)N(C)C)c3)nc2N1C1CCN(C(=O)OC(C)(C)C)CC1] | tert-butyl 4-[(3R)-6-{[3- (dimethylsulphamoyl)- phenyl]amino}-1,3- dimethyl-2-oxo-2,3- dihydropyrido[2,3- b]pyrazin-4(1H)- yl]piperidine-1- carboxylate | Intermediate 52, Amine 2, General Synthesis Method B | (300 MHz, DMSO-d6): δ = 1.04 (d, 3H); 1.41 (s, 9H); 1.52-1.75 (m, 3H); 1.97 (d, 1H); 2.60 (s, 6H); 2.78-3.01 (m, 2H); 3.20 (s, 3H); 4.05 (bs, 2H); 4.20 (q, J = 6.53 Hz, 1H); 4.39 (t, 1H); 6.27 (d, 1H); 7.13 (d, 1H); 7.29 (d, 1H); 7.49 (t, 1H); 7.85 (s, 1H); 8.03 (d, 1H); 9.23 (s, 1H). |
| 62 | 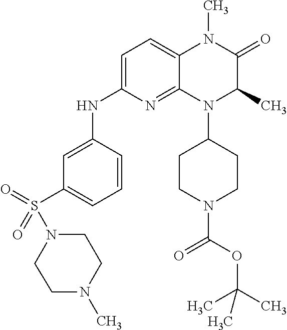 [SMILES: C[C@@H]1C(=O)N(C)c2ccc(Nc3cccc(S(=O)(=O)N4CCN(C)CC4)c3)nc2N1C1CCN(C(=O)OC(C)(C)C)CC1] | tert-butyl 4-[(3R)-1,3- dimethyl-6-({3-[(4- methylpiperazin-1- yl)sulphonyl]phenyl}- amino)-2-oxo-2,3- dihydropyrido[2,3- b]pyrazin-4(1H)- yl]piperidine-1- carboxylate | Intermediate 52, Intermediate 110, General Synthesis Method B | (300 MHz, DMSO-d6): δ = 1.05 (d, 3H); 1.41 (s, 9H); 1.49-1.80 (m, 3H); 1.90- 2.05 (m, 1H); 2.12 (s, 3H); 2.34 (bs, 4H); 2.86 (bs, 6H); 3.20 (s, 3H); 3.96-4.13 (m, 2H); 4.20 (q, 1H); 4.38 (br. t, 1H); 6.27 (d, 1H); 7.12 (d, 1H); 7.29 (d, 1H); 7.49 (t, 1H); 7.84 (br. s, 1H); 8.01 (br. d, 1H); 9.23 (s, 1H). |
| 63 | 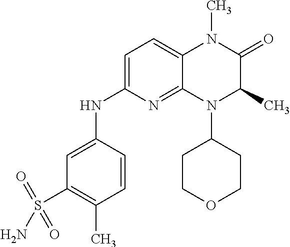 [SMILES: Cc1ccc(Nc2ccc3c(n2)N(C2CCOCC2)[C@H](C)C(=O)N3C)cc1S(N)(=O)=O] | 5-{[(3R)-1,3-dimethyl-2- oxo-4-(tetrahydro-2H- pyran-4-yl)-1,2,3,4- tetrahydropyrido[2,3- b]pyrazin-6-yl]amino}-2- methylbenzene- sulphonamide | Intermediate 10, Amine 12, General Synthesis Method B | (400 MHz, DMSO-d6): δ = 1.07 (d, 3H); 1.53-1.61 (m, 1H); 1.67-1.79 (m, 1H); 1.81-1.90 (m, 1H); 1.90- 1.98 (m, 1H); 2.50-2.54 (m, 2H); 3.19 (s, 3H); 3.46-3.56 (m, 2H); 3.87-3.98 (m, 2H); 4.21 (q, 1H); 4.46 (tt, 1H); 6.24 (d, 1H); 7.18-7.22 (m, 3H); 7.25 (d, 1H); 7.89 (dd, 1H); 7.95 (d, 1H); 9.00 (s, 1H). |
| 64 | 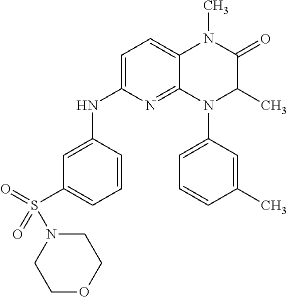 [SMILES: Cc1cccc(N2c3nc(Nc4cccc(S(=O)(=O)N5CCOCC5)c4)ccc3N(C)C(=O)C2C)c1] | 1,3-dimethyl-4-(3- methylphenyl)-6-{[3- (morpholin-4- ylsulphonyl)phenyl]- amino}-3,4- dihydropyrido[2,3- b]pyrazin-2(1H)-one | Intermediate 77, Amine 6, General Synthesis Method B | (400 MHz. DMSO-d6): δ = 1.30 (d, 3H); 2.33 (s, 3H); 2.63-2.87 (m, 4H); 3.33 (s, 3H); 3.60 (t, 4H); 4.55 (q, 1H); 6.42 (d, 1H); 7.01- 7.11 (m, 3H); 7.15 (d, 1H); 7.19 (s, 1H); 7.33 (t, 1H); 7.37 (br. s, 1H); 7.45 (d, 1H); 7.99 (d, 1H); 9.30 (s, 1H). |
| 65 | 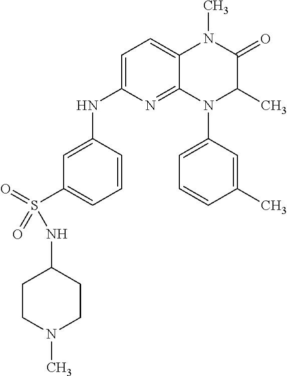 [SMILES: Cc1cccc(N2c3nc(Nc4cccc(S(=O)(=O)NC5CCN(C)CC5)c4)ccc3N(C)C(=O)C2C)c1] | 3-{[1,3-dimethyl-4-(3- methylphenyl)-2-oxo- 1,2,3,4-tetrahydropyrido- [2,3-b]pyrazin-6- yl]amino}-N-(1- methylpiperidin-4- yl)benzenesulphonamide | Intermediate 77, Intermediate 92, General Synthesis Method B | (300 MHz, DMSO-d6): δ = 1.28 (d, 3H); 2.11 (s, 3H); 2.31 (s, 7H); 2.79 (d, 4H); 3.29 (s, 3H); 4.54 (d, 1H); 6.40 (d, 1H); 6.97-7.08 (m, 3H); 7.10-7.20 (m, 2H); 7.28-7.37 (m, 2H); 7.43 (d, 1H); 7.96 (d, 1H); 9.27 (s, 1H). |
| 66 | 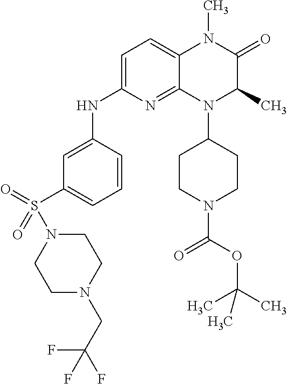 [SMILES: C[C@@H]1C(=O)N(C)c2ccc(Nc3cccc(S(=O)(=O)N4CCN(CC(F)(F)F)CC4)c3)nc2N1C1CCN(C(=O)OC(C)(C)C)CC1] | tert-butyl 4-[(3R)-1,3- dimethyl-2-oxo-6-[(3-{[4- (2,2,2-trifIuoroethyl)- piperazin-1-yl]sulphonyl}- phenl)amino]-2,3- dihydropyrido[2,3- b]pyrazin-4(1H)- yl]piperidine-1- carboxylate | Intermediate 52, Intermediate 108, General Synthesis Method B | (400 MHz, DMSO-d6): δ = 1.06 (d, 3H); 1.42 (s, 9H); 1.51-1.66 (m, 2H); 1.67- 1.80 (m, 1H); 1.92-2.06 (m, 1H); 2.61-2.77 (m, 4H); 2.80-3.02 (m, 6H); 3.14- 3.26 (m, 5H); 3.99-4.14 (m, 2H); 4.22 (q, 1H); 4.40 (br. t, 1H); 6.29 (d, 1H); 7.13 (d, 1H); 7.31 (d, 1H); 7.51 (s, 1H); 7.85 (br. s, 1H); 8.05 (br. d, 1H); 9.25 (s, 1H). |
| 67 | 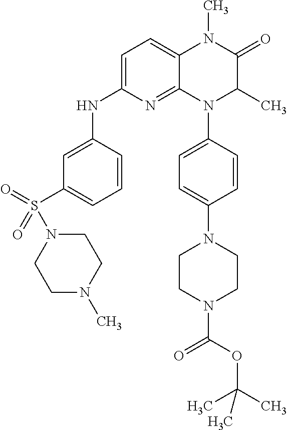 [SMILES: CC1C(=O)N(C)c2ccc(Nc3cccc(S(=O)(=O)N4CCN(C)CC4)c3)nc2N1c1ccc(N2CCN(C(=O)OC(C)(C)C)CC2)cc1] | tert-butyl 4-{4-[1,3- dimethyl-6-({3-[(4- methylpiperazin-1- yl)sulphonyl]phenl}- amino)-2-oxo-2,3- dihydropyrido[2,3- b]pyrazin-4(1H)- yl]phenyl}piperazine-1- carboxylate | Intermediate 84, Intermediate 110, General Synthesis Method B | (400 MHz, DMSO-d6): δ = 1.28 (d, 3H); 1.44 (s, 10H); 2.13 (d, 6H); 3.13-3.19 (m, 4H); 3.29 (s, 3H); 3.48-3.54 (m, 4H); 4.45 (q, 1H); 6.30 (d, 1H); 6.78 (ddd, 1H); 6.82 (ddd, 1H); 6.89 (t, 1H); 6.99- 7.03 (m, 2H); 7.03-7.08 (m, 2H); 7.19-7.27 (m, 3H); 7.29 (br. s, 1H); 7.37 (d, 1H); 7.93-7.98 (m, 1H); 9.22 (s, 1H). |
| 68 | 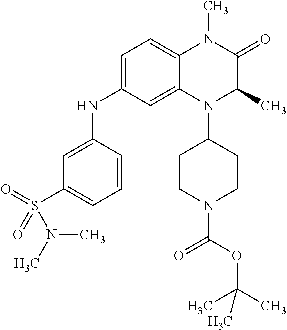 [SMILES: C[C@@H]1C(=O)N(C)c2ccc(Nc3cccc(S(=O)(=O)N(C)C)c3)cc2N1C1CCN(C(=O)OC(C)(C)C)CC1] | tert-butyl 4-[(2R)-7-{[3- (dimethylsulphamoyl)- phenyl]amino}-2,4- dimethyl-3-oxo-3,4- dihydroquinoxalin-1(2H)- yl]piperidine-1- carboxylate | Intermediate 52, Amine 2, General Synthesis Method B | (300 MHz, CDCl3): δ = 1.20 (d, 3H); 1.49 (s, 9H); 1.53- 1.83 (m, 5H); 1.94-2.03 (m, 1H); 2.76 (s, 9H); 3.45-3.58 (m, 1H); 4.14 (q, 1H); 4.18- 4.34 (m, 3H); 6.81 (d, 2H); 6.95 (d, 1H); 7.19 (s, 1H); 7.26 (d, 1H); 7.41 (d, 1H); 7.44 (s, 1H). |
| 69 | 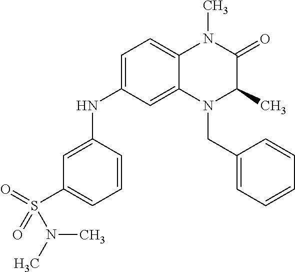 [SMILES: C[C@@H]1C(=O)N(C)c2ccc(Nc3cccc(S(=O)(=O)N(C)C)c3)cc2N1Cc1ccccc1] | 3-{[(3R)-4-benzyl-1,3- dimethyl-2-oxo-1,2,3,4- tetrahydroquinoxalin-6- yl]amino}-N,N-dimethyl- benzenesulphonamide | Intermediate 86, Amine 2, General Synthesis Method B | (400 MHz, CDCl3): δ = 1.19 (d, 3H); 2.72-2.76 (m, 6H); 3.43 (s, 3H); 4.06 (q, 1H); 4.18 (d, 1H); 4.50 (d, 1H); 5.80 (bs, 1H); 6.45 (d, 1H); 6.64 (dd, 1H); 6.92 (d, 2H); 7.17-7.27 (m, 2H); 7.30-7.40 (m, 6H). |
| 70 | 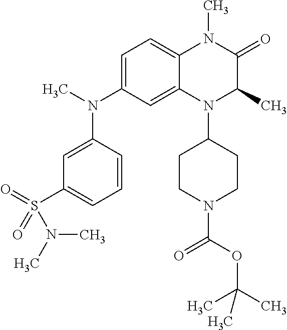 [SMILES: C[C@@H]1C(=O)N(C)c2ccc(N(C)c3cccc(S(=O)(=O)N(C)C)c3)cc2N1C1CCN(C(=O)OC(C)(C)C)CC1] | tert-butyl 4-[(2R)-7-{[3- (dimethylsulphamoyl)- phenyl](methyl)amino}- 2,4-dimethyl-3-oxo-3,4- dihydroquinoxalin-1(2H)- yl]piperidine-1- carboxylate | Intermediate 88, Amine 3, General Synthesis Method B | (300 MHz, CDCl3): δ = 1.14 (d, 3H); 1.48 (s, 9H); 1.58- 1.75 (m, 4H); 1.87-1.98 (m, 1H); 2.71-2.77 (m, 7H); 3.37 (s, 3H); 3.40 (s, 3H); 3.41-3.49 (m, 1H); 4.07- 4.15 (m, 1H); 4.15-4.32 (m, 2H); 6.70 (s, 1H); 6.71-6.76 (m, 1H); 6.97 (d, 1H); 7.02 (dd, 1H); 7.18 (d, 1H); 7.22 (d, 1H); 7.32-7.40 (m, 1H). |
| 71 | 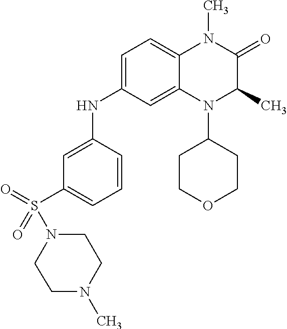 [SMILES: C[C@@H]1C(=O)N(C)c2ccc(Nc3cccc(S(=O)(=O)N4CCN(C)CC4)c3)cc2N1C1CCOCC1] | (3R)-1,3-dimethyl-6-({3- [(4-methylpiperazin-1- yl)sulphonyl]phenl}- amino)-4-(tetrahydro-2H- pyran-4-yl)-3,4- dihydroquinoxalin-2(1H)- one | Intermediate 14, Intermediate 110, General Synthesis Method B | (400 MHz, DMSO-d6): δ = 0.97 (d, 3H); 1.54-1.61 (m, 1H); 1.63-1.80 (m, 2H); 1.81-1.88 (m, 1H); 2.13 (s, 3H); 2.35 (t, 4H); 2.88 (bs, 4H); 3.24 (s, 3H); 3.36-3.43 (m, 2H); 3.60 (tt, 1H); 3.85- 3.96 (m, 2H); 4.06 (q, 1H); 6.67 (dd, 1H); 6.72 (d, 1H); 7.00-7.04 (m, 2H); 7.24 (dd, 1H); 7.29 (t, 1H); 7.43 (t, 1H); 8.46 (s, 1H). |

TABLE 3
The following examples were prepared in accordance with General Synthesis Method A
described in Example 1 from the respective intermediates and amines (Table 1):

| | | | Intermediate/ | |
|---|---|---|---|---|
| Ex. | Structure | Name | Amine | Analytical data ¹H NMR |
| 72 | 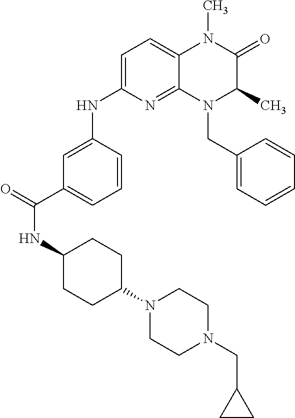 | 3-{[(3R)-4-benzyl-1,3- dimethyl-2-oxo-1,2,3,4- tetrahydropyrido[2,3- b]pyrazin-6-yl]amino}-N- {trans-4-[4-(cyclo- propylmethyl)piperazin-1- yl]cyclohexyl}benzamide | Intermediate 43, Amine 13 | (400 MHz, DMSO-d6): δ = 0.01-0.06 (m, 2H); 0.40- 0.46 (m, 2H); 0.72-0.84 (m, 1H); 1.10 (d, 3H); 1.15- 1.29 (m, 4H); 1.69-1.82 (m, 4H); 2.03-2.09 (m, 1H); 2.12 (d, 2H); 2.40 (bs, 4H); 2.47 (bs, 4H); 3.22 (s, 3H); 3.55-3.67 (m, 1H); 3.93 (q, 1H); 4.23 (d, 1H); 5.26 (d, 1H); 6.26 (d, 1H); 7.14- 7.21 (m, 2H); 7.23-7.37 (m, 6H); 7.56 (dt, 1H); 8.03 (d, 1H); 8.08 (s, 1H); 8.91 (s, 1H). |
| 73 | 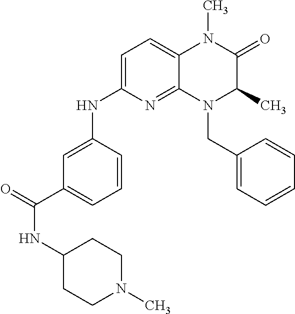 [SMILES: C[C@@H]1C(=O)N(C)c2ccc(Nc3cccc(C(=O)NC4CCN(C)CC4)c3)nc2N1Cc1ccccc1] | 3-{[(3R)-4-benzyl-1,3- dimethyl-2-oxo-1,2,3,4- tetrahydropyrido[2,3- b]pyrazin-6-yl]amino}-N- (1-methylpiperidin-4- yl)benzamide | Intermediate 43, Amine 14 | (400 MHz, DMSO-d6): δ = 1.11 (d, 3H); 1.42-1,57 (m, 2H); 1.58-1.70 (m, 2H); 1.83-1.94 (m, 2H); 2.13 (s, 3H); 2.65-2.74 (m, 2H); 3.22 (s, 3H); 3.60-3.69 (m, 1H); 3.97 (q, 1H); 4.24 (d, 1H); 5.24 (d, 1H); 6.26 (d, 1H); 7.12-7.36 (m, 8H); 7.61 (dt, 1H); 8.03 (br. s, 1H); 8.08 (d, 1H); 8.90 (s, 1H). |
| 74 | 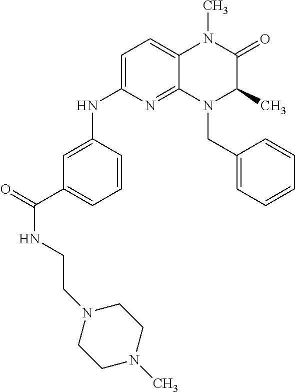 [SMILES: C[C@@H]1C(=O)N(C)c2ccc(Nc3cccc(C(=O)NCCN4CCN(C)CC4)c3)nc2N1Cc1ccccc1] | 3-{[(3R)-4-benzyl-1,3- dimethyl-2-oxo-1,2,3,4- tetrahydropyrido[2,3- b]pyrazin-6-yl]amino}-N- [2-(4-methylpiperazin-1- yl)ethyl]benzamide | Intermediate 43, Amine 15 | (400 MHz, DMSO-d6): δ = 1.11 (d, 3H); 2.12 (s, 3H); 2.19-2.32 (m, 4H); 2.37 (t, 5H); 3.22 (s, 3H); 3.25-3.31 (m, 3H); 3.98 (q, 1H); 4.25 (d, 1H); 5.26 (d, 1H); 6.26 (d, 1H); 7.14-7.21 (m, 2H); 7.21-7.37 (m, 6H); 7.62 (dt, 1H); 8.02-8.05 (m, 1H); 8.18 (t, 1H); 8.91 (s, 1H). |
| 75 | 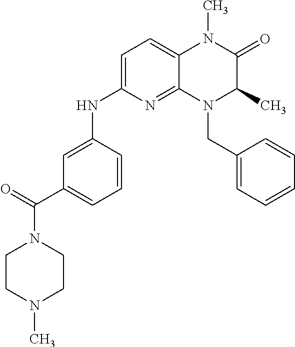 [SMILES: C[C@@H]1C(=O)N(C)c2ccc(Nc3cccc(C(=O)N4CCN(C)CC4)c3)nc2N1Cc1ccccc1] | (3R)-4-benzyl-1,3- dimethyl-6-({3-[(4- methylpiperazin-1- yl)carbonyl]phenyl {amino)-3,4- dihydropyrido[2,3- b]pyrazin-2(1H)-one | Intermediate 43, Amine 16 | (400 MHz, DMSO-d6): δ = 1.08-1.13 (m, 3H); 2.09- 2.12 (m, 3H); 2.14-2.28 (m, 4H); 3.22 (s, 3H); 3.24-3.31 (m, 2H); 3.40-3.55 (m, 2H); 3.97 (q, 1H); 4.25 (d, 1H); 5.20 (d, 1H); 6.25 (d, 1H); 6.72 (br. d, 1H); 7.16 (t, 1H); 7.22-7.29 (m, 2H); 7.31- 7.35 (m, 4H); 7.46 (dd, 1H); 7.66 (t, 1H); 8.92 (s, 1H). |
| 76 | 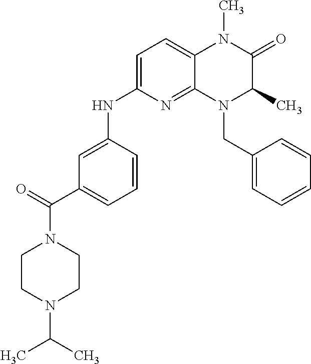 [SMILES: CC(C)N1CCN(C(=O)c2cccc(Nc3ccc4c(n3)N(Cc3ccccc3)[C@H](C)C(=O)N4C)c2)CC1] | (3R)-4-benzyl-6-({3-[(4- isopropylpiperazin-1- yl)carbonyl]phenyl}- amino)-1,3-dimethyl-3,4- dihydropyrido[2,3- b]pyrazin-2(1H)-one | Intermediate 43, Amine 17 | (400 MHz, DMSO-d6): δ = 0.92 (d, 6H); 1.11 (d, 3H); 2.25-2.43 (m, 4H); 2.50- 2.54 (m, 1H); 2.55-2.63 (m, 1H); 3.22 (s, 3H); 3.24-3.29 (m, 1H); 3.36-3.59 (m, 2H); 3.99 (q, 1H); 4.26 (d, 1H); 5.20 (d, 1H); 6.25 (d, 1H); 6.72 (br. d, 1H); 7.15 (t, 1H): 7.20-7.36 (m, 6H); 7.49 (dd, 1H); 7.60 (t, 1H); 8.91 (s, 1H). |
| 77 | 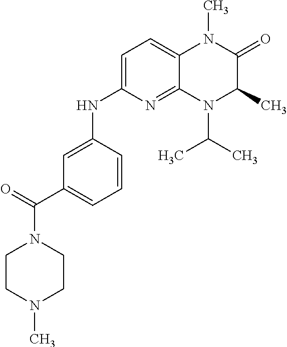 [SMILES: CC(C)N1c2nc(Nc3cccc(C(=O)N4CCN(C)CC4)c3)ccc2N(C)C(=O)[C@H]1C] | (3R)-4-isopropl-1,3- dimethyl-6-({3-[(4- methylpiperazin-1- yl)carbonyl]phenyl}- amino)-3,4-dihydro- pyrido[2,3-b]pyrazin- 2(1H)-one | Intermediate 35, Amine 17 | (400 MHz, DMSO-d6): δ = 1.08 (d, 3H); 1.23 (d, 3H); 1.29 (d, 3H); 2.17 (s, 3H); 2.19-2.39 (m, 4H); 3.19 (s, 3H); 3.34 (d, 2H); 3.49- 3.66 (m, 2H); 4.23 (q, 1H); 4.57 (spt, 1H); 6.22 (d, 1H); 6.75 (d, 1H); 7.25 (t, 2H); 7.46 (dd, 1H); 7.92 (t, 1H); 8.92 (s, 1H). |
| 78 | 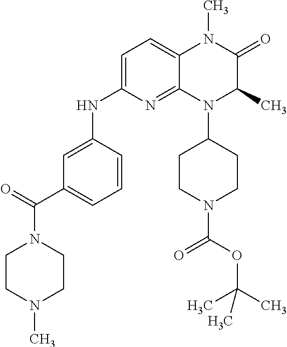 [SMILES: C[C@@H]1C(=O)N(C)c2ccc(Nc3cccc(C(=O)N4CCN(C)CC4)c3)nc2N1C1CCN(C(=O)OC(C)(C)C)CC1] | tert-butyl 4-[(3R)-1,3- dimethyl-6-({3-[(4- methylpiperazin-1- yl)carbonyl]phenyl}- amino)-2-oxo-2,3- dihydropyrido[2,3- b]pyrazin-4(1H)- yl]piperidine-1- carboxylate | Intermediate 54, Amine 16 | (300 MHz, DMSO-d6): δ = 1.05 (d, 3H); 1.41 (s, 9H); 1.45-1.64 (m, 2H); 1.64- 1.81 (m, 1H); 1.90-2.00 (m, 1H); 2.18 (s, 3H); 2.29 (bs, 4H); 2.66-2.90 (m, 2H); 3.19 (s, 3H); 3.35 (bs, 2H); 3.56 (bs, 2H); 4.06 (bs, 2H); 4.20 (q, 1H); 4.36 (br. t, 1H); 6.25 (d, 1H); 6.76 (d, 1H); 7.22-7.29 (m, 2H); 7.41 (br. d, 1H); 7.97 (br. s, 1H); 8.99 (s, 1H). |
| 79 | 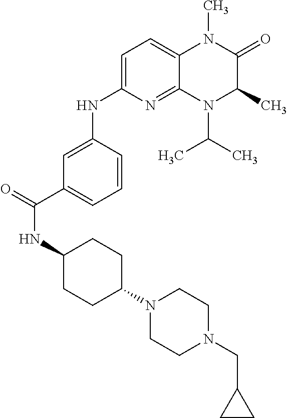 [SMILES: CC(C)N1c2nc(Nc3cccc(C(=O)N[C@H]4CC[C@H](N5CCN(CC6CC6)CC5)CC4)c3)ccc2N(C)C(=O)[C@H]1C] | N-{trans-4-[4-(cyclo- propylmethyl)piperazin-1- yl]cyclohexyl}-3-{[(3R)-4- isopropyl-1,3-dimethyl-2- oxo-1,2,3,4- tetrahydropyrido[2,3- b]pyrazin-6- yl]amino}benzamide | Intermediate 35, Amine 13 | (400 MHz, DMSO-d6): δ = 0.00-0.07 (m, 2H); 0.40- 0.45 (m, 2H); 0.72-0.84 (m, 1H); 1.08 (d, 3H); 1.22 (d, 3H); 1.26-1.39 (m, 6H); 2.12 (d, 2H); 2.14-2.22 (m, 1H); 2.33-2.45 (m, 3H); 3.19 (s, 3H); 3.62-3.73 (m, 1H); 4.22 (q, 1H); 4.61 (spt, 1H); 6.21 (d, 1H); 7.20- 7.27 (m, 3H); 7.72 (dt, 1H); 8.01 (br. s, 1H); 8.04 (d, 1H); 8.86 (s, 1H). |
| 80 | 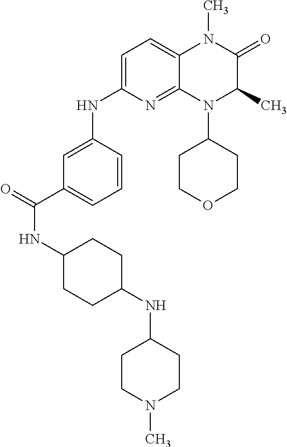 [SMILES: C[C@@H]1C(=O)N(C)c2ccc(Nc3cccc(C(=O)NC4CCC(NC5CCN(C)CC5)CC4)c3)nc2N1C1CCOCC1] | 3-{[(3R)-1,3-dimethyl-2- oxo-4-(tetrahydro-2H- pyran-4-yl)-1,2,3,4- tetrahydropyrido[2,3- b]pyrazin-6-yl]amino}-N- {4-[(1-methylpiperidin-4- yl)amino]cyclohexyl} benzamide | Intermediate 18, Intermediate 118 | (300 MHz, DMSO-d6): δ = 0.95-1.04 (m, 1H); 1.07 (d, 3H); 1.11-1.41 (m, 4H); 1.42-1.64 (m, 3H); 1.71 (d, 3H); 1.84 (t, 8H); 2.10 (s, 3H); 2.67 (d, 2H); 3.19 (s, 3H); 3.38-3.53 (m, 4H); 3.60-3.77 (m, 1H); 3.90 (br. d, 2H); 4.21 (q, 1H); 4.43 (br. t, 1H); 6.23 (d, 1H); 7.25 (d, 3H); 7.71-7.78 (m, 1H); 7.96 (br. s, 1H); 8.08 (br. d, 1H); 8.93 (s, 1H). |
| 81 | 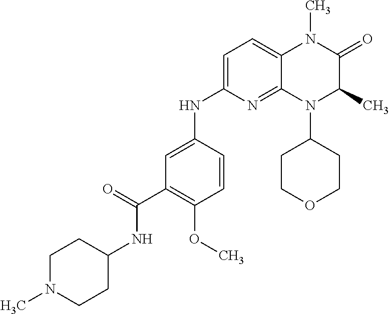 | 5-{[(3R)-1,3-dimethyl-2- oxo-4-(tetrahydro-2H- pyran-4-yl)-1,2,3,4- tetrahydropyrido[2,3- b]pyrazin-6-yl]amino}-2- methoxy-N-(1- methylpiperidin-4- yl)benzamide | Intermediate 79, Amine 14 | (400 MHz, DMSO-d6): δ = 1.06 (d, 3H); 1.55 (d, 2H); 1.62-1.75 (m, 1H); 1.83 (d, 3H); 1.87-1.94 (m, 1H); 2.08-2.17 (m, 2H); 2.20 (s, 3H); 2.63-2.74 (m, 2H); 3.18 (s, 3H); 3.37-3.53 (m, 4H); 3.84 (s, 3H); 3.88 (bs, 2H); 4.19 (q, 1H); 4.44 (tt, 1H); 6.16 (d, 1H); 7.02 (d, 1H); 7.21 (d, 1H); 7.63 (dd, 1H); 7.99-8.04 (m, 2H); 8.71 (s, 1H). |
| 82 | 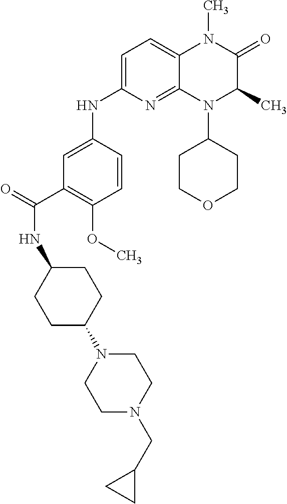 [SMILES: COc1ccc(Nc2ccc3c(n2)N(C2CCOCC2)[C@H](C)C(=O)N3C)cc1C(=O)N[C@H]1CC[C@H](N2CCN(CC3CC3)CC2)CC1] | N-{trans-4-[4-(cyclo- propylmethyl)piperazin-1- yl]cyclohexyl}-5-{[(3R)- 1,3-dimethyl-2-oxo-4- (tetrahydro-2H-pyran-4- yl)-1,2,3,4- tetrahydropyrido[2,3- b]pyrazin-6-yl]amino}-2- methoxybenzamide | Intermediate 79, Amine 13 | (400 MHz, DMSO-d6): δ = 0.01-0.08 (m, 2H); 0.40- 0.48 (m, 2H); 0.80 (bs, 1H); 1.06 (d, 3H); 1.23-1.35 (m, 4H); 1.52-1.59 (m, 1H); 1.62-1.75 (m, 1H); 1.79- 1.90 (m, 3H); 1.93 (d, 2H); 2.16 (d, 2H); 2.19-2.28 (m, 1H); 3.18 (s, 3H); 3.35-3.53 (m, 4H); 3.54-3.74 (m, 8H); 3.83 (s, 3H); 3.89 (dd, 2H); 4.19 (q, 1H); 4.43 (tt, 1H); 6.15 (d, 1H); 7.00 (d, 1H); 7.21 (d, 1H); 7.64 (dd, 1H); 7.92 (d, 1H); 7.98 (d, 1H); 8.70 (s, 1H). |
| 83 | 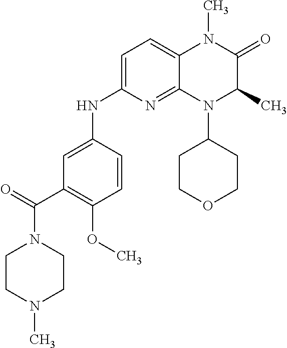 [SMILES: COc1ccc(Nc2ccc3c(n2)N(C2CCOCC2)[C@H](C)C(=O)N3C)cc1C(=O)N1CCN(C)CC1] | (3R)-6-({4-methoxy-3-[(4- methylpiperazin-1- yl)carbonyl]phenyl}- amino)-1,3-dimethyl-4- (tetrahydro-2H-pyran-4- yl)-3,4-dihydropyrido[2,3- b]pyrazin-2(1H)-one | Intermediate 79, Amine 16 | (400 MHz, DMSO-d6): δ = 1.06 (d, 3H); 1.51-1.59 (m, 1H); 1.67-1.80 (m, 1H); 1.81-1.92 (m, 2H); 2.15- 2.19 (m, 4H); 2.23-2.36 (m, 3H); 3.11-3.15 (m, 1H); 3.18 (s, 3H); 3.41-3.48 (m, 3H); 3.54-3.63 (m, 2H); 3.73 (s, 3H); 3.88-3.98 (m, 2H); 4.20 (q, 1H); 4.41 (tt, 1H); 6.17 (d, 1H); 6.96 (d, 1H); 7.22 (d, 1H); 7.38- 7.48 (m, 1H); 7.64-7.70 (m, 1H); 8.69 (s, 1H). |
| 84 | 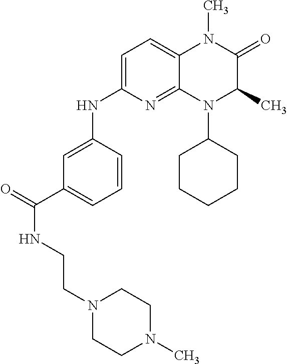 [SMILES: C[C@@H]1C(=O)N(C)c2ccc(Nc3cccc(C(=O)NCCN4CCN(C)CC4)c3)nc2N1C1CCCCC1] | 3-{[(3R)-4-cyclohexyl-1,3- dimethyl-2-oxo-1,2,3,4- tetrahydropyrido[2,3- b]pyrazin-6-yl]amino}-N- [2-(4-methlpiperazin-1- yl)ethyl]benzamide | Intermediate 30, Amine 15 | (300 MHz, DMSO-d6): δ = 1.06 (d, 3H); 1.09-1.51 (m, 5H); 1.54-1.68 (m, 3H); 1.75 (d, 2H); 1.99 (bs, 1H); 2.12 (s, 3H); 2.29 (bs, 4H); 2.38-2.46 (m, 5H); 3.19 (s, 3H); 3.34 (bs, 2H); 4.15- 4.29 (m, 2H); 6.20 (d, 1H); 7.18-7.29 (m, 3H); 7.82 (br. d, 1H); 7.94 (s, 1H); 8.23 (t, 1H); 8.91 (s, 1H). |
| 85 | 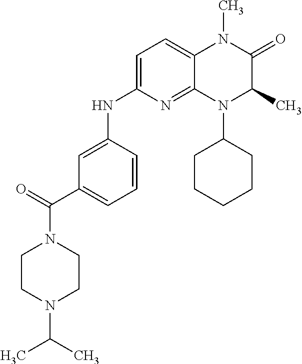 [SMILES: CC(C)N1CCN(C(=O)c2cccc(Nc3ccc4c(n3)N(C3CCCCC3)[C@H](C)C(=O)N4C)c2)CC1] | (3R)-cyclohexyl-6-({3- [(4-isopropylpiperazin-1- yl)carbonyl]phenyl}- amino)-1,3-dimethyl-3,4- dihydropyrido[2,3- b]pyrazin-2(1H)-one | Intermediate 30, Amine 17 | (400 MHz, DMSO-d6): δ = 0.95 (d, 6H); 1.06 (d, 3H); 1.10-1.24 (m, 1H); 1.27- 1.51 (m, 3H); 1.60 (d, 3H); 1.77 (bs, 2H); 1.96-2.04 (m, 1H); 2.33-2.47 (m, 4H); 2.66 (spt, 1H); 3.19 (s, 3H); 3.31-3.41 (m, 2H); 3.48- 3.64 (m, 2H); 4.17-4.26 (m, 2H); 6.21 (d, 1H); 6.76 (br. d, 1H); 7.21-7.26 (m, 2H); 7.57 (dd, 1H); 7.80 (t, 1H); 8.92 (s, 1H). |
| 86 | 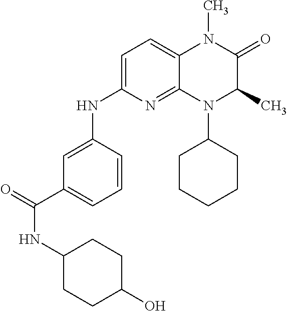 [SMILES: C[C@@H]1C(=O)N(C)c2ccc(Nc3cccc(C(=O)NC4CCC(O)CC4)c3)nc2N1C1CCCCC1] | 3-{[(3R)-4-cyclohexyl-1,3- dimethyl-2-oxo-1,2,3,4- tetrahydropyrido[2,3- b]pyrazin-6-yl]amino}-N- (4-hydroxycyclohexyl)- benzamide | Intermediate 30, Amine 14 | (400 MHz, DMSO-d6): δ = 1.12-1.28 (m, 3H); 1.28- 1.46 (m, 5H); 1.54-1.70 (m, 3H); 1.72-1.87 (m, 6H); 1.97-2.05 (m, 1H); 3.18 (s, 3H); 3.32-3.41 (m, 1H); 3.61-3.74 (m, 1H); 4.16- 4.24 (m, 2H); 4.52 (d, 1H); 6.20 (d, 1H); 7.19-7.26 (m, 3H); 7.84-7.90 (m, 2H); 8.02 (d, 1H); 8.88 (s, 1H). |
| 87 | 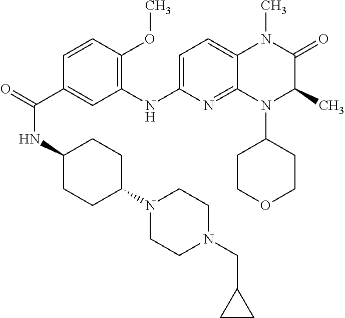 [SMILES: COc1ccc(C(=O)N[C@H]2CC[C@H](N3CCN(CC4CC4)CC3)CC2)cc1Nc1ccc2c(n1)N(C1CCOCC1)[C@H](C)C(=O)N2C] | N-{trans-4-[4-(cyclo- propylmethyl)piperazin-1- yl]cyclohexyl}-3-{[(3R)- 1,3-dimethyl-2-oxo-4- (tetrahydro-2H-pyran-4- yl)-1,2,3,4- tetrahydropyrido[2,3- b]pyrazin-6-yl]amino}-4- methoxybenzamide | Intermediate 20, Amine 13 | (400 MHz, DMSO-d6): δ = 0.22-0.27 (m, 2H); 0.34- 0.39 (m, 2H); 1.06 (d, 3H); 1.21-1.39 (m, 5H); 1.47- 1.64 (m, 4H); 1.73-1.89 (m, 7H); 2.12-2.22 (m, 1H); 2.43 (bs, 4H); 3.19 (s, 3H); 3.21-3.27 (m, 2H); 3.39- 3.47 (m, 1H); 3.62-3.71 (m, 1H); 3.75 (d, 1H); 3.79- 3.85 (m, 1H); 3.88 (s, 3H); 4.18 (q, 1H); 4.42 (t, 1H); 6.43 (d, 1H); 6.97 (d, 1H); 7.23 (d, 1H); 7.36 (dd, 1H); 7.86 (s, 1H); 7.94 (d, 1H); 8.51 (d, 1H). |
| 88 | 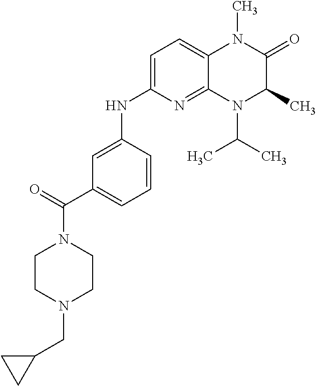 [SMILES: CC(C)N1c2nc(Nc3cccc(C(=O)N4CCN(CC5CC5)CC4)c3)ccc2N(C)C(=O)[C@H]1C] | (3R)-6-[(3-{[4-(cyclo- propylmethyl)piperazin-1- yl]carbonyl}phenyl) amino]-4-isopropyl-1,3- dimethyl-3,4- dihydropyrido[2,3- b]pyrazin-2(1H)-one | Intermediate 35, Amine 22 | (400 MHz, DMSO-d6): δ = 0.05 (d, 2H); 0.41-0.47 (m, 2H); 0.75-0.87 (m, 1H); 1.08 (d, 3H); 1.23 (d, 3H); 1.29 (d, 3H); 1.43-1.54 (m, 1H); 2.19 (d, 2H); 2.28- 2.45 (m, 4H); 3.19 (s, 3H); 3.49-3.69 (m, 3H); 4.23 (q, 1H); 4.57 (spt, 1H); 6.22 (d, 1H); 6.75 (br. d, 1H); 7.22- 7.28 (m, 2H); 7.47 (dd, 1H); 7.91 (br. s, 1H); 8.92 (s, 1H). |
| 89 | 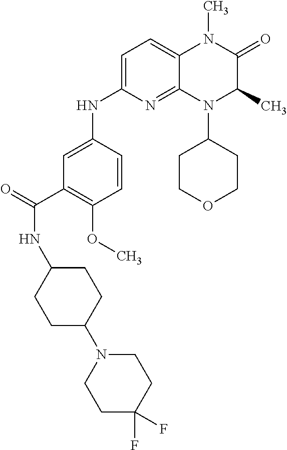 [SMILES: COc1ccc(Nc2ccc3c(n2)N(C2CCOCC2)[C@H](C)C(=O)N3C)cc1C(=O)NC1CCC(N2CCC(F)(F)CC2)CC1] | N-[4-(4,4- difluoropiperidin-1- yl)cyclohexyl]-5-{[(3R)- 1,3-dimethyl-2-oxo-4- (tetrahydro-2H-pyran-4- yl-1,2,3,4- tetrahydropyrido[2,3- b]pyrazin-6-yl]amino}-2- methoxybenzamide | Intermediate 79, Intermediate 120 | (400 MHz, DMSO-d6): δ = 1.22-1.44 (m, 2H); 1.48- 1.63 (m, 5H); 1.65-1.84 (m, 4H); 1.86-1.99 (m, 6H); 2.38-2.45 (m, 1H); 2.56- 2.64 (m, 4H); 3.18 (s, 3H); 3.36-3.57 (m, 2H); 3.65- 3.74 (m, 1H*); 3.83 (s, 1H); 3.85-3.92 (m, 4H); 4.01- 4.10 (m, 1H); 4.19 (q, 1H); 4.38-4.50 (m, 1H); 6.15 (d, 1H*); 6.16 (d, 1H); 7.00 (d, 1H*); 7.05 (d, 1H); 7.21 (d, 1H*); 7.22 (d, 1H); 7.62 (dd, 1H); 7.63 (dd, 1H*); 7.92 (d, 1H*); 7.98 (d, 1H*); 8.08 (d, 1H); 8.17 (d, 1H); 8.70 (s, 1H*); 8.72 (s, 1H); Diastereomere (2:1), *) Nebendiastereomer. |
| 90 | 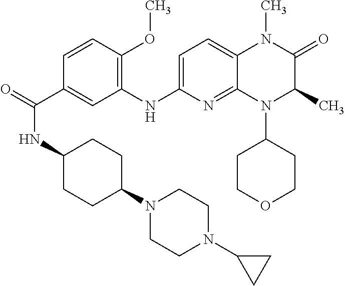 [SMILES: COc1ccc(C(=O)N[C@H]2CC[C@@H](N3CCN(C4CC4)CC3)CC2)cc1Nc1ccc2c(n1)N(C1CCOCC1)[C@H](C)C(=O)N2C] | N-[cis-4-(4- cyclopropylpiperazin-1- yl)cyclohexyl]-3-{[(3R)- 1,3-dimethyl-2-oxo-4- (tetrahydro-2H-pyran-4- yl)-1,2,3,4- tetrahydropyrido[2,3- b]pyrazin-6-yl]amino}-4- methoxybenzamide | Intermediate 20, Intermediate 122 | (400 MHz, DMSO-d6): δ = 0.23-0.28 (m, 2H); 0.34- 0.41 (m, 2H); 1.04-1.08 (m, 3H); 1.10 (s, 2H); 1.12-1.66 (m, 9H); 1.68-1.89 (m, 7H); 2.06-2.12 (m, 1H); 2.35- 2.43 (m, 3H); 3.19 (s, 3H); 3.22-3.28 (m, 2H); 3.39- 3.50 (m, 1H); 3.72-3.78 (m, 1H); 3.79-3.85 (m, 1H); 3.88 (s, 3H); 4.18 (q, 1H); 4.43 (tt, 1H); 6.44 (d, 1H); 6.97 (d, 1H); 7.23 (d, 1H); 7.40 (dd, 1H); 7.84-7.87 (m, 1H); 7.90 (d, 1H); 8.53 (d, 1H). |
| 91 | 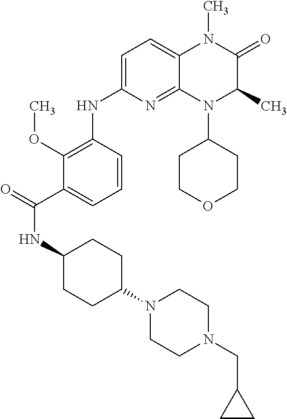 | N-{trans-4-[4-(cyclo- propylmethyl)piperazin-1- yl]cyclohexyl}-3-{[(3R)- 1,3-dimethyl-2-oxo-4- (tetrahydro-2H-pyran-4- yl)-1,2,3,4- tetrahydropyrido[2,3- b]pyrazin-6-yl]amino}-2- methoxybenzamide | Intermediate 81, Amine 13 | (400 MHz, DMSO-d6): δ = 0.05 (q, 2H); 0.41-0.46 (m, 2H); 0.75-0.84 (m, 1H); 1.08 (d, 3H); 1.23-1.36 (m, 4H); 1.59 (br. d, 1H); 1.69- 1.98 (m, 7H); 2.15 (d, 2H); 2.19 (d, 1H); 2.44 (bs, 3H); 2.53 (bs, lH); 3.19 (s, 3H); 3.32-3.46 (m, 5H); 3.52 (bs, 2H); 3.71 (s, 3H); 3.91- 4.01 (m, 2H); 4.22 (q, 1H); 4.33 (tt, 1H); 6.55 (d, 1H); 6.93 (dd, 1H); 7.04 (t, 1H); 7.25 (d, 1H); 8.02 (d, 1H); 8.08 (s, 1H); 8.35 (dd, 1H). |
| 92 | 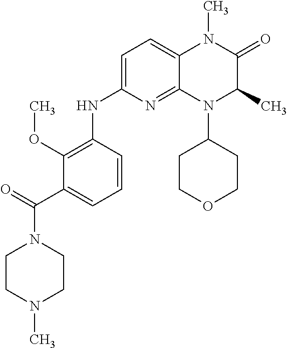 [SMILES: COc1c(Nc2ccc3c(n2)N(C2CCOCC2)[C@H](C)C(=O)N3C)cccc1C(=O)N1CCN(C)CC1] | (3R)-6-({2-methoxy-3-[(4- methylpiperazin-1- yl)carbonyl]phenyl}- amino)-1,3-dimethyl-4- (tetrahydro-2H-pyran-4- yl)-3,4-dihydropyrido[2,3- b]pyrazin-2(1H)-one | Intermediate 81, Amine 16 | (400 MHz, DMSO-d6): δ = 1.08 (d, 3H); 1.60 (d, 1H); 1.70-1.83 (m, 1H); 1.88- 2.00 (m, 2H); 2.40 (s, 3H); 2.52-2.59 (m, 2H); 2.60- 2.72 (m, 2H); 3.20 (s, 3H); 3.27 (bs, 2H); 3.33-3.49 (m, 4H); 3.67-3.76 (m, 5H); 3.97 (bs, 2H); 4.23 (q, 1H); 4.35 (bs, 1H); 6.57 (d, 1H); 6.71 (dd, 1H); 7.06 (t, 1H); 7.23-7.29 (m, 1H); 8.11 (s, 1H); 8.39 (dd, 1H). |
| 93 | 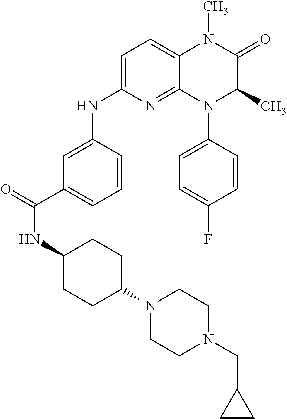 | N-{trans-4-[4-(cyclo- propylmethyl)piperazin-1- yl]cyclohexyl}-{[(3R)-4- (4-fluorophenyl)-1,3- dimethyl-2-oxo-1,2,3,4- tetrahydropyrido[2,3- b]pyrazin-6- yl]amino}benzamide | Intermediate 74, Amine 13 | (400 MHz, DMSO-d6): δ = 0.01-0.06 (m, 2H); 0.40- 0.46 (m, 2H); 0.73-0.86 (m, 2H); 0.87-1.17 (m, 2H); 1.18-1.38 (m, 10H); 1.83 (bs, 4H); 2.12 (d, 2H); 2.13- 2.22 (m, 1H); 2.33-2.47 (m, 6H); 3.60-3.73 (m, 1H); 4.49 (d, 1H); 6.36 (d, 1H); 6.89 (t, 1H); 7.14 (br. d, 1H); 7.22 (t, 2H); 7.34-7.40 (m, 3H); 7.43 (s, 1H); 7.58 (dd, 1H); 8.91 (s, 1H). |
| 94 | 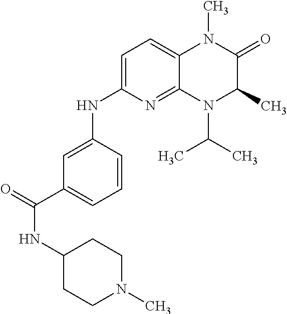 [SMILES: CC(C)N1c2nc(Nc3cccc(C(=O)NC4CCN(C)CC4)c3)ccc2N(C)C(=O)[C@H]1C] | 3-{[(3R)-4-isopropyl-1,3- dimethyl-2-oxo-1,2,3,4- tetrahydropyrido[2,3- b]pyrazin-6-yl]amino}-N- (1-methylpiperidin-4- yl)benzamide | Intermediate 35, Amine 14 | (400 MHz, DMSO-d6): δ = 1.08 (d, 3H); 1.22 (d, 3H); 1.28 (d, 3H); 1.51-1.63 (m, 2H); 1.75 (d, 2H); 2.02 (t, 2H); 2.19 (s, 3H); 2.80 (d, 2H); 3.19 (s, 3H); 3.66-3.76 (m, 1H); 4.22 (q, 1H); 4.62 (spt, 1H); 6.22 (d, 1H); 7.20- 7.28 (m, 3H); 7.72 (dt, 1H); 8.03 (br. s, 1H); 8.10 (d, 1H): 8.87 (s, 1H). |
| 95 | 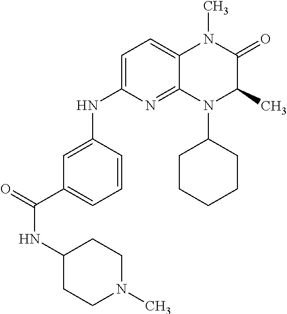 [SMILES: C[C@@H]1C(=O)N(C)c2ccc(Nc3cccc(C(=O)NC4CCN(C)CC4)c3)nc2N1C1CCCCC1] | 3-{[(3R)-4-cyclohexyl-1,3- dimethyl-2-oxo-1,2,3,4- tetrahydropyrido[2,3- b]pyrazin-6-yl]amino}-N- (1-methylpiperidin-4- yl)benzamide | Intermediate 30, Amine 14 | (400 MHz, DMSO-d6): δ = 1.06 (d, 3H); 1.09-1.23 (m, 1H); 1.28-1.48 (m, 3H); 1.50-1.66 (m, 5H); 1.67- 1.81 (m, 4H); 1.86-1.96 (m, 2H); 2.01 (d, 1H); 2.14 (s, 3H); 2.76 (s, 1H); 2.73 (s, 1H); 3.18 (s, 3H); 3.63-3.75 (m, 1H); 4.15-4.26 (m, 2H); 6.20 (d, 1H); 7.18-7.28 (m, 3H); 7.82-7.92 (m, 2H); 8.10 (d, 1H); 8.89 (s, 1H). |
| 96 | 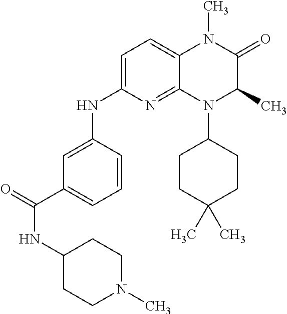 [SMILES: C[C@@H]1C(=O)N(C)c2ccc(Nc3cccc(C(=O)NC4CCN(C)CC4)c3)nc2N1C1CCC(C)(C)CC1] | 3-{[(3R)-4-(4,4- dimethylcyclohexyl)-1,3- dimethyl-2-oxo-1,2,3,4- tetrahydropyrido[2,3- b]pyrazin-6-yl]amino}-N- (1-methylpiperidin-4- yl)benzamide | Intermediate 57, Intermediate 124, General Synthesis Method B | (400 MHz, DMSO-d6): δ = 0.93 (d, 6H); 1.07 (d, 3H); 1.35 (bs, 1H); 1.40 (bs, 4H); 1.45 (bs, 1H); 1.49-1.65 (m, 3H); 1.68-1.85 (m, 4H); 1.90 (t, 2H); 2.14 (s, 3H); 2.76 (s, 1H); 2.73 (s, 1H); 3.19 (s, 3H); 3.65-3.78 (m, 1H); 4.15-4.29 (m, 2H); 6.20 (d, 1H); 7.18-7.28 (m, 3H); 7.78 (dt, 1H); 7.92- 7.98 (m, 1H); 8.08 (d, 1H); 8.88 (s, 1H). |
| 97 | 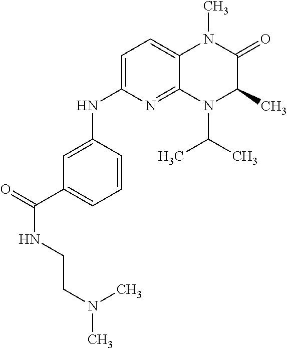 [SMILES: CC(C)N1c2nc(Nc3cccc(C(=O)NCCN(C)C)c3)ccc2N(C)C(=O)[C@H]1C] | N-[2- (dimethylamino)ethyl]-3- {[(3R)-4-isopropyl-1,3- dimethyl-2-oxo-1,2,3,4- tetrahydropyrido[2,3- b]pyrazin-6- yl]amino}benzamide | Intermediate 35, Amine 19 | (400 MHz, DMSO-d6): δ = 1.08 (d, 3H); 1.22 (d, 3H); 1.29 (d, 3H); 2.20 (s, 6H); 2.43 (t, 2H); 3.19 (s, 3H); 3.30-3.37 (m, 2H); 4.22 (d, 1H); 4.65 (spt, 1H); 6.22 (d, 1H); 7.19-7.29 (m, 3H); 7.71 (d, 1H); 8.09 (br. s, 1H); 8.15-8.23 (m, 1H); 8.88 (s, 1H). |
| 98 | 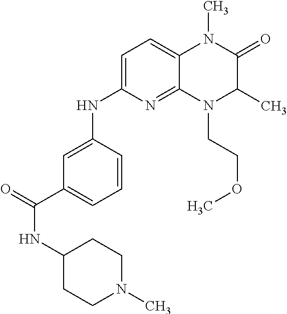 [SMILES: COCCN1c2nc(Nc3cccc(C(=O)NC4CCN(C)CC4)c3)ccc2N(C)C(=O)C1C] | 3-{[4-(2-methoxyethyl)- 1,3-dimethyl-2-oxo- 1,2,3,4- tetrahydropyrido[2,3- b]pyrazin-6-yl]amino}-N- (1-methylpiperidin-4- yl)benzamide | Intermediate 49, Amine 14 | (300 MHz, DMSO-d6): δ = 1.13 (d, 3H); 1.49-1.64 (m, 2H); 1.67-1.80 (m, 2H); 1.86-2.00 (m, 2H); 2.15 (s, 3H); 2.75 (d, 2H); 3.20 (s, 3H); 3.21 (s, 3H); 3.23-3.28 (m, 1H); 3.50-3.60 (m, 2H); 3.62-3.78 (m, 1H); 4.04- 4.20 (m, 2H); 6.21 (d, 1H); 7.18-7.28 (m, 3H); 7.65- 7.72 (m, 1H); 8.04 (s, 1H); 8.09 (d, 1H); 8.88 (s, 1H). |
| 99 | 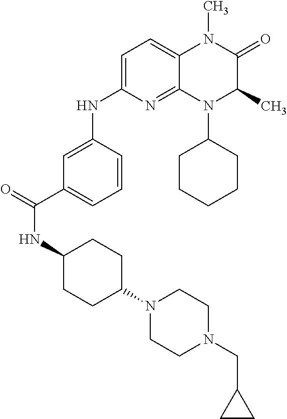 | 3-{[(3R)-4-cyclohexyl-1,3- dimethyl-2-oxo-1,2,3,4- tetrahydropyrido[2,3- b]pyrazin-6-yl]amino}-N- {trans-4-[4-(cyclopropyl- methyl)piperazin-1- yl]cyclohexyl}benzamide | Intermediate 30, Amine 13 | (300 MHz, DMSO-d6): δ = −0.01-0.08 (m, 2H); 0.37- 0.47 (m, 2H); 0.71-0.84 (m, 1H); 1.06 (d, 3H); 1.19- 1.38 (m, 8H); 1.52-1.68 (m, 3H); 1.69-1.92 (m, 6H); 2.00 (t, 2H); 2.11 (d, 2H); 2.39 (bs, 4H); 2.53 (s, 4H); 3.18 (s, 3H); 3.67 (d, 1H); 4.20 (q, 2H); 6.20 (d, 1H); 7.18-7.26 (m, 3H); 7.82- 7.92 (m, 2H); 8.07 (d, 1H); 8.90 (s, 1H). |
| 100 | 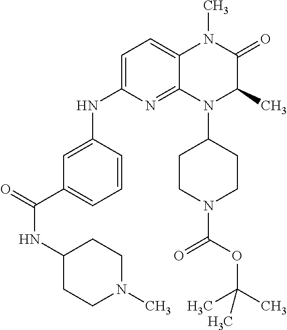 [SMILES: C[C@@H]1C(=O)N(C)c2ccc(Nc3cccc(C(=O)NC4CCN(C)CC4)c3)nc2N1C1CCN(C(=O)OC(C)(C)C)CC1] | tert-butyl 4-[(3R)-1,3- dimethyl-6-({3-[(1- methylpiperidin-4- yl)carbamoyl]phenyl} amino)-2-oxo-2,3- dihydropyrido[2,3- b]pyrazin-4(1H)- yl]piperidine-1- carboxylate | Intermediate 54, Amine 14 | (300 MHz, DMSO-d6): δ = 1.04 (d, 3H); 1.40 (s, 9H); 1.46-1.63 (m, 4H); 1.65- 1.78 (m, 3H); 1.83-2.01 (m, 3H); 2.13 (s, 3H); 2.75 (s, 2H); 2.71 (s, 2H); 2.88 (bs, 1H); 3.19 (s, 3H); 3.59-3.76 (m, 1H); 4.00 (bs, 2H); 4.19 (q, 1H); 4.38 (t, 1H); 6.23 (d, 1H); 7.19-7.29 (m, 3H); 7.57 (d, 1H); 8.09-8.19 (m, 2H); 8.94 (s, 1H). |
| 101 | 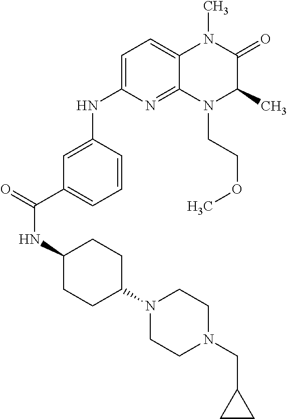 | N-{trans-4-[4- (cyclopropylmethyl) piperazin-1- yl]cyclohexyl}-3- {[(3R)-4-(2-methoxyethyl)- 1,3-dimethyl-2-oxo- 1,2,3,4-tetrahydropyrido- [2,3-b]pyrazin-6- yl]amino}benzamide | Intermediate 49, Amine 13 | (300 MHz, DMSO-d6): δ = 0.04 (q, 2H); 0.38-0.48 (m, 2H); 0.72-0.87 (m, 2H); 1.12 (d, 3H); 1.20-1.43 (m, 5H); 1.74-1.96 (m, 4H); 2.12 (d, 2H); 2.18 (bs, 2H); 2.40 (bs, 4H); 3.17-3.23 (m, 6H); 3.23-3.30 (m, 2H); 3.55 (t, 2H); 3.69 (bs, 1H); 4.03-4.11 (m, 1H); 4.16 (q, 1H); 6.21 (d, 1H); 7.17- 7.29 (m, 3H); 7.69 (dt, 1H); 8.03 (br. s, 1H); 8.07 (d, 1H); 8.90 (s, 1H). |
| 102 | 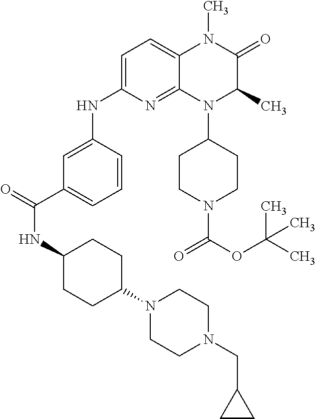 | tert-butyl 4-[(3R)-6-{[3- ({trans-4-[4-(cyclopropl- methyl)piperazin-1- yl]cyclohexyl}carbamoyl) phenyl]amino}-1,3- dimethyl-2-oxo-2,3- dihydropyrido[2,3- b]pyrazin-4(1H)- yl]piperidine-1- carboxylate | Intermediate 54, Amine 13 | (300 MHz, DMSO-d6): δ = −0.01-0.07 (m, 3H); 0.44 (bs, 3H); 0.71-0.86 (m, 2H); 1.04 (d, 3H); 1.13- 1.36 (m, 7H); 1.40 (s, 9H); 1.45-1.70 (m, 4H); 1.76- 1.89 (m, 6H); 1.90-2.01 (m, 2H); 2.11 (d, 3H); 3.19 (s, 3H); 3.58-3.73 (m, 1H); 3.94-4.10 (m, 2H); 4.19 (q, 1H); 4.38 (br. t, 1H); 6.22 (d, 1H); 7.20-7.30 (m, 3H); 7.53-7.60 (m, 1H); 8.10 (d, 1H); 8.14 (br. s, 1H); 8.90- 8.97 (m, 1H). |
| 103 | 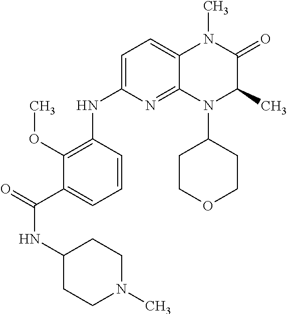 [SMILES: COc1c(Nc2ccc3c(n2)N(C2CCOCC2)[C@H](C)C(=O)N3C)cccc1C(=O)NC1CCN(C)CC1] | 3-{[(3R)-1,3-dimethyl-2- oxo-4-(tetrahydro-2H- pyran-4-yl)-1,2,3,4- tetrahydropyrido[2,3- b]pyrazin-6-yl]amino}-2- methoxy-N-(1- melhylpiperidin-4- yl)benzamide | Intermediate 81, Amine 14 | (400 MHz, DMSO-d6): δ = 1.08 (d, 3H); 1.60 (d, 1H); 1.65-1.80 (m, 3H); 1.87- 2.02 (m, 4H); 2.63 (s, 3H); 2.89 (bs, 2H); 3.20 (s, 3H); 3.24 (d, 2H); 3.34-3.46 (m, 4H); 3.72 (s, 3H); 3.92-4.01 (m, 3H); 4.22 (q, 1H); 4.34 (tt, 1H); 6.56 (d, 1H); 6.91 (dd, lH); 7.06 (t, 1H); 7.26 (d, 1H); 8.08 (s, 1H); 8.27 (d, 1H); 8.38 (dd, 1H). |
| 104 | 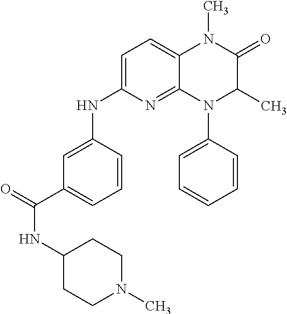 [SMILES: CC1C(=O)N(C)c2ccc(Nc3cccc(C(=O)NC4CCN(C)CC4)c3)nc2N1c1ccccc1] | 3-[(1,3-dimethyl-2-oxo-4- phenyl-1,2,3.4- tetrahydropyrido[2,3- b]pyrazin-6-yl)amino]-A- (1-methylpiperidin-4- yl)benzamide | Intermediate 69, Intermediate 124, General Synthesis Method B | (300 MHz, DMSO-d6): δ = 1.27 (d, 3H); 1.42-1.64 (m, 2H); 1.71 (d, 2H); 1.91 (t, 2H); 2.14 (s, 3H); 2.74 (d, 2H); 3.34 (bs, 3H); 3.61- 3.74 (m, 1H); 4.54 (q, 1H); 6.40 (d, 1H); 6.88 (t, 1H); 7.10-7.22 (m, 2H); 7.30- 7.45 (m, 6H); 7.71 (dd, 1H); 8.07 (d, 1H); 8.96 (s, 1H). |

TABLE 4
The following examples were prepared in accordance with the named general synthesis
methods from the respective intermediates and amines (Table 1):

| | | | Intermediate/ | |
|---|---|---|---|---|
| | | | Amine, | |
| | | | General | |
| | | | Synthesis | |
| Ex. | Structure | Name | Method | Analytical data: ¹H NMR |
| 105 | 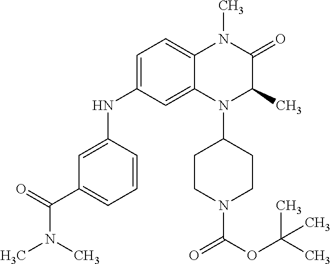 [SMILES: C[C@@H]1C(=O)N(C)c2ccc(Nc3cccc(C(=O)N(C)C)c3)cc2N1C1CCN(C(=O)OC(C)(C)C)CC1] | tert-butyl 4-[(2R)-7-{[3- (dimethylcarbamoyl)phenyl] amino}-2,4-dimethyl-3- oxo-3,4- dihydroquinoxalin-1(2H)- yl]piperidine-1- carboxylate | Intermediate 88, Amine 21, General Synthesis Method B | (400 MHz, DMSO-d6): δ = 0.97 (d, 3H); 1.41 (s, 9H); 1.61 (bs, 3H); 1.85-1.94 (m, 1H); 2.71-2.88 (m, 2H); 2.94 (bs, 6H); 3.24 (s, 3H); 3.47-3.58 (m, 1H); 3.95- 4.11 (m, 3H); 6.65 (dd, 1H); 6.68 (d, 1H); 6.75 (br. d, 1H); 6.97-7.01 (m, 2H); 7.03 (dd, 1H); 7.25 (t, 1H); 8.17 (s, 1H). |
| 106 | 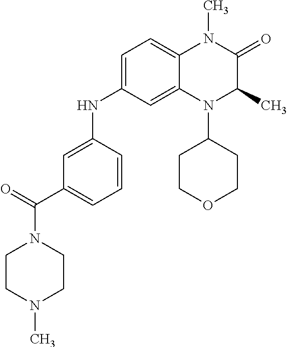 [SMILES: C[C@@H]1C(=O)N(C)c2ccc(Nc3cccc(C(=O)N4CCN(C)CC4)c3)cc2N1C1CCOCC1] | (3R)-1,3-dimethyl-6-({3- [(4-methylpiperazin-1- yl)carbonyl]phenyl}amino)- 4-(tetrahydro-2H-pyran- 4-yl)-3,4- dihydroquinoxalin-2(1H)- one | Intermediate 90, Amine 16, General Synthesis Method A | (300 MHz, DMSO-d6): δ = 0.97 (d, 3H); 1.54-1.88 (m, 5H); 2.18 (s, 3H); 2.29 (bs, 4H); 3.23 (s, 3H); 3.31-3.44 (m, 5H); 3.83-3.96 (m, 3H); 4.05 (q, 1H); 6.61-6.66 (m, 1H); 6.67 (s, 1H); 6.71 (d, 1H); 6.94-7.00 (m, 2H); 7.01-7.07 (m, 1H); 7.19- 7.28 (m, 1H); 8.18 (s, 1H). |
| 107 | 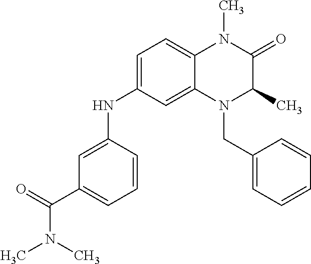 [SMILES: C[C@@H]1C(=O)N(C)c2ccc(Nc3cccc(C(=O)N(C)C)c3)cc2N1Cc1ccccc1] | 3-{[(3R)-4-benzyl-1,3- dimethyl-2-oxo-1,2,3,4- tetrahydroquinoxalin-6- yl]amino}-N,N- dimethylbenzamide | Intermediate 86, Amine 21, General Synthesis Method C | (400 MHz, CDCl3): δ = 1.17 (d, 3H); 1.64 (bs, 1H); 3.00 (bs, 3H); 3.11 (bs, 3H); 3.42 (s, 3H); 4.04 (q, 1H); 4.16 (d, 1H); 4.49 (d, 1H); 6.43 (d, 1H); 6.61 (dd, 1H); 6.79 (dd, 1H); 6.86 (d, 1H); 6.89 (d, 1H); 6.95 (s, 1H); 7.13 (t, 1H); 7.30-7.39 (m, 5H). |
| 108 | 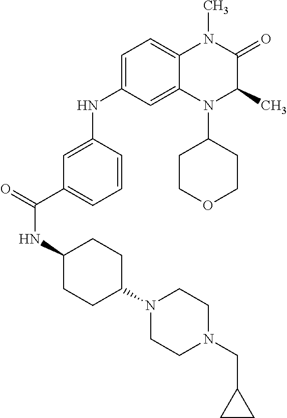 | N-{trans-4-[4- (cyclopropylmethyl)piperazin- 1-yl]cyclohexyl}-3- {[(3R)-1,3-dimethyl-2- oxo-4-(tetrahydro-2H- pyran-4-yl)-1,2,3,4- tetrahydroquinoxalin-6- yl]amino}benzamide | Intermediate 90, Amine 13, General Synthesis Method A | (400 MHz, DMSO-d6): δ = 0.01-0.06 (m, 2H), 0.39- 0.46 (m, 2H); 0.72-0.83 (m, 1H); 0.97 (d, 3H); 1.19- 1.41 (m, 4H); 1.57-1.64 (m, 1H); 1.64-1.77 (m, 2H); 1.77-1.90 (m, 5H); 2.12 (d, 2H); 2.14-2.21 (m, 1H); 2.29-2.45 (m, 4H); 3.23 (s, 3H); 3.32-3.41 (m, 4H); 3.56 (br. t, 1H); 3.62-3.72 (m, 1H); 3.85-3.95 (m, 2H); 4.05 (q, 1H); 6.63 (dd, 1H); 6.68 (d, 1H); 6.96 (d, 1H); 7.09 (br. d, 1H); 7.20 (br. d, 1H); 7.24 (t, 1H); 7.50 (br. s, 1H); 8.06 (d, 1H); 8.14 (s, 1H). |
| 109 | 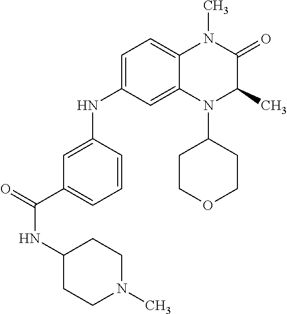 [SMILES: C[C@@H]1C(=O)N(C)c2ccc(Nc3cccc(C(=O)NC4CCN(C)CC4)c3)cc2N1C1CCOCC1] | 3-{[(3R)-1,3-dimethyl-2- oxo-4-(tetrahydro-2H- pyran-4-yl)-1,2,3,4- tetrahydroquinoxalin-6- yl]amino}-N-(1- methylpiperidin-4- yl)benzamide | Intermediate 90, Amine 14, General Synthesis Method A | (300 MHz, DMSO-d6): δ = 0.97 (d, 3H); 1.60 (d, 4H); 1.67-1.90 (m, 5H); 1.90- 2.02 (m, 2H); 2.16 (s, 3H); 2.76 (d, 2H); 3.23 (s, 3H); 3.30-3.42 (m, 2H); 3.65- 3.76 (m, 1H); 3.90 (bs, 2H); 4.05 (q, 1H); 6.63 (dd, 1H); 6.68 (d, 1H); 6.96 (d, 1H); 7.10 (dt, 1H); 7.19-7.29 (m, 2H); 7.51 (br. s, 1H); 8.11 (d, 1H); 8.15 (s, 1H). |
| 110 | 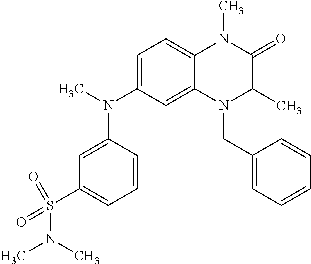 [SMILES: CC1C(=O)N(C)c2ccc(N(C)c3cccc(S(=O)(=O)N(C)C)c3)cc2N1Cc1ccccc1] | 3-[(4-benzyl-1,3-dimethyl- 2-oxo-1,2,3,4- tetrahydroquinoxalin-6- yl)(methyl)amino]-N,N- dimethylbenzenesulphon- amide | Intermediate 86, Amine 3, General Synthesis Method C | (400 MHz, CHCl3): δ = 1.19 (d, 3H); 2.71 (s, 6H); 3.25 (s, 3H); 3.44 (s, 3H); 4.04 (q, 1H); 4.13 (d, 1H); 4.47 (d, 1H); 6.49 (d, 1H); 6.68 (dd, 1H); 6.87 (ddd, 1H); 6.95 (d, 1H); 7.14 (br. d, 1H); 7.17 (t, 1H); 7.22 (t, 1H); 7.29-7.36 (m, 5H). |

TABLE 5
The following examples were obtained by separating the corresponding racemic example
compounds:

| | | | Racemic | Column; eluent; flow rate; |
|---|---|---|---|---|
| Ex. | Structure | Name | example No. | analysis |
| 111 | 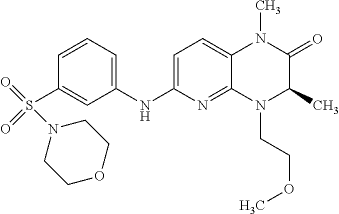 [SMILES: COCCN1c2nc(Nc3cccc(S(=O)(=O)N4CCOCC4)c3)ccc2N(C)C(=O)[C@H]1C] | (3R)-4-(2-methoxyethyl)- 1,3-dimethyl-6-{[3- (morpholin-4-ylsulphonyl) phenyl]amino}-3,4- dihydropyrido[2,3- b]pyrazin-2(1H)-one | Example 49 | Chiralpak IA 5 μm 250 × 20 mm; hexane/ethanol/diethylamine 70:30:0.1 flow rate 20 ml/min; Rt = 8.8-9.9 min Yield: 14 mg |
| 112 | 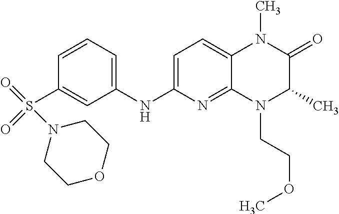 [SMILES: COCCN1c2nc(Nc3cccc(S(=O)(=O)N4CCOCC4)c3)ccc2N(C)C(=O)[C@@H]1C] | (3S)-4-(2-methoxyethyl)- 1,3-dimethyl-6-{[3- (morpholin-4-ylsulphonyl) phenyl]amino}-3,4- dihydropyrido[2,3- b]pyrazin-2(1H)-one | Example 49 | Chiralpak IA 5 μm 250 × 20 mm; hexane/ethanol/diethylamine 70:30:0.1 flow rate 20 ml/min; Rt = 5.2-6.7 min Yield: 10 mg |
| 113 | 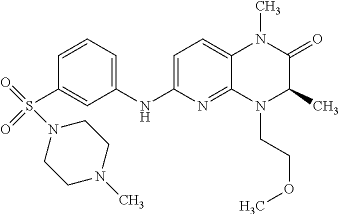 [SMILES: COCCN1c2nc(Nc3cccc(S(=O)(=O)N4CCN(C)CC4)c3)ccc2N(C)C(=O)[C@H]1C] | (3R)-4-(2-methoxyethyl)- 1,3-dimethyl-6-({3-[(4- methylpiperazin-1- yl)sulphonyl]phenyl}amino)- 3,4-dihydropyrido[2,3- b]pyrazin-2(1H)-one | Example 53 | Chiralpak IA 5 μm 250 × 30 mm; hexane/2- propanol/diethylamine 70:30:0.1 flow rate 50 ml/min; Rt = 7.8-11.0 min Yield: 55 mg |
| 114 | 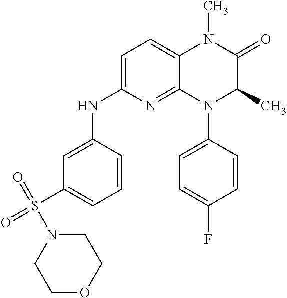 [SMILES: C[C@@H]1C(=O)N(C)c2ccc(Nc3cccc(S(=O)(=O)N4CCOCC4)c3)nc2N1c1ccc(F)cc1] | (3R)-4-(4-fluorophenyl)- 1,3-dimethyl-6-{[3- (morpholin-4- ylsulphonyl)phenyl]amino}- 3,4-dihydropyrido[2,3- b]pyrazin-2(1H)-one | Example 135 | Chiralpak IA 5 μm 250 × 30 mm; hexane/2- propanol/diethylamine 70:30:0.1 flow rate 40 ml/min; Rt = 15.2-23.5 min (400 MHz, 25° C., DMSO-d6): δ = 1.29 (d, 3H); 2.75-2.84 (m, 4H); 3.30 (s, 3H); 3.57.3.65 (m, 4H); 4.52 (q, 1H); 6.38 (d, 1H); 7.01-7.10 (m, 2H); 7.26-7.32 (m, 2H); 7.36 (t, 1H); 7.38-7.44 (m, 3H); 7.84-7.88 (m, 1H); 9.26 (s, 1H). Yield: 88 mg |
| 115 | 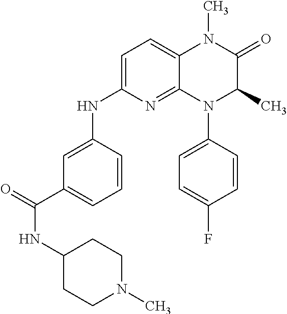 [SMILES: C[C@@H]1C(=O)N(C)c2ccc(Nc3cccc(C(=O)NC4CCN(C)CC4)c3)nc2N1c1ccc(F)cc1] | 3-{[(3R)-4-(4- fluorophenyl)-1,3- dimethyl-2-oxo-1,2,3,4- tetrahydropyrido[2,3- b]pyrazin-6-yl]amino}-N- (1-methylpiperidin-4- yl)benzamide | Example 136 | Chiralpak IC 5 μm 250 × 30 mm; hexane/ethanol/diethylamine 70:30:0.1 flow rate 50 ml/min; Rt = 13.7-15.3 min (400 MHz, DMSO-d6): δ = 1.26 (d, 3H); 1.49-1.61 (m, 2H); 1.68-1.77 (m, 2H); 1.91- 2.02 (m, 2H); 2.17 (s, 3H); 2.73- 2.81 (m, 2H); 3.28 (s, 3H); 3.64-3.76 (m, 1H); 4.50 (q, 1H); 6.36 (d, 1H); 6.90 (t, 1H); 7.15 (br. d, 1H); 7.22 (t, 2H); 7.35-7.41 (m, 3H); 7.44 (t, 1H); 7.58 (dd, 1H); 8.05 (d, 1H); 8.92 (s, 1H). Yield: 26 mg |
| 116 | 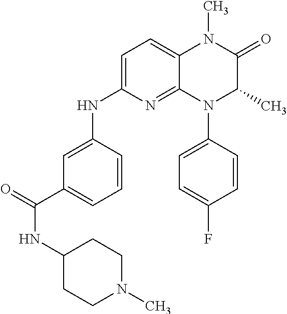 [SMILES: C[C@H]1C(=O)N(C)c2ccc(Nc3cccc(C(=O)NC4CCN(C)CC4)c3)nc2N1c1ccc(F)cc1] | 3-{[(3S)-4-(4- fluorophenyl)-1,3- dimethyl-2-oxo-1,2,3,4- tetrahydropyrido[2,3- b]pyrazin-6-yl]amino}-N- (1-methylpiperidin-4- yl)benzamide | Example 136 | Chiralpak IC 5 μm 250 × 30 mm; hexane/ethanol/diethylamine 70:30:0.1 flow rate 50 ml/min; Rt = 21.2-23.4 min (400 MHz, DMSO-d6): δ = 1.26 (d, 3H); 1.49-1.61 (m, 2H); 1.68-1.77 (m, 2H); 1.91- 2.02 (m, 2H); 2.17 (s, 3H); 2.73- 2.81 (m, 2H); 3.28 (s, 3H); 3.64-3.76 (m, 1H); 4.50 (q, 1H); 6.36 (d, 1H); 6.90 (t, 1H); 7.15 (br. d, 1H); 7.22 (t, 2H); 7.35-7.41 (m, 3H); 7.44 (t, 1H); 7.58 (dd, 1H); 8.05 (d, 1H); 8.92 (s, 1H). Yield: 32 mg |
| 117 | 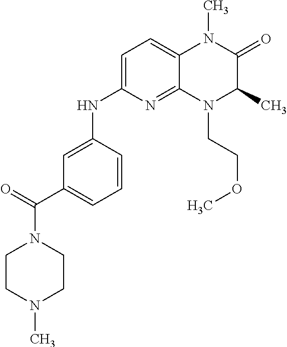 [SMILES: COCCN1c2nc(Nc3cccc(C(=O)N4CCN(C)CC4)c3)ccc2N(C)C(=O)[C@H]1C] | (3R)-4-(2-methoxyethyl)- 1,3-dimethyl-6-({3-[(4- methylpiperazin-1- yl)carbonyl]phenyl}amino)- 3,4-dihydropyrido[2,3- b]pyrazin-2(1H)-one | Example 137 | Chiralpak AS-H 5 μm 250 × 20 mm; methanol/ethanol/diethylamine 50:50:0.1 flow rate 31 ml/min; Rt = 2.5-4.25 min (400 MHz, DMSO-d6): δ = 1.12 (d, 3H); 2.18 (s, 3H); 2.21- 2.37 (m, 4H); 3.19 (s, 3H); 3.22-3.26 (m, 4H); 3.33-3.41 (m, 2H); 3.52-3.63 (m, 4H); 3.97-4.08 (m, 1H); 4.12-4.19 (m, 2H); 6.21 (d, 1H); 7.22 (d, 1H); 7.24-7.27 (m, 1H); 7.54 (dd, 1H); 7.69 (s, 1H); 8.91 (s, 1H). Yield: 6 mg |
| 118 | 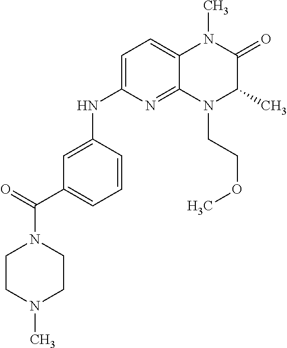 [SMILES: COCCN1c2nc(Nc3cccc(C(=O)N4CCN(C)CC4)c3)ccc2N(C)C(=O)[C@@H]1C] | (3S)-4-(2-methoxyethyl)- 1,3-dimethyl-6-({3-[(4- methylpiperazin-1- yl)carbonyl]phenyl}amino)- 3,4-dihydropyrido[2,3- b]pyrazin-2(1H)-one | Example 137 | Chiralpak AS-H 5 μm 250 × 20 mm; methanol/ethanol/diethylamine 50:50:0.1 flow rate 31 ml/min; Rt = 5.2-9.0 min (400 MHz, DMSO-d6): δ = 1.12 (d, 3H); 2.18 (s, 3H); 2.21- 2.37 (m, 4H); 3.19 (s, 3H); 3.22-3.26 (m, 4H); 3.33-3.41 (m, 2H); 3.52-3.63 (m, 4H); 3.97-4.08 (m, 1H); 4.12-4.19 (m, 2H); 6.21 (d, 1H); 7.22 (d, 1H); 7.24-7.27 (m, 1H); 7.54 (dd, 1H); 7.69 (s, 1H); 8.91 (s, 1H). Yield: 2 mg |
| 119 | 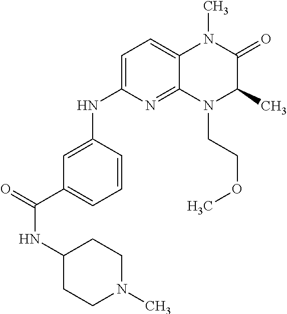 [SMILES: COCCN1c2nc(Nc3cccc(C(=O)NC4CCN(C)CC4)c3)ccc2N(C)C(=O)[C@H]1C] | 3-{[(3R)-4-(2- methoxyethyl)-1,3- dimethyl-2-oxo-1,2,3,4- tetrahydropyrido[2,3- b]pyrazin-6-yl]amino}-N- (1-methylpiperidin-4- yl)benzamide | Example 98 | Chiralpak AD-H 5 μm 250 × 30 mm; hexane/2- propanol/diethylamine 70:30:0.1 flow rate 240 ml/min; Rt = 14.5-18.0 min (300 MHz, DMSO-d6): δ = 1.12 (d, 3H); 1.23 (d, 1H); 1.56 (d, 2H); 1.73 (d, 2H); 1.93 (t, 2H); 2.15 (s, 3H); 2.75 (d, 2H); 3.19 (s, 3H); 3.20-3.22 (m, 3H); 3.55 (t, 2H); 3.63-3.78 (m, 1H); 4.02-4.21 (m, 3H); 6.21 (d, 1H); 7.18-7.27 (m, 3H); 7.65-7.72 (m, 1H); 8.04 (br. s, 1H); 8.11 (d, 1H); 8.90 (s, 1H). Yield: 60 mg |
| 120 | 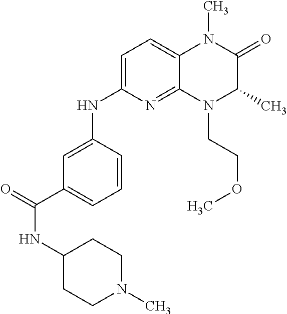 [SMILES: COCCN1c2nc(Nc3cccc(C(=O)NC4CCN(C)CC4)c3)ccc2N(C)C(=O)[C@@H]1C] | 3-{[(3S)-4-(2- methoxyethyl)-1,3- dimethyl-2-oxo-1,2,3,4- tetrahydropyrido[2,3- b]pyrazin-6-yl]amino}-N- (1-methylpiperidin-4- yl)benzamide | Example 98 | Chiralpak AD-H 5 μm 250 × 30 mm; hexane/2- propanol/diethylamine 70:30:0.1 flow rate 240 ml/min; Rt = 19.0-23.0 min (300 MHz, DMSO-d6): δ = 1.12 (d, 3H); 1.23 (d, 1H); 1.56 (d, 2H); 1.73 (d, 2H); 1.93 (t, 2H); 2.15 (s, 3H); 2.75 (d, 2H); 3.19 (s, 3H); 3.20-3.22 (m, 3H); 3.55 (t, 2H); 3.63-3.78 (m, 1H); 4.02-4.21 (m, 3H); 6.21 (d, 1H); 7.18-7.27 (m, 3H); 7.65-7.72 (m, 1H); 8.04 (br. s, 1H); 8.11 (d, 1H); 8.90 (s, 1H). Yield: 45 mg |
| 121 | 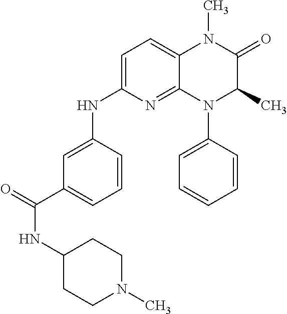 [SMILES: C[C@@H]1C(=O)N(C)c2ccc(Nc3cccc(C(=O)NC4CCN(C)CC4)c3)nc2N1c1ccccc1] | 3-{[(3R)-1,3-dimethyl-2- oxo-4-phenyl-1,2,3,4- tetrahydropyrido[2,3- b]pyrazin-6-yl]amino}-N- (1-methylpiperidin-4- yl)benzamide | Example 104 | Chiralpak IC 5 μm 250 × 20 mm; hexane/ethanol/diethylamine 75:25:0.1 flow rate 31 ml/min; Rt = 8.8-10.2 min (300 MHz, DMSO-d6): δ = 1.10 (s, 1H); 1.28 (d, 3H); 1.31- 1.62 (m, 3H); 1.66-1.77 (m, 2H); 1.84-2.04 (m, 3H); 2.14 (s, 3H); 2.69-2.78 (m, 2H); 3.59-3.76 (m, 1H); 4.54 (q, 1H); 6.40 (d, 1H); 6.88 (t, 1H); 7.11-7.23 (m, 2H); 7.31-7.45 (m, 6H); 7.70 (dd, 1H); 8.04 (d, 1H); 8.93 (s, 1H). Yield: 30 mg |

Column; eluent; flow rate; temperature: 25° C.; DAD 996 scan: 280 nm.

### Example 122, General Synthesis Method D

### (3R)-6-{[3-(2-Azaspiro[3.3]hept-2-ylsulphonyl)phenyl]amino}-1,3-dimethyl-4-(piperidin-4-yl)-3,4-dihydropyrido[2,3-b]pyrazin-2(1H)-one

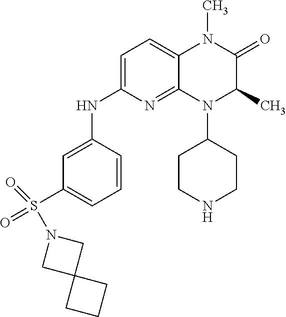 [SMILES: C[C@@H]1C(=O)N(C)c2ccc(Nc3cccc(S(=O)(=O)N4CC5(CCC5)C4)c3)nc2N1C1CCNCC1]

A solution of 150 mg of Example 60 in 17 ml of dichloromethane and 0.25 ml of trifluoroacetic acid was stirred at RT for 14 hours. With addition of toluene, the solvent was removed under reduced pressure and the residue was purified by RP-HPLC (Waters SQD autopurification system; column: Waters XBridge C18 5μ 100×30 mm; eluent A: water+0.1% by vol. of formic acid (99%), eluent B: acetonitrile; gradient: 0-8.0 min 1-100% B, 8.0-10.0 min 100% B; flow rate 50.0 ml/min; temperature: RT; injection: 2500 μl; DAD scan: 210-400 nm). This gave 85 mg of (3R)-6-{[3-(2-azaspiro[3.3]hept-2-ylsulphonyl)phenyl]amino}-1,3-dimethyl-4-(piperidin-4-yl)-3,4-dihydropyrido[2,3-b]pyrazin-2(1H)-one.

¹H NMR (300 MHz, DMSO-d6): δ=1.08 (d, 3H); 1.63 (q, 2H); 1.74-2.00 (m, 6H); 2.12 (d, 1H); 3.01 (t, 3H); 3.21 (s, 3H); 3.26-3.40 (m, 3H); 3.57-3.70 (m, 4H); 4.16 (q, 1H); 4.52 (br. t, 1H); 6.32 (d, 1H); 7.20 (d, 1H); 7.34 (d, 1H); 7.51 (t, 1H); 7.76 (dd, 1H); 8.25 (br. s, 1H); 9.32 (s, 1H).

TABLE 6
The following examples were obtained in accordance with General Synthesis Method D from
the named preparation examples:

| | | | From | |
|---|---|---|---|---|
| Ex. | Structure | Name | Example | Analytical data: ¹H NMR |
| 123 | 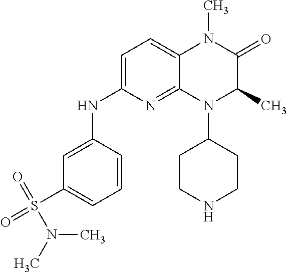 [SMILES: C[C@@H]1C(=O)N(C)c2ccc(Nc3cccc(S(=O)(=O)N(C)C)c3)nc2N1C1CCNCC1] | 3-{[(3R)-1,3-dimethyl-2- oxo-4-(piperidin-4-yl)- 1,2,3,4- tetrahydropyrido[2,3- b]pyrazin-6-yl]amino}- N,N-dimethylbenzene- sulphonamide | Example 61 | (300 MHz, DMSO-d6): δ = 1.07 (d, 3H); 1.48-1.60 (m, 2H); 1.60-1.77 (m, 1H); 1.95 (d, 1H); 2.56-2.70 (m, 9H); 3.03 (t, 2H); 3.20 (s, 3H); 4.20 (q, 1H); 4.32 (t, 1H); 6.25 (d, 1H); 7.13 (d, 1H); 7.28 (d, 1H); 7.45 (t, 1H); 7.73 (s, 1H); 8.22 (d, 1H); 9.22 (s, 1H). |
| 124 | 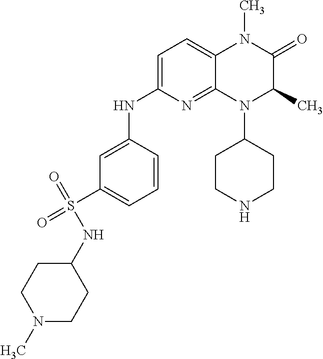 [SMILES: C[C@@H]1C(=O)N(C)c2ccc(Nc3cccc(S(=O)(=O)NC4CCN(C)CC4)c3)nc2N1C1CCNCC1] | 3-{[(3R)-1,3-dimethyl-2- oxo-4-(piperidin-4-yl)- 1,2,3,4- tetrahydropyrido[2,3- b]pyrazin-6-yl]amino}-N- (1-methylpiperidin-4- yl)benzenesulphonamide | Example 57 | (400 MHz, DMSO-d6): δ = 1.07 (d, 3H); 1.30-1.42 (m, 2H); 1.47-1.64 (m, 5H); 1.74 (d, 3H); 1.94 (br. d, 1H); 2.04 (s, 3H); 2.54-2.68 (m, 3H); 2.89 (s, 1H); 2.96- 3.07 (m, 3H); 3.20 (s, 3H); 4.19 (q, 1H); 4.30 (br. t, 1H); 6.25 (d, 1H); 7.23 (br. d, 1H); 7.26 (d, 1H); 7.39 (t, 1H); 7.59-7.70 (m, 1H); 7.86 (s, 1H); 8.02 (d, 1H); 9.13 (s, 1H). |
| 125 | 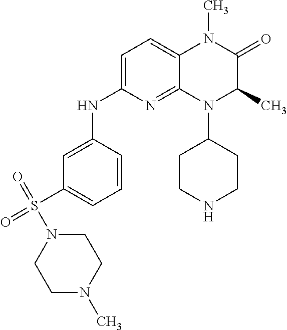 [SMILES: C[C@@H]1C(=O)N(C)c2ccc(Nc3cccc(S(=O)(=O)N4CCN(C)CC4)c3)nc2N1C1CCNCC1] | (3R)-1,3-dimethyl-6-({3- [(4-methylpiperazin-1- yl)sulphonyl]phenyl}amino)- 4-(piperidin-4-yl)-3,4- dihydropyrido[2,3- b]pyrazin-2(1H)-one | Example 62 | (400 MHz, DMSO-d6): δ = 1.09 (d, 3H); 1.55 (bs, 3H); 1.92-2.00 (m, 1H); 2.14 (s, 4H); 2.34-2.39 (m, 4H); 2.63 (d, 2H); 2.89 (bs, 4H); 3.03 (bs, 2H); 3.21 (s, 3H); 4.21 (q, 1H); 4.33 (tt, 1H); 6.26 (d, 1H); 7.13 (br. d, 1H); 7.29 (d, 1H); 7.47 (t, 1H); 7.73 (t, 1H); 8.22 (dd, 1H); 9.23 (s, 1H). |
| 126 | 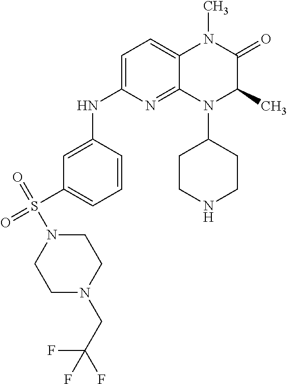 [SMILES: C[C@@H]1C(=O)N(C)c2ccc(Nc3cccc(S(=O)(=O)N4CCN(CC(F)(F)F)CC4)c3)nc2N1C1CCNCC1] | (3R)-1,3-dimethyl-4- (piperidin-4-yl)-6-[(3-{[4- (2,2,2- trifluoroethyl)piperazin-1- yl]sulphonyl}phenyl)amino]- 3,4-dihydropyrido[2,3- b]pyrazin-2(1H)-one | Example 66 | (400 MHz, DMSO-d6): δ = 1.10 (d, 3H); 1.85 (d, 1H); 1.90-2.04 (m, 1H); 2.18 (br. d, 1H); 2.68 (br. t, 4H); 2.89 (bs, 4H); 2.98-3.12 (m, 2H); 3.13-3.21 (m, 2H); 3.22-3.24 (m, 3H); 3.33- 3.42 (m, 4H); 4.17 (q, 1H); 4.53 (tt, 1H); 6.33 (d, 1H); 7.16 (br. d, 1H); 7.35 (d, 1H); 7.51 (t, 1H); 7.80 (dd, 1H); 8.12 (t, 1H); 9.33 (s, 1H). |
| 127 | 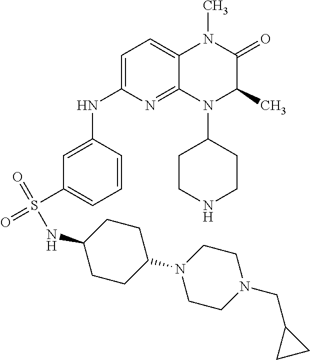 | N-{trans-4-[4- (cyclopropylmethyl)piperazin- 1-yl]cyclohexyl}-3- {[(3R)-1,3-dimethyl-2- oxo-4-(piperidin-4-yl)- 1,2,3,4- tetrahydropyrido[2,3- b]pyrazin-6-yl]amino}- benzenesulphonamide | Example 52 | (400 MHz, DMSO-d6): δ = 0.25 (bs, 2H); 0.56 (d, 2H); 0.83-1.05 (m, 2H); 1.09 (d, 3H); 1.19 (bs, 4H); 1.62- 1.90 (m, 6H); 1.90-2.08 (m, 2H); 2.09-2.29 (m, 2H); 2.72 (bs, 2H); 2.88 (bs, 3H); 2.99-3.30 (m, 10H); 4.15 (q, 1H); 4.57 (br. t, 1H); 6.31 (d, 1H); 7.25 (d, 1H); 7.33 (d, 1H); 7.38-7.46 (m, 1H); 7.50 (d, 1H); 7.66 (br. d, 1H); 8.26-8.42 (m, 2H); 8.76 (br. d, 1H); 9.25 (s, 1H). |
| 128 | 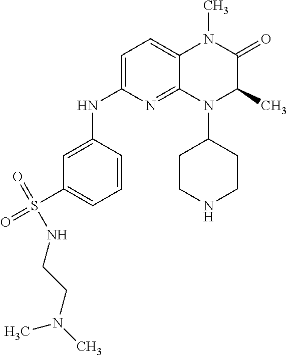 [SMILES: C[C@@H]1C(=O)N(C)c2ccc(Nc3cccc(S(=O)(=O)NCCN(C)C)c3)nc2N1C1CCNCC1] | N-[2- (dimethylamino)ethyl]-3- {[(3R)-1,3-dimethyl-2- oxo-4-(piperidin-4-yl)- 1,2,3,4- tetrahydropyrido[2,3- b]pyrazin-6-yl]amino}- benzenesulphonamide | Example 59 | (400 MHz, DMSO-d6): δ = 1.09 (d, 3H); 1.50-1.64 (m, 2H); 1.65-1.78 (m, 1H); 1.92-1.99 (m, 1H); 2.05 (s, 7H); 2.24 (t, 2H); 2.56-2.70 (m, 3H); 2.82 (d, 2H); 2.97- 3.07 (m, 2H); 3.21 (s, 3H); 4.21 (q, 1H); 4.32 (br. t, 1H); 6.26 (d, 1H); 7.23 (br. d, 1H); 7.28 (d, 1H); 7.41 (t, 1H); 7.86 (s, 1H); 8.06 (br. d, 1H); 9.16 (s, 1H). |
| 129 | 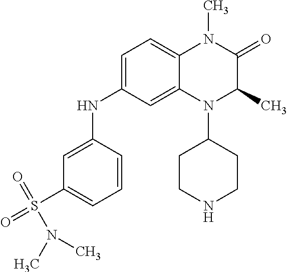 [SMILES: C[C@@H]1C(=O)N(C)c2ccc(Nc3cccc(S(=O)(=O)N(C)C)c3)cc2N1C1CCNCC1] | 3-{[(3R)-1,3-dimethyl-2- oxo-4-(piperidin-4-yl)- 1,2,3,4- tetrahydroquinoxalin-6- yl]amino}-N,N- dimethylbenzenesulphon- amide | Example 68 | (600 MHz, DMSO-d6): δ = 0.98 (d, 3H); 1.47-1.63 (m, 3H); 1.82-1.88 (m, 1H); 2.52 (d, 2H); 2.62 (s, 6H); 2.95-3.03 (m, 2H); 3.25 (s, 3H); 3.37-3.45 (m, 1H); 4.04 (q, 1H); 5.75 (s, 1H); 6.67 (dd, 1H); 6.68 (br. s, 1H); 7.01 (d, 1H); 7.05 (br. d, 1H); 7.24 (dd, 1H); 7.32 (t, 1H); 7.43 (dd, 1H); 8.46 (s, 1H). |
| 130 | 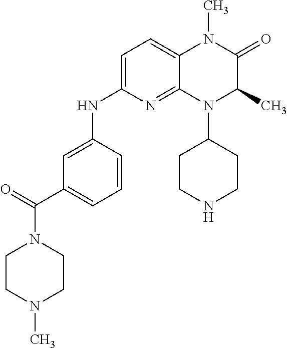 [SMILES: C[C@@H]1C(=O)N(C)c2ccc(Nc3cccc(C(=O)N4CCN(C)CC4)c3)nc2N1C1CCNCC1] | (3R)-1,3-dimethyl-6-({3- [(4-methylpiperazin-1- yl)carbonyl]phenyl}amino)- 4-(piperidin-4-yl)-3,4- dihydropyrido[2,3- b]pyrazin-2(1H)-one | Example 78 | (400 MHz, DMSO-d6): δ = 1.09 (d, 3H); 1.48-1.60 (m, 2H); 1.61-1.74 (m, 1H); 1.89-1.96 (m, 1H); 2.19 (s, 3H); 2.23-2.38 (m, 4H); 2.52-2.60 (m, 4H); 2.98- 3.07 (m, 2H); 3.21 (s, 3H); 3.52-3.69 (m, 2H); 4.16- 4.23 (m, 2H); 4.30 (tt, 1H); 6.24 (d, 1H); 6.78 (d, 1H); 7.26 (d, 2H); 7.62 (dd, 1H); 7.77 (br. s, 1H); 8.96 (s, 1H). |
| 131 | 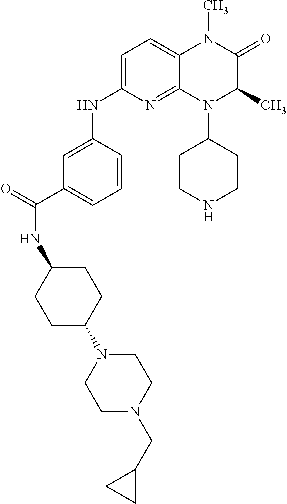 [SMILES: C[C@@H]1C(=O)N(C)c2ccc(Nc3cccc(C(=O)N[C@H]4CC[C@H](N5CCN(CC6CC6)CC5)CC4)c3)nc2N1C1CCNCC1] | N-{trans-4-[4-(cyclo- propylmethyl)piperazin-1- yl]cyclohexyl}-3-{[(3R)- 1,3-dimethyl-2-oxo-4- (piperidin-4-yl)-1,2,3,4- tetrahydropyrido[2,3- b]pyrazin-6- yl]amino}benzamide | Example 102 | (400 MHz, DMSO-d6): δ = −0.01-0.07 (m, 2H); 0.39- 0.46 (m, 2H); 0.73-0.83 (m, 1H); 1.07 (d, 3H); 1.19- 1.40 (m, 4H); 1.45-1.57 (m, 2H); 1.68 (dd, 1H); 1.78- 1.91 (m, 4H); 1.95 (d, 1H); 2.12 (d, 2H); 2.17 (t, 1H); 2.39 (bs, 4H); 2.51-2.64 (m, 3H); 3.00 (d, 2H); 3.19 (s, 3H); 3.21-3.24 (m, 2H); 3.68 (bs, 1H); 4.17 (q, 1H); 4.29 (t, 1H); 6.21 (d, 1H); 7.20-7.28 (m, 3H); 7.82 (s, 1H); 7.90 (d, 1H); 8.06 (d, 1H); 8.90 (s, 1H). |
| 132 | 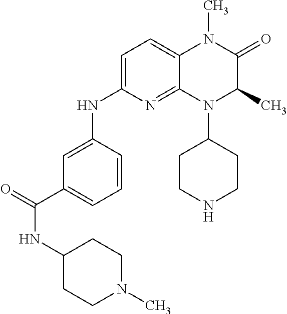 [SMILES: C[C@@H]1C(=O)N(C)c2ccc(Nc3cccc(C(=O)NC4CCN(C)CC4)c3)nc2N1C1CCNCC1] | 3-{[(3R)-1,3-dimethyl-2- oxo-4-(piperidin-2-yl)- 1,2,3,4- tetrahydropyrido[2,3- b]pyrazin-6-yl]amino}-N- (1-methylpiperidin-4- yl)benzamide | Example 100 | (300 MHz, DMSO-d6): δ = 1.07 (d, 3H); 1.45-1.64 (m, 4H); 1.65-1.79 (m, 3H); 1.83-2.00 (m, 3H); 2.14 (s, 3H); 2.56 (d, 4H); 2.74 (d, 2H); 3.00 (d, 2H); 3.19 (s, 3H); 4.17 (q, 1H); 4.29 (br. t, 1H); 6.22 (d, 1H); 7.20- 7.28 (m, 3H); 7.84 (br. s, 1H); 7.86-7.94 (m, 1H); 8.12 (d, 1H); 8.93 (s, 1H). |
| 133 | 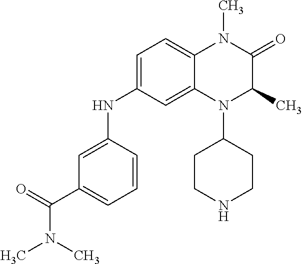 [SMILES: C[C@@H]1C(=O)N(C)c2ccc(Nc3cccc(C(=O)N(C)C)c3)cc2N1C1CCNCC1] | 3-{[(3R)-1,3-dimethyl-2- oxo-4-(piperidin-4-yl)- 1,2,3,4- tetrahydroquinoxalin-6- yl]amino}-N,N- dimethylbenzamide | Example 105 | (400 MHz, DMSO-d6): δ = 0.98 (d, 3H); 1.51-1.70 (m, 3H); 1.89 (d, 1H); 2.53- 2.63 (m, 2H); 2.94 (bs, 6H); 3.01-3.12 (m, 2H); 3.24 (s, 3H); 3.37-3.51 (m, 2H); 4.02 (q, 1H); 6.62-6.68 (m, 2H); 6.75 (br. d, 1H); 6.96- 7.01 (m, 2H); 7.04 (dd, 1H); 7.25 (t, 1H); 8.16 (s, 1H). |

### Example 134

### (3R)-1,3-Dimethyl-6-{[3-(5-methyl-1,3,4-oxadiazol-2-yl)phenyl]amino}-4-(tetrahydro-2H-pyran-4-yl)-3,4-dihydropyrido[2,3-b]pyrazin-2(1H)-one

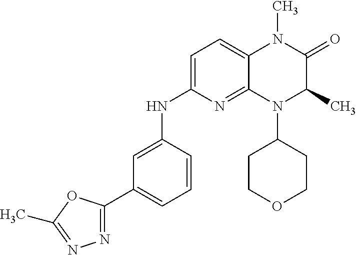

A mixture of 200 mg of Intermediate 10, 225 mg of 3-(5-methyl-1,3,4-oxadiazol-2-yl)-phenylaniline (CAS 122733-40-8), 29 mg of palladium(II) acetate (CAS 3375-31-3), 1.05 g of caesium carbonate and 80 mg of (+)-BINAP in 14.3 ml of toluene was stirred at 120° C. under an argon atmosphere for 5 hours. The mixture was added to water and extracted twice with ethyl acetate. The organic phase was washed with saturated sodium chloride solution and dried over sodium sulphate, and the solvent was removed under reduced pressure. The residue was purified by RP-HPLC chromatography (column: X-Bridge C18 5 μm 100×30 mm, mobile phase: acetonitrile/water (0.1% by vol. of formic acid) gradient). This gave 31 mg of (3R)-1,3-dimethyl-6-{[3-(5-methyl-1,3,4-oxadiazol-2-yl)phenyl]amino}-4-(tetrahydro-2H-pyran-4-yl)-3,4-dihydropyrido[2,3-b]pyrazin-2(1H)-one.

¹H NMR: (400 MHz, 25° C., DMSO-d6): δ=1.09 (d, 3H); 1.56-1.64 (m, 1H); 1.75 (qd, 1H); 1.89 (qd, 1H); 1.93-2.01 (m, 1H); 2.58 (s, 3H); 3.22 (s, 3H); 3.35 (dt, signal partly beneath water peak, 1H); 3.46 (dt, 1H); 3.83-3.94 (m, 2H); 4.24 (q, 1H); 4.49 (tt, 1H); 6.28 (d, 1H); 7.30 (d, 1H); 7.38 (td, 1H); 7.43 (t, 1H); 7.80 (td, 1H); 8.27 (t, 1H); 9.11 (s, 1H).

### Example 135

### 4-(4-Fluorophenyl)-1,3-dimethyl-6-{[3-(morpholin-4-ylsulphonyl) phenyl]amino}-3,4-dihydropyrido[2,3-b]pyrazin-2(1H)-one

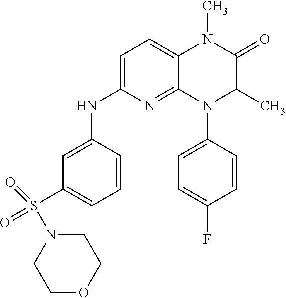 [SMILES: CC1C(=O)N(C)c2ccc(Nc3cccc(S(=O)(=O)N4CCOCC4)c3)nc2N1c1ccc(F)cc1]

A mixture of 400 mg of Intermediate 72, 451 mg of 3-(morpholin-4-ylsulphonyl)aniline (Amine 6), 17.1 mg of tris(dibenzylideneacetone)dipalladium(0) (CAS 51364-51-3), 607 mg of caesium carbonate and 41.6 mg of Xanthphos (CAS 161265-03-8) in 35 ml of dioxane was stirred under an argon atmosphere at 120° C. for 20 hours. The mixture was added to water and extracted twice with ethyl acetate. The organic phase was washed with saturated sodium chloride solution and dried over sodium sulphate, and the solvent was removed under reduced pressure. The residue was purified by RP-HPLC chromatography (column: X-Bridge C18 5 μm 100×30 mm, mobile phase: acetonitrile/water (0.1% by vol. of formic acid) gradient). This gave 900 mg of 3-{[4-(4-fluorophenyl)-1,3-dimethyl-2-oxo-1,2,3,4-tetrahydropyrido[2,3-b]pyrazin-6-yl]amino}N-(tetrahydro-2H-pyran-4-yl)benzenesulphonamide as a crude product.

UPLC-MS: Instrument: Waters Acquity UPLC-MS SQD; column: Acquity UPLC BEH C18 1.7 50×2.1 mm; eluent A: water+0.1% by vol. of formic acid (99%), eluent B: acetonitrile; gradient: 0-1.6 min 1-99% B, 1.6-2.0 min 99% B; flow rate 0.8 ml/min; temperature: 60° C.; injection: 2 μl;

DAD scan: 210-400 nm

Rt=1.22 min (M⁺+1=511)

### Example 136

### 3-{[4-(4-fluorophenyl)-1,3-dimethyl-2-oxo-1,2,3,4-tetrahydropyrido[2,3-b]pyrazin-6-yl]amino}-N-(1-methylpiperidin-4-yl)benzamide

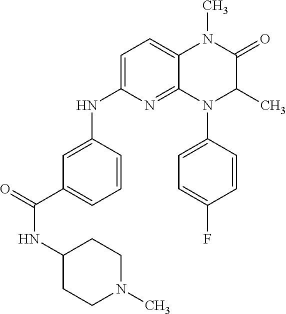 [SMILES: CC1C(=O)N(C)c2ccc(Nc3cccc(C(=O)NC4CCN(C)CC4)c3)nc2N1c1ccc(F)cc1]

A solution of 78 mg of Intermediate 74, 46 mg of 4-amino-1-methylpiperidine, 0.11 ml of triethylamine and 109 mg of HATU in 3 ml of DMF was stirred at RT for 16 hours. The mixture was added to saturated sodium chloride solution and extracted three times with ethyl acetate. The combined organic phases were washed with saturated sodium chloride solution and dried over sodium sulphate, and the solvent was removed under reduced pressure. This gave 200 mg of 3-{[4-(4-fluorophenyl)-1,3-dimethyl-2-oxo-1,2,3,4-tetrahydropyrido[2,3-b]pyrazin-6-yl]amino}-N-(1-methylpiperidin-4-yl)benzamide as a crude product.

UPLC-MS: Instrument: Waters Acquity UPLC-MS SQD; column: Acquity UPLC BEH C18 1.7 50×2.1 mm; eluent A: water+0.1% by vol. of formic acid (99%), eluent B: acetonitrile; gradient: 0-1.6 min 1-99% B, 1.6-2.0 min 99% B; flow rate 0.8 ml/min; temperature: 60° C.; injection: 2 μl;

DAD scan: 210-400 nm

Rt=0.86 min (M⁺+1=503)

### Example 137

### 4-(2-Methoxyethyl)-1,3-dimethyl-6-({3-[(4-methylpiperazin-1-yl)carbonyl]phenyl}amino)-3,4-dihydropyrido[2,3-b]pyrazin-2(1H)-one

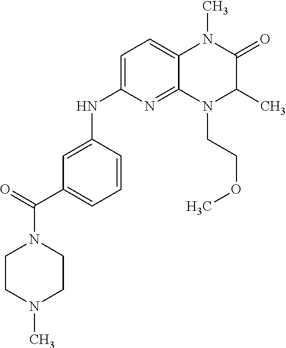 [SMILES: COCCN1c2nc(Nc3cccc(C(=O)N4CCN(C)CC4)c3)ccc2N(C)C(=O)C1C]

A solution of 100 mg of Intermediate 49, 46 mg of 1-methylpiperazine, 0.30 ml of triethylamine and 307 mg of HATU in 22 ml of DMF was stirred at RT for 48 hours. The mixture was added to semisaturated sodium chloride solution and extracted three times with ethyl acetate. The combined organic phases were washed with saturated sodium chloride solution and dried over sodium sulphate, and the solvent was removed under reduced pressure. This gave 100 mg of 4-(2-methoxyethyl)-1,3-dimethyl-6-({3-[(4-methylpiperazin-1-yl)carbonyl]phenyl}amino)-3,4-dihydropyrido[2,3-b]pyrazin-2(1H)-one as a crude product.

UPLC-MS: Instrument: Waters Acquity UPLC-MS SQD; column: Acquity UPLC BEH C18 1.7 50×2.1 mm; eluent A: water+0.1% by vol. of formic acid (99%), eluent B: acetonitrile; gradient: 0-1.6 min 1-99% B, 1.6-2.0 min 99% B; flow rate 0.8 ml/min; temperature: 60° C.; injection: 2 μl;

DAD scan: 210-400 nm

Rt=0.95 min (M⁺+1=453)

### Biological Efficacy of the Inventive Compounds

Protein-Protein Interaction Assay: BRD4/Acetylated Peptide H4 Binding Assay

### 1. Assay Description for BRD4 Bromo Domain 1 [BRD4(1)]

To assess the BRD4(1) binding strength of the substances described in this application, the ability thereof to inhibit the interaction between BRD4(1) and acetylated histone H4 in a dose-dependent manner was quantified.

For this purpose, a time-resolved fluorescence resonance energy transfer (TR-FRET) assay was used, which measures the binding between N-terminally His6-tagged BRD4(1) (amino acids 67-152) and a synthetic acetylated histone H4 (Ac-H4) peptide with sequence GRGK(Ac)GGK(Ac)GLGK(Ac)GGAK(Ac)RHGSGSK-biotin. The recombinant BRD4(1) protein produced in-house according to Filippakopoulos et al., Nature, 2010, 468:1119-1123 and Cell, 2012, 149:214-231, was expressed in E. coli and purified by means of (Ni-NTA) affinity and (Sephadex G-75) size exclusion chromatography. The Ac-H4 peptide can be purchased, for example, from Biosyntan (Berlin, Germany).

In the assay, typically 11 different concentrations of each substance (0.1 nM, 0.33 nM, 1.1 nM, 3.8 nM, 13 nM, 44 nM, 0.15 μM, 0.51 μM, 1.7 μM, 5.9 μM and 20 μM) were analysed as duplicates on the same microtitre plate. For this purpose, 100-fold concentrated solutions in DMSO were prepared by serial dilutions (1:3.4) of a 2 mM stock solution into a clear, 384-well microtitre plate (Greiner Bio-One, Frickenhausen, Germany). From this, 50 nl were transferred into a black test plate (Greiner Bio-One, Frickenhausen, Germany). The test was started by the addition of 2 μl of a 2.5-fold concentrated BRD4(1) solution (final concentration typically 10 nM in the 5 μl of reaction volume) in aqueous assay buffer [50 mM HEPES pH 7.5, 50 mM sodium chloride (NaCl), 0.25 mM CHAPS and 0.05% serum albumin (BSA)] to the substances in the test plate. This was followed by a 10-minute incubation step at 22° C. for the pre-equilibration of putative complexes between BRD4(1) and the substances. Subsequently, 3 μl of a 1.67-fold concentrated solution (in assay buffer) consisting of Ac-H4 peptide (83.5 nM) and TR-FRET detection reagents [16.7 nM anti-6His-XL665 and 3.34 nM streptavidin cryptate (both from Cisbio Bioassays, Codolet, France), and 668 mM potassium fluoride (KF)] were added.

The mixture was then incubated in the dark at 22° C. for one hour and then at 4° C. for at least 3 hours and for no longer than overnight. The formation of BRD4(1)/Ac-H4 complexes was determined by the measurement of the resonance energy transfer from the streptavidin-Eu cryptate to the anti-6His-XL665 antibody present in the reaction. For this purpose, the fluorescence emission was measured at 620 nm and 665 nm after excitation at 330-350 nm in a TR-FRET measuring instrument, for example a Rubystar or Pherastar (both from BMG Lab Technologies, Offenburg, Germany) or a Viewlux (Perkin-Elmer). The ratio of the emissions at 665 nm and at 622 nm was taken as an indicator of the amount of BRD4(1)/Ac-H4 complexes formed.

The data (ratios) obtained were normalized, with 0% inhibition corresponding to the mean from the measurements for a set of controls (typically 32 data points) in which all the reagents were present. In these, in place of test substances, 50 nl of DMSO (100%) were used. Inhibition of 100% corresponded to the mean from the measurements for a set of controls (typically 32 data points) in which all the reagents except BRD4(1) were present. The IC₅₀ was determined by regression analysis based on a 4-parameter equation (minimum, maximum, IC₅₀, Hill; Y=max+(min−max)/(1+(X/IC₅₀)Hill)).

### 2. Assay Description for BRD4 Bromo Domain 2 [BRD4(2)]

To assess the BRD4(2) binding strength of the substances described in this application, the ability thereof to inhibit the interaction between BRD4(2) and acetylated histone H4 in a dose-dependent manner was quantified.

For this purpose, a time-resolved fluorescence resonance energy transfer (TR-FRET) assay was used, which measures the binding between N-terminally His6-tagged BRD4(2) (amino acids 357-445) and a synthetic acetylated histone H4 (Ac-H4) peptide with sequence SGRGK(Ac)GGK(Ac)GLGK(Ac)GGAK(Ac)RHRKVLRDNGSGSK-biotin. The recombinant BRD4(2) protein produced in-house according to Filippakopoulos et al., Nature, 2010, 468:1119-1123 and Cell, 2012, 149:214-231, was expressed in E. coli and purified by means of (Ni-NTA) affinity and (Sephadex G-75) size exclusion chromatography. The Ac-H4 peptide can be purchased, for example, from Biosyntan (Berlin, Germany).

In the assay, typically 11 different concentrations of each substance (0.1 nM, 0.33 nM, 1.1 nM, 3.8 nM, 13 nM, 44 nM, 0.15 μM, 0.51 μM, 1.7 μM, 5.9 μM and 20 μM) were analysed as duplicates on the same microtitre plate. For this purpose, 100-fold concentrated solutions in DMSO were prepared by serial dilutions (1:3.4) of a 2 mM stock solution into a clear, 384-well microtitre plate (Greiner Bio-One, Frickenhausen, Germany). From this, 50 nl were transferred into a black test plate (Greiner Bio-One, Frickenhausen, Germany). The test was started by the addition of 2 μl of a 2.5-fold concentrated BRD4(2) solution (final concentration typically 100 nM in the 5 μl of reaction volume) in aqueous assay buffer [50 mM HEPES pH 7.5, 50 mM sodium chloride (NaCl); 50 mM potassium fluoride (KF); 0.25 mM CHAPS and 0.05% serum albumin (BSA)] to the substances in the test plate. This was followed by a 10-minute incubation step at 22° C. for the pre-equilibration of putative complexes between BRD4(2) and the substances. Subsequently, 3 μl of a 1.67-fold concentrated solution (in assay buffer) consisting of Ac-H4 peptide (83.5 nM) and TR-FRET detection reagents [83.5 nM anti-6His-XL665 (Cisbio Bioassays, Codolet, France) and 12.52 nM streptavidin-Eu), (Perkin Elmer, #W1024)] in assay buffer were added.

The mixture was then incubated in the dark at 22° C. for one hour and then at 4° C. for at least 3 hours and for no longer than overnight. The formation of BRD4(2)/Ac-H4 complexes was determined by the measurement of the resonance energy transfer from the streptavidin-Eu chelate to the anti-6His-XL665 antibody present in the reaction. For this purpose, the fluorescence emission was measured at 620 nm and 665 nm after excitation at 330-350 nm in a TR-FRET measuring instrument, for example a Rubystar or Pherastar (both from BMG Lab Technologies, Offenburg, Germany) or a Viewlux (Perkin-Elmer). The ratio of the emissions at 665 nm and at 622 nm was taken as an indicator of the amount of BRD4(2)/Ac-H4 complexes formed.

The data (ratios) obtained were normalized, with 0% inhibition corresponding to the mean from the measurements for a set of controls (typically 32 data points) in which all the reagents were present. In these, in place of test substances, 50 nl of DMSO (100%) were used. Inhibition of 100% corresponded to the mean from the measurements for a set of controls (typically 32 data points) in which all the reagents except BRD4(2) were present. The IC₅₀ was determined by regression analysis based on a 4-parameter equation (minimum, maximum, IC₅₀, Hill; Y=max+(min−max)/(1+(X/IC₅₀)Hill)).

### 3. Cell Assay

### Cell Proliferation Assay

In accordance with the invention, the ability of the substances to inhibit cell proliferation was determined. Cell viability was determined by means of the alamarBlue® reagent (Invitrogen) in a Victor X3 Multilabel Reader (Perkin Elmer). The excitation wavelength was 530 nm and the emission wavelength 590 nM.

The MOLM-13 cells (DSMZ, ACC 554) were sown at a concentration of 4000 cells/well in 100 μl of growth medium (RPMI1640, 10% FCS) on 96-well microtitre plates.

The MOLP-8 cells (DSMZ, ACC 569) were sown at a concentration of 4000 cells/well in 100 μl of growth medium (RPMI1640, 20% FCS) on 96-well microtitre plates.

The B16F10 cells (ATCC, CRL-6475) were sown at a concentration of 300-500 cells/well in 100 μl of growth medium (DMEM with phenol red, 10% FCS) on 96-well microtitre plates.

The CHL-1 cells (ATCC, CRL-9446) were sown at a concentration of 1000 cells/well in 100 μl of growth medium (DMEM with glutamine, 10% FCS) on 96-well microtitre plates.

After overnight incubation at 37° C., the fluorescence values were determined (CI values). Then the plates were treated with various substance dilutions (1E-5 M, 3E-6 M, 1E-6 M, 3E-7 M, 1E-7 M, 3E-8 M, 1E-8 M) and incubated at 37° C. over 96 hours (MOLM-13, B16F10, CHL-1 cells) or 120 hours (MOLP-8 cells). Subsequently, the fluorescence values were determined (CO values). For the data analysis, the CI values were subtracted from the CO values and the results were compared between cells which had been treated with various dilutions of the substance or only with buffer solution. The IC50 values (substance concentration needed for 50% inhibition of cell proliferation) were calculated therefrom.

The substances were tested in the cell lines in Table 7, which represent the indications specified by way of example:

TABLE 7

| Cell line | Source | Indication |
|---|---|---|
| MOLM-13 | DSMZ | acute myeloid leukaemia |
| MOLP-8 | DSMZ | multiple myeloma |
| B16F10 | ATCC | Melanoma (BRAF wild-type) |
| CHL-1 | ATCC | Melanoma (BRAF wild-type) |

### 4. Results:

### 4.1 Binding Assay

Table 8 shows the results from the BRD4(1) binding assay.

TABLE 8

| Example | IC₅₀ [BRD4(1)] (nmol/l) |
|---|---|
| 1 | 67 |
| 2 | 94 |
| 3 | 91 |
| 4 | 144 |
| 5 | 99 |
| 6 | 27 |
| 7 | 46 |
| 8 | 79 |
| 9 | 28 |
| 10 | 159 |
| 11 | 208 |
| 12 | 56 |
| 13 | 322 |
| 14 | 144 |
| 15 | 108 |
| 16 | 234 |
| 17 | 129 |
| 18 | 149 |
| 19 | 234 |
| 20 | 58 |
| 21 | 48 |
| 22 | 708 |
| 23 | 15 |
| 24 | 16 |
| 25 | 16 |
| 26 | 17 |
| 27 | 17 |
| 28 | 26 |
| 29 | 29 |
| 30 | 44 |
| 31 | 40 |
| 32 | 22 |
| 33 | 22 |
| 34 | 23 |
| 35 | 26 |
| 36 | 727 |
| 37 | 28 |
| 38 | 19 |
| 39 | 38 |
| 40 | 43 |
| 41 | 20 |
| 42 | 36 |
| 43 | 49 |
| 44 | 50 |
| 45 | 51 |
| 46 | 51 |
| 47 | 21 |
| 48 | 75 |
| 49 | 112 |
| 50 | 76 |
| 51 | 77 |
| 52 | 80 |
| 53 | 151 |
| 54 | 106 |
| 55 | 112 |
| 56 | 112 |
| 57 | 114 |
| 58 | 139 |
| 59 | 156 |
| 60 | 200 |
| 61 | 208 |
| 62 | 256 |
| 63 | 163 |
| 64 | 271 |
| 65 | 386 |
| 66 | 1110 |
| 67 | 4380 |
| 68 | 433 |
| 69 | 496 |
| 70 | 749 |
| 71 | 232 |
| 72 | 62 |
| 73 | 77 |
| 74 | 87 |
| 75 | 118 |
| 76 | 143 |
| 77 | 144 |
| 78 | 151 |
| 79 | 193 |
| 80 | 196 |
| 81 | 210 |
| 82 | 218 |
| 83 | 218 |
| 84 | 268 |
| 85 | 314 |
| 86 | 322 |
| 87 | 351 |
| 88 | 382 |
| 89 | 399 |
| 90 | 430 |
| 91 | 439 |
| 92 | 452 |
| 93 | 466 |
| 94 | 467 |
| 95 | 475 |
| 96 | 501 |
| 97 | 503 |
| 98 | 531 |
| 99 | 551 |
| 100 | 555 |
| 101 | 617 |
| 102 | 788 |
| 103 | 810 |
| 104 | 956 |
| 105 | 208 |
| 106 | 357 |
| 107 | 385 |
| 108 | 575 |
| 109 | 521 |
| 110 | 1290 |
| 111 | 75 |
| 112 | 5300 |
| 113 | 84 |
| 114 | 286 |
| 115 | 379 |
| 116 | 6750 |
| 117 | 489 |
| 118 | 18200 |
| 119 | 742 |
| 120 | 6870 |
| 121 | 447 |
| 122 | 21 |
| 123 | 18 |
| 124 | 30 |
| 125 | 37 |
| 126 | 38 |
| 127 | 47 |
| 128 | 66 |
| 129 | 45 |
| 130 | 206 |
| 131 | 251 |
| 132 | 306 |
| 133 | 124 |
| 134 | 179 |

Table 9 shows the results from the BRD4(2) binding assay.

TABLE 9

| Example | IC₅₀ [BRD4(2)] (nmol/l) |
|---|---|
| 1 | 55 |
| 2 | 89 |
| 3 | 91 |
| 4 | 101 |
| 5 | 59 |
| 6 | 32 |
| 7 | 71 |
| 8 | 89 |
| 9 | 54 |
| 10 | 62 |
| 11 | 48 |
| 12 | 68 |
| 13 | 218 |
| 14 | 170 |
| 15 | 196 |
| 16 | 174 |
| 17 | 60 |
| 18 | 66 |
| 19 | 132 |
| 20 | 52 |
| 21 | 53 |
| 22 | 157 |
| 23 | 39 |
| 24 | 55 |
| 25 | 56 |
| 26 | 31 |
| 27 | 40 |
| 28 | 53 |
| 29 | 64 |
| 30 | 58 |
| 32 | 39 |
| 33 | 76 |
| 34 | 125 |
| 35 | 69 |
| 36 | 4230 |
| 37 | 16 |
| 38 | 48 |
| 39 | 135 |
| 40 | 52 |
| 41 | 43 |
| 43 | 96 |
| 44 | 88 |
| 45 | 148 |
| 46 | 69 |
| 47 | 59 |
| 48 | 101 |
| 49 | 159 |
| 50 | 121 |
| 51 | 99 |
| 52 | 86 |
| 53 | 186 |
| 54 | 62 |
| 55 | 149 |
| 56 | 77 |
| 57 | 177 |
| 58 | 103 |
| 59 | 77 |
| 60 | 265 |
| 61 | 166 |
| 62 | 133 |
| 63 | 158 |
| 64 | 197 |
| 65 | 589 |
| 66 | 308 |
| 67 | 5190 |
| 68 | 76 |
| 69 | 63 |
| 70 | 384 |
| 71 | 315 |
| 72 | 256 |
| 73 | 86 |
| 74 | 91 |
| 75 | 79 |
| 76 | 108 |
| 77 | 118 |
| 78 | 157 |
| 79 | 116 |
| 80 | 247 |
| 81 | 227 |
| 82 | 337 |
| 83 | 227 |
| 84 | 155 |
| 85 | 189 |
| 86 | 127 |
| 87 | 339 |
| 88 | 177 |
| 89 | 497 |
| 90 | 332 |
| 91 | 325 |
| 92 | 579 |
| 93 | 2450 |
| 94 | 115 |
| 95 | 277 |
| 96 | 573 |
| 97 | 179 |
| 98 | 216 |
| 99 | 274 |
| 100 | 462 |
| 101 | 195 |
| 102 | 687 |
| 103 | 518 |
| 104 | 471 |
| 105 | 598 |
| 106 | 817 |
| 107 | 117 |
| 108 | 520 |
| 109 | 1120 |
| 110 | 320 |
| 111 | 115 |
| 112 | 57 |
| 113 | 189 |
| 114 | 143 |
| 115 | 706 |
| 116 | >40 000 |
| 117 | 434 |
| 118 | >40 000 |
| 119 | 428 |
| 120 | 4460 |
| 121 | 430 |
| 122 | 94 |
| 123 | 62 |
| 124 | 46 |
| 125 | 44 |
| 126 | 86 |
| 127 | 90 |
| 128 | 137 |
| 129 | 97 |
| 130 | 178 |
| 131 | 188 |
| 132 | 252 |
| 133 | 990 |
| 134 | 191 |

### 4.2 Cell Proliferation Assay

Table 10 shows the results from the cell proliferation assays.

TABLE 10

| | | | IC₅₀ | IC₅₀ |
|---|---|---|---|---|
| | IC₅₀ [MOLM- | IC₅₀ [MOLP-8] | [B16F10] | [CHL-1] |
| Example | 13] (nmol/l) | (nmol/l) | (nmol/l) | (nmol/l) |
| 1 | 613 | 337 | 661 | |
| 2 | 352 | 114 | 442 | |
| 3 | 296 | 187 | 695 | |
| 4 | 535 | 249 | 778 | |
| 5 | 326 | 136 | 506 | |
| 6 | 53 | 37 | 71 | |
| 7 | 149 | | 178 | |
| 8 | 271 | | 258 | |
| 9 | 66 | 40 | 78 | |
| 10 | 233 | 82 | 122 | |
| 12 | 195 | 126 | 544 | |
| 13 | 697 | 306 | 840 | |
| 14 | 343 | 240 | 809 | |
| 15 | 511 | 313 | 1080 | |
| 16 | 679 | 306 | 1140 | 346 |
| 20 | 175 | 190 | 556 | |
| 21 | 237 | 175 | 626 | |
| 23 | 47 | 37 | 44 | 50 |
| 24 | 20 | 11 | 112 | 13 |
| 25 | 18 | 11 | 43 | 16 |
| 26 | 32 | 24 | 25 | 31 |
| 27 | 41 | 30 | 60 | 23 |
| 28 | 21 | 18 | 30 | 14 |
| 29 | 64 | 41 | 230 | 36 |
| 30 | 152 | 96 | 165 | 80 |
| 33 | 16 | 12 | 157 | 13 |
| 34 | 55 | 20 | 202 | |
| 35 | 140 | 23 | 146 | |
| 37 | 74 | 65 | 93 | 53 |
| 38 | 20 | 12 | 190 | 19 |
| 39 | 90 | 91 | 149 | 63 |
| 40 | 98 | 73 | 314 | 56 |
| 41 | 22 | 17 | 52 | 21 |
| 43 | 196 | 144 | 537 | |
| 44 | 253 | 335 | 944 | 362 |
| 45 | 126 | 67 | 393 | 81 |
| 46 | 45 | 33 | 135 | 64 |
| 47 | 51 | 25 | 188 | 20 |
| 48 | 240 | 197 | 586 | 111 |
| 49 | 269 | 163 | 399 | 153 |
| 50 | 213 | 140 | 727 | 118 |
| 51 | 97 | 58 | 423 | |
| 52 | 180 | 133 | 569 | 147 |
| 53 | 565 | 365 | 1300 | 179 |
| 54 | 233 | 247 | 164 | 156 |
| 55 | 78 | 48 | 312 | |
| 56 | 239 | 214 | 488 | 181 |
| 57 | 193 | 151 | 531 | 175 |
| 58 | 378 | 268 | 613 | 299 |
| 59 | 516 | 297 | 1140 | 204 |
| 72 | 250 | 122 | 396 | 106 |
| 73 | 110 | 63 | 468 | 80 |
| 74 | 157 | 82 | 491 | 83 |
| 75 | 197 | 133 | 381 | 125 |
| 77 | 1010 | 494 | 973 | |
| 79 | 287 | 77 | 478 | |
| 88 | 2080 | 876 | 1620 | |
| 93 | 410 | 800 | 1180 | 539 |
| 104 | 1440 | 826 | 2430 | |
| 111 | 223 | 106 | 316 | 85 |
| 113 | 329 | 240 | 674 | 251 |
| 122 | 396 | 493 | 1840 | 712 |
| 123 | 140 | 169 | 2500 | 218 |
| 124 | >20 000 | >20 000 | >20 000 | >20 000 |
| 125 | 383 | 719 | >10 000 | 2260 |
| 126 | 84 | 130 | 1160 | 202 |
| 127 | >10 000 | >10 000 | >20 000 | >20 000 |
| 129 | 536 | 567 | 2160 | 945 |
| 131 | >10 000 | >20 000 | >20 000 | >20 000 |
| 132 | >20 000 | >20 000 | >20 000 | >20 000 |
| 133 | >10 000 | 2010 | >20 000 | >10 000 |
| 134 | 546 | 453 | 768 | |

1. A compound of the formula (I) 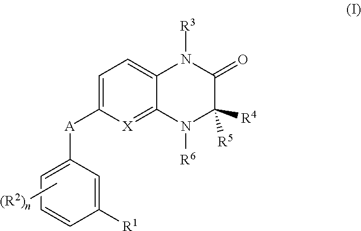
in which
A is —NH—, —N(C₁-C₃-alkyl)- or —O—,
X is —N— or —CH—,
n is 0, 1 or 2,
R¹ is a —C(═O)NR⁷R⁸ or —S(═O)₂NR⁷R⁸ group,
or
is 5-membered monocyclic heteroaryl- which may optionally be mono-, di- or trisubstituted identically or differently by halogen, cyano, C₁-C₄-alkyl-, C₂-C₄-alkenyl-, C₂-C₄-alkynyl-, halo-C₁-C₄-alkyl-, C₁-C₄-alkoxy-, halo-C₁-C₄-alkoxy-, C₁-C₄-alkylthio-, halo-C₁-C₄-alkylthio-,
—NR⁹R¹⁰, —C(═O)OR¹¹, —C(═O)N⁹R¹⁰, —C(═O)R¹¹, —S(═O)₂R¹¹, or —S(═O)₂NR⁹R¹⁰,
R² is hydrogen, halogen, cyano, C₁-C₃-alkyl-, C₂-C₄-alkenyl-, C₂-C₄-alkynyl-, halo-C₁-C₄-alkyl-, C₁-C₄-alkoxy-, halo-C₁-C₄-alkoxy-, C₁-C₄-alkylthio- or halo-C₁-C₄-alkylthio-, and, if n is 2, R² may be the same or different, or
R¹ and R² together are a *—S(═O)₂—NR⁸—CH₂—**, *—S(═O)₂—NR⁸—CH₂—CH₂—**, *—C(═O)—NR⁸—CH₂—** or *—C(═O)—NR⁸—CH₂—CH₂—** group in which “*” denotes the attachment point of R¹ to the phenyl ring in formula (I), and in which “**” denotes a carbon atom of this phenyl ring adjacent to this attachment point,
R³ is methyl- or ethyl-,
R⁴ is hydrogen or C₁-C₃-alkyl-,
R⁵ is hydrogen or C₁-C₃-alkyl-,
or
R⁴ and R⁵ together with the carbon atom to which they are bonded are C₃-C₆-cycloalkylene,
R⁶ is C₁-C₆-alkyl- which may optionally be monosubstituted by C₁-C₃-alkoxy-, phenyl-, C₃-C₈-cycloalkyl-, or 4- to 8-membered heterocycloalkyl-,
in which phenyl- may itself optionally be mono-, di- or trisubstituted identically or differently by: halogen, cyano, C₁-C₄-alkyl-, C₂-C₄-alkenyl-, C₂-C₄-alkynyl-, C₁-C₄-alkoxy-, halo-C₁-C₄-alkyl-, or halo-C₁-C₄-alkoxy-, and
in which C₃-C₈-cycloalkyl- and 4- to 8-membered heterocycloalkyl- may themselves optionally be mono- or disubstituted identically or differently by C₁-C₃-alkyl-,
or
is C₃-C₈-cycloalkyl- or 4- to 8-membered heterocycloalkyl-, which may optionally be mono- or disubstituted identically or differently by C₁-C₃-alkyl- or C₁-C₄-alkoxycarbonyl-,
or
is phenyl which may optionally be mono- or disubstituted identically or differently by halogen, C₁-C₃-alkyl- or 4-8-membered heterocycloalkyl-,
in which the 4-8-membered heterocycloalkyl- may itself optionally be mono- or disubstituted identically or differently by C₁-C₃-alkyl or C₁-C₄-alkoxycarbonyl-,
R⁷ is hydrogen,
or
is C₁-C₆-alkyl- which may optionally be mono-, di- or trisubstituted identically or differently by: hydroxyl, oxo, fluorine, cyano, C₁-C₄-alkoxy-, halo-C₁-C₄-alkoxy-, —NR⁹R¹⁰, C₃-C₈-cycloalkyl-, C₄-C₈-cycloalkenyl-, 4- to 8-membered heterocycloalkyl-, 4-bis 8-membered heterocycloalkenyl-, C₅-C₁₁-spirocycloalkyl-, C₅-C₁₁-heterospirocycloalkyl-, bridged C₆-C₁₂-cycloalkyl-, bridged C₆-C₁₂-heterocycloalkyl-, C₆-C₁₂-bicycloalkyl-, C₆-C₁₂-heterobicycloalkyl-, phenyl-, or 5- to 6-membered heteroaryl-,
in which C₃-C₈-cycloalkyl-, C₄-C₈-cycloalkenyl-, 4- to 8-membered heterocycloalkyl-, 4- to 8-membered heterocycloalkenyl-, C₅-C₁₁-spirocycloalkyl-, C₅-C₁₁-heterospirocycloalkyl-, bridged C₆-C₁₂-cycloalkyl-, bridged C₆-C₁₂-heterocycloalkyl-, C₆-C₁₂-bicycloalkyl-, and C₆-C₁₂-heterobicycloalkyl- may itself optionally be mono- or disubstituted identically or differently by: hydroxyl, fluorine, oxo, cyano, C₁-C₃-alkyl-, fluoro-C₁-C₃-alkyl-, C₃-C₆-cycloalkyl-, cyclopropylmethyl-, C₁-C₃-alkylcarbonyl-, C₁-C₄-alkoxycarbonyl- or —NR⁹R¹⁰, and
in which phenyl and 5- to 6-membered heteroaryl may optionally be mono- or disubstituted identically or differently by: halogen, cyano, trifluoromethyl-, C₁-C₃-alkyl-, or C₁-C₃-alkoxy-,
or
is C₃-C₆-alkenyl- or C₃-C₆-alkynyl-,
or
is C₃-C₈-cycloalkyl-, C₄-C₈-cycloalkenyl-, C₅-C₁i-spirocycloalkyl-, bridged C₆-C₁₂-cycloalkyl- or C₆-C₁₂-bicycloalkyl-, which may optionally be mono- or disubstituted identically or differently by: hydroxyl, oxo, cyano, fluorine, C₁-C₃-alkyl, C₁-C₃-alkoxy, trifluoromethyl, or —NR⁹R¹⁰,
or
is 4- to 8-membered heterocycloalkyl-, 4- to 8-membered heterocycloalkenyl-, C₅-C₁₁-heterospirocycloalkyl-, bridged C₆-C₁₂-heterocycloalkyl- or C₆-C₁₂-heterobicycloalkyl-, which may optionally be mono- or disubstituted identically or differently by: hydroxyl, fluorine, oxo, cyano, C₁-C₃-alkyl-, fluoro-C₁-C₃-alkyl-, C₃-C₆-cycloalkyl-, cyclopropylmethyl-, C₁-C₃-alkylcarbonyl-, C₁-C₄-alkoxycarbonyl- or —NR⁹R¹⁰,
R⁸ is hydrogen or optionally singly or doubly, identically or differently hydroxyl-, oxo- or C₁-C₃-alkoxy-substituted C₁-C₃-alkyl-, or fluoro-C₁-C₃-alkyl,
or
R⁷ and R⁸ together with the nitrogen atom to which they are bonded are 4- to 8-membered heterocycloalkyl, 4- to 8-membered heterocycloalkenyl-, C₅-C₁-heterospirocycloalkyl-, bridged C₆-C₁₂-heterocycloalkyl- or C₆-C₁₂-heterobicycloalkyl-, which may optionally be mono- or disubstituted identically or differently by: hydroxyl, fluorine, oxo, cyano, C₁-C₃-alkyl-, fluoro-C₁-C₃-alkyl-, C₃-C₆-cycloalkyl-, cyclopropylmethyl-, C₁-C₃-alkylcarbonyl-, C₁-C₄-alkoxycarbonyl- or —NR⁹R¹⁰,
R⁹ and R¹⁰ are each independently hydrogen or optionally singly or doubly, identically or differently hydroxyl-, oxo- or C₁-C₃-alkoxy-substituted C₁-C₃-alkyl, or fluoro-C₁-C₃-alkyl, or 4- to 8-membered heterocycloalkyl,
in which the 4- to 8-membered heterocycloalkyl- may itself optionally be mono- or disubstituted identically or differently by C₁-C₃-alkyl,
or
R⁹ and R¹⁰ together with the nitrogen atom to which they are attached are 4- to 8-membered heterocycloalkyl- which may optionally be mono- or disubstituted identically or differently by: hydroxyl, fluorine, oxo, cyano, C₁-C₃-alkyl-, fluoro-C₁-C₃-alkyl-, C₃-C₆-cycloalkyl-, cyclopropylmethyl-, C₁-C₃-alkylcarbonyl- or C₁-C₄-alkoxycarbonyl-,
R¹¹ is C₁-C₆-alkyl or phenyl-C₁-C₃-alkyl,
or a diastereomer, racemate, polymorph or physiologically acceptable salt thereof.

2. A compound of the formula (I) according to claim 1, in which
A is —NH— or —N(C₁-C₃-alkyl)-,
X is —N— or —CH—,
n is 0, 1 or 2,
R¹ is a —C(═O)NR⁷R⁸ or —S(═O)₂NR⁷R⁸ group,
or
is oxazolyl-, thiazolyl-, oxadiazolyl- or thiadiazolyl-, which may optionally be mono- or disubstituted identically or differently by halogen, cyano, C₁-C₃-alkyl-, or trifluoromethyl-,
C₁-C₃-alkoxy-, trifluoromethoxy- or —NR⁹R¹⁰,
R² is hydrogen, fluorine, chlorine, cyano, methyl-, methoxy-, ethyl- or ethoxy-, and if n is 2, R² may be the same or different,
R¹ and R² together are a *—S(═O)₂—NR⁸—CH₂—** or *—C(═O)—NR⁸—CH₂—** group in which “*” denotes the attachment point of R¹ to the phenyl ring in formula (I), and in which “**” denotes a carbon atom of this phenyl ring adjacent to this attachment point,
R³ is methyl- or ethyl-,
R⁴ is hydrogen, methyl- or ethyl-,
R⁵ is hydrogen, methyl- or ethyl-,
R⁶ is C₂-C₅-alkyl-,
or
is methyl- or ethyl-monosubstituted by
C₁-C₃-alkoxy-, phenyl- or 4- to 8-membered heterocycloalkyl-,
in which phenyl- may itself optionally be mono-, di- or trisubstituted identically or differently by: fluorine, chlorine, bromine, cyano, C₁-C₃-alkyl-, or C₁-C₃-alkoxy-, and
in which the 4- to 8-membered heterocycloalkyl- may itself optionally be mono- or disubstituted identically or differently by methyl-,
or
is C₃-C₈-cycloalkyl- or 4- to 8-membered heterocycloalkyl-, which may optionally be mono- or disubstituted identically or differently by C₁-C₃-alkyl- or C₁-C₄-alkoxycarbonyl-,
or
is phenyl which may optionally be mono- or disubstituted identically or differently by fluorine, chlorine, methyl- or 6-membered heterocycloalkyl-,
in which the 6-membered heterocycloalkyl- may itself optionally be monosubstituted by methyl- or tert-butoxycarbonyl-,
R⁷ is hydrogen,
or
is C₁-C₆-alkyl- which may optionally be mono-, di- or trisubstituted identically or differently by: hydroxyl, oxo, fluorine, cyano, C₁-C₃-alkoxy-, fluoro-C₁-C₃-alkoxy-, —NR⁹R¹⁰, 4- to 8-membered heterocycloalkyl-, phenyl-, or 5- to 6-membered heteroaryl-,
in which the 4- to 8-membered heterocycloalkyl- may itself optionally be monosubstituted by: hydroxyl, oxo, C₁-C₃-alkyl-, fluoro-C₁-C₃-alkyl-, cyclopropyl-, cyclopropylmethyl-, acetyl- or tert-butoxycarbonyl-,
or
is C₃-C₆-cycloalkyl- which may optionally be mono- or disubstituted identically or differently by: hydroxyl, oxo, cyano, fluorine, or —NR⁹R¹⁰,
or
is 4- to 8-membered heterocycloalkyl-, C₆-C₈-heterospirocycloalkyl-, bridged C₆-C₁₀-heterocycloalkyl- or C₆-C₁₀-heterobicycloalkyl-, which may optionally be mono- or disubstituted identically or differently by: hydroxyl, oxo, C₁-C₃-alkyl-, fluoro-C₁-C₃-alkyl-, cyclopropyl-, cyclopropylmethyl-, acetyl- or tert-butoxycarbonyl-,
R⁸ is hydrogen or C₁-C₃-alkyl-,
or
R⁷ and R⁸ together with the nitrogen atom to which they are bonded are 4- to 8-membered heterocycloalkyl-, C₆-C₈-heterospirocycloalkyl-, bridged C₆-C₁₀-heterocycloalkyl- or C₆-C₁₀-heterobicycloalkyl-,
which may optionally be mono- or disubstituted identically or differently by: hydroxyl, fluorine, oxo, C₁-C₃-alkyl-, fluoro-C₁-C₃-alkyl-, cyclopropyl-, cyclopropylmethyl-, acetyl- or tert-butoxycarbonyl-,
R⁹ and R¹⁰ are each independently hydrogen or optionally mono-hydroxyl- or -oxo-substituted C₁-C₃-alkyl- or trifluoromethyl-, or 6-membered heterocycloalkyl-, in which the 6-membered heterocycloalkyl- may itself optionally be mono- or disubstituted identically or differently by C₁-C₃-alkyl,
or
R⁹ and R¹⁰ together with the nitrogen atom to which they are bonded are 4- to 7-membered heterocycloalkyl- which may optionally be mono- or disubstituted identically or differently by: hydroxyl, fluorine, oxo, C₁-C₃-alkyl-, fluoro-C₁-C₃-alkyl-, cyclopropyl-, cyclopropylmethyl-, acetyl- or tert-butoxycarbonyl-,
or a diastereomer, racemate, polymorph or physiologically acceptable salt thereof.

3. A compound of the formula (I) according to claim 1, in which
A is —NH— or —N(methyl)-,
X is —N— or —CH—,
n is 0 or 1,
R¹ is a —C(═O)NR⁷R⁸ or —S(═O)₂NR⁷R⁸ group,
or
is oxazolyl- or oxadiazolyl- which may optionally be mono- or disubstituted identically or differently by C₁-C₃-alkyl-,
R² is hydrogen, fluorine, chlorine, methyl- or methoxy-,
or
R¹ and R² together are a *—S(═O)₂—NR⁸—CH₂—** group in which “*” denotes the attachment point of R¹ to the phenyl ring in formula (I), and in which “**” denotes a carbon atom of this phenyl ring adjacent to this attachment point,
R³ is methyl-,
R⁴ is methyl- or ethyl-,
R⁵ is hydrogen,
R⁶ is C₃-C₅-alkyl- or 2-methoxyethyl-,
or
is methyl-monosubstituted by phenyl- or 4- to 6-membered heterocycloalkyl-, in which phenyl- may itself optionally be mono- or disubstituted identically or differently by: fluorine, chlorine, cyano, methyl-, or methoxy-, and
in which the 4- to 6-membered heterocycloalkyl- may itself optionally be monosubstituted by methyl-,
or
is C₃-C₈-cycloalkyl- or is 4- to 6-membered heterocycloalkyl-, which may optionally be mono- or disubstituted identically or differently by C₁-C₃-alkyl- or C₁-C₄-alkoxycarbonyl-,
or
is phenyl which may optionally be mono- or disubstituted identically or differently by fluorine, chlorine, methyl- or N-tert-butoxycarbonylpiperazinyl-,
R⁷ is hydrogen,
or
is C₁-C₄-alkyl which may optionally be monosubstituted by —NR⁹R¹⁰ or 4- to 8-membered heterocycloalkyl,
in which the 4- to 8-membered heterocycloalkyl- may itself optionally be monosubstituted by: oxo, C₁-C₃-alkyl-, fluoro-C₁-C₃-alkyl-, cyclopropyl- or cyclopropylmethyl-,
or
is C₃-C₆-cycloalkyl- which may optionally be monosubstituted by hydroxyl, fluorine or NR⁹R¹⁰,
or
is 4- to 8-membered heterocycloalkyl- which may optionally be mono- or disubstituted identically or differently by: oxo, C₁-C₃-alkyl-, fluoro-C₁-C₃-alkyl-, cyclopropyl- or cyclopropylmethyl-,
R⁸ is hydrogen, methyl- or ethyl-,
or
R⁷ and R⁸ together with the nitrogen atom to which they are bonded are 4- to 6-membered heterocycloalkyl- or C₆-C₈-heterospirocycloalkyl-, which may optionally be mono- or disubstituted identically or differently by: fluorine, oxo, C₁-C₃-alkyl-, fluoro-C₁-C₃-alkyl-, cyclopropyl- or cyclopropylmethyl-,
R⁹ and R¹⁰ are each independently hydrogen or optionally mono-hydroxyl- or -oxo-substituted C₁-C₃-alkyl-, trifluoromethyl-, or if N-methylpiperidinyl-,
or
R⁹ and R¹⁰ together with the nitrogen atom to which they are bonded are 4- to 7-membered heterocycloalkyl- which may optionally be mono- or disubstituted identically or differently by: fluorine, oxo, C₁-C₃-alkyl-, fluoro-C₁-C₃-alkyl-, cyclopropyl- or cyclopropylmethyl-,
or a diastereomer, racemate, polymorph or physiologically acceptable salt thereof.

4. A compound of the formula (I) according to claim 1, in which
A is —NH— or —N(methyl)-,
X is —N—,
n is 0 or 1,
R¹ is a —C(═O)NR⁷R⁸ or —S(═O)₂NR⁷R⁸ group,
or
is oxazolyl- or oxadiazolyl- which may optionally be mono- or disubstituted identically or differently by C₁-C₃-alkyl-,
R² is hydrogen, fluorine, chlorine, methyl- or methoxy-,
or
R¹ and R² together are a *—S(═O)₂—NR⁸—CH₂—** group in which “*” denotes the attachment point of R¹ to the phenyl ring in formula (I), and in which “**” denotes a carbon atom of this phenyl ring adjacent to this attachment point,
R³ is methyl-,
R⁴ is methyl- or ethyl-,
R⁵ is hydrogen,
R⁶ is C₃-C₅-alkyl- or 2-methoxyethyl-,
or
is methyl-monosubstituted by phenyl- or 4- to 6-membered heterocycloalkyl-, in which phenyl- may itself optionally be mono- or disubstituted identically or differently by: fluorine, chlorine, cyano, methyl-, or methoxy-, and
in which the 4- to 6-membered heterocycloalkyl- may itself optionally be monosubstituted by methyl-,
or
is C₃-C₈-cycloalkyl- or is 4- to 6-membered heterocycloalkyl-, which may optionally be mono- or disubstituted identically or differently by C₁-C₃-alkyl- or C₁-C₄-alkoxycarbonyl-,
or
is phenyl which may optionally be mono- or disubstituted identically or differently by fluorine, chlorine, methyl- or N-tert-butoxycarbonylpiperazinyl-,
R⁷ is hydrogen,
or
is C₁-C₄-alkyl which may optionally be monosubstituted by —NR⁹R¹⁰ or 4- to 8-membered heterocycloalkyl,
in which the 4- to 8-membered heterocycloalkyl- may itself optionally be monosubstituted by: oxo, C₁-C₃-alkyl-, fluoro-C₁-C₃-alkyl-, cyclopropyl- or cyclopropylmethyl-,
or
is C₃-C₆-cycloalkyl- which may optionally be monosubstituted by hydroxyl, fluorine or NR⁹R¹⁰,
or
is 4- to 8-membered heterocycloalkyl- which may optionally be mono- or disubstituted identically or differently by: oxo, C₁-C₃-alkyl-, fluoro-C₁-C₃-alkyl-, cyclopropyl- or cyclopropylmethyl-,
R⁸ is hydrogen, methyl- or ethyl-,
or
R⁷ and R⁸ together with the nitrogen atom to which they are bonded are 4- to 6-membered heterocycloalkyl- or C₆-C₈-heterospirocycloalkyl-, which may optionally be mono- or disubstituted identically or differently by: fluorine, oxo, C₁-C₃-alkyl-, fluoro-C₁-C₃-alkyl-, cyclopropyl- or cyclopropylmethyl-,
R⁹ and R¹⁰ are each independently hydrogen or optionally mono-hydroxyl- or -oxo-substituted C₁-C₃-alkyl-, trifluoromethyl-, or N-methylpiperidinyl-,
or
R⁹ and R¹⁰ together with the nitrogen atom to which they are bonded are 4- to 7-membered heterocycloalkyl- which may optionally be mono- or disubstituted identically or differently by: fluorine, oxo, C₁-C₃-alkyl-, fluoro-C₁-C₃-alkyl-, cyclopropyl- or cyclopropylmethyl-,
or a diastereomer, racemate, polymorph or physiologically acceptable salt thereof.

5. A compound of the formula (I) according to claim 1, in which
A is —NH— or —N(methyl)-,
X is —CH—,
n is 0 or 1,
R¹ is a —C(═O)NR⁷R⁸ or —S(═O)₂NR⁷R⁸ group,
or
is oxazolyl- or oxadiazolyl- which may optionally be mono- or disubstituted identically or differently by C₁-C₃-alkyl-,
R² is hydrogen, fluorine, chlorine, methyl- or methoxy-,
or
R¹ and R² together are a *—S(═O)₂—NR⁸—CH₂—** group in which “*” denotes the attachment point of R¹ to the phenyl ring in formula (I), and in which “**” denotes a carbon atom of this phenyl ring adjacent to this attachment point,
R³ is methyl-,
R⁴ is methyl- or ethyl-,
R⁵ is hydrogen,
R⁶ is C₃-C₅-alkyl- or 2-methoxyethyl-,
or
is methyl-monosubstituted by phenyl- or 4- to 6-membered heterocycloalkyl-, in which phenyl- may itself optionally be mono- or disubstituted identically or differently by: fluorine, chlorine, cyano, methyl-, or methoxy-, and
in which the 4- to 6-membered heterocycloalkyl- may itself optionally be monosubstituted by methyl-,
or
is C₃-C₈-cycloalkyl- or is 4- to 6-membered heterocycloalkyl-, which may optionally be mono- or disubstituted identically or differently by C₁-C₃-alkyl- or C₁-C₄-alkoxycarbonyl-,
or
is phenyl which may optionally be mono- or disubstituted identically or differently by fluorine, chlorine, methyl- or N-tert-butoxycarbonylpiperazinyl-,
R⁷ is hydrogen,
or
is C₁-C₄-alkyl which may optionally be monosubstituted by —NR⁹R¹⁰ or 4- to 8-membered heterocycloalkyl,
in which the 4- to 8-membered heterocycloalkyl- may itself optionally be monosubstituted by: oxo, C₁-C₃-alkyl-, fluoro-C₁-C₃-alkyl-, cyclopropyl- or cyclopropylmethyl-,
or
is C₃-C₆-cycloalkyl- which may optionally be monosubstituted by hydroxyl, fluorine or NR⁹R¹⁰,
or
is 4- to 8-membered heterocycloalkyl- which may optionally be mono- or disubstituted identically or differently by: oxo, C₁-C₃-alkyl-, fluoro-C₁-C₃-alkyl-, cyclopropyl- or cyclopropylmethyl-,
R⁸ is hydrogen, methyl- or ethyl-,
or
R⁷ and R⁸ together with the nitrogen atom to which they are bonded are 4- to 6-membered heterocycloalkyl- or C₆-C₈-heterospirocycloalkyl-, which may optionally be mono- or disubstituted identically or differently by: fluorine, oxo, C₁-C₃-alkyl-, fluoro-C₁-C₃-alkyl-, cyclopropyl- or cyclopropylmethyl-,
R⁹ and R¹⁰ are each independently hydrogen or optionally mono-hydroxyl- or -oxo-substituted C₁-C₃-alkyl-, trifluoromethyl-, or N-methylpiperidinyl-,
or
R⁹ and R¹⁰ together with the nitrogen atom to which they are bonded are 4- to 7-membered heterocycloalkyl- which may optionally be mono- or disubstituted identically or differently by: fluorine, oxo, C₁-C₃-alkyl-, fluoro-C₁-C₃-alkyl-, cyclopropyl- or cyclopropylmethyl-,
or a diastereomer, racemate, polymorph or physiologically acceptable salt thereof.

6. A compound of the formula (I) according to claim 1, in which
A is —NH— or —N(methyl)-,
X is —N— or —CH—,
n is 0 or 1,
R¹ is a —C(═O)NR⁷R⁸ or —S(═O)₂NR⁷R⁸ group,
or is oxazolyl- or oxadiazolyl- which may optionally be mono- or disubstituted by methyl-,
R² is hydrogen, methyl- or methoxy-,
or
R¹ and R² together are a *—S(═O)₂—NH—CH₂—** group in which “*” denotes the attachment point of R¹ to the phenyl ring in formula (I), and in which “**” denotes a carbon atom of this phenyl ring adjacent to this attachment point,
R³ is methyl-,
R⁴ is methyl-,
R⁵ is hydrogen,
R⁶ is isopropyl-, isobutyl- or 2-methoxyethyl-,
or
is benzyl wherein the phenyl moiety may optionally be mono- or disubstituted identically or differently by: fluorine, or methoxy-,
or
is C₅-C₇-cycloalkyl- which may optionally be mono- or disubstituted by methyl-,
or
is tetrahydrofuranyl-, tetrahydropyranyl- or piperidinyl-,
in which piperidinyl- may optionally be monosubstituted by methyl- or tert-butoxycarbonyl-,
or
is phenyl which may optionally be monosubstituted by fluorine, methyl- or N-tert-butoxycarbonylpiperazinyl-,
R⁷ is hydrogen,
or
is C₁-C₃-alkyl which may optionally be monosubstituted by —NR⁹R¹⁰ or N-methylpiperidinyl-,
or
is cyclopropyl-, or is cyclohexyl-,
in which cyclohexyl- may optionally be monosubstituted by hydroxyl- or —NR⁹R¹⁰,
or
is 4- to 6-membered heterocycloalkyl which may optionally be monosubstituted by methyl-,
R⁸ is hydrogen, methyl- or ethyl-,
or
R⁷ and R⁸ together with the nitrogen atom to which they are bonded are 4- to 6-membered heterocycloalkyl- which may optionally be mono- or disubstituted by fluorine, or which may optionally be monosubstituted by methyl-, isopropyl-, 2,2,2-trifluoroethyl- or cyclopropylmethyl-,
or
are 6-azaspiro[3.3]heptyl- or are 2-oxa-6-azaspiro[3.3]heptyl-,
R⁹ and R¹⁰ are each independently hydrogen, C₁-C₃-alkyl- or N-methylpiperidinyl-,
or
R⁹ and R¹⁰ together with the nitrogen atom to which they are bonded are 6-membered heterocycloalkyl- which may optionally be mono- or disubstituted by fluorine, or which may optionally be monosubstituted by methyl-, 2,2,2-trifluoroethyl-, cyclopropyl- or cyclopropylmethyl-,
or a diastereomer, racemate, polymorph or physiologically acceptable salt thereof.

7. A compound of the formula (I) according to claim 1, in which
A is —NH— or —N(methyl)-,
X is —N— or —CH—,
n is 0 or 1,
R¹ is a —C(═O)NR⁷R⁸ or —S(═O)₂NR⁷R⁸ group,
or
is 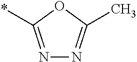 [SMILES: *c1nnc(C)o1]
in which “*” denotes the attachment point to the rest of the molecule,
R² is hydrogen, methyl- or methoxy-,
or
R¹ and R² together with the phenyl ring to which they are bonded are 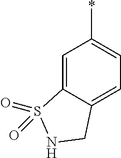 [SMILES: *c1ccc2c(c1)S(=O)(=O)NC2]
in which “*” denotes the attachment point to the rest of the molecule,
R³ is methyl-,
R⁴ is methyl-,
R⁵ is hydrogen,
R⁶ is isopropyl-, isobutyl-, 2-methoxyethyl-, benzyl-, 4-methoxybenzyl-, 2,6-difluorobenzyl-, cyclopentyl-, cyclohexyl-, cycloheptyl-, tetrahydropyran-4-yl-, phenyl-, 3-methylphenyl- or 4-fluorophenyl-,
or
is 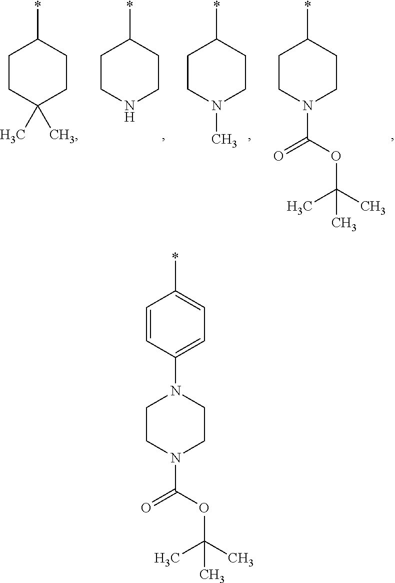
in which “*” denotes the attachment point to the rest of the molecule,
R⁷ is hydrogen, methyl-, ethyl-, isopropyl- or cyclopropyl-,
or
is 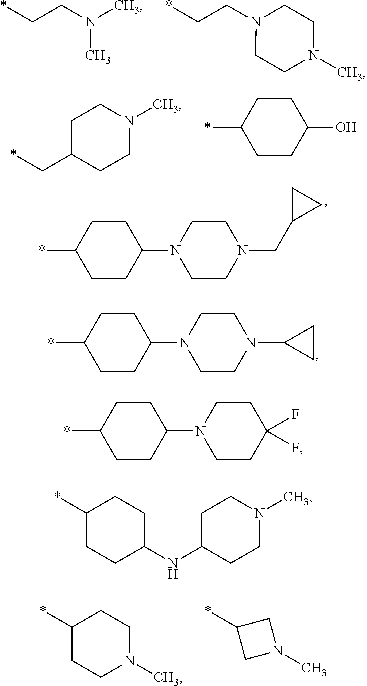
where “*” in each case denotes the attachment point to the rest of the molecule,
R⁸ is hydrogen, methyl- or ethyl-,
or
R⁷ and R⁸ together with the nitrogen atom to which they are bonded are 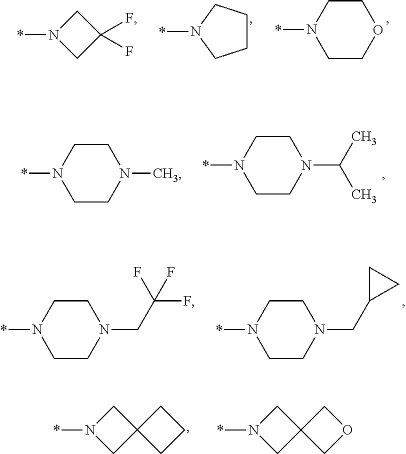
where “*” in each case denotes the attachment point to the rest of the molecule,
or a diastereomer, racemate, polymorph and or physiologically acceptable salt thereof.

8. A compound of the formula I according to claim 1, selected from:
(3R)-4-cyclopentyl-1,3-dimethyl-6-({3-[(4-methylpiperazin-1-yl)carbonyl]phenyl}amino)-3,4-dihydropyrido[2,3-b]pyrazin-2(1H)-one;
3-{[(3R)-4-cyclopentyl-1,3-dimethyl-2-oxo-1,2,3,4-tetrahydropyrido[2,3-b]pyrazin-6-yl]amino}-N-(1-methylpiperidin-4-yl)benzamide;
3-{[(3R)-4-cyclopentyl-1,3-dimethyl-2-oxo-1,2,3,4-tetrahydropyrido[2,3-b]pyrazin-6-yl]amino}-N-(1-methylazetidin-3-yl)benzamide;
3-{[(3R)-4-cyclopentyl-1,3-dimethyl-2-oxo-1,2,3,4-tetrahydropyrido[2,3-b]pyrazin-6-yl]amino}-N-[2-(dimethylamino)ethyl]benzamide;
3-{[(3R)-4-cyclopentyl-1,3-dimethyl-2-oxo-1,2,3,4-tetrahydropyrido[2,3-b]pyrazin-6-yl]amino}-N-{trans-4-[4-(cyclopropylmethyl)piperazin-1-yl]cyclohexyl}benzamide;
3-{[(3R)-1,3-dimethyl-2-oxo-4-(tetrahydro-2H-pyran-4-yl)-1,2,3,4-tetrahydropyrido[2,3-b]pyrazin-6-yl]amino}-N,N-dimethylbenzenesulphonamide;
3-{[(3R)-1,3-dimethyl-2-oxo-4-(tetrahydro-2H-pyran-4-yl)-1,2,3,4-tetrahydroquinoxalin-6-yl]amino}-N,N-dimethylbenzenesulphonamide;
(3R)-1,3-dimethyl-6-{[3-(morpholin-4-ylsulphonyl)phenyl]amino}-4-(tetrahydro-2H-pyran-4-yl)-3,4-dihydroquinoxalin-2(1H)-one;
(3R)-1,3-dimethyl-6-{[3-(morpholin-4-ylsulphonyl)phenyl]amino}-4-(tetrahydro-2H-pyran-4-yl)-3,4-dihydropyrido[2,3-b]pyrazin-2(1H)-one;
3-{[(3R)-4-(4-methoxybenzyl)-1,3-dimethyl-2-oxo-1,2,3,4-tetrahydroquinoxalin-6-yl]amino}-N,N-dimethylbenzenesulphonamide;
(3R)-4-(4-methoxybenzyl)-1,3-dimethyl-6-{[3-(morpholin-4-ylsulphonyl)phenyl]amino}-3,4-dihydroquinoxalin-2(1H)-one;
(3R)-1,3-dimethyl-6-({3-[(4-methylpiperazin-1-yl)sulphonyl]phenyl}amino)-4-(tetrahydro-2H-pyran-4-yl)-3,4-dihydropyrido[2,3-b]pyrazin-2(1H)-one;
3-{[(3R)-1,3-dimethyl-2-oxo-4-(tetrahydro-2H-pyran-4-yl)-1,2,3,4-tetrahydropyrido[2,3-b]pyrazin-6-yl]amino}-N-(1-methylpiperidin-4-yl)benzamide;
N-{trans-4-[4-(cyclopropylmethyl)piperazin-1-yl]cyclohexyl}-3-{[(3R)-1,3-dimethyl-2-oxo-4-(tetrahydro-2H-pyran-4-yl)-1,2,3,4-tetrahydropyrido[2,3-b]pyrazin-6-yl]amino}benzamide;
(3R)-1,3-dimethyl-6-({3-[(4-methylpiperazin-1-yl)carbonyl]phenyl}amino)-4-(tetrahydro-2H-pyran-4-yl)-3,4-dihydropyrido[2,3-b]pyrazin-2(1H)-one;
3-{[(3R)-1,3-dimethyl-2-oxo-4-(tetrahydro-2H-pyran-4-yl)-1,2,3,4-tetrahydropyrido[2,3-b]pyrazin-6-yl]amino}-4-methoxy-N-(1-methylpiperidin-4-yl)benzamide;
(3R)-6-({2-methoxy-5-[(4-methylpiperazin-1-yl)carbonyl]phenyl}amino)-1,3-dimethyl-4-(tetrahydro-2H-pyran-4-yl)-3,4-dihydropyrido[2,3-b]pyrazin-2(1H)-one;
N-{trans-4-[4-(cyclopropylmethyl)piperazin-1-yl]cyclohexyl}-3-{[(3R)-1,3-dimethyl-2-oxo-4-(tetrahydro-2H-pyran-4-yl)-1,2,3,4-tetrahydropyrido[2,3-b]pyrazin-6-yl]amino}-4-methoxybenzamide;
N-[2-(dimethylamino)ethyl]-3-{[(3R)-1,3-dimethyl-2-oxo-4-(tetrahydro-2H-pyran-4-yl)-1,2,3,4-tetrahydropyrido[2,3-b]pyrazin-6-yl]amino}-4-methoxybenzamide;
3-{[(3R)-1,3-dimethyl-2-oxo-4-(tetrahydro-2H-pyran-4-yl)-1,2,3,4-tetrahydropyrido[2,3-b]pyrazin-6-yl]amino}-N-(1-methylpiperidin-4-yl)benzenesulphonamide;
N-[2-(dimethylamino)ethyl]-3-{[(3R)-1,3-dimethyl-2-oxo-4-(tetrahydro-2H-pyran-4-yl)-1,2,3,4-tetrahydropyrido[2,3-b]pyrazin-6-yl]amino}benzenesulphonamide;
N-{trans-4-[4-(cyclopropylmethyl)piperazin-1-yl]cyclohexyl}-3-{[4-(2,6-difluorobenzyl)-1,3-dimethyl-2-oxo-1,2,3,4-tetrahydroquinoxalin-6-yl]amino}benzamide;
3-{[(3R)-1,3-dimethyl-2-oxo-4-(tetrahydro-2H-pyran-4-yl)-1,2,3,4-tetrahydropyrido[2,3-b]pyrazin-6-yl]amino}-N-ethylbenzenesulphonamide;
(3R)-1,3-dimethyl-4-(1-methylpiperidin-4-yl)-6-{[3-(pyrrolidin-1-ylsulphonyl)phenyl]amino}-3,4-dihydropyrido[2,3-b]pyrazin-2(1H)-one;
(3R)-6-{[2-methoxy-5-(morpholin-4-ylsulphonyl)phenyl]amino}-1,3-dimethyl-4-(1-methylpiperidin-4-yl)-3,4-dihydropyrido[2,3-b]pyrazin-2(1H)-one;
N-cyclopropyl-3-{[(3R)-1,3-dimethyl-2-oxo-4-(tetrahydro-2H-pyran-4-yl)-1,2,3,4-tetrahydropyrido[2,3-b]pyrazin-6-yl]amino}benzenesulphonamide;
(3R)-1,3-dimethyl-6-{[3-(pyrrolidin-1-ylsulphonyl)phenyl]amino}-4-(tetrahydro-2H-pyran-4-yl)-3,4-dihydropyrido[2,3-b]pyrazin-2(1H)-one;
(3R)-6-{[2-methoxy-5-(morpholin-4-ylsulphonyl)phenyl]amino}-1,3-dimethyl-4-(tetrahydro-2H-pyran-4-yl)-3,4-dihydropyrido[2,3-b]pyrazin-2(1H)-one;
(3R)-6-({3-[(3,3-difluoroazetidin-1-yl)sulphonyl]phenyl}amino)-1,3-dimethyl-4-(1-methylpiperidin-4-yl)-3,4-dihydropyrido[2,3-b]pyrazin-2(1H)-one;
(3R)-6-{[2-methoxy-5-(2-oxa-6-azaspiro[3.3]hept-6-ylsulphonyl)phenyl]amino}-1,3-dimethyl-4-(tetrahydro-2H-pyran-4-yl)-3,4-dihydropyrido[2,3-b]pyrazin-2(1H)-one;
(3R)-6-({3-[(3,3-difluoroazetidin-1-yl)sulphonyl]phenyl}amino)-1,3-dimethyl-4-(tetrahydro-2H-pyran-4-yl)-3,4-dihydropyrido[2,3-b]pyrazin-2(1H)-one;
3-{[(3R)-1,3-dimethyl-2-oxo-4-(tetrahydro-2H-pyran-4-yl)-1,2,3,4-tetrahydropyrido[2,3-b]pyrazin-6-yl]amino}-N,N-diethylbenzenesulphonamide;
3-{[(3R)-4-benzyl-1,3-dimethyl-2-oxo-1,2,3,4-tetrahydropyrido[2,3-b]pyrazin-6-yl]amino}-N-(1-methylpiperidin-4-yl)benzenesulphonamide;
N-{trans-4-[4-(cyclopropylmethyl)piperazin-1-yl]cyclohexyl}-3-{[(3R)-4-isopropyl-1,3-dimethyl-2-oxo-1,2,3,4-tetrahydropyrido[2,3-b]pyrazin-6-yl]amino}benzenesulphonamide;
N-{trans-4-[4-(cyclopropylmethyl)piperazin-1-yl]cyclohexyl}-3-{[(3R)-1,3-dimethyl-2-oxo-4-(tetrahydro-2H-pyran-4-yl)-1,2,3,4-tetrahydropyrido[2,3-b]pyrazin-6-yl]amino}benzenesulphonamide;
N-{trans-4-[4-(cyclopropylmethyl)piperazin-1-yl]cyclohexyl}-3-{[(3S)-1,3-dimethyl-2-oxo-4-(tetrahydro-2H-pyran-4-yl)-1,2,3,4-tetrahydropyrido[2,3-b]pyrazin-6-yl]amino}benzenesulphonamide;
(3R)-1,3-dimethyl-4-(tetrahydro-2H-pyran-4-yl)-6-[(3-{[4-(2,2,2-trifluoroethyl)piperazin-1-yl]sulphonyl}phenyl)amino]-3,4-dihydropyrido[2,3-b]pyrazin-2(1H)-one;
3-{[(3R)-4-benzyl-1,3-dimethyl-2-oxo-1,2,3,4-tetrahydropyrido[2,3-b]pyrazin-6-yl]amino}-N-{trans-4-[4-(cyclopropylmethyl)piperazin-1-yl]cyclohexyl}benzenesulphonamide;
3-{[(3R)-1,3-dimethyl-2-oxo-4-(tetrahydro-2H-pyran-4-yl)-1,2,3,4-tetrahydropyrido[2,3-b]pyrazin-6-yl]amino}-N-methylbenzenesulphonamide;
(3R)-6-({2-methoxy-5-[(4-methylpiperazin-1-yl)sulphonyl]phenyl}amino)-1,3-dimethyl-4-(tetrahydro-2H-pyran-4-yl)-3,4-dihydropyrido[2,3-b]pyrazin-2(1H)-one;
3-{[(3R)-1,3-dimethyl-4-(1-methylpiperidin-4-yl)-2-oxo-1,2,3,4-tetrahydropyrido[2,3-b]pyrazin-6-yl]amino}-N,N-dimethylbenzenesulphonamide;
3-{[(3R)-1,3-dimethyl-2-oxo-4-(tetrahydro-2H-pyran-4-yl)-1,2,3,4-tetrahydropyrido[2,3-b]pyrazin-6-yl]amino}-N-isopropylbenzenesulphonamide;
(3R)-4-isopropyl-1,3-dimethyl-6-({3-[(4-methylpiperazin-1-yl)sulphonyl]phenyl}amino)-3,4-dihydropyrido[2,3-b]pyrazin-2(1H)-one;
3-{[(3R)-1,3-dimethyl-2-oxo-4-(tetrahydro-2H-pyran-4-yl)-1,2,3,4-tetrahydropyrido[2,3-b]pyrazin-6-yl]amino}-N-[(1-methylpiperidin-4-yl)methyl]benzenesulphonamide;
N-{trans-4-[4-(cyclopropylmethyl)piperazin-1-yl]cyclohexyl}-3-{[(3R)-4-isobutyl-1,3-dimethyl-2-oxo-1,2,3,4-tetrahydropyrido[2,3-b]pyrazin-6-yl]amino}benzenesulphonamide;
(3R)-6-({2-methoxy-5-[(4-methylpiperazin-1-yl)sulphonyl]phenyl}amino)-1,3-dimethyl-4-(1-methylpiperidin-4-yl)-3,4-dihydropyrido[2,3-b]pyrazin-2(1H)-one;
(3R)-1,3-dimethyl-4-(1-methylpiperidin-4-yl)-6-{[3-(morpholin-4-ylsulphonyl)phenyl]amino}-3,4-dihydropyrido[2,3-b]pyrazin-2(1H)-one;
3-{[(3R)-4-cycloheptyl-1,3-dimethyl-2-oxo-1,2,3,4-tetrahydropyrido[2,3-b]pyrazin-6-yl]amino}-N-(1-methylpiperidin-4-yl)benzenesulphonamide;
4-(2-methoxyethyl)-1,3-dimethyl-6-{[3-(morpholin-4-ylsulphonyl)phenyl]amino}-3,4-dihydropyrido[2,3-b]pyrazin-2(1H)-one;
(3R)-1,3-dimethyl-6-({3-[(4-methylpiperazin-1-yl)sulphonyl]phenyl}amino)-4-(1-methylpiperidin-4-yl)-3,4-dihydropyrido[2,3-b]pyrazin-2(1H)-one;
3-{[(3R)-4-isopropyl-1,3-dimethyl-2-oxo-1,2,34-tetrahydropyrido[2,3-b]pyrazin-6-yl]amino}-N-(1-methylpiperidin-4-yl)benzenesulphonamide;
tert-butyl 4-[(3R)-6-{[3-({trans-4-[4-(cyclopropylmethyl)piperazin-1-yl]cyclohexyl}sulphamoyl)phenyl]amino}-1,3-dimethyl-2-oxo-2,3-dihydropyrido[2,3-b]pyrazin-4(1H)-yl]piperidine-1-carboxylate;
4-(2-methoxyethyl)-1,3-dimethyl-6-({3-[(4-methylpiperazin-1-yl)sulphonyl]phenyl}amino)-3,4-dihydropyrido[2,3-b]pyrazin-2(1H)-one;
3-{[(3R)-1,3-dimethyl-2-oxo-4-(tetrahydro-2H-pyran-4-yl)-1,2,3,4-tetrahydropyrido[2,3-b]pyrazin-6-yl](methyl)amino}-N,N-dimethylbenzenesulphonamide;
N-[2-(dimethylamino)ethyl]-3-{[(3R)-4-isopropyl-1,3-dimethyl-2-oxo-1,2,3,4-tetrahydropyrido[2,3-b]pyrazin-6-yl]amino}benzenesulphonamide;
(3R)-6-[(1,1-dioxido-2,3-dihydro-1,2-benzothiazol-6-yl)amino]-1,3-dimethyl-4-(tetrahydro-2H-pyran-4-yl)-3,4-dihydropyrido[2,3-b]pyrazin-2(1H)-one;
tert-butyl 4-[(3R)-1,3-dimethyl-6-({3-[(1-methylpiperidin-4-yl)sulphamoyl]phenyl}amino)-2-oxo-2,3-dihydro-pyrido[2,3-b]pyrazin-4(1H)-yl]piperidine-1-carboxylate;
3-{[(3R)-1,3-dimethyl-2-oxo-4-(tetrahydro-2H-pyran-4-yl)-1,2,3,4-tetrahydropyrido[2,3-b]pyrazin-6-yl]amino}benzenesulphonamide;
tert-butyl 4-[(3R)-6-[(3-{[2-(dimethylamino)ethyl]sulphamoyl}phenyl)amino]-1,3-dimethyl-2-oxo-2,3-dihydropyrido[2,3-b]pyrazin-4(1H)-yl]piperidine-1-carboxylate;
tert-butyl 4-[(3R)-6-{[3-(2-azaspiro[3.3]hept-2-ylsulphonyl)phenyl]amino}-1,3-dimethyl-2-oxo-2,3-dihydropyrido[2,3-b]pyrazin-4(1H)-yl]piperidine-1-carboxylate;
tert-butyl 4-[(3R)-6-{[3-(dimethylsulphamoyl)phenyl]amino}-1,3-dimethyl-2-oxo-2,3-dihydropyrido[2,3-b]pyrazin-4(1H)-yl]piperidine-1-carboxylate;
tert-butyl 4-[(3R)-1,3-dimethyl-6-({3-[(4-methylpiperazin-1-yl)sulphonyl]phenyl}amino)-2-oxo-2,3-dihydropyrido[2,3-b]pyrazin-4(1H)-yl]piperidine-1-carboxylate;
5-{[(3R)-1,3-dimethyl-2-oxo-4-(tetrahydro-2H-pyran-4-yl)-1,2,3,4-tetrahydropyrido[2,3-b]pyrazin-6-yl]amino}-2-methylbenzenesulphonamide;
1,3-dimethyl-4-(3-methylphenyl)-6-{[3-(morpholin-4-ylsulphonyl)phenyl]amino}-3,4-dihydropyrido[2,3-b]pyrazin-2(1H)-one;
3-{[1,3-dimethyl-4-(3-methylphenyl)-2-oxo-1,2,3,4-tetrahydropyrido[2,3-b]pyrazin-6-yl]amino}-N-(1-methylpiperidin-4-yl)benzenesulphonamide;
tert-butyl 4-[(3R)-1,3-dimethyl-2-oxo-6-[(3-{[4-(2,2,2-trifluoroethyl)piperazin-1-yl]sulphonyl}phenyl)amino]-2,3-dihydropyrido[2,3-b]pyrazin-4(1H)-yl]piperidine-1-carboxylate;
tert-butyl 4-{4-[1,3-dimethyl-6-({3-[(4-methylpiperazin-1-yl)sulphonyl]phenyl}amino)-2-oxo-2,3-dihydropyrido[2,3-b]pyrazin-4(1H)-yl]phenyl}piperazien-1-carboxylate;
tert-butyl 4-[(2R)-7-{[3-(dimethylsulphamoyl)phenyl]amino}-2,4-dimethyl-3-oxo-3,4-dihydroquinoxalin-1(2H)-yl]piperidine-1-carboxylate;
3-{[(3R)-4-benzyl-1,3-dimethyl-2-oxo-1,2,3,4-tetrahydroquinoxalin-6-yl]amino}-N,N-dimethylbenzenesulphonamide;
tert-butyl 4-[(2R)-7-{[3-(dimethylsulphamoyl)phenyl](methyl)amino}-2,4-dimethyl-3-oxo-3,4-dihydroquinoxalin-1(2H)-yl]piperidine-1-carboxylate;
(3R)-1,3-dimethyl-6-({3-[(4-methylpiperazin-1-yl)sulphonyl]phenyl}amino)-4-(tetrahydro-2H-pyran-4-yl)-3,4-dihydroquinoxalin-2(1H)-one;
3-{[(3R)-4-benzyl-1,3-dimethyl-2-oxo-1,2,3,4-tetrahydropyrido[2,3-b]pyrazin-6-yl]amino}-N-{trans-4-[4-(cyclopropylmethyl)piperazin-1-yl]cyclohexyl}benzamide;
3-{[(3R)-4-benzyl-1,3-dimethyl-2-oxo-1,2,3,4-tetrahydropyrido[2,3-b]pyrazin-6-yl]amino}-N-(1-methylpiperidin-4-yl)benzamide;
3-{[(3R)-4-benzyl-1,3-dimethyl-2-oxo-1,2,3,4-tetrahydropyrido[2,3-b]pyrazin-6-yl]amino}-N-[2-(4-methylpiperazin-1-yl)ethyl]benzamide;
(3R)-4-benzyl-1,3-dimethyl-6-({3-[(4-methylpiperazin-1-yl)carbonyl]phenyl}amino)-3,4-dihydropyrido[2,3-b]pyrazin-2(1H)-one;
(3R)-4-benzyl-6-({3-[(4-isopropylpiperazin-1-yl)carbonyl]phenyl}amino)-1,3-dimethyl-3,4-dihydropyrido[2,3-b]pyrazin-2(1H)-one;
(3R)-4-isopropyl-1,3-dimethyl-6-({3-[(4-methylpiperazin-1-yl)carbonyl]phenyl}amino)-3,4-dihydropyrido[2,3-b]pyrazin-2(1H)-one;
tert-butyl 4-[(3R)-1,3-dimethyl-6-({3-[(4-methylpiperazin-1-yl)carbonyl]phenyl}amino)-2-oxo-2,3-dihydropyrido[2,3-b]pyrazin-4(1H)-yl]piperidine-1-carboxylate;
N-{trans-4-[4-(cyclopropylmethyl)piperazin-1-yl]cyclohexyl}-3-{[(3R)-4-isopropyl-1,3-dimethyl-2-oxo-1,2,3,4-tetrahydropyrido[2,3-b]pyrazin-6-yl]amino}benzamide;
3-{[(3R)-1,3-dimethyl-2-oxo-4-(tetrahydro-2H-pyran-4-yl)-1,2,3,4-tetrahydropyrido[2,3-b]pyrazin-6-yl]amino}-N-{4-[(1-methylpiperidin-4-yl)amino]cyclohexyl}benzamide;
5-{[(3R)-1,3-dimethyl-2-oxo-4-(tetrahydro-2H-pyran-4-yl)-1,2,3,4-tetrahydropyrido[2,3-b]pyrazin-6-yl]amino}-2-methoxy-N-(1-methylpiperidin-4-yl)benzamide;
N-{trans-4-[4-(cyclopropylmethyl)piperazin-1-yl]cyclohexyl}-5-{[(3R)-1,3-dimethyl-2-oxo-4-(tetrahydro-2H-pyran-4-yl)-1,2,3,4-tetrahydropyrido[2,3-b]pyrazin-6-yl]amino}-2-methoxybenzamide;
(3R)-6-({4-methoxy-3-[(4-methylpiperazin-1-yl)carbonyl]phenyl}amino)-1,3-dimethyl-4-(tetrahydro-2H-pyran-4-yl)-3,4-dihydropyrido[2,3-b]pyrazin-2(1H)-one;
3-{[(3R)-4-cyclohexyl-1,3-dimethyl-2-oxo-1,2,3,4-tetrahydropyrido[2,3-b]pyrazin-6-yl]amino}-N-[2-(4-methylpiperazin-1-yl)ethyl]benzamide;
(3R)-4-cyclohexyl-6-({3-[(4-isopropylpiperazin-1-yl)carbonyl]phenyl}amino)-1,3-dimethyl-3,4-dihydropyrido[2,3-b]pyrazin-2(1H)-one;
3-{[(3R)-4-cyclohexyl-1,3-dimethyl-2-oxo-1,2,3,4-tetrahydropyrido[2,3-b]pyrazin-6-yl]amino}-N-(4-hydroxycyclohexyl)benzamide;
N-{trans-4-[4-(cyclopropylmethyl)piperazin-1-yl]cyclohexyl}-3-{[(3R)-1,3-dimethyl-2-oxo-4-(tetrahydro-2H-pyran-4-yl)-1,2,3,4-tetrahydropyrido[2,3-b]pyrazin-6-yl]amino}-4-methoxybenzamide;
(3R)-6-[(3-{[4-(cyclopropylmethyl)piperazin-1-yl]carbonyl}phenyl) amino]-4-isopropyl-1,3-dimethyl-3,4-dihydropyrido[2,3-b]pyrazin-2(1H)-one;
N-[4-(4,4-difluoropiperidin-1-yl)cyclohexyl]-5-{[(3R)-1,3-dimethyl-2-oxo-4-(tetrahydro-2H-pyran-4-yl)-1,2,3,4-tetrahydropyrido[2,3-b]pyrazin-6-yl]amino}-2-methoxybenzamide;
N-[cis-4-(4-cyclopropylpiperazin-1-yl)cyclohexyl]-3-{[(3R)-1,3-dimethyl-2-oxo-4-(tetrahydro-2H-pyran-4-yl)-1,2,3,4-tetrahydropyrido[2,3-b]pyrazin-6-yl]amino}-4-methoxybenzamide;
N-{trans-4-[4-(cyclopropylmethyl)piperazin-1-yl]cyclohexyl}-3-{[(3R)-1,3-dimethyl-2-oxo-4-(tetrahydro-2H-pyran-4-yl)-1,2,3,4-tetrahydropyrido[2,3-b]pyrazin-6-yl]amino}-2-methoxybenzamide;
(3R)-6-({2-methoxy-3-[(4-methylpiperazin-1-yl)carbonyl]phenyl}amino)-1,3-dimethyl-4-(tetrahydro-2H-pyran-4-yl)-3,4-dihydropyrido[2,3-b]pyrazin-2(1H)-one;
N-{trans-4-[4-(cyclopropylmethyl)piperazin-1-yl]cyclohexyl}-3-{[(3R)-4-(4-fluorophenyl)-1,3-dimethyl-2-oxo-1,2,3,4-tetrahydropyrido[2,3-b]pyrazin-6-yl]amino}benzamide;
3-{[(3R)-4-isopropyl-1,3-dimethyl-2-oxo-1,2,34-tetrahydropyrido[2,3-b]pyrazin-6-yl]amino}-N-(1-methylpiperidin-4-yl)benzamide;
3-{[(3R)-4-cyclohexyl-1,3-dimethyl-2-oxo-1,2,3,4-tetrahydropyrido[2,3-b]pyrazin-6-yl]amino}-N-(1-methylpiperidin-4-yl)benzamide;
3-{[(3R)-4-(4,4-dimethylcyclohexyl)-1,3-dimethyl-2-oxo-1,2,3,4-tetrahydropyrido[2,3-b]pyrazin-6-yl]amino}-N-(1-methylpiperidin-4-yl)benzamide;
N-[2-(dimethylamino)ethyl]-3-{[(3R)-4-isopropyl-1,3-dimethyl-2-oxo-1,2,3,4-tetrahydropyrido[2,3-b]pyrazin-6-yl]amino}benzamide;
3-{[4-(2-methoxyethyl)-1,3-dimethyl-2-oxo-1,2,3,4-tetrahydropyrido[2,3-b]pyrazin-6-yl]amino}-N-(1-methylpiperidin-4-yl)benzamide;
3-{[(3R)-4-cyclohexyl-1,3-dimethyl-2-oxo-1,2,3,4-tetrahydropyrido[2,3-b]pyrazin-6-yl]amino}-N-{trans-4-[4-(cyclopropylmethyl)piperazin-1-yl]cyclohexyl}benzamide;
tert-butyl 4-[(3R)-1,3-dimethyl-6-({3-[(1-methylpiperidin-4-yl)carbamoyl]phenyl}amino)-2-oxo-2,3-dihydropyrido[2,3-b]pyrazin-4(1H)-yl]piperidine-1-carboxylate;
N-{trans-4-[4-(cyclopropylmethyl)piperazin-1-yl]cyclohexyl}-3-{[(3R)-4-(2-methoxyethyl)-1,3-dimethyl-2-oxo-1,2,3,4-tetrahydropyrido[2,3-b]pyrazin-6-yl]amino}benzamide;
tert-butyl 4-[(3R)-6-{[3-({trans-4-[4-(cyclopropylmethyl)piperazin-1-yl]cyclohexyl}carbamoyl)phenyl]amino}-1,3-dimethyl-2-oxo-2,3-dihydropyrido[2,3-b]pyrazin-4(1H)-yl]piperidine-1-carboxylate;
3-{[(3R)-1,3-dimethyl-2-oxo-4-(tetrahydro-2H-pyran-4-yl)-1,2,34-tetrahydropyrido[2,3-b]pyrazin-6-yl]amino}-2-methoxy-N-(1-methylpiperidin-4-yl)benzamide;
3-[(1,33-dimethyl-2-oxo-4-phenyl-1,2,3,4-tetrahydropyrido[2,3-b]pyrazin-6-yl)amino]-N-(1-methylpiperidin-4-yl)benzamide;
tert-butyl 4-[(2R)-7-{[3-(dimethylcarbamoyl)phenyl]amino}-2,4-dimethyl-3-oxo-3,4-dihydroquinoxalin-1(2H)-yl]piperidine-1-carboxylate;
(3R)-1,3-dimethyl-6-({3-[(4-methylpiperazin-1-yl)carbonyl]phenyl}amino)-4-(tetrahydro-2H-pyran-4-yl)-3,4-dihydroquinoxalin-2(1H)-one;
3-{[(3R)-4-benzyl-1,3-dimethyl-2-oxo-1,2,3,4-tetrahydroquinoxalin-6-yl]amino}-N,N-dimethylbenzamide;
N-{trans-4-[4-(cyclopropylmethyl)piperazin-1-yl]cyclohexyl}-3-{[(3R)-1,3-dimethyl-2-oxo-4-(tetrahydro-2H-pyran-4-yl)-1,2,3,4-tetrahydroquinoxalin-6-yl]amino}benzamide;
3-{[(3R)-1,3-dimethyl-2-oxo-4-(tetrahydro-2H-pyran-4-yl)-1,2,3,4-tetrahydroquinoxalin-6-yl]amino}-N-(1-methylpiperidin-4-yl)benzamide;
3-[(4-benzyl-1,3-dimethyl-2-oxo-1,2,3,4-tetrahydroquinoxalin-6-yl)(methyl)amino]-N,N-dimethylbenzamide;
(3R)-4-(2-methoxyethyl)-1,3-dimethyl-6-{[3-(morpholin-4-ylsulphonyl) phenyl]amino}-3,4-dihydropyrido[2,3-b]pyrazin-2(1H)-one;
(3S)-4-(2-methoxyethyl)-1,3-dimethyl-6-{[3-(morpholin-4-ylsulphonyl) phenyl]amino}-3,4-dihydropyrido[2,3-b]pyrazin-2(1H)-one;
(3R)-4-(2-methoxyethyl)-1,3-dimethyl-6-({3-[(4-methylpiperazin-1-yl)sulphonyl]phenyl}amino)-3,4-dihydropyrido[2,3-b]pyrazin-2(1H)-one;
(3R)-4-(4-fluorophenyl)-1,3-dimethyl-6-{[3-(morpholin-4-ylsulphonyl)phenyl]amino}-3,4-dihydropyrido[2,3-b]pyrazin-2(1H)-one;
3-{[(3R)-4-(4-fluorophenyl)-1,3-dimethyl-2-oxo-1,2,3,4-tetrahydropyrido[2,3-b]pyrazin-6-yl]amino}-N-(1-methylpiperidin-4-yl)benzamide;
3-{[(3S)-4-(4-fluorophenyl)-1,3-dimethyl-2-oxo-1,2,3,4-tetrahydropyrido[2,3-b]pyrazin-6-yl]amino}-N-(1-methylpiperidin-4-yl)benzamide;
(3R)-4-(2-methoxyethyl)-1,3-dimethyl-6-({3-[(4-methylpiperazin-1-yl)carbonyl]phenyl}amino)-3,4-dihydropyrido[2,3-b]pyrazin-2(1H)-one;
(3S)-4-(2-methoxyethyl)-1,3-dimethyl-6-({3-[(4-methylpiperazin-1-yl)carbonyl]phenyl}amino)-3,4-dihydropyrido[2,3-b]pyrazin-2(1H)-one;
3-{[(3R)-4-(2-methoxyethyl)-1,3-dimethyl-2-oxo-1,2,3,4-tetrahydropyrido[2,3-b]pyrazin-6-yl]amino}-N-(1-methylpiperidin-4-yl)benzamide;
3-{[(3S)-4-(2-methoxyethyl)-1,3-dimethyl-2-oxo-1,2,3,4-tetrahydropyrido[2,3-b]pyrazin-6-yl]amino}-N-(1-methylpiperidin-4-yl)benzamide;
3-{[(3R)-1,3-dimethyl-2-oxo-4-phenyl-1,2,3,4-tetrahydropyrido[2,3-b]pyrazin-6-yl]amino}-N-(1-methylpiperidin-4-yl)benzamide;
(3R)-6-{[3-(2-azaspiro[3.3]hept-2-ylsulphonyl)phenyl]amino}-1,3-dimethyl-4-(piperidin-4-yl)-3,4-dihydropyrido[2,3-b]pyrazin-2(1H)-one;
3-{[(3R)-1,3-dimethyl-2-oxo-4-(piperidin-4-yl)-1,2,3,4-tetrahydropyrido[2,3-b]pyrazin-6-yl]amino}-N,N-dimethylbenzenesulphonamide;
3-{[(3R)-1,3-dimethyl-2-oxo-4-(piperidin-4-yl)-1,2,3,4-tetrahydropyrido[2,3-b]pyrazin-6-yl]amino}-N-(1-methylpiperidin-4-yl)benzenesulphonamide;
(3R)-1,3-dimethyl-6-({3-[(4-methylpiperazin-1-yl)sulphonyl]phenyl}amino)-4-(piperidin-4-yl)-3,4-dihydropyrido[2,3-b]pyrazin-2(1H)-one;
(3R)-1,3-dimethyl-4-(piperidin-4-yl)-6-[(3-{[4-(2,2,2-trifluoroethyl)piperazin-1-yl]sulphonyl}phenyl)amino]-3,4-dihydropyrido[2,3-b]pyrazin-2(1H)-one;
N-{trans-4-[4-(cyclopropylmethyl)piperazin-1-yl]cyclohexyl}-3-{[(3R)-1,3-dimethyl-2-oxo-4-(piperidin-4-yl)-1,2,3,4-tetrahydropyrido[2,3-b]pyrazin-6-yl]amino}benzenesulphonamide;
N-[2-(dimethylamino)ethyl]-3-{[(3R)-1,3-dimethyl-2-oxo-4-(piperidin-4-yl)-1,2,3,4-tetrahydropyrido[2,3-b]pyrazin-6-yl]amino}benzenesulphonamide;
3-{[(3R)-1,3-dimethyl-2-oxo-4-(piperidin-4-yl)-1,2,3,4-tetrahydroquinoxalin-6-yl]amino}-N,N-dimethylbenzenesulphonamide;
(3R)-1,3-dimethyl-6-({3-[(4-methylpiperazin-1-yl)carbonyl]phenyl}amino)-4-(piperidin-4-yl)-3,4-dihydropyrido[2,3-b]pyrazin-2(1H)-one;
N-{trans-4-[4-(cyclopropylmethyl)piperazin-1-yl]cyclohexyl}-3-{[(3R)-1,3-dimethyl-2-oxo-4-(piperidin-4-yl)-1,2,3,4-tetrahydropyrido[2,3-b]pyrazin-6-yl]amino}benzamide;
3-{[(3R)-1,3-dimethyl-2-oxo-4-(piperidin-2-yl)-1,2,3,4-tetrahydropyrido[2,3-b]pyrazin-6-yl]amino}-N-(1-methylpiperidin-4-yl)benzamide;
3-{[(3R)-1,3-dimethyl-2-oxo-4-(piperidin-4-yl)-1,2,3,4-tetrahydroquinoxalin-6-yl]amino}-N,N-dimethylbenzamide;
(3R)-1,3-dimethyl-6-{[3-(5-methyl-1,3,4-oxadiazol-2-yl)phenyl]amino}-4-(tetrahydro-2H-pyran-4-yl)-3,4-dihydropyrido[2,3-b]pyrazin-2(1H)-one;
4-(4-fluorophenyl)-1,3-dimethyl-6-{[3-(morpholin-4-ylsulphonyl) phenyl]amino}-3,4-dihydropyrido[2,3-b]pyrazin-2(1H)-one;
3-{[4-(4-fluorophenyl)-1,3-dimethyl-2-oxo-1,2,3,4-tetrahydropyrido[2,3-b]pyrazin-6-yl]amino}-N-(1-methylpiperidin-4-yl)benzamide
and
4-(2-methoxyethyl)-1,3-dimethyl-6-({3-[(4-methylpiperazin-1-yl)carbonyl]phenyl}amino)-3,4-dihydropyrido[2,3-b]pyrazin-2(1H)-one.

9. (canceled)

10. (canceled)

11. (canceled)

12. (canceled)

13. (canceled)

14. (canceled)

15. (canceled)

16. A pharmaceutical composition comprising a compound according to claim 1 in combination with one or more further pharmacologically active substances.

17. (canceled)

18. (canceled)

19. (canceled)

20. A compound of the formulae (IX) or (XVI) 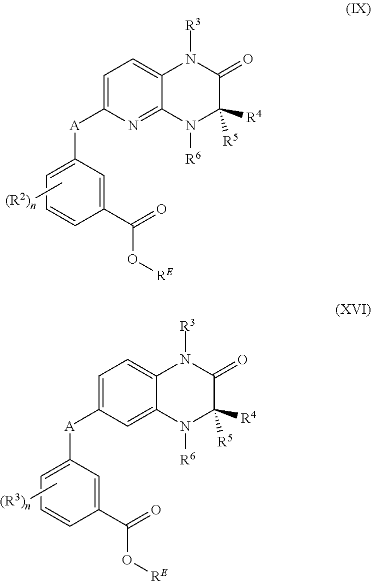
in which A, R², R³, R⁴, R⁵, R⁶ and n are each as defined in claim 1 and R^{E} is C₁-C₆-alkyl.

21. A compound of the formulae (IX) or (XVI) according to claim 20, in which R^{E} is methyl or ethyl.

22. A compound of the formulae (IX) or (XVI) according to claim 20, selected from:
methyl 3-{[(3R)-4-cyclopentyl-1,3-dimethyl-2-oxo-1,2,3,4-tetrahydropyrido[2,3-b]pyrazin-6-yl]amino}benzoate;
methyl 3-{[(3R)-1,3-dimethyl-2-oxo-4-(tetrahydro-2H-pyran-4-yl)-1,2,3,4-tetrahydropyrido[2,3-b]pyrazin-6-yl]amino}benzoate;
methyl 3-{[(3R)-1,3-dimethyl-2-oxo-4-(tetrahydro-2H-pyran-4-yl)-1,2,3,4-tetrahydropyrido[2,3-b]pyrazin-6-yl]amino}-4-methoxybenzoate;
ethyl 3-{[4-(2,6-difluorobenzyl)-1,3-dimethyl-2-oxo-1,2,3,4-tetrahydroquinoxalin-6-yl]amino}benzoate;
methyl 3-{[(3R)-4-cyclohexyl-1,3-dimethyl-2-oxo-1,2,3,4-tetrahydropyrido[2,3-b]pyrazin-6-yl]amino}benzoate;
methyl 3-{[(3R)-1,3-dimethyl-2-oxo-4-(propan-2-yl)-1,2,3,4-tetrahydropyrido[2,3-b]pyrazin-6-yl]amino}benzoate;
methyl 3-{[(3R)-4-benzyl-1,3-dimethyl-2-oxo-1,2,3,4-tetrahydropyrido[2,3-b]pyrazin-6-yl]amino}benzoate;
methyl 3-{[4-(2-methoxyethyl)-1,3-dimethyl-2-oxo-1,2,3,4-tetrahydropyrido[2,3-b]pyrazin-6-yl]amino}benzoate;
tert-butyl 4-[(3R)-6-{[3-(ethoxycarbonyl)phenyl]amino}-1,3-dimethyl-2-oxo-2,3-dihydropyrido[2,3-b]pyrazin-4(1H)-yl]piperidine-1-carboxylate;
methyl 3-{[4-(4-fluorophenyl)-1,3-dimethyl-2-oxo-1,2,3,4-tetrahydropyrido[2,3-b]pyrazin-6-yl]amino}benzoate;
methyl 5-{[(3R)-1,3-dimethyl-2-oxo-4-(tetrahydro-2H-pyran-4-yl)-1,2,3,4-tetrahydropyrido[2,3-b]pyrazin-6-yl]amino}-2-methoxybenzoate;
Methyl 3-{[(3R)-1,3-dimethyl-2-oxo-4-(tetrahydro-2H-pyran-4-yl)-1,2,3,4-tetrahydropyrido[2,3-b]pyrazin-6-yl]amino}-2-methoxybenzoate
and
methyl 3-{[(3R)-1,3-dimethyl-2-oxo-4-(tetrahydro-2H-pyran-4-yl)-1,2,3,4-tetrahydroquinoxalin-6-yl]amino}benzoate.

23. A compound of the formulae (X) or (XVII) 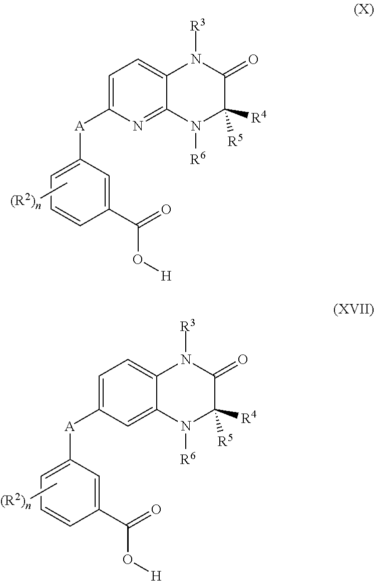
in which A, R², R³, R⁴, R⁵, R⁶ and n are each as defined in claim 1.

24. A compound of the formulae (X) or (XVII) according to claim 23, selected from:
3-{[(3R)-4-cyclopentyl-1,3-dimethyl-2-oxo-1,2,3,4-tetrahydropyrido[2,3-b]pyrazin-6-yl]amino}benzoic acid;
3-{[(3R)-1,3-dimethyl-2-oxo-4-(tetrahydro-2H-pyran-4-yl)-1,2,3,4-tetrahydropyrido[2,3-b]pyrazin-6-yl]amino}benzoic acid;
3-{[(3R)-1,3-dimethyl-2-oxo-4-(tetrahydro-2H-pyran-4-yl)-1,2,3,4-tetrahydropyrido[2,3-b]pyrazin-6-yl]amino}-4-methoxybenzoic acid;
3-{[4-(2,6-difluorobenzyl)-1,3-dimethyl-2-oxo-1,2,3,4-tetrahydroquinoxalin-6-yl]amino}benzoic acid;
3-{[(3R)-4-cyclohexyl-1,3-dimethyl-2-oxo-1,2,3,4-tetrahydropyrido[2,3-b]pyrazin-6-yl]amino}benzoic acid;
3-{[(3R)-1,3-dimethyl-2-oxo-4-(propan-2-yl)-1,2,3,4-tetrahydropyrido[2,3-b]pyrazin-6-yl]amino}benzoic acid;
3-{[(3R)-4-benzyl-1,3-dimethyl-2-oxo-1,2,3,4-tetrahydropyrido[2,3-b]pyrazin-6-yl]amino}benzoic acid;
3-{[4-(2-methoxyethyl)-1,3-dimethyl-2-oxo-1,2,3,4-tetrahydropyrido[2,3-b]pyrazin-6-yl]amino}benzoic acid;
3-({(3R)-4-[1-(tert-butoxycarbonyl)piperidin-4-yl]-1,3-dimethyl-2-oxo-1,2,3,4-tetrahydropyrido[2,3-b]pyrazin-6-yl}amino)benzoic acid;
3-{[4-(4-fluorophenyl)-1,3-dimethyl-2-oxo-1,2,3,4-tetrahydropyrido[2,3-b]pyrazin-6-yl]amino}benzoic acid;
5-{[(3R)-1,3-dimethyl-2-oxo-4-(tetrahydro-2H-pyran-4-yl)-1,2,3,4-tetrahydropyrido[2,3-b]pyrazin-6-yl]amino}-2-methoxybenzoic acid;
3-{[(3R)-1,3-Dimethyl-2-oxo-4-(tetrahydro-2H-pyran-4-yl)-1,2,3,4-tetrahydropyrido[2,3-b]pyrazin-6-yl]amino}-2-methoxybenzoic acid
and
3-{[(3R)-1,3-dimethyl-2-oxo-4-(tetrahydro-2H-pyran-4-yl)-1,2,3,4-tetrahydroquinoxalin-6-yl]amino}benzoic acid.

25. A method for the treatment of a neoplastic disorder comprising administering to a subject in need thereof a therapeutically effective amount of a compound according to claim 1.

26. A method for the treatment of a hyperproliferative disorder comprising administering to a subject in need thereof a therapeutically effective amount of a compound according to claim 1.

27. A method for the treatment of a viral infection, neurodegenerative disorder, inflammation disorder, atherosclerotic disorder or for controlling male fertility comprising administering to a subject in need thereof a therapeutically effective amount of a compound according to claim 1.

28. A method for the treatment of a neoplastic disorder comprising administering to a subject in need thereof a therapeutically effective amount of a compound according to claim 1.


SEQUENCE LISTING (2 sequences)

SEQ ID NO: 1 - protein, 30 aa, Artificial Sequence
  Synthetic acetylated histone H4 (Ac-H4) Peptid; acetylated on the lysine in positions 4, 7, 11 and 15
GRGKACGGKACGLGKACGGAKACRHGSGSK

SEQ ID NO: 2 - protein, 38 aa, Artificial Sequence
  Synthetic acetylated histone H4 (Ac-H4) Peptid; acetylated on the lysine in positions 5, 8, 12 and 16
SGRGKACGGKACGLGKACGGAKACRHRKVLRDNGSGSK